(19) United States
(12) Patent Application Publication
(10) Pub. No.: US 2022/0211761 A1
(43) Pub. Date: Jul. 7, 2022

(54) GENOMIC SAFE HARBORS FOR TRANSGENE INTEGRATION

(71) Applicant: MEMORIAL SLOAN-KETTERING CANCER CENTER, New York, NY (US)

(72) Inventors: Michel Sadelain, New York, NY (US); Ashlesha Odak, Mumbai (IN)

(73) Assignee: MEMORIAL SLOAN-KETTERING CANCER CENTER, New York, NY (US)

(21) Appl. No.: 17/697,044

(22) Filed: Mar. 17, 2022

Related U.S. Application Data
(63) Continuation of application No. PCT/US2020/051286, filed on Sep. 17, 2020.
(60) Provisional application No. 62/901,475, filed on Sep. 17, 2019.

(51) Int. Cl. A61K 35/17; C07K 14/725; C07K 14/705; A61P 35/00; C12N 15/62; C07K 16/28

(52) U.S. Cl. CPC A61K 35/17; C07K 14/7051; C07K 14/70521; C07K 14/70578; A61P 35/00; A61K 2039/505; C07K 16/2803; C07K 2319/30; C07K 2319/33; C07K 2319/02; C12N 15/625

(57) ABSTRACT

The present disclosure provides genomic safe harbors (GSHs) at which transgenes can be integrated for stable and reliable expression, without disrupting the expression or regulation of the endogenous genes. The present disclosure further provides genetically modified cells comprising a transgene that is integrated within the GSHs disclosed herein. The present disclosure further provides compositions, kits, and formulations comprising genetically modified cells disclosed herein.

### CROSS-REFERENCE TO RELATED APPLICATIONS

This application is a continuation of International Application No. PCT/US2020/051286, filed Sep. 17, 2020, which claims priority to U.S. Provisional Application No. 62/901,475 filed Sep. 17, 2019, the contents of each of which are incorporated by reference in their entireties herein, and to which each of which priority is claimed.

### SEQUENCE LISTING

This application contains a Sequence Listing which has been submitted in ASCII format via EFS-Web and is hereby incorporated by reference in its entirety. Said ASCII copy, created on Mar. 17, 2022, is named 0727341334_ST25 and is 69,195 bytes in size.

### 1. TECHNICAL FIELD

The present disclosure provides novel genomic safe harbors (GSHs) at which transgenes can be integrated for stable and reliable expression.

### 2. BACKGROUND

Modification of genomes by the stable insertion of functional transgenes is of great value in biomedical research and medicine. Genetically modified cells are also valuable for the study of gene function, and for creating reporter systems. The reliable function of the introduced transgenes is important for the applications of the genetically modified cells. However, randomly inserted transgenes, i.e., random integration, are subject to position effects and silencing, making their expression unreliable and unpredictable. Reciprocally, newly integrated transgenes may alter the expression of the endogenous genes near the integration site, potentially affecting cell behavior or promoting cellular transformation.

Thus, there remain needs for genomic locations at which transgenes can integrate and function in a predictable and reliable manner without disrupting the expression or regulation of the endogenous genes.

### 3. SUMMARY OF THE INVENTION

The present disclosure provides GSHs at which transgenes can be integrated for stable and reliable expression, without disrupting the expression or regulation of the endogenous genes.

The present disclosure provides a cell including an exogenous composition, wherein the exogenous composition is integrated up to about 10 kb upstream or downstream or within a locus of the genome of the cell, wherein the locus comprises or consists of a nucleotide sequence that is at least about 80% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75.

In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75.

In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, or SEQ ID NO: 6.

In certain embodiments, the locus comprises or consists of the nucleotide sequence set forth in SEQ ID NO: 6.

In certain embodiments, the cell is an immunoresponsive cell. In certain embodiments, the cell is a cell of the lymphoid lineage. In certain embodiments, the cell of the lymphoid lineage is selected from the group consisting of T cells, Natural Killer (NK) cells, B cells, dendritic cells, and, stem cell from which lymphoid cells may be differentiated. In certain embodiments, the cell is a T cell.

In certain embodiments, the exogenous composition comprises a transgene. In certain embodiments, the transgene encodes a molecule. In certain embodiments, the molecule is an antigen-recognizing receptor that binds to an antigen. In certain embodiments, the antigen is a tumor antigen or a pathogen antigen. In certain embodiments, the antigen is a tumor antigen. In certain embodiments, the tumor antigen is CD19.

In certain embodiments, the antigen-recognizing receptor is selected from the group consisting of a chimeric antigen receptor (CAR), a T-cell receptor (TCR), a chimeric co-stimulating receptor (CCR), and a TCR like fusion molecule. In certain embodiments, the antigen-recognizing receptor is a CAR. In certain embodiments, the CAR comprises an extracellular antigen binding domain, and an intracellular signaling domain. In certain embodiments, the intracellular signaling domain comprises a CD3ζ polypeptide. In certain embodiments, the CD3ζ polypeptide comprises a CD3ζ chain signaling domain. In certain embodiments, the intracellular signaling domain further comprises a co-stimulatory signaling region. In certain embodiments, the co-stimulatory signaling region comprises a signaling domain of CD28 or a portion thereof. In certain embodiments, the co-stimulatory signaling region comprises a signaling domain of 4-1BB or a portion thereof.

In certain embodiments, the exogenous composition further comprises a promoter, a transcription factor, and/or an inducible element. In certain embodiments, the transgene is operably linked to the promoter, the transcription factor, and/or the inducible element. In certain embodiments, the promoter is an inducible promoter. In certain embodiments, the inducible promoter is selected from the group consisting of a tetracycline-responsive element promoter (TRE) promoter, a CD69 promoter, a CD25 promoter, an IL-2 promoter, a 4-1 BB promoter, a hypoxia responsive promoter, and a beta globin promoter. In certain embodiments, the promoter is an endogenous promoter. In certain embodiments, the promoter is a constitutive promoter. In certain embodiments, the constitutive promoter is selected from the group consisting of an elongation factor (EF) 1 promoter, a cytomegalovirus immediate-early promoter (CMV) promoter, a simian virus 40 early promoter (SV40) promoter, a phosphoglycerate kinase (PGK) promoter, a CAG promoter, and a metallothionein promoter. In certain embodiments, the exogenous composition further comprises a polyadenylation signal.

In certain embodiments, the exogenous composition further comprises at least one insulator. In certain embodiments, the at least one insulator comprises a CTCF binding site consisting of the nucleotide sequence set forth in SEQ ID NO:76. In certain embodiments, the at least one insulator comprises or consists of the nucleotide sequence set forth in SEQ ID NO: 77. In certain embodiments, the at least one insulator comprises a CTCF binding site consisting of the nucleotide sequence set forth in SEQ ID NO:78. In certain embodiments, the at least one insulator comprises or consists of the nucleotide sequence set forth in SEQ ID NO: 79, SEQ ID NO: 80, SEQ ID NO: 81, SEQ ID NO: 82, or SEQ ID NO: 83.

In certain embodiments, the exogenous composition further comprises two insulators. In certain embodiments, each of the two insulators comprises or consists of the nucleic acid sequence set forth in SEQ ID NO: 77.

In certain embodiments, the exogenous composition is integrated by a gene editing system into the cell. In certain embodiments, the gene editing system is selected from the group consisting of a CRISPR-Cas system, a zinc-finger nuclease (ZFN), a meganuclease, and a transcription activator-like effector nuclease (TALEN). In certain embodiments, the gene editing system is a CRISPR-Cas system.

The present disclosure further provides compositions comprising a presently disclosed cell. In certain embodiments, the composition is a pharmaceutical composition that comprises a pharmaceutically acceptable carrier.

The cell or the composition disclosed herein can be used for reducing tumor burden, treating and/or preventing a tumor or a neoplasm, treating and/or preventing a pathogen infection, and/or treating and/or preventing an infectious disease.

The present disclosure also provides methods of reducing tumor burden in a subject. In certain embodiments, the method comprises administering to the subject the cells disclosed herein or the composition disclosed herein.

In certain embodiments, the method reduces the number of tumor cells, reduces tumor size, and/or eradicates the tumor in the subject.

Furthermore, the present disclosure provides methods of treating and/or preventing a tumor or neoplasm in a subject. In certain embodiments, the method comprises administering to the subject the cells disclosed herein or the composition disclosed herein.

In certain embodiments, the neoplasm or tumor is selected from the group consisting of blood cancers and solid tumors.

In certain embodiments, the blood cancer is selected from the group consisting of B cell leukemia, multiple myeloma, acute lymphoblastic leukemia (ALL), chronic lymphocytic leukemia, and non-Hodgkin's lymphoma.

The present disclosure further provides methods of treating and/or preventing a pathogen infection in a subject. In certain embodiments, the method comprises administering to the subject the cells disclosed herein or the composition disclosed herein.

The present disclosure also provides methods of treating and/or preventing an infectious disease in a subject. In certain embodiments, the method comprises administering to the subject the cells disclosed herein or the composition disclosed herein.

Furthermore, the present disclosure provides methods of producing a cell disclosed herein. In certain embodiments, the method comprises integrating the exogenous composition up to about 10 kb upstream or downstream or within a locus of the genome of the cell, wherein the locus comprises or consists of a nucleotide sequence that is at least about 80% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75.

In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75. In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, or SEQ ID NO: 6. In certain embodiments, the locus comprises or consists of the nucleotide sequence set forth in SEQ ID NO: 6.

In certain embodiments, the method comprises integrating the exogenous composition to the cell by a gene editing system.

In certain embodiments, the gene editing system is selected from the group consisting of a CRISPR-Cas system, a zinc-finger nuclease (ZFN), a meganuclease, and a transcription activator-like effector nuclease (TALEN). In certain embodiments, the gene editing system is a CRISPR-Cas system.

### 4. BRIEF DESCRIPTION OF THE DRAWINGS

FIG. 1 depicts the experimental scheme to obtain ATAC-seq atlas for human T cell genome.

FIG. 2 depicts the experimental scheme to identify candidate genomic safe harbors (GSHs).

FIG. 3 depicts the flowchart for identification of candidate Genomic safe harbors (GSHs) for testing. GSH atlas without pseudogene i.e. where pseudogenes were excluded from GSH and considered as genes, comprised 233 Mbp while with pseudogene atlas comprised 312 Mbp. The T-cell ATAC-seq atlas comprised of 21566 ATAC-seq peaks that were reproducible across all cell types and donors tested. GSH atlas with pseudogene and T cell ATAC-seq atlas were overlaid to identify ATAC-seq peaks that had a GSH within 5 kb. 379 such GSH peaks were identified which were then scored for their signal intensities as reads per million averaged across all cell types and donors and then ranked based on the average peak signal intensities. The top 6 highest intensity sites highlighted by inner box were tested for their cleavage efficiencies and transgene expression. The top 20 sites are highlighted by outer box.

FIG. 4 depicts a zoomed view of a candidate GSH peak spanning 860 bp (in black) and the 4 guide RNAs (gRNAs) indicated in flash signs tested for the GSH lying within the peak boundary and at the summit of the peak.

FIG. 5 provides cleavage efficiencies for all six selected GSHs. CRISPR/Cas9 cleavage efficiencies of four independent gRNAs represented by each independent symbol at each of the 6 top GSHs. Cleavage efficiencies were determined through analysis of the sequencing data after PCR amplification of the site and sequencing of the amplicon via deep sequencing or Sanger sequencing. Results are shown for all 24 gRNAs tested with the peripheral blood derived human T cells from one donor.

FIG. 6 depicts CAR knock-in construct at first three GSHs. CRISPR/Cas9-targeted CAR gene cassette for integration into the first three top GSHs. The top part illustrates representative GSH peak with gRNA cleavage site is indicated by flash signs; the bottom part illustrates rAAV6 donor cassette containing a 1928z1xx CAR (shown on the right) driven by an Elongation factor 1 alpha (EF1α) flanked by homology arms for the GSH peak.

FIG. 7 depicts the experimental scheme for CAR integration and preparation of CAR integrated T cells for proliferation assay.

FIGS. 8A and 8B provide CAR expression at GSHs overtime in culture upon multiple antigenic stimulation. FIG. 8A illustrates the experimental scheme for weekly antigenic stimulation of CAR+ T cells. CAR+ T cells were plated onto 3T3 cells expressing CD19 at day 7 after transduction and profiled for CAR expression at day 0, 4, 7 and 14 days after initial stimulation. Flow cytometry for CAR expression on day 0, 7 and 14 was performed just before plating onto 3T3 cells. FIG. 8B provides CAR expression profile (MFI) of CAR+ T cells with CAR integrated at GSH 1, 2 and 3 and TRAC over two weekly stimulations.

FIG. 9 depicts CAR knock-in construct with chromatin insulator element C1 incorporated. Chromatin insulator element (C.I.) C1 was incorporated into the CAR gene cassette shown in FIG. 4 by flanking the gene cassette with the C.I. C1 on both sides in an opposing, convergent orientation in order to rescue CAR expression levels overtime in culture possibly occurring as a result of heterochromatinization of the locus.

FIG. 10 provides CAR expression after introduction of chromatin insulator (C.I.) C1. Purified CAR⁺ T cells with CAR integrated at GSH 1 without and with C.I. C1 were exposed to antigenic stimulation for 3 weeks.

FIG. 11 provides CAR expression after introduction of chromatin insulator (C.I.) C1. Purified CAR⁺ T cells with CAR integrated at GSH 4, 5, and 6 with C.I. C1 were exposed to antigenic stimulation for 3 weeks.

FIGS. 12A-12C provide cell proliferation in response to weekly antigenic stimulation. FIG. 12A depicts the experimental scheme to stimulate CAR⁺ T cells with antigen weekly. The number of CAR⁺ T cells were counted on day 7, 14, and 21. The cumulative fold change was calculated to reflect the proliferation of CAR⁺ T cells without C1 insulator (FIG. 12B) and with C1 insulator (FIG. 12C).

FIGS. 13A-13C depict visualization of genomic features at GSHs 1, 4, and 6. Zoomed in views of a 1000 kb region centered on the gRNA cleavage site for each of sites 1 (FIG. 13A), 4 (FIG. 13B) and 6 (FIG. 13C). Next to “RefSeq Genes”, Annotations of Ref-Seq genes; next to “Activated”, ATAC-seq data in CD3, CD4 and CD8 cells from the data generated in this study; next to “Resting”, ATAC-seq data from non-activated CD4 and CD8 cells taken from Corces et al., Nat. Genet. 2016; 48(10):1193-1203; next to “Corces_Erythro”, ATAC-seq data from erythroid cells taken from Corces et al., Nat. Genet. 2016; 48(10):1193-1203; next to “H3K4me1_ENCODE_T_cell”, H3K4me1 marks in T cells from ENCODE; next to “H3K27ac_ENCODE_T_cell”, H3K27ac marks in T cells from ENCODE.

FIGS. 14A and 14B provide the tumor control ability offered by CAR⁺ T cells with different integration sites. FIG. 14A depicts the experimental schema for in vivo assessment of tumor burden using CD19-CAR stress test model for B-ALL. FIG. 14B provides median tumor burden for 4 mice per group administered T cells with CAR and C.I. C1 integrated at GSH 1, 4 and 6 as well as TRAC-CAR monitored for 60 days post T cell injection.

FIG. 15 provides total number of CAR⁺ T cells with different integration sites in mouse bone marrow at day 10 post injection.

FIG. 16 provides flow cytometry analysis of CAR⁺ T cells with different integration sites at day 10 post injection.

FIGS. 17A and 17B provide CAR expression of CAR⁺ T cells with different integration sites on re-exposure to antigen. FIG. 17A depicts the experimental scheme of re-exposing CAR⁺ T cells to antigen. FIG. 17B depicts CAR expressions in CAR⁺ T cells with CAR integrated at GSH6 with C.I. C1 and with TRAC-CAR.

FIG. 18 provides the cytotoxic activity of CAR⁺ T cells with different integration sites.

FIG. 19 provides the tumor control ability of GSH 6-CAR with and without C.I. C1 for at least 45 days in vivo.

FIGS. 20A and 20B provide the cytotoxic activity of CAR⁺ T cells with different integration sites upon re-exposure to antigen. Cytotoxic activity was determined by 18 h luciferase assay at day 10 (FIG. 20A) and day 17 (FIG. 20B) post T cell injection using cells taken from the bone marrow of mice. Each group contained cells taken from 4 independent mice of the same group and pooled together. Different CAR: NALM6=Effector: Target (E:T) ratios were analyzed based on cell numbers available.

FIGS. 21A-21E provide identification and targeting of Genomic Safe Harbors (GSH). Left panel of FIG. 21A depicts the flowchart used for identification of accessible candidate GSHs and right, mean signal intensities of ATAC-seq peaks associated with the 379 GSHs (without pseudogene list) ranked by their signal intensities; RPM, reads per million. The top 6 highest intensity GSHs highlighted by the black box were tested for their cleavage efficiencies and transgene expression. Error bars are ±s.d. of n=7 cell replicates. FIG. 21B is a volcano plot depicting the 379 GSHs centered on the GSH peak with a 5 kb region on each side of the peak in each of the 7 cell samples. Peaks are arranged in decreasing order of their highest (peak summit) signal intensities. The gray-shades indicate the value of signal intensities as given in the key to the right. The GSH coverage column depicts the region that falls under GSH criteria 1-6 in light gray and the region that falls outside the criteria in dark gray. FIG. 21C is an analysis of cleavage efficiency at top 6 candidate GSHs. Top, A zoomed-in view of an example candidate GSH peak spanning 1865 bps and the 4 gRNAs tested for the GSH at the summit of the peak. Bottom, CRISPR/Cas9 cleavage efficiencies of 4 independent gRNAs (each independent symbol) at the 6 top GSHs. Cleavage efficiencies were determined through analysis of the sequencing data after PCR amplification of the site and sequencing of the amplicon via deep sequencing or Sanger sequencing. Results are shown for all 24 gRNAs tested with peripheral blood derived human T cells from one donor. See FIG. 26B for data from additional donors for one selected gRNA per GSH. FIG. 21D is analysis of cleavage efficiency within vs outside a GSH peak. 4.5 kb genomic region within and around GSH 1 peak showing gRNAs targeted and their respective cleavage efficiencies. Distances from the edge of the peak are given at the top along with the name of each gRNA; R.B.: right boundary of peak; L.B.: left boundary of peak. Cleavage efficiency values are shown as symbols for two independent T cell donors, Dotted line represents mean of the two values. Numbers on x axis indicate distance in basepairs for the entire 4.5 kb region. FIG. 21E depicts the cytotoxicity assay for the CD19-CAR targeted at GSHs 1, 2, 3 and TRAC locus using firefly luciferase (FFL)-expressing NALM-6 as targets cells. Data is shown as mean±s.d of 3 technical replicates from the same donor.

FIGS. 22A-22H provide in vitro assessment of GSH-CAR functionality. FIG. 22A depicts experimental schema for weekly antigenic stimulation of purified CAR+ T cells at day7 after transduction. Flow cytometry for CAR expression on day 0, 7 and 14 was performed just before plating onto CD19-aAPCs; aAPCs: artificial Antigen presenting cells. FIG. 22B depicts CAR expression profile of CAR⁺ T cells with CAR integrated at GSH 1, 2, 3 and TRAC locus; UT: Untransduced cells used as controls. Right, Median fluorescence intensity of CAR expression for all histograms. FIG. 22C depicts proliferation in response to weekly antigenic stimulation for the CAR T cells in FIG. 22B shown as cumulative fold change in T cell numbers. FIG. 22D, Top, rAAV6 cassette design incorporating chromatin insulator element C1 flanking the gene cassette on both sides in an opposing, convergent orientation. Bottom left, Proliferation of GSH CAR+ cells without insulator C1 for 3 weeks in culture. Bottom middle, Proliferation of GSH 1, 4, 5 and 6 CAR′ cells with insulator C1 for 3 weeks in culture. Bottom right, table showing cumulative fold change in T cell numbers as mean±s.d. from two technical replicates for all constructs shown in the graphs. All T cells were obtained from the same donor (different from FIG. 22C) and experiments were performed with all constructs simultaneously. FIG. 22E depicts CAR expression at GSHs with and without insulator C1 at day 3 post transduction before CAR purification. All CARs were transduced at the same MOIs (multiplicities of infection) and experiments were performed simultaneously. MFI, median fluorescence intensity. See FIG. 26D for GSH 2 and 3. FIG. 22F depicts CAR expression profile of all CARs in FIG. 22D at the indicated time points over the three weeks. FIGS. 22G-22H depict cytotoxicity assay for all CARs shown in FIG. 22F at day 0 in g and at day 21 in h. Data is shown as mean±s.d of 3 technical replicates from the same donor. See FIGS. 26G and 26H for data from additional donors.

FIGS. 23A-23G provide in vivo assessment of GSH-CARs. FIG. 23A depicts experimental schema for in vivo assessment of tumor burden using CD19-CAR stress test model for B-ALL. The image shown is a representative image of the BLI at day 0, before T cell injection. FIG. 23B depicts Kaplan Meier tumor-free survival curves for mice administered with GSH 1, 4 and 6 CARs±insulator C1 and TRAC-CARs. Control refers to untreated mice. Combined results from two experiments with 2 independent T cell donors (n=7-12). FIG. 23C depicts tumor burden curves over 90 d in the group of mice from FIG. 23B. Some mice with no tumor burden had to be euthanized owing to severe GvHD. FIG. 23D depicts total CAR+ T cell number in the bone marrow of mice 10 d post-infusion (results from one representative donor, n=4-8 mice per group). Data is shown as mean±s.d. FIG. 23E depicts that mouse cells were depleted from the bone marrow cells of all mice illustrated in FIG. 23D, and the remaining cells were pooled by group and an 18 hr cytotoxicity assay was performed with CD19-NALM6 cells at a ratio of 3:1 T-cells:NALM6 cells. Cell number calculation was done based on flow cytometry data after mouse cell depletion (See FIG. 28B). Data is not shown for GSH 1 CARs since measurement of GFP was skewed due to the presence of NALM6+ cells (not eliminated by mouse cell depletion) present in bone marrow at day 10. Data is shown as mean±s.d of 3 technical replicates from the pooled cells. FIG. 23F depicts tumor burden in tumor bearing mice administered with GSH 6 CARs without insulator C1 and TRAC CARs at doses 2×10⁵, 1×10⁵ and 5×10⁴ monitored over a period of 80 d post T cell injection, n=5. FIG. 23G depicts tumor burden of mice comparing the in vivo efficacy of GSH 6-CAR and TRAC-CAR following 5 tumor re-challenges 10 days apart starting at day 17 post T cell injection, versus no further re-challenge followed for 80 days post-CAR administration, n=5 for both re-challenged and non-re-challenged groups. The NALM6 only group represents treatment naïve, age-matched mice injected with NALM6 cells at the re-challenge timepoints. 2 mice each were injected for the first 3 time points and 1 each for the last two time points. Statistics: *P<0.05, **P<0.01, ***P<0.001, ****P<0.0001; Mann-Whitney U-test (FIG. 23D); Welch's t-test (FIG. 23E).

FIGS. 24A-24C provide characterization of GSHs and association with function. FIG. 24A depicts 1 Mbp region centered on the GSH peak for GSHs 1-6 as well as GSHs 7, 12, 20 and 30 are shown. Refseq coding genes are GAP43, LSAMP, TSPAN13, AGR2, AGR3, AHR, TMEM161B, NECTIN3, TSC22D1, NUFIP1, GPALPP1, GTF2F2, TPT1, SLC24A30, ZNF425, ZNF398, ZNF282, ZND212, ZNF777, ZNF746, ZNF467, ZNF862, ACTR3C, SIPA1L2, MAP10, NTPCR, PCNX2, ZNF338, BMS2, noncoding genes are KCCAT333, NR_110013, NR_039993, NR_105020, LINC00461, NECTIN3-AS1, TSC22D1-AS1, LINC0030, NR_120424, LINC01745, LINC00839, LINC01518, LOC283028, NR_134479, LINC01264, NR_125822, pseudogenes are BRWD1P3, RPSAP29, ZNF767P, SSPO, LOC441666, CCNYL2, and ZNF378P, RPSAP74, KRT8P4. ATAC-seq peaks in activated cells obtained from the presently disclosed data (donor 2 is used as a representative) are next to “Activated”. ATAC-seq peaks in resting cells, obtained from data in Corces et al., are next to “Resting”. The signal intensity for both sets of data were scaled to the same range for all panels. FIG. 24B is a summary of CAR expression over multiple weekly stimulations, surrounding ATAC-seq peaks, gene presence and expression at all 10 GSHs given in FIG. 24A. Column 2: expression in the immediate (Imm) or day 0, early or day 7 and late or day 14 stages of multiple stimulation as per data in FIG. 22E and FIG. 31C; Column 3: Number of ATAC-seq peaks within 250 kb in activated(A) or resting(R) state; Column 4: Presence of ATAC-seq peaks in activated(A) or resting(R) state; Column 5: Peak signal intensity of neighboring ATACseq peaks. Peaks are characterized with a peak signal intensity of Hi:=/>1.5; Med:1-1.5; Lo:<1; Column 9: Gene expression in T cells (activated or resting); Column 10: Activated vs resting state gene expression. NS, non-significant; DE, differentially expressed; NA, not applicable (See also FIG. 32A). The GSHs are highlighted by shades based on their functionality with respect to expression over-time. GSHs 2-4 and 30, immediate expression only; GSHs 1, 5, 7, 12, 20, immediate and early expression; GSH 6, immediate, early and late expression. FIG. 24C depicts the presently disclosed criteria for GSH selection.

FIGS. 25A-25C provide analysis of correlation between cleavage efficiency and chromatin accessibility. FIG. 25A depicts cleavage efficiencies of Multiple target site specific (MTSS) gRNAs in K562 cells (data taken from Van Overbeek et al.) plotted vs maximum ATAC-seq peak signal intensities in K562 cells (data taken from ENCODE) within 200 bp of the gRNA target. The first panel represents all 127 MTSS gRNA targets and subsequent panels show MTSS gRNA targets grouped by the respective gRNA. The no. of target sites and the Spearman's correlation co-efficient between cleavage efficiency and signal intensity for the associated ATAC-seq peak for each group are given in the enclosed box in each panel. A target RPM=1>0.2 signifies presence of an ATACseq peak at site. FIGS. 25B-25C depict mean signal intensities in 7 cell replicates as in FIG. 21B and cleavage efficiencies at FIG. 25B, Low intensity GSH peaks with 2gRNAs per site (each symbol of the same sign) and at 3 GSHs with 4gRNAs/site identified in Papapetrou et al. not associated with an ATAC-seq peak and FIG. 25C, MTSS Group 3 gRNA targets. All were simultaneously analyzed in 3 independent T cell donors and 2 replicates of one of these donors (Donor 4_1 and 4_2), represented as different symbols. Target sites are ordered by their signal intensities. SH2 has a lncRNA gene located 1 kb away from the gRNA target; Sites 3b,c,d,h are located within a gene or very close to a gene (<1 kb away); 3g,j have a gene ˜5 kb away from the gRNA target while sites 3a,e and f are non-genic and do not have a gene located within 5 kb from the target.

FIGS. 26A-26H provide in vitro analysis of top 6 GSHs. FIG. 26A depicts an analysis of cleavage efficiency within vs outside a GSH peak. 4.5 kb genomic region within and around GSH 5 peak showing gRNAs targeted and their respective cleavage efficiencies. Distances from the edge of the peak are given at the top along with the name of each gRNA; R.B.: right boundary of peak; L.B.: left boundary of peak. Cleavage efficiency values are shown as symbols for two independent T cell donors, Dotted line represents mean of the two values. Numbers on x axis indicate distance in basepairs for the entire 4.5 kb region. FIG. 26B depicts CRISPR/Cas9 cleavage efficiencies with the gRNA for each of the top 6 GSHs that was used for CAR targeting with peripheral blood derived human T cells from 2 or 3 independent donors different from FIG. 21D (each independent symbol). FIGS. 26C-26E depict flow plots of CAR expression from T cells transduced with GSH-CARs at day 3 after transduction before CAR purification indicative of integration efficiency in 3 independent experiments with n=3 different T cell donors. MFI, median fluorescence intensity. FIG. 26C depicts data from cells used in FIGS. 21G, 22B, 22C; FIG. 26D depicts data from cells used in FIGS. 22D, 22F, 22G and 22H. FIG. 26E depicts data from an independent donor. FIG. 26F depicts CAR expression profile of all CARs in FIG. 26E, at the indicated time points over three weeks of antigenic stimulation. FIG. 26G depicts proliferation in response to weekly antigenic stimulation over 2 or 3 weeks as in FIGS. 22C and 22D with additional donors. Each panel illustrates data from all constructs performed simultaneously with an independent donor. Data is shown as cumulative fold change in T cell numbers, mean±s.d of 2 technical replicates or one sample in panels 2 and 4. FIG. 26H depicts cytotoxicity assay performed at day 7 after CAR transduction (see schema in FIG. 21E). Each panel illustrates data from all constructs performed simultaneously with an independent donor. Data is shown as mean±s.d of 3 technical replicates.

FIG. 27 provides relapses seen with GSHs 1 and 4 CARs occur at distant sites other than bone marrow and result in death at a low tumor burden. BLI images of tumor burden in mice treated with GSH 1-CAR, GSH 1+C1-CAR and GSH 4±C1-CAR from FIG. 23C that show the location of tumor relapse. The day of imaging is shown to the left of each set of images. The gray box around a mouse indicates death occurred at that time from tumor burden.

FIGS. 28A-28C provide gating strategy to analyze CAR T cells obtained from bone marrow of treated mice. FIGS. 28A and 28B depicts representative flow cytometric analysis of TRAC-CAR T cells on day 10 post CAR infusion with (FIG. 28A) fraction of cells used for complete immunophenotyping and total cell count determination, (FIG. 28B) cells obtained after mouse cell depletion and (FIG. 28C) cells after 18 hour co-culture with NALM6 cells at 3:1 ratio. Placement of gating was based on FMO controls shown.

FIGS. 29A-29F provide immunophenotypic analysis of CAR T cells in bone marrow of mice. FIGS. 29A and 29B depict total CAR+ CD4 and CD8 T cell number. FIGS. 29C and 29D depict phenotype of CAR+ CD4 and CD8 T cells based on CD45RA and CD62L expression. Naïve: CD45RA−CD62L−, Central Memory: CM: CD45RA−CD62L+; Effector Memory, EM: CD45RA+CD62L+; Effector: CD45RA+CD62L−. FIGS. 29E and 29F depict exhaustion marker expression in CAR+ CD4 and CD8 T cells, in the bone marrow of mice 10 d post-infusion (results from one representative donor, n=4-8 mice per group). All data is shown as mean±s.d. Statistics: *P<0.05, **P<0.01, ***P<0.001, ****P<0.0001; Mann-Whitney U-test (FIGS. 29A and 29B); Two-way Anova with Dunnett's multiple comparison test (FIGS. 29C-29F).

FIGS. 30A-30G provide in vivo analysis of CAR expression and functionality of GSH-CARs. FIG. 30A depicts CAR expression profiles of CART cells before infusion into mice (day 0), at day 10 post CAR T cell infusion in one representative mouse (0 hr) and after 18 hour co-culture with NALM6 cells (18 hr). Numbers in the box for day 0 indicate the CAR+ cell percentage used to calculate the number of T cells to be injected and dotted line indicates the CAR+ gate used. Refer to FIG. 28 for gating strategy. FIG. 30B depicts relative CAR MFI (1=MFI at 0 hour) of CART cells after stimulation with CD19-aAPCs over a week in culture (n=3 independent experiments on different donors). Data from 2 weekly stimulations is overlaid. Data is shown as mean±s.d. FIG. 30C depicts representative flow plots depicting CAR expression after stimulation with CD19+ aAPCs. FIG. 30D depicts tumor burden curves over 80 d using CAR T cells from an independent donor, n=5. FIG. 30E depicts tumor burden in tumor bearing mice administered with GSH 6 CARs with insulator C1 at doses 1×10⁵ and 5×10⁴ monitored over a period of 80 d post T cell injection, n=5. FIG. 30F depicts tumor burden over 40 days after re-challenge with 1e6 NALM6 cells in mice surviving at day 120 post CAR T-cell administration (from FIG. 23C). Some mice had to be euthanized because of Graft vs host disease, none had tumor. FIG. 30G depicts total CAR⁺ T cell number in the bone marrow of mice surviving till day 40 after re-challenge. Some mice had to be euthanized due to GvHD presentations. Each symbol represents a mouse. The mouse with the lowest cell number in the GSH 6+C1-CAR group corresponds to the mouse with tumor.

FIGS. 31A-31F provide in vitro efficacy characterization of GSHs 7, 12, 20 and 30. FIG. 31A depicts CRISPR/Cas9 cleavage efficiencies of 2 independent gRNAs at the peak summit (each independent symbol) at the GSHs 7, 12, 20 and 30. FIG. 31B depicts Flow plots of CAR expression from T cells transduced with GSH-CARs at day 3 after transduction before CAR purification indicative of integration efficiency in one representative T cell donor. FIG. 31C depicts CAR expression profile of GSH-CARs over three weeks in 2 independent T cell donors shown in the 2 adjoining panels for GSHs 7, 12, 20 and 30, day 0 is 7 days after T cell purification i.e. day 10 as per schema in FIG. 21F. FIGS. 31D and 31E depict two vertical panels show cytotoxicity assay data for all CARs shown in both panels in FIG. 31C depicts at day 0 (d) and at day 21 (e). Data is shown as mean±s.d of 3 technical replicates. FIG. 31F depicts two vertical panels show proliferation in response to weekly antigenic stimulation for the cells in both panels in FIG. 31C.

FIGS. 32A-32C provide analysis of gene expression around GSHs. FIG. 32A depicts plot of log 2fold change between stimulated and resting T cells vs respective −log 10adjusted p-values for all genes within 250 kb of the 10 GSHs tested (see FIG. 24A). Only genes with significant p-values are included. Genes including LSAMP, SIPA1L2, and GAP43 have very low expression (see FIG. 33 for read counts). Genes in the gray box having a log 2fold change ±0.5 i.e. a fold change of ±1.5 are not considered to be differentially expressed for FIG. 24B. The GSH corresponding to each gene is mentioned in the bracket next to the gene name. FIG. 32B depicts the 263 kb genomic region around GSH 6 is shown encompassing the two closest genes on either side of GSH6. The corresponding PCR amplicons used for qRTPCR for each gene (FIG. 32C) are shown next to “PCR” along with their names below. The arrows at the center indicates the gRNA cut site. FIG. 32C depicts RNA expression of genes around GSH 6 represented as ΔCt in comparison to 18srRNA (see methods). Two primer pairs were used for ZNF746 and one each for KRBA1 and ZNF767P. Data are mean±s.d. of 5-9 technical replicates from RNA of CART cells at day 0 and day 7 after stimulation on CD19+ aAPCs (CAR protein expression of the cells is shown in FIG. 22F). Dotted line indicates non-template control values for ΔCt i.e. no expression as can be seen with the CAR expression in untransduced cells; Statistics: *P<0.05, **P<0.01, ***P<0.001, ****P<0.0001; Two-way Anova with Dunnett's multiple comparison test.

FIG. 33 provides a tabula showing RNA-seq data for genes in the neighborhood of GSHs. baseMean=absolute expression in resting T cells; log 2FoldChange=fold change in comparison with activated cells; padj=p adjusted value; Highlighted in yellow genes with low/no expression.

### 5. DETAILED DESCRIPTION

The present disclosure provides genomic safe harbors (GSHs) at which transgenes can be integrated for stable and reliable expression, without disrupting the expression or regulation of the endogenous genes. The present disclosure further provides cells comprising a transgene that is integrated within the GSHs disclosed herein. Furthermore, the present disclosure provides compositions, kits, and formulations comprising cells disclosed herein. The cells can be genetically modified cells. It is based, at least in part, on the discovery that cells comprising a therapeutic transgene that is integrated within the GSH disclosed herein have stable and reliable expression of the transgene, and the cells are therapeutically effective in vivo.

Non-limiting embodiments of the present disclosure are described by the present specification and Examples.

For purposes of clarity of disclosure and not by way of limitation, the detailed description is divided into the following subsections:
5.1 Definitions;
5.2 Genomic Safe Harbors (GSHs);
5.3 Cells and Compositions;
5.4 Transgenes;
5.5 Exogenous Compositions;
5.6 Vectors and Gene Editing Systems;
5.7 Method of Producing Cells;
5.8 Administration;
5.9 Formulations;
5.10 Methods of Treatment; and
5.11 Kits.

### 5.1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, a “genomic safe harbor” or “GSH” refers to a chromosome location where an integrated transgene can be predictably expressed without adversely affecting endogenous gene structure or expression. In certain embodiments, integrating a transgene at the GSH does not alter cell behavior and/or promote malignant transformation of the host cell or the organism. In certain embodiments, the GSH permits sufficient transgene expression to yield desirable levels of protein or non-coding RNA encoded by the transgene.

As used herein, a “transgene” refers to an exogenous DNA sequence that is introduced into the genome of a cell, including a genetically modified cell. In certain embodiment, the transgene encodes a non-coding RNA. In certain embodiment, the transgene encodes a polypeptide. In certain embodiments, the polypeptide is a therapeutic polypeptide. In certain embodiments, the polypeptide is not expressed in the genetically modified cell. In certain embodiments, the polypeptide is endogenously expressed in the genetically modified cell in an amount that does not have an intended biological or therapeutic effect.

As used herein, the term “locus” refers to the specific physical location of a DNA sequence (e.g., a genomic safe harbor, a gene, a pseudogene, an extragenic region) on a chromosome.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that comprises clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40 and ICOS. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain V region fragments (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as VH) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imuno12009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immuno11997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also comprises “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

By “substantially identical” or “substantially homologous” is meant a polypeptide or nucleic acid molecule exhibiting at least about 50% homologous or identical to a reference amino acid sequence (for example, any one of the amino acid sequences described herein) or nucleic acid sequence (for example, any one of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% homologous or identical to the sequence of the amino acid or nucleic acid used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

As used herein, the term “endogenous” refers to a nucleic acid molecule or polypeptide that is normally expressed in a cell or tissue.

As used herein, the term “exogenous” refers to a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

The term “constitutive expression” or “constitutively expressed” as used herein refers to expression or expressed under all physiological conditions.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “neoplasm” is meant a disease characterized by the pathological proliferation of a cell or tissue and its subsequent migration to or invasion of other tissues or organs. Neoplastic growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells).

By “recognize” is meant selectively binds to a target. A T cell that recognizes a tumor can expresses a receptor (e.g., a TCR or CAR) that binds to a tumor antigen.

By “specifically binds” is meant a polypeptide or fragment thereof that recognizes and binds to a biological molecule of interest (e.g., a polypeptide), but which does not substantially recognize and bind other molecules in a sample, for example, a biological sample, which naturally comprises a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen comprises any polypeptide expressed by a tumor that is capable of activating or inducing an immune response via an antigen recognizing receptor (e.g., CD19, MUC-16) or capable of suppressing an immune response via receptor-ligand binding (e.g., CD47, PD-L1/L2, B7.1/2).

The terms “comprises”, “comprising”, and are intended to have the broad meaning ascribed to them in U.S. Patent Law and can mean “comprises”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

Other aspects of the presently disclosed subject matter are described in the following disclosure and are within the ambit of the presently disclosed subject matter.

### 5.2. Genomic Safe Harbors (GSHs)

The present disclosure provides GSHs in a genome (e.g., a human genome) at which a transgene can be integrated.

In certain embodiments, the GSH disclosed herein is located in an extragenic region of the genome, thus avoiding disrupting at least one endogenous gene. In certain embodiments, the GSH disclosed herein is not located in close proximity from the 5′ end of each gene of the genome. In certain embodiments, the GSH disclosed herein is located at a distance of at least about 50 kb, at least about 60 kb, at least about 70 kb, at least about 80 kb, at least about 90 kb, or at least about 100 kb from the 5′ end of each gene of the genome. In certain embodiments, the GSH disclosed herein is located at a distance of more than about 50 kb from the 5′ end of each gene of the genome.

In certain embodiments, the GSH disclosed herein is located outside of each non-coding RNA region of the genome. In certain embodiments, a non-coding RNA (ncRNA) is a functional RNA molecule that is transcribed from DNA but not translated into proteins. Non-limiting examples of ncRNAs include microRNAs (miRNAs), small interference RNAs (siRNAs), PIWI-interacting RNAs (piRNAs), long non-coding RNAs (lncRNAs), Mt_rRNA, Mt tRNA, misc.RNA, rRNA, scRNA, snRNA, snoRNA, ribozyme, sRNA, and scaRNA.

In certain embodiments, the GSH disclosed herein is not located in close proximity from each miRNA gene of the genome. In certain embodiments, the GSH disclosed herein is located at a distance of at least about 300 kb, at least about 320 kb, more than about 350 kb, more than about 380 kb, or more than about 400 kb from each miRNA gene of the genome. In certain embodiments, the GSH disclosed herein is located at a distance of more than about 300 kb from each miRNA gene of the genome.

A major risk posed by transgene integration is that of malignant transformation, in which transgene integration may activate expression of an oncogene, and thus may cause or facilitate cancer. In certain embodiments, the GSH disclosed herein is not located in proximity to at least one cancer-related gene. In certain embodiments, the GSH disclosed herein is located at least about 300 kb, at least about 350 kb, at least about 400 kb, at least about 450 kb, at least about 500 kb, at least about 550 kb, at least about 600 kb, at least about 650 kb, or at least about 700 kb from each cancer-related gene of the genome. In certain embodiments, cancer-related genes include oncogenes or any genes that are known to play a role in cancer initiation, growth, metastasis, or any aspects of cancer in humans or non-humans.

In certain embodiments, the GSH disclosed herein is located outside transcription units, to avoid disruption of the expression of at least one endogenous coding gene. In certain embodiments, the GSH disclosed herein is located outside each gene transcription unit of the genome. A transcription unit refers to a segment of DNA that is transcribed into an RNA molecule. In certain embodiments, the transcription unit comprises at least one gene. In certain embodiments, the transcription unit comprises at least two genes.

In certain embodiments, the GSH disclosed herein is located outside of each ultra-conserved region of the genome. An ultra-conserved element or an ultra-conserved region is a segment of DNA that is over about 100 bps in length, and is over about 95% conserved in human, rat, mouse, chicken and dog genomes and significantly conserved in the fish genome. In certain embodiments, the ultra-conserved element or the ultra-conserved region is a class of genetic elements that are more highly conserved among human, rat, mouse, chicken, dog, and fish than proteins. In certain embodiments, these genetic elements may be essential for the ontogeny of mammals and other vertebrates. Altering the copy number of ultra-conserved elements can be deleterious and can be associated with cancer. Thus, integrating a transgene into a GSH that is located outside of each ultra-conserved region of the genome can avoid disruption of ultra-conserved regions and any adverse effects associated with the disruption.

In certain embodiments, the GSH disclosed herein is: (a) located at a distance of more than about 50 kb from the 5′ end of each gene of the genome; (b) located at a distance of more than about 300 kb from each cancer-related gene of the genome; (c) located outside each gene transcription unit of the genome; (d) located outside of each ultra-conserved region of the genome; (e) located outside of each non-coding RNA region of the genome; and (f) located at a distance more than about 300 kb from each microRNA (miRNA) of the genome. In certain embodiments, the GSHs disclosed herein comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75.

In certain embodiments, the GSH disclosed herein comprises a pseudogene. In certain embodiments, pseudogenes are segments of DNA that have homology to protein coding genes but generally suffer from a disrupted coding sequence. An active homologous gene of a pseudogene can be found at another locus. In certain embodiments, the pseudogenes have an intact coding sequence or an open but truncated ORF, in which case other evidence is used (for example genomic polyA stretches at the 3′ end) to classify them as a pseudogene. In certain embodiments, pseudogenes are similar or substantially similar to a functional gene but are non-functional. In certain embodiments, a pseudogene is an allele of a functional gene that has become non-functional due to the accumulation of mutations. For example, the protein coding region of the pseudogene may contain a premature stop codon, or a frameshift mutation, or an internal deletion or insertion relative to the functional gene. Because pseudogenes are by definition non-functional but can support gene expression, selecting a pseudogene region that conforms to the presently disclosed GSH criteria allows the expression of transgenes of interest at therapeutic levels but without adversely impacting the functionality of cells.

In certain embodiments, the GSH disclosed herein has high DNA accessibility such that the locus has higher chromatin accessibility than about 90% of the loci screened. High DNA accessibility is associated with reliable and stable expression of a transgene, which may be important for the downstream application of a genetically modified cell.

In certain embodiments, DNA accessibility is indicated by the cleavage efficiency of a gene editing system at the GSH. In certain embodiments, the cleavage efficiency of the gene editing system at the GSH disclosed herein is at least about 90%. In certain embodiments, the cleavage efficiency of the gene editing system at the GSH disclosed herein is or at least about 95%.

Any gene editing system known in the art for targeted integration of a transgene to a predetermined chromosomal location can be used with the presently disclosed subject matter (e.g., disclosed in Section 5.6). Non-limiting examples of gene editing systems include CRISPR/Cas systems, zinc-finger nuclease (ZFN) systems, meganucleases, and transcription activator-like effector nuclease (TALEN) systems.

In certain embodiments, DNA accessibility is indicated by the expression level of a transgene that is integrated at a GSH. In certain embodiments, the expression of the transgene that is integrated at the GSH disclosed herein is detectable. In certain embodiments, the expression of a transgene that is integrated at the GSH disclosed herein is measure by genetically modifying a cell to integrate a transgene at the GSH, culturing the cell under conditions that favor the expression of the transgene, and measuring the transgene expression of the cell.

In certain embodiments, the expression of a transgene that is integrated at the GSH disclosed herein is sustainable, for example, the transgene is expressed consistently or stably for a period of time. In certain embodiments, the expression of the transgene that is integrated at the GSH disclosed herein is detectable for at least about 1 week, at least about 2 weeks, at least about 3 weeks, at least about 4 weeks, at least about 5 weeks, at least about 6 weeks, at least about 7 weeks, or at least about 8 weeks after its integration to the cell. In certain embodiments, the expression of the transgene is inducible, in which the expression of the transgene is only initiated upon contacting the cell with a stimuli that induces the expression of the transgene. In certain embodiments, the expression of the inducible transgene that is integrated at the GSH disclosed herein is detectable for at least about 1 week, at least about 2 weeks, at least about 3 weeks, at least about 4 weeks, at least about 5 weeks, at least about 6 weeks, at least about 7 weeks, or at least about 8 weeks after contacting the cell with the stimuli that induces the expression of the transgene.

In certain embodiments, the GSH disclosed herein has high chromatin accessibility. Chromatin accessibility of a GSH is important for the cleavage efficiency of editing system as well as expression of the transgene integrated at the GSH. Low chromatin accessibility of a GSH can result in lower efficiency of editing at the locus and low expression of the transgene integrated at the GSH.

Non-limiting methods for evaluating chromatin accessibility include micrococcal nuclease (MNase)-assisted isolation of nucleosomes sequencing (MNase-seq), DNase I hypersensitive sites sequencing (DNase-seq), formaldehyde-assisted isolation of regulatory elements seqencing (FAIRE-seq), and assay for transposase-accessible chromatin using sequencing (ATAC-seq). Tsompana et al., Epigenetics Chromatin (2014); 7:33 reviews tools for evaluating chromatin accessibility, content of which is incorporated herein by reference.

In certain embodiments, the chromatin accessibility of the loci is evaluated by ATAC-seq. In certain embodiments, the GSH disclosed herein is located at a distance of up to about 10 kb, up to about 9 kb, up to about 8 kb, up to about 7 kb, up to about 6 kb, up to about 5 kb, up to about 4 kb, up to about 3 kb, up to about 2 kb, or up to about 1 kb from an ATAC-seq peak. In certain embodiments, the ATAC-seq peak is present in both resting and activated states of cells (e.g., T cells). In certain embodiments, the ATAC-seq peak is present in either resting or activated states of cells (e.g., T cells). In certain embodiments, the methods disclosed herein comprise selecting a locus as a GSH if the locus is located within an ATAC-seq peak.

In certain embodiments, the chromatin accessibility of the loci is evaluated by the presence of and expression of surrounding genes in resting and activated state of a cell (e.g., a T cell). In certain embodiments, the GSH disclosed herein is located at a distance of up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb, from at least one gene that is activated and expressed in both resting and activated states of cells (e.g., T cells). In certain embodiments, the GSH disclosed herein is located at a distance of up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb, from at least one gene that is activated and expressed in either resting or activated states of cells (e.g., T cells). In certain embodiments, the GSH disclosed herein is located at a distance of up to about 250 kb from at least one gene that is activated and expressed in both resting and activated states of cells (e.g., T cells). In certain embodiments, the GSHs disclosed comprises or consists of the nucleotide sequence set forth in SEQ ID NO: 6.

In certain embodiments, the chromatin accessibility of the loci is evaluated by the presence of ATAC-seq peaks surrounding the targeted site on one or both sides. In certain embodiments, the chromatin accessibility of the loci is evaluated by the presence of ATAC-seq peaks surrounding the targeted site on both sides. In certain embodiments, the GSH disclosed herein is located up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb from ATAC-seq peaks that are present in both the activated and resting states of cells (e.g., T cells). In certain embodiments, the GSH disclosed herein is located up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb from ATAC-seq peaks that are present in either the activated or resting states of cells (e.g., T cells). In certain embodiments, the GSH disclosed herein is located up to about 250 kb from ATAC-seq peaks that are present in both the activated and resting states of cells (e.g., T cells). In certain embodiments, the GSHs disclosed comprises or consists of the nucleotide sequence set forth in SEQ ID NO: 6. In certain embodiments, the GSH disclosed herein comprises a nucleotide sequence that is at least about 80%, at least about 85%, at least about 90%, at least about 95%, or at least about 99% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75. In certain embodiments, the GSHs disclosed herein comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75. SEQ ID Nos: 1-20 and 75 are provided below.

[SEQ ID NO: 1]
GGGGCGGAATCGCAACAAGACCCCAGGACCCCGTGTGCGCGCGCGTGTCGGGGGAGCTGACCTATGACAAACAT
GTGCAGAGCTCTAGTACCCAGCCACGCGGAGGCTGCCGGTTGACAGAAGCGTTGCCGCCCCCTGCCGCCGAGTT
CACCCTGCCGTCACTGCTGGAAGCCGCACCCCTAGGAAAGGCTGCAAACCCACCCTGCCCCCAACACACCCGCG
CTCCCTCTCTGCAGGCCGGCTCCATCCCCGCCCCCAGGTGGCCAAGCAACACCGCCTTCTAGCTTCCTCCCGAC
CGGGCGGCGCCTGCAGCTGCCTGGGGAAGGAGCCCCGCAGCAGCGCGAGCGGGTGGGCGGGGGGTGCGCGGGCC
GCAGGGCAGGAGGGAGGGCGCAGGTAGGCGGGCGGGGAGAAGCTAGGCGGCCGGGCCGAGCGGTCGCGCCCAGC
CCTGCCTCTCTCCCTTCTCGACTCACAGGCGGCCTGACTCACTCCTTTGTTTCTGGAAAAGATCTATCTGTACT
AGCCCGAGGCGGAATTCACCCCGCCCGCACTCATCAGAAATCGTTTGCTTATTCTCTGCGCCCCTCCGTGCTCG
CCTGGGACGCGGGCGGAATCCTGACGCAGGCTGTCAGCCGCGGTGACGGGTGACACCGGGTGAGCAGCCGTCCG
CGCCGCCTTCCCTGCGCCGACGGGCAGCGCGGCGTTCGGCGCGCACTGCTGCCCCCTCGTGGCAGGAGCAAGCA
TCGCAGCAGCGCCGACACCACCGCCCACCTGCAGCCCGGGGAGCGTCACTTCTGGGTGTTTTTCAAAGGGAGCA
TTTTTACAGCTCCATTCAACTGCTGGGATTAGGAGATGT
[SEQ ID NO: 2]
ATATTTTTATGTTGCTTGATAAAAGATGATTGTCAAAAGAGATCCACACAGACAGTGGCTATAAGAGTGAAGCT
GCACAGGTCCCTTTGTAGCAGTCAGTCTCTCTCCTACTCCCTGTCTCATCTCCTTCCCCTGCCACAAGGACCCA
GAGAAGCAGGCAGGCTGGCAGTGTGATGACGGTATCTTACCCTGGCTCTGAGGTTGACACAGATTTCACAGGTC
ACTTCTATCACTGCAAAAGCACATTGGTCTGGAGTTTATGAAGAACATACAATTTCTGTTGGCATGTTAGGTAC
ATTATATAAAATGCCCCGCATAAAGAAAGCAGTGTAGTTAAAAGCAAATGAAAGATAAAAAGAGTAACAACGTG
GTTTCTCAGTATTTCATGGTACCGGTAGATATGACCTTCACAGACAAACCTAAATCTTTCTTATGTAACTGACA
TTTAGTCCGTTAGCCATTTTTAATAAAAGAGTAGAGAAGTTACTCTACTGCGGCTTTTGCTGGTACTTTTTATA
GCCGTCGTGTGGTAGGAAGTAGGCCCCAAGTTCCCTTGGCAACAAGTAGAGTAGAAGCGATACACAGAAAACTG
AGTCATCGGTGAATCATCTGAGGATTTCTGTTTCCAATCCTTGGAAATAATTTACCGATTGTTTTTTATTTGTG
CAGTGTTTGCTGACTCTGAGTGGACACCTCAGAAACTGTAACATCTTTGGTCAATTTGGTCAATCGTTTAAGAA
ACATTCTGGATGCTCCAAGTAGACCTTAAGATTGATATCCAAACAAAACAAACCTCTGCTTGCTTTAAGATGAC
ACTCACTCCCCATCTGAGGAGGAACAGATAAGTGAGTCAAGGTATTGGCAATGGGGAAGTCCCCTTTCTACAAA
GAAATCTGATGTCCCCAGAAAAATGTTTCATGACATTGTCACATTTAAAATGTTAGTCACAACAAATTTTCAGA
CACCTAAAAAGAGAACAAATATGGTCTCATACATTTTCTCTCTCTCACTCTGTCTGAGACTTATGTTCATCAGT
TACTCAATTCCCCCATTTTTTTTTTTACAAATACATCTCTTATATAATCCCTTTATCTCCACAATTGTTGCTTC
TACCTTATATCAAGGTTTACCATTTCATTTATCTAGGCTCAGATTGTCCTTTACTTCATTGCGTGTACAAGGAG
AAAGAAAAAACCTCCCCTAAAAATGCTTTTTGGCCAGGTGCGGTAGCTCAAACATGTAATCCCAGCACTTTGGG
AGGCTGAGGTGGGTGGATCACCTGAGGTCAGAAGCTTGAGACCAGCATGGCCAACACGGCGAAACCCCATCTGT
ACTAAAAATGCAAAAATTAGCTGGGTGTAGTGTTGCACTCCTGTAATCCCAGCTACTCAGGAGG
[SEQ ID NO: 3]
TAGGGGGAGAAAGGCATTTCCAACCAGTCTCCGTGCAGAACCTTCCCGCAAACCCTGTAGCGGCATTTTTCGAC
ACTTTTCTCCTCCTTTCGGCAAGCCTCAAACTTGACCTTCGAGAGGATTTTAAGGGTAGAGCCTTCCTCCCTTT
CCCTCGTCTGCTCCAAGAGCTCACGTAGGCCTTCTTCTGGGAAACAGCGGCTCAGGACCAGAGCTCGGAGAACT
GCGCGTCCAGGAGCGCGGGCGCGGCCGGGGAAAAGGGAGGAGGCGGCAGGAGGGGGCAGGAGGTGGAGGGAGGA
GGAGGATCGGTCTGAATATGCATGAGGCACCCAGCACTGCCCTCTTCCCCGGCAGCTGCTCGGGCGGGGGCGAA
GGAGGGAAGGGGAGGGGAGGGGAGGAAATGAGGCTGGGGCGGGGTGGCCAGAGCAGCCGCAGAGGCTGACGCGG
GTTTGGAGAACGGAAGAGATGATTTGCATGGCGCCTGGTGATTGGCTGATGGCCGGGGGCTGGGTCTGAGAGGC
GGCCCCTGAATGGTTTAACGGAGGAAATTACAAGATTCATTCGACTAAAGAAAGCCGGGGCGGGCCTGGTCGGC
GCAATATGCAGCCTGTCCGCAAGGGGGCGCGATGGCCTATCTTTGTTCTGGTTAAAGGCTCCTTTTCCCTCCTC
CCCCGCCCCCGCCTCCGCCCCCGCCCCCGCCCCCACCCCCCACACACAGACACACACGCAGAAAGTGCAGAGAA
ACCCTGTGATCCAGTCAACTGGCTCTGCCCCATCTATTAAGAAAGATGAACACTCCTTTCTCTTCCATCTCCTT
CATGGGAGACCACTTTTCATTTCAGATT
[SEQ ID NO: 4]
ATTTCCCTACTCACTCACCACACAATCATTTCCTAATTGCTGTCTGTGTTTGACTTGCACCTGTATTAGGTAAT
AAAGATTCAGAATTTTATTCACACTGGTTCCACTACCAATTTTACTATTCAATTGAGAAACAGATACCATTAGC
TGCTAATTCCAAAATCATCAGTTTGGTGACTATAACTTGGGTCAGAAAGATTTAGCACTGCTTCTCCTTCCACA
TCCACAACTTCTAGCTCACAGTTTGCCCCTTAGAAGTATCTGTCTAACATTCATAGTTTAGCCTGCAAGCTGTT
GCTTAGTCTAGTAATGACACATACAGAATGGTTTTATGGTCAAGCACTGAGTCATTCCCCAAGCTGCTTGTTTT
AGCTATATTCTCCAAATGTATACAATATAGATTTATAAAAATGATTATGCTGAGGTTATAATTTCAAAATGAGC
CACAAAGTATGTTTGAGGTACTCATCTGGCCTTTTCATTTTACAGATGAGGAAATACCCACAGGCGGTTGAGCA
AATTCCTGAAGAGCATACAACTTGTAAGATGATGACTTCAAAGCTCTTTGAGTGCTATCAGAATTTGTGAGTAA
CAGAGGCTTCAGCCTATGGATGGGGATGGAGGCTACACAATTTGAGGTAGGGAAGGGTGGGAGTTTGGGAACCA
AAAGTAGTTACTAGCTTGTTACCAAAGATAGATTTTGGTTGCAAAGAGGCAAAGTAAACTGCCAGTGACCTCAC
CACAAGGCAGGGGCAGACTTCCTGCTAAGGCATTAAGACCTTCTCAGTCATCTGGGGAACAAAGTGGCATTCAC
AAGGAACCGCCTCTGACCGCAGACCTACTGGGTAGTGGAGGTTTTTCATTCTTAAGTTTGTTCTTTCATTCCTA
TCAACTTGCAGATTTTCTACTGTCATCACCCACCTCCTCTCAAACCCTCATTTCTATTCAGCTTAGATCTCCTC
TTTTCCTTTTGTTTTCTCCTCGATTATTTTTATCTTATTTTATGCCATCATGCCCCATTTCAAAGAAATGTTCT
TGTTTTCTTAAAGCCTTCTACGTAAGATCCATTTAGCTCCTCATTC
[SEQ ID NO: 5]
AAAAGAGCACTCTTTTGGTCATTTGCTCATCTTCACAGTACAAGGAGAAAATACTGGAGAAAAACCAGCGGGTG
TATTTCGGCATGCGACCGCTTGGTTCCCCCGCTTTCCTTCACCTCTGACCTAATCTCTCACTTCAAAAACTGAC
TTCAAGCTGTTGCCAGATACGTCCCTTCCGCCGCCTGCCGCAGGTGGCTAGGGTCTGAGCACACTTGAACCTCA
CACCCGCCCCAGGGGTAGCTCCTTGGTTCCCTTCAGCCCCGATGTGTCCCTCGTGCTTTGAAATGGAATTACAG
TTTTGGTTAAAAACATGCCTTTTCCGAGTTAGGAAGAATCTAAATCGACTGAACGCCAGTCTAAAATTTCGGCG
TTCCCACACCGGGAGTCGAACCCGGGCCGCCTGGGTGAAAACCAGGAATCCTAACCGCTAGACCATGTGGGAGA
CGGCAATAGCGACTCCAAGCCTAGACAAATTGAGTCTTCTCGGTCGGCTTCCGCCCACTCCATCGCGTTCATCC
GTAGGCGTCAAACCTGCTCCTGCGCCTGCGCGGAGTCTGCAGCGGTTTAAACCGTTCAGGTTCGCATTCTACTG
TTTCTCTCCTTGCAGGGGCCCTTGAATCTTTCTTCAATCTACTCTCGCGTCTTTCGTGATCTCATCCAGTCTCC
TGGTGCTAGGTACCATGTCGACACCAATGTAGACACTAGATTGGATGAGTCATTGTTCACAATTCCTCACAAAA
CTCCTGCAAAGTCCAAAAGAACTGGGGTTTGAATGTTAGAGGCTGGTGAAGAAGTGAGGACCACGGGGCGTCAT
AACACCGCGCAGTGGAGGCCACGCTGCACTGGCCGGTGTCCGCCGACAGGGTGTGTGCCACAGACGCGCAGTGC
CTTGCCCGAATCTGTCCCTCCCCTCAGCGCTGGTCCAGGCTGGCCACAGGCACCACCCCGTCCCTGAAAGTGGA
TCTTTTCCTCTGAAAAAACAGGAATGAGAACTGCCCGGAAGCCTGGAACATTCCAGGTTCTGATGACTCAGCGG
CTGAAGGCAGGATTTGTGTCACCGAAAGCAGCCAGAGTGGCGGTCTGTAAATGTAGCTTAGAAAAGTGGAGAAA
GTCTCTTACTGCAGGCGTTCATGATAGATGGAAACTCCGTGTGGTTTCCAAGGCCCTCCTCGATTTGAAGCATT
TCAAGGGAGCGGTTGATGCCTTAGCAGTCTACTGTTTGAATTAGGGAAATGCAAAGAGTCAGCCATTGGGGATA
GCCGGCGGAGAGGAGCTGTGGCTCTGGTGTCCTCAGTGGCTTAA
[SEQ ID NO: 6]
ATATTCCCTTCCGCGCCGACCCTGGGGAGCTGGGCACGTAAGGGGTGTCGGTATACGCGGATTCGAAGCCTCAC
AACTCCCAGGTACCACTAAGCCGCTGGGGGGAGCTGGGGGAGGGACCCACAGGCCGAGCGCCAAGCAGAGAAAA
GAGCCTCGGGTGGGGGGACGCAGATGTAGGGTTCAGCGGCTGGGGCGGGGGCAGGAAGCCCCGGAGGGGCCAAG
GATCTTGGCGTGGGGCGGCCCGGGGCGGGGACAACCGTTCCGCCAGCGCTCGGGCTCGGCCCGGAGTGGGGGGC
CCGGCTGGCAAGCGCAGTAGGCCCCGGCAGACGCCGCGGCCCTGTTCCCCGGAGCCCCAGGCGCGGTAGTACGG
CTCGGCTCCCGCAGCCCCCGTGTGCCGGGCCCGGGTCGCCCGGGGAATCCAGGCCGCGAGTTCGTGCGCGCGCT
GGTCGCCCTTACCGGAGCTGCGGCCGCCTCCTCCATGGCCCTGCGCTGTCCCCCGCGGCCCAGATAAAGTCGTC
GCCGCTGCTGCGCGCGGCCCCACGCAGGGCCCGCCTCCCGGTGCTCTCCGCAGGCGGCGCCCGCCCAGCCCTGT
CTCTGCCGCCGCTGCTCCCTGACTGCCCCTGCCCTGCGCCGCCCGCAGTTGCGTCAACGCCTTTAGCGCCCGGC
CGGTCCGCACTGTATCCTGGGAGCCGGCGCGGCCGACGAAGGCACATGAGGCTTCCTGGAGGCAGGCAGCCGCA
CAGCCCCACCTGCAGCTCCGGAGGCGAACTGCAGGCGCGACCCCGCCTGGCCCGCGCCCGGGAGCTGACCCCGC
GACCCCTGGCCGACCCCTCGCCCCTTCCCCGACTGCCGCGTGTGCGCGCGCAGTCACTGCGCTGGCCCCGCGAC
CCAGGCCGAGGGACTCGGTTACCGAAAGAGTCCCCCTCATAAGACCCGCCTGGCCGCGCCCGCGGGCAAGCAGG
GGCTTCCAGGCAGGGCCCTCCCCGGTGACCGCGGGCAGTGTGGCCCCCTCGCCCAGCTGGGCCCCGGCGCGGCC
GAACAGGCACTTCTGCCGCCCGAGGTCCTGGGCCTGGAGGTTTGGGCTGTTTTCGGGGGGCAGCTGTAACTCAT
CCCCATCTGTGTTTGAAGTGGAATAAATACTTCGAACAATAGGAATATACCGGTAGATGCTACTTAGCATTTTC
[SEQ ID NO: 7]
CGTCAGCCCCCCAAAGCGCCCCGAAGGGCATCCCCTTCCCCTTCGGGACAGTCCCCGCCTGGCCGCAGGCATCC
CGAACCGACCCTCGCCACGCTGCCCGTCGGATTCCCCGACACGAACCTTCGCAACGCCGTCCGCCGGAACCTGC
TACAGCCTGTGCGCGCCGGGCGGCGCGTACCCGGGCTGCCGCCTCGCCGCCCGCCGCTGCCCGGGGCTGCCCAT
CGGCCCGGACTCTCCCCGCGCCGGGCTCCGGCGGAGGCGGCCCGGACCCGCTGGCTGCGGCTGGAGCTCTCGGC
CTGCGCTCGGGCGGCCGGCGGGGGCGCGGTGCTCCTCCTCCGTCCTCCTCCTCCTCTCGCTCCGCCAGCTCCTC
CCGGGCTCCCAGTCTGCCGCGCCGGCTCCCGATGCCACCGCCCGCCCGCCCAGCGGCACCGCCCCCCGCCCGCT
CTGCGGGCTCCCATTGGTCGCTGCCTGCAGGCCCCCTCGGCCCCGCCCCACGATTGGGCGCGGACGCCGTCAGT
CTCACCGTCCGCCCGGCTCCGGGCGCAGCCGGTGCGCTGTTCCCAGGCCCCGCGGCGCGCGGGAGCTGCGCGTC
CAGGAGGGGCCCGGACGCCCTTCTTGGGGAGGGGGCTGCGGGGCGCATCATGAGCGGCGCTGGGCGAGCTCGAG
GGCCGGGCAGCCTACCTGGACCCCGGCTTCTAGAGAGCCGTCGCCGTCCTTGTCGAGACTCGCTTTCGCCCCTC
TGAGGAATAAGTTAAAAACCGCGAGTCTGGTTCTGAGCCGCCCCCTGGCGCAGGCGCGACCCCACCCGGCCCTC
CCGTCTCCCAACTTGGGACAGACGCGGCGGCCGGGCGGTCGGATCCGTGAGCCTCGGCCGCTGGGGCTACATAC
ACAGCCCAGGACGCCACAGAATCGCCGAGGCCCCGCGAACGCCCAAACGGCCGCAAGTCGAAGTGATCCCATCT
GAAGAAGACAGCGCGAGGAAGCGAGACCCTGCTGTGTGCCGGCCCCCGGAGGGCGCCCCGGGCCCTGTGGGTCG
GAGGGTGGGGAAGCACAGAGAAACACGCAGGCAGTAAAACGTTAGGAGGACAGGGGGTGCCAGCGTGCAGGAAA
TGCTGCTAAGAGACCTTTAGGAGCCAAGGGGAAAACGGAGAGATTGGCAGTTCCAGGCACCCACATCCCAGCGC
GGTCCGCTCACCCCTTCCTCCCTG
[SEQ ID NO: 8]
TGAAAATAAAATCAAAAATTTGCTTTTTGTCATTCTGGTAATGCTGTTCTTCATCATAGACAATAACTTGGAGA
TAACTTCAAGCCAGAGATGGTAGCTTTGCTCATAGTTGCTTTGGATGGTAATCAAATGAGACTTTCGTTTATGT
CACAGATGGAAGCTTGCAAGTCAAAACATTCAAAAGAAATGAAGATTTCTACATACCCAAGAACTAATGAGCTA
GCCAGTGAGTGAGTCATTATCCATTTATGTCACAGATGGAAGCTTCTAAGTCAAAACATTCAGAAGAAATGAAG
ATTTCTACATACCCAAGAACCACTCAGCTAGCCAGTGAGTGAGTCATCATCCCTCACTTGGCCTGCTCTTTGGC
TCCATACTGGCTTCTTGGCTCCTGTTCTTTACCTGCTACAGTTTTTGTCCACATAGTAGCCAGAATGATTCTAT
TGAAATGAAACAGACTATGTCATTTAAGCTCAAAAACCTCGAACAACTTTTCATCTCACTCAGAATACTATGAT
TGTGTGGAACCCATATTATATGACCTCTGGCTAACTTGATTGAATTTATACCTCACCATTCTTTCCTTCTCAAA
GTTCACTCCATCAGCCCTGACTTTTTGGTGGTGAAACCAAGTATGCTTCCATTCACTGCCTCTGCACCTACATA
GCTTCTGCTTGTGAAGCTCTTCCTGGAGACATTTACTTGGCTCACACTCTCCAATCAAGTCTTTGTTAGAATGT
CCCTTATTTGCGTGTTGCTCCTACCTAATCTTTATAAAATGGCAC
[SEQ ID NO: 9]
AAGGAAGAGAAGGAAATATTGCTAACGCAGAGCCTAGACCAACTGGGAGGGATGTGATATAGAGCCCAGAGGAG
CAATCACTTCAAATGGGAACATGGATGGCTCATTTATAGGCCCAAGTGGGAAGGCAAAAGTTGTGAGTGGGAAT
GGTGGTAAGTGAGTACAAGTCGTGGTGGAAGACTATGCCACTTATGTATTGCTACAGTTTTTAAGAAATATAGG
AAGCAAGGCCATTGGCTAAGAGTGATGATAGAGGAGATTTGGAGGTGTGAGAAGAGAGTCTTTTAAGGGGATGC
TTCCCAAACTTAAATACACTATGCAGAAGAATCTCCTAGTATCTTGATAAAACTCAGGTTCGTATTCAGTAGGT
CTAGGATGGGGTCTGAGATCTGTATTTCTCAGAATATCTAGGTAATATCAATAGTGTTGGCCCATGGGCTACAC
ATGGAGTAGCAAAGTTCTATGAGAAATGAGAAAGTTGAGTGAATCAGGAAAGTACTGAGTGCGTAACAGCATTA
AACATCTACTGGAGTTCATTGCTGTTAACTGAAAGTGAAACTAATCAGAATATTTGTGTGTTTTGTTCCAACTA
CTCTGGGCTTAAATGAGGCCAAATGTGGCTGGAAGTGAAAGTGAAAATGATGAAGGAAGAGAGATATGAGTCAA
GGGTGTGTGCACTGGAGAGGTTATAATTGGTAAATTTAAGCTGGGTTAGGAGGGAGAAGTGTTACAGAGTGAAT
ATATCCTGCCGCAACTCATTTGTTGAAGCTCTAACCCCCAGTGCAATGGAATTTAGAGGTAAGGCCTAAGGGAG
ATAATTAGAGCTAGATGGGGTCATGTAAGTGGGTCCCTCATGATGAGATTAGTGGCCTTATAAATAGAGTATGA
GAGAGACCTCTCCTCTCTTTACCCAAAAAAAAAAAAAAAACAAAA
[SEQ ID NO: 10]
TTTTCATTCCACTTACCATGACAGTTAGCTCAGGTCTCTACAATGCCCTTTCCACCAACAAGTTGAATTTCTAA
GTCTCTTGGCTGAAAAGGGCAAAGAGCTTTCCTGTATGATGAAACAGGCTGCATGGCCCAAAGGCAGCCTGCTA
TAAAGGTTGACTGTGGCTGCCTCCTCTGTCTCAAGCTTTGCTGGGAAGCCACATTTTTCACAATTGAGTTGTTG
CAAAAAGTTATGTGTGTTACACTCTATGTGTTCTTGAAAACTATATTTGTCAAAATGTTCATGGTAGGAAACTG
TTTTCCCTTCAAGTTGTAACTGAGGTTTACCTTGGAGGCTACTTTGACCACTGTGATATTTAACTCTATGACCA
CCAACTCTTGATCTGGTGCTAATGAACTCAAACAGCATTAAAGACTATGGGGATATTCTTAACATTGGGAACAC
AGCTAAAGGCAGCTCTTTCTGGGGGGAAAAATGGTTTACAAGTAAATAAATAAACAGTGACTCTAACACATTAA
CTTCAAAAACATCATACAGAATCTCAACTTGCCATGACATCGTTCTGTGGTCTTGGGCCAGTCATAATGTCTAT
GAGGCTTAGTTTCAACTTCTGTCACGATGTGTGTGGCTCAGCCTTCCCTCATCAGTGCTGTGAGGAGTAAATGA
GGAGACTTGTGCGAAGAGATCCACACAGAGCTGAGCACGCGGTGAGCACCCAGCAACACCAACATAGAGGGGAG
AGCCTTCTCTCACACGCCTCGAGCTTCCACAGAAGAAGGAACTTCTGCTCTACTCACCGAGATGGACAAGGATA
GGAGAAACACCCTCCCAGTTTCTTCCCCACATACTACCCTCTCTCACAGAAGAAGCCAAGCAGAGGGGTGGGCC
TACAACACACCCTGGGCCACGGCGCACCTGAAGGAGGGCCGCTG
[SEQ ID NO: 11]
AATCATCATGTTTCAAGGGATAGCAGAGTTGATATTGGTAGGAAGAGTGGAGGCAGCCAGATGAAGGAGGTGGG
AAGTGAGAAGAGCACCCCAGGCAGAGGTGCCAGCATAAGAACGTCGTGTGAAAAGGCAGTGACCTGCTGCTCCG
TGTCCTGAGCTTGGAAACCCTAGTGAGACATTAGGAGAGAGTGCAGGCAAGAGGATCCTGCAGGCCTTGAGACA
GGGCAGAAAACAAGGCCTCCAGAAATGAGGCAGCAGGAGGAAGTGTATGAGAAAGCAGAATAGATCTAGAAAGA
GCCAGCAGATCAGAGGATAAGATGGGCAAGTACCATGTCACAGAAGCAAAGAGAGGAAGTGGAGTTTCAACAGC
GGCCTTTAACAGAGAGAGTGCGAAGGTCAGAGACCCAGAAGAAGGAGGGCTTGGCTAGAAGGTCACTAGTAGGC
TCTGGCTTCCATCTCCAGATAGGCTAAGATTAAGCCAATTCACCATTTTCCCACAACTGTCAACTCTGCAGAAT
TGCCTTTTCTTCCTCTCAGACAGAAAGGGAAATCCCAGCTGCCTGAGCAACCGGTTTTTCACTTTCCTCCTTGT
TTGCAGAGTCTAGTGGGGCTGGCCCTGGTAACCTCTGGTGATTCATCCAGCACACCCTATGACCACATCCCCAT
TGTGTGTGTGTAGAGAGGATGAGAGTTTATGTGTTCCTCTGAACGTTATTACAAAAGAAAAGAACATTGTTCTC
TAGAAATATGAAAGAAAAAGAAAAGAACAAGGTGTTACTCTAGCATAGTTTTTTCTCAATTATCTGAGTTGTGA
AAAAAAAAGTAATGAATAGTGGAGCTTCCTCAAAACAGATTCTGAGCGCTGTCAGTAGTTAACAGTAACTACTG
TGGTGGAATTCCTGTGTGGATCTATTTTGGCACTGTGTTACGTAATTATTTTTATCTTAGGATCAGATCTGAAT
CATTCAAAGCCAATGTCTTCAGGGCACTGGTGTGACTCCAGGTGTTCTCACATGCCTGTCTTTCTTCTCAGATC
ACTGCCCAACACTGAATCTCAAGTGATGGACCTCAGTGCATATGAATTCGGGGCCTATATGTT
[SEQ ID NO: 12]
TCTTTCTCTTTTTCAAAATATCTTGTCGTACTGCACACTTAGATGTGTAGTATAATAAGATATTTCCTTGTTAT
GATGTGAGATGACACAATGCCTATGTGATGTGATGAAGTGAGGTGAATGAGGTAGGCATTGTAGCATTAGGTTA
CCAGTCACCTTCTTACAATAGTCACAAAGAGATCATTTGCTTTGAGTGATCCTGCATCATCGAGCTGTGATGAT
GTTGATGATGCGGATGACTAATAGGCAAGTAATGTACGCAGCATGGATACACTAAATGAAAGAATGACTCATGT
CTCGGGTGGGACAAAACACCCACGACATGAATCATCACATGGTACAGTGTTTCTTCAGGGTATTCAGAATGACA
CACAATTTAAAACTTACAAATTGTTTATTTATTGAATTTTCTATTTAAAAATTTCAAACTGCAGTTGACTGTGG
TAAACTTAAACTGGAGAAAGTGAAACTGGGTAAGAATACGTTTTGCATCTGTGTATCCTGACAATTTCAAGGAA
CACACATTAAAATTAGCTTATTAAGGTATGCACATGTTTAACTTTATTGGATATTATCTTAGTATGTTCAGGCT
GCTGTTAAAAGAAAAACAAAAGCTGGGTAGCTTACAAATAACATACATTTATTTTTTACAGTTCTGGATGCTGG
GAAGCCTAAGATCATGGGAAAGGTAAATTTAAAGTCTGGTTAGAGTTCACTTTCAGGTTTATAGATGGTGCCTT
CTAGCTGTGTCCTCACATGATGAAAGGGGTTAGTGGTCTCTTTGGGGCCTTTTCAAAAGGCACTATGGAACCAC
ACACACAAACACACACACACACACACACACACACACACACCCTGGAATAGCCAAGGCACTTGTGAGAAAAAGAA
CAAAGAGTTA
[SEQ ID NO: 13]
CGGGGACACACTCAGAGTCTGGATCTGATTCACCAAGGCGCTAACACTGAAACAGTGGTCCAGAGTGCCAGCTT
TTGTTCATGTCTGGTCAGACACCTCCATCCTCTGGGCAATCTCACTCACTCTCCTGGGACCTAAAAGGAGAGCC
TCACCTGGGGTGCCACAAAATGTTACCAGCATCATGGATTCTTGTGATTGCCCAGGATAGAAACCAAAAGAGAT
AACCAAACATAGCAGCAAAGGAAAGTTTATTCCGCTTGTGCACAGGGGAGTCAGCACCAAGAAGGGAAAAGAAA
TGGGTTGCTTATCGAGGGTAGTCTGTGAGTTAGGTTTACAGGAGTTTTCTACAGGAAAGTATTACATTCGAGCA
TGTATAGGAGTAGGAGGGATTTTTCTAGTGCTTGTGCATTGGCTCAACATGCTTTTTCATACATCATATGTAGA
ATTAGCATTTTACATCTCCACCTCTGGGCATGATTTTTAACATTAAAATAAGGAAGGGGTAACTGTAGTTCGAA
GTTTAACTCTACTGCACATGCAGGGCCCTGGGGAAGTCCCTAGCCCCCGAAGCAGGAATTTGTGGTTAACAGC
TTCTTGGGTCTTTTATTACTGATTGGTTGAGAGTTAGGTAAGCTACAGCTTGAGTAAGGAACTTGCATTCTTTT
ACTCCAGACCATATTAAAACAGGGAACCAACCAGCCTGCCTGTCTCATAGCTAGTTTGCTTTAATCTGTTCCCA
GCTTACCTTCCTTTTCTCAGATGAAATGAGACTCATGTTAACCAAAATGCCATCCTACCTTTTGTCTTTTGACT
AGACACATTGTAGAGGTTTGGATTTGTTAACCTGGAATGGGAAAGGCTGAGAGGGGAAATGATCCTGGGTATGC
TAATGAGCAGAAAGAAAACAAGGATGGCACGGATACCTCTCTCTCCCCCTTCTATGTGTGTCCTCTACCTGTTG
TACATTCGTATGAGATAGGTATTAATATAACCTACACTTCGTAGGTGACGAAACAGTCTCAGAGAGTGAAAGGG
ACTTGACCAAAGTCACAGAGCCAGTGAGTGACAGGGTCAGGGAGAGAAGCCACGTCTGCATGACTCAAAGCTTA
TGTCTCTAATTACCCTACATACTACCCCGCCGCCTTTGCAAGTTATAAATAGGTCATGCTGGCTTTACTCATTA
AGTCCTGGGTTGCTAAAGTGAAAGAAAAACTTCCTTCCCAGTATGCCTTGTACACATCCTGAAACTGAAAGAAA
GCACTTTAGAACAAATTAAAGGCAGTTGTCTTCTGTGTCTTAGGGAACAAAAATACTAATTGTATTTTTAACCT
CAAATTGTAGAGCCTGAAAAGACTGGCTTGAGAAGGAGCCTAGGTCATTCCCTGGACACCTAGAGGTCTAGAGG
GCATAATAGAGGTCCACAAGTTTTTCTCAATGTGAAACAAACAATAATCCTCCCAACCAGACAGAGAATTTACA
TACGCTCCCTTTCAGACCGGGCAGGCAGACAAAGTCACAAGTGCTTTGACTTTGGGCTAAGGATTCTGTCAGGT
CCATCAATCCTTTAACCTCACCTTCACAGCCATGATAGCTAACCCTGACTGAACACTTTCTGTATGCCAGCCAC
TGTTCCACTTCCTTTACTCCTCATAACAACCCTGAGGTTATGACTAGTATCACCTCTATTTTGCAGAAACTTGA
GGGCATCTCATGCTGACAAGAAACTAATTGGGAGTT
[SEQ ID NO: 14]
AAACCAGGACATACAGAGATAGGAGCTGAAGGGACATGGTGAGAAGTGACCAGAAGACTAGTGTGAGCCCTCTG
TCACACCCGGACAGGGCCACTAGAGGGCTCCTTGGTCTAGTGGTAACGCTAGTGCCTGGGAAGGCACCCGTTAC
TTAGCAGATCGGGAAAGGGAGTCTCCCTTTCCCCGGTGGAGTTAGAGAAGACTCTGCTCCACCACCTCTTGTGG
AAGGCCTGACATCAGTCAGGCCCGCCCGCAGCCATCCGGAGACCTAAACGTCTCGCTGTGATGCTGTGCTTCAG
AGGTCACGCTCCTGTTTCACTTTCATGTTCTGCTCTGTACACCTGGCTCCGCCTTCTAGATAGCAATAGCAGAA
TTAGTGAAAGTATTAAAGTCTTTGATCTTTCTGAGAAGAGCATAGAAGAAATAATGACGTAAGCTGTCCTCTCT
CCAGCTCGGCTACCTAAAAGGGAAAGGCCCCCTGTCCGGTGGACACGTGACTCACATGACCTTATTAATCACTG
GAGATGACTCACACTCCTTACCCTGCCCCTTTGCCTTGTACACAATAAATAACAGCGCGACCAGGCATTCGGGG
CCACTGCCAGTCTCCACATCTAGGTGGTAGTGGTCCCCCGGGCCCAGCTGTCTTTTCTTCTATCTCTTTGTCTT
GTGTCTTTATTTCTACGACCTCTCATCTCCGCACACCAGGAGAAAAACCCACAGACCCAGTAGGGCTGGACCCT
ACAGCTCTATGCAAATCAGATACTGCCTCCTCCAGCCTCCCATATAACCGACCACTTTTCTGCCCTCCC
[SEQ ID NO: 15]
TTTTAAGTGTGTTTTTAAAAAAAAGTCTGTGCTACTAGAAAGAAAAAGAAAACAACTGTTCTTGCTATCATACA
TTACATGGTTAAAGGGGCACCTCCCTTAACTGACTGGAGGAAGTTAGCTGTTGGTTTCAAAATCACTCTTTCCA
GAAAAGCAGGGTGTGTGACTCAGATTTCCACCCGAAGGTGGTCCTTGAGAGAAGGTCTTAGATGTTTAGAAGAG
GTTATGTTACATTCTTTGGTATCACTTATTATTTCCTTCAGGCAAACCACATGTCAGAGGACTTCAATCTGAAA
ATGACGTGAGTTACATGGGTTCCTTCCTCTTCGTTTTTTAAAGGTCCTCTTGTCTTTCTGTTTTCATGTGTGCT
CTTCACTGGCAGCTTTCTATAAACAAATGCACTTATCAGAATAAAAGAGTCATTTCACTTCTCTAGGGAGAGAT
TTTTATATCCATCTTTTGAAGAGATGAGATGAAACGTAGTTTGAAATTGAATGTATACAAATTAGTGTATAGAT
CTCCACATTTTAAAAGACCCTGAGTGGTCAAAATGATGAAACAAATGAGAAAGAAAAATAAGAGGACAAGAACT
AACATTAATTGGAAACCCAGTGTGTATCTCACAACCTTATAGGTATTTTGCACACAGCATTTAATTTGATCCTC
CTATTAAGGATTAAATAGACATTTTATCTCCATGTTAT
[SEQ ID NO: 16]
GTCTCAAGTTTCTAGAGAGCCAGGCTCTCGCAGGCTGAGGTGGCTTCCACACTGTTCATGAACTGCTCTTGGCG
GGCTGGTGGCCTGTTTGATATTCTGAGGTCTAGCAAAGATTTAGCTCCAGGGGGTCATCAAGAGGTCATGGTCT
TGATCATTTCCTCTGTTATTTATCTTTGGCTTGTGGTACTGGAGCCTGCCTTTGATACAGGGCATTTCTACCAC
AGCTGCACAGAATCAAGCATTTCAGTGATTTGGTTTGAGGTGAAGGAGGCTTGGCTTCTAATGCTGCGAAAATG
TCACTGGGTACATCTGAGAATATCTAGCTCTGTCCTTCACTGATGAGCAAATTCATTTCTGGGGGTTTACCAGT
GGCACTTCCCAGACTTGGGTCAGGAAAGGAATCCTCCTGGTGAGGACAGCAGTTTTCAGTTTGAGTAATTCCAT
TTTCAGTTCCTTCTGAGACATTTTTCTTGCTCTGAGCTGGAGATAAAAGCAGCATGTCTCAGGCCTCTAGGGTG
GATCTCTACTTTTCAACACTGTTTATGACTCACCTGTGACTGCCCTCAGTGCAGCCAAGGGGACCATTTATGCT
GAATTAGCCTTTGGATTTTTCTTCTTAGTGACAGCAAAACTTCCGTGACAGCAAACAAGTAAACCACAGGAACA
TCCCTGGTCTGCTCAGCACAGCAGGCTCTTGGGTTCTCAGAGAGCCTGGGAGGACTTTTCAGCTTGTCTTATGT
GGGTTTGTTCACCCCGTCCGGTTTCATTTTTATTTAAAACAATTGGTATCTTTCATCTTGGTTGCCAAGTATGG
GTTGCATATGGTTTTGACCTGATATGAAGGGCCCAAATCAAATACCCAGGACTTGGCCTGGAAAATTGGTTGTA
TAAGACCTGCTCTCTTGTTCCCCTAAAAAGCAGCCTAGCAAATGGAAGCAATGGGTCCCAGGAGTTCGTTTCTG
TATGCGGCTCTAACTCTGCCACGAAGGCCAGGACAGATAGTGTGCAGTGCTCTGTCATGTCTACCAGAGAAAAT
GATGCAACATGTGCTGGAGACTTTACCATCTGTGCAAGAGTGCAGCCCTAGGCTCAATCCAAAAGAAAAAGGAA
GTG
[SEQ ID NO: 17]
ATTGGCATACAGCTGTTTCCTGCTACTGCCTTACTCTAGAGAGAGGATGACTTATCACTCAACTGAAGCTCCCA
AAAGAAGAGCTGTAGGAGGCCTTGTTACCTCACACAGCTGGCTAAACTCATAACCTGCCCTGTCATGATTGCTA
TTCTGGGGCTTGATCGATAAAGACTCGGGTGTTCTCAATTCTGTTCTTTTCTTTGCAATCGCAACACTCAACAA
TTTCTTGCTTGATAACTATATGCAGACACTGCCCTCACTTCCCCAAGGAATTGCACTGTGCCTGCACATCCCTG
TCACTTTCACAAAGAGGCAGATAATTTCCTCTCACTGGATAGGGTGCTATGTGGAAATGACGTGAAATCCGCTC
AACCTGATTTCCAACATGTGCGTTCGTTTTCTCCCTATTCAGGTCATAGTGATTCATGAGAGAGAGAGGCAGAG
CGGGAACTGAAGCAGTTTCAGCCTCACCAAAGTACCTTTCCCCTAGTTCCTCTTACTGTGCTCATTATCCTTTC
TTTCCTGCTTACCATACATCGTTTACCAACCCTTTGAAAATTTAAACTACCAGGTTCATCTAGCTCTGGCACTG
CTAGAACCGAGACTACAGAAGATGTCTGAGTCTTCCAAGCTATTTGGGTCAAACAAAACAAACAAAACCACACA
CAGCCACCGAGTTGGGTTATTTTAAGGACTTAGAGCTACAGAAGGCAATCTGTCAGCATCCGAGGCTGTTTCAA
AACCACCTCCTCCCTCCGTGCTTGAGTCAAGAGCTGCAGTCTTTCTATCGCCCATAGCCATGCTGCAGCACTTC
CTCAATCACCCTCTTCTCGCTGCACGCATTGACTTTCGGTTTCTTCACAGAGTCACTGACAGCTTGCAGCTTTG
GCTTATCGATAGCCCCGTGGTTCTCTCCATCCATGACCTACCAGATGGCATTTTAGTTTAGTTCCCACCACTCA
CTGCCTTATTTAATTGGTATTTCTTGCCCCACATTTCCAAAGCAAGCCCGGTCTGGATTCTGTTTGGTTCAGCC
ATCTCATGGGTCTCCAGCTGCTTCCCTGTCCTGTAGGCGGCCTGCGTGAGGCGCAGTTCCTACTTCCAAGGTAT
TGTGGGGCAGTGGCTATTTTTGCAGGCTATTGTGAGGCAGCAGAGGTAAAGTATGAGTTGAAACATACAAACAA
AATATGGCCCCTCAGCATTGTAGAACTTTTGCAGATAATGAATTTTAGAACCAAATAGAGCAGGACATTTGTTT
TCACAGTGAGTCTTATGGCTCTACTTAGAATCTTGTCACTCTGTTTTGCAGTCACCTCTCCAAGTCATTGTCTT
TTATTGTACCCTTGAGAAACAATACTGTTTCCATTCCAACCACTTTACTGAGTATTTTTACTCAGTGGTCAGGT
CACAAATAATCTGTTAAGTTTTAAATTCAGTTGTCTTGTATTGCTGATTTCTCTGTCTTATTTAATGTATTAT
[SEQ ID NO: 18]
CAGCAAAGAGAACAAACTTTTGCAGATGCCCTGTGTGATAAGAAGCACACTCATTCAACAAGTTTAGAACATCC
ATTTAATCCTGCTCTTTTCTTGCTAATATTCAAATTTCTTTAGTTGACTTGTATTTCAGGACCTTGTTGAAAAA
ATAACAATATTCTAGATTCTAGAGTCTAGAAAAGTAGGATCAACCTTTGACATATATCAGAATTGAGGGACTAA
GTGGCTAAGTTGGCTGGACTTCCTGGGTCAACAGGGACTTCCCCAGGGGGACTTTCCCCTAAGCCAAAATGAGT
CATAGCTGCAAGGTACGGGATTGAAACTTCACCCAATCAACAGGGACTTTCCCCTAAGCCAAAATGAGTCATAG
CTGCAAGCGAAGGGATTAAAACTTCAACCAATGAAAGGGGACTTTCCCCTAAACCAAAATGAGTCATAGCTGCA
AGCTAAGGGATTGAAATTTCAAGCAATCAAAGGGGACTTTCTTCTAAGCCAAAATGAGTCACAGCTGCAAACTA
AGGCATTGAAACTTCAACCAATCATATAGGGAGTTTAAGCTCTAGCTGCAGCCTGATGTTTTTAACCAAGCAGG
CCCACCAACCCACAAGCGGATAGAAAATAAGCTAATCCTACAGGACAGGAAAAGGAAAAGGGGAGGAGTCATAA
GGGGACATAAGCCTAAGACACCCAAGCCAGAAACGGCAGCCATTCCGGATCCCCATGGAAGCTTTACTTTCTCT
TTTGCTTTACTTTCGCTTTTGCTTTACTTCCGCTTTTGCTTTACTTTCGCTTTTGCTTTACTTTCACTTTCGCT
TTAATAAATCTTGCCGCAGCACACTCTTTGGGTCTGCGCGTTTCTCTAATCGAGCT
[SEQ ID NO: 19]
ATAGTAAAGAAAAAGTTTTGTTTTCAGATATAATTGAATACCGAAGAAAATCAGAAAATTCAACTGAAAACCTA
TTTAAAGTAAAAAGAAAACTTACTAAGATGGCTGCATACAAAAATCAAGAGTTTTCCTTTTATAGTAGTATAAC
AGGATGTGAAAATGAAAGCTTTGACTGAACTCTGAATTACAGTGTGCTATGAGTCAGATCAACAAGCTGAAATT
TCAAAGAAGGTGTTTGTGAACCTGCTAGATGAATCATCTACATCAAGGATCTAAAACGGTCAGGTGGCTGGTGA
TTCAACAGGATTTGGTGAAGAGGACTGGTTTGGAAAATCTGTGTTCTTAATCCTGTCTTCCATCCCAGAGAAAG
GGAGGGGGGATGATTGACCCTTCTTTCTCAAATCTAGGGAGAAAGTTTCATGAGGGCTGCTTGCAGAGCTTGAT
ATATGTTTCTTGTAGTCGTGGGGCATTGTGAATTGGTTTCTGATTCATGCCTGTGAAATTTGAGACAAAAAGAT
CATGGCTGGAAGTTTCGTGTGACTAGGTGACTAAGTTCCTTCCAATAAGAAATAATGAATGCAGATGTCTTGTT
CTCCTTTTCTCTCCTTCTTCCTGTTTCTAGTTGGCCCTGGAACAGTGGCCTCCAGGCCTGAGATAATCGAAGCA
ACTGGAGAAGCTTGAAGGAGTCTGGGTCCCTGATCCTGACAAAGCCGCTATTCCAC
[SEQ ID NO: 20]
TCACCCCACAACAGATGTTTTGCTCCAAGACAGCTGGGCCTAAGCCCTTCAGTTCGAATCCTCATCCAAGACTG
AAAGCTCCTACATCTTCCTGTCACTTTAGCCACAAGCTGCACCGTCTGGAAATGCCCACTGCTACTAGGGACAC
AGGCGAGGCCGGACAGTGCCGGAGGACCCGGGGTGCCTCCCTCCACGTGCGCGGCCTCACAGGTCAGTGGAAAC
AAGAAATCCCCCAATCCACAGCCACGATGACTTCAAAATACTTCCTGAAGGCATCAGGCCATGAAGCTGCGACC
TGCGAGGACACAGCAAGCGGGGTGACGCTGAGCAGAGGCTTGGTGTGGAGTCCGCGCCCGCAGGGTCCCTCCCC
GCCCCGCGGGTGCTTCCCGCCACCTCGCTCGCCTCTGCGTCGCAGTGACAACGCGGGGATGCCGACCACAGGTG
GTTGGAAGGAGACAGCGAGATTCTGAGGGGAAAGAATAAGGAGAGAAGCAGCATCTTTTGGGCAGAAGGCCCGT
TTCCGCACGGTCCTGCCGAGGCCTCCGGGCATCCTTCACCTCAGGGAAGAGAAAATCCAACCTTACTTATCACA
GGCGCGGGACCTACGTTCCCTTCGGGAGCAGAAGAACCAGGACAGGATTAGGACGCAGGCCAGGCGGCAAAGCC
GCTCCCTCTCTCCCACCTCCAGGACAAGCGGCACCTCCAGTCCACCCTCGCTCCAGCCCTCCCGTACTCTACCC
CCGGCATCCTCTGCCCCGAAGCAGGGAGATGCCGGGAGCCCTCCCTACCAGGCTGTACCGGAGAAAAACAAACC
TGGAGCCACACCGCGCACATGCGCAGAAAGCCCAAAGCGGCTCCCGCGACCGGGGCCAAAAAGTGCACACTACA
TTTCCCACAAGTCCTCGCGCCGCTCCCTTAGAAAGTACACGCTACATTTCCCACAAGCCCTCGCGCCGCTCCCT
GAGAAAGTGAAGACTACATTTCCCAGAAGTCCTCGCGCCGCTCCTTTAGAAACGGCCTGAGGTTTCAGCGCCGT
TCGGTAGTTAGTTCCCAGATGCACCGAGTGCAATACTGCAATTTTATTCGTTAGCTGAAATAAATTATGAGTAT
TTAAATTACACAGGTTTCTTATGAATTACATCTCCCTGATGATTTGGCTGTCCTCAACACTTCCTCGTTTGGCA
GAATTCAGGAGTGGAATTTCTCCAGACACGGTCGGTTCCTGTGGCCCCGCTGCGCCGCGGCGGCACCACCTCCA
CTTGCGCCTGTGGCTCCACTTGCTTGGTTCTGGTCCACTGGACGCTCACCCTGAGAAAACACGCTGGACATGAG
CATTACCAGCCTGTACACTGTTGTAAAGATAACTCGGAATTGCTCTTCAAATTTTATT
[SEQ ID NO: 75]
TTCACTCAAATTCCTCCCAAATAGTTTTGAGAGTCTCCAGATGTCTCACATCTACTTTACATACAAGTTCCCTC
AGATCTCCTTCTCTCATGATCCCATTTCAGCTCTCTGTGGTGAATCTGATACCCCACTACTGATGCCAGGATCT
ACAATGGTTAAGGTTCCACTGCCTGACAAACATGTTAGTGATACTGGTGCTTGCTGTTGTCTGCATTGAGGCTT
CTGATATCTGTTACTGAGGATATTGAGGTTTACCCACAGAGAACTCTTGCCTGGTGCTGCTGAAGCTCTTCGTA
TACCATCTCCTCTGAACAGAGTCAACATATGACTTGTCTTCAATTATAGCCAAAGCTGTAAGTCCCAATATCAC
TAATGCTTTTCTTTGAAGCTCTTAAATGCATTTAATTCTTAATGCATAGCAGTTCTATTTGACTGTACACCCTA
GTACTCAAAATTGATTCTAAGAAAGCTGTATGGGAATACAAACTAAAACAATGAATGCTATACCACCTTAAGAA
CCAGTTGATCCCTCCATCCTTGGCCACTGTTCTTGAAAACAAAAGTGAATTGACTCCCTTTTTCTCTAGAATGA
TTTGCTCCAGAACAAAGTTTAATTCTTTATGTATGAAGACCCATAATTGTTTCTATCACCCTGCCTACATATTC
CTAACTAGTAAGGTAAATAAACTAAATTT

SEQ ID NOS: 1-20 and 75 correspond to the chromosome coordinates in human genome assemblies hg19 and hg38 as shown in Table 1.

TABLE 1

| SEQ ID | | |
|---|---|---|
| NO. | Hg19 coordinates | Hg38 coordinates |
| 1 | chr3: 115502796-115503649 | chr3: 115783949-115784802 |
| 2 | chr7: 17273689-17275085 | chr7: 17234065-17235461 |
| 3 | chr5: 87436882-87437724 | chr5: 88141065-88141907 |
| 4 | chr3: 110532783-110533865 | chr3: 110813936-110815018 |
| 5 | chr13: 45491691-45492993 | chr13: 44917556-44918858 |
| 6 | chr7: 149321176-149322360 | chr7: 149624085-149625269 |
| 7 | chr1: 232765494-232766702 | chr1: 232629748-232630956 |
| 8 | chr3: 106147345-106148130 | chr3: 106428498-106429283 |
| 9 | chr7: 12296845-12297778 | chr7: 12257219-12258152 |
| 10 | chr3: 137784804-137785736 | chr3: 138065962-138066894 |
| 11 | chr5: 90458122-90459221 | chr5: 91162305-91163404 |
| 12 | chr6: 141647521-141648419 | chr6: 141326384-141327282 |
| 13 | chrX: 147461745-147463483 | chrX: 148380225-148381963 |
| 14 | chr12: 31004108-31004917 | chr12: 30851174-30851983 |
| 15 | chr3: 105895555-105896259 | chr3: 106176708-106177412 |
| 16 | chr10: 90030671-90031784 | chr10: 88270914-88272027 |
| 17 | chr13: 105791495-105792974 | chr13: 105139144-105140623 |
| 18 | chr18: 36520619-36521489 | chr18: 38940655-38941525 |
| 19 | chr5: 44875678-44876400 | chr5: 44875576-44876298 |
| 20 | chr10: 43047433-43048823 | chr10: 42551985-42553375 |
| 75 | chr8: 90253578-90254272 | chr8: 89241348-89242043 |

### 5.3. Cells and Compositions

The present disclosure provides cells comprising an exogenous composition, wherein the exogenous composition is integrated up to about 10 kb upstream or downstream or within a locus of the genome of the cells. In certain embodiments, the locus comprises a GSH disclosed herein. In certain embodiments, the locus comprises or consists of a nucleotide sequence that is at least about 80% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75. In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75. In certain embodiments, the locus comprises or consists of the nucleotide sequence set forth in SEQ ID NO: 1, SEQ ID NO: 2, SEQ ID NO: 3, SEQ ID NO: 4, SEQ ID NO: 5, or SEQ ID NO: 6. In certain embodiments, the locus comprises or consists of the nucleotide sequence set forth in SEQ ID NO: 6.

In certain embodiments, the cell is a human cell. In certain embodiments, the cell is a non-human cell. Non-limiting examples of non-human cells include animal cells, plant cells, fungal cells, and yeast cells. Non-limiting examples of animal cells that can be used with the presently disclosed subject matter include mammal cells, bird cells, reptile cells, fish cells, and insect cells. Non-limiting examples of mammal cells that can be used with the presently disclosed subject matter include mouse cells, rat cells, hamster cells, guinea pig cells, rabbit cells, dog cells, cat cells, sheep cells, pig cells, goat cells, cattle cells, horse cells, monkey cells, and ape cells.

The presently disclosed cell can be from any tissue, including, but not limited to, exocrine secretory epithelial cells, hormone-secreting cells, keratinizing epithelial cells, wet stratified barrier epithelial cells, sensory transducer cells, autonomic neuron cells, sense organ and peripheral neuron supporting cells, central nervous system neurons and glial cells, lens cells, metabolism and storage cells (e.g., adipocytes, liver lipocytes), kidney cells, lung cells, gut cells, exocrine gland cells, urogenital tract cells, extracellular matrix cells, contractile cells (skeletal muscle cells, heart muscle cells, smooth muscle cells), stem cells, blood cells, immune cells, interstitial cells, germ cells, and nurse cells (e.g., ovarian follicle cells).

In certain embodiments, the cell is a human stem cell (HSC). In certain embodiments, the cell is an erythroid cell.

In certain embodiments, the cell is an immunoresponsive cell. In certain embodiments, the cells are selected from cells of the lymphoid lineage and cells of the myeloid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R.A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

The cells (e.g., T cells) can be autologous, non-autologous (e.g., allogeneic), or derived in vitro from engineered progenitor or stem cells.

In certain embodiments, the presently disclosed cells are capable of modulating the tumor microenvironment. Tumors have a microenvironment that is hostile to the host immune response involving a series of mechanisms by malignant cells to protect themselves from immune recognition and elimination. This “hostile tumor microenvironment” comprises a variety of immune suppressive factors including infiltrating regulatory CD4⁺ T cells (Tregs), myeloid derived suppressor cells (MDSCs), tumor associated macrophages (TAMs), immune suppressive cytokines including TGF-β, and expression of ligands targeted to immune suppressive receptors expressed by activated T cells (CTLA-4 and PD-1). These mechanisms of immune suppression play a role in the maintenance of tolerance and suppressing inappropriate immune responses, however within the tumor microenvironment these mechanisms prevent an effective anti-tumor immune response.

Collectively these immune suppressive factors can induce either marked anergic or apoptosis of adoptively transferred CAR modified T cells upon encounter with targeted tumor cells.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

The presently discloses subject matter further provides compositions comprising a presently disclosed cell or cells. In certain embodiments, the composition is a pharmaceutical composition that further comprises a pharmaceutically acceptable carrier.

### 5.4. Transgene

In certain embodiments, the exogenous composition comprises a transgene. In certain embodiment, the transgene encodes a non-coding RNA. In certain embodiments, the non-coding RNA is a microRNA (miRNA), a small interference RNA (siRNA), a piwi-interacting RNA (piRNA), a small nucleolar RNAs (snoRNAs), a small nuclear RNA (snRNA), a small hairpin RNA (shRNA), an extracellular RNA (exRNA), or a long non-coding RNA (lncRNA),

In certain embodiment, the transgene encodes a polypeptide. In certain embodiments, the polypeptide is a therapeutic polypeptide. In certain embodiments, the polypeptide is not expressed endogenously in the cell. In certain embodiments, the polypeptide is endogenously expressed in the cell in an amount that does not have an intended biological or therapeutic effect.

In certain embodiments, the transgene is a β-globin transgene, e.g., a transgene that encodes a β-globin molecule. In certain embodiments, the β-globin transgene further comprises a β-globin promoter. In certain embodiments, the β-globin transgene further comprises a human β-globin 3′ enhancer.

In certain embodiments, the transgene encodes an antigen-recognizing receptor. In certain embodiments the antigen-recognizing receptor binds to an antigen of interest. In certain embodiments, the antigen is a tumor antigen or a pathogen antigen.

In certain embodiments, the antigen is a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. Non-limiting examples of tumor antigens include CD19, carbonic anhydrase IX (CAIX), carcinoembryonic antigen (CEA), CD8, CD7, CD10, CD20, CD22, CD30, CD33, CLL1, CD34, CD38, CD41, CD44, CD49f, CD56, CD74, CD133, CD138, CD123, CD99, CD70, CD44V6, an antigen of a cytomegalovirus (CMV) infected cell (e.g., a cell surface antigen), epithelial glycoprotein-2 (EGP-2), epithelial glycoprotein-40 (EGP-40), epithelial cell adhesion molecule (EpCAM), receptor tyrosine-protein kinases, folate-binding protein (FBP), fetal acetylcholine receptor (AChR), folate receptor-α, Ganglioside G2 (GD2), Ganglioside G3 (GD3), human Epidermal Growth Factor Receptor 2 (HER-2), human telomerase reverse transcriptase (hTERT), Interleukin-13 receptor subunit alpha-2 (IL-13Rα2), κ-light chain, kinase insert domain receptor (KDR), Lewis Y (LeY), L1 cell adhesion molecule (L1CAM), melanoma antigen family A, 1 (MAGE-A1), Mucin 16 (MUC16), Mucin 1 (MUC1), Mesothelin (MSLN), ERBB2, MAGEA3, p53, MART1, GP100, Proteinase03 (PR1), Tyrosinase, Survivin, hTERT, EphA2, NKG2D ligands, cancer-testis antigen NY-ESO-1, oncofetal antigen (h5T4), prostate stem cell antigen (PSCA), prostate-specific membrane antigen (PSMA), ROR1, tumor-associated glycoprotein 72 (TAG-72), vascular endothelial growth factor R2 (VEGF-R2), Wilms tumor protein (WT-1), BCMA, NKCS1, EGF1R, EGFR-VIII, ADGRE2, CCR1, LILRB2, PRAME, and ERBB (e.g., Erb-B2, Erb-B3, and Erb-B4). In certain embodiments, the antigen-recognizing receptor binds to CD19. In certain embodiments, the antigen-recognizing receptor binds to the extracellular domain of a CD19 protein.

In certain embodiments, the antigen-recognizing receptor binds to a pathogen antigen, e.g., for use in treating and/or preventing a pathogen infection or other infectious disease, for example, in an immunocompromised subject. Non-limiting examples of pathogen comprises a virus, bacteria, fungi, parasite and protozoa capable of causing disease.

Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses). Non-limiting examples of bacteria include Pasteurella, Staphylococci,

Streptococcus, Escherichia coli, Pseudomonas species, and Salmonella species. Specific examples of infectious bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtherias, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

Non-limiting examples of antigen-recognizing receptors include a chimeric antigen receptor (CAR), a T-cell receptor (TCR), a chimeric co-stimulating receptor (CCR), and a TCR like fusion molecule.

In certain embodiments, the antigen-recognizing receptor is a chimeric antigen receptor (CAR). CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., a scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40, CD27, CD40 and NKGD2) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the CAR is a second-generation CAR. In certain embodiments, the CAR comprises an extracellular antigen-binding domain that binds to an antigen, a transmembrane domain, and an intracellular signaling domain, wherein the intracellular signaling domain comprises a co-stimulatory signaling region. In certain embodiments, the CAR further comprises a hinger/spacer region.

In certain embodiments, the extracellular antigen-binding domain of the CAR (for example, an scFv) binds to an antigen with a dissociation constant (K_{d}) of about 2×10⁻⁷ M or less. In certain embodiments, the K_{d} is about 2×10⁻⁷ M or less, about 1×10⁻⁷ M or less, about 9×10⁻⁸M or less, about 1×10⁻⁸M or less, about 9×10⁻⁹ M or less, about 5×10⁻⁹M or less, about 4×10⁻⁹ M or less, about 3×10⁻⁹M or less, about 2×10⁻⁹M or less, about 1×10⁻⁹ M or less, about 1×10⁻¹⁰ M or less, or about 1×10⁻¹¹M or less. In certain embodiments, the K_{d} is about 1×10⁻⁸M or less.

Binding of the extracellular antigen-binding domain (for example, in a scFv or an analog thereof) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (MA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can be an scFv, a F(ab)_{2, or} a Fab (which is optionally crosslinked). In certain embodiments, the extracellular antigen-binding domain is an scFv, which can be a human scFv, or a murine scFv. Any of the foregoing molecules can be comprised in a fusion protein with a heterologous sequence to form the extracellular antigen-binding domain.

In certain embodiments, the extracellular antigen-binding domain comprises an scFv that binds to CD19 (e.g., human CD19). In certain embodiments, the extracellular antigen-binding domain comprises the amino acid sequence set forth in SEQ ID NO: 21 and specifically binds to human CD19. In certain embodiments, the nucleotide sequence encoding the amino acid sequence of SEQ ID NO: 21 is set forth in SEQ ID NO: 22. In certain embodiments, the scFv is from a clone designated as “SJ25C1”.

In certain embodiments, the extracellular antigen-binding domain comprises a heavy chain variable region (V_{H}) comprising a CDR1 comprising the amino acid sequence set forth in SEQ ID NO: 23 or a conservative modification thereof, a CDR2 comprising the amino acid sequence set forth in SEQ ID NO: 24 or a conservative modification thereof, and a CDR3 comprising the amino acid sequence set forth in SEQ ID NO: 25 or a conservative modification thereof. In certain embodiments, the extracellular antigen-binding domain comprises a V_{H} comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25.

In certain embodiments, the extracellular antigen-binding domain comprises a light chain variable region (V_{L}) comprising a CDR1 comprising the amino acid sequence set forth in SEQ ID NO: 26 or a conservative modification thereof, a CDR2 comprising the amino acid sequence set forth in SEQ ID NO: 27 or a conservative modification thereof, and a CDR3 comprising the amino acid sequence set forth in SEQ ID NO: 28 or a conservative modification thereof. In certain embodiments, the extracellular antigen-binding domain comprises a V_{L} comprising a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28.

In certain embodiments, the extracellular antigen-binding domain comprises a V_{H} comprising a CDR1 comprising the amino acid sequence set forth in SEQ ID NO: 23 or a conservative modification thereof, a CDR2 comprising the amino acid sequence set forth in SEQ ID NO: 24 or a conservative modification thereof, and a CDR3 comprising the amino acid sequence set forth in SEQ ID NO: 25, a conservative modification thereof; and a V_{L} comprising a CDR1 comprising the amino acid sequence set forth in SEQ ID NO: 26 or a conservative modification thereof, a CDR2 comprising the amino acid sequence set forth in SEQ ID NO: 27 or a conservative modification thereof, and a CDR3 comprising the amino acid sequence set forth in SEQ ID NO: 28 or a conservative modification thereof. In certain embodiments, the extracellular antigen-binding domain comprises a V_{H} comprising a CDR1 comprising amino acids having the sequence set forth in SEQ ID NO: 23, a CDR2 comprising the amino acid sequence set forth in SEQ ID NO: 24, and a CDR3 comprising the amino acid sequence set forth in SEQ ID NO: 25; and a V_{L} comprising a CDR1 comprising the amino acid sequence set forth in SEQ ID NO: 26, a CDR2 comprising the amino acid sequence set forth in SEQ ID NO: 27, and a CDR3 comprising the amino acid sequence set forth in SEQ ID NO: 28.

In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 29. For example, the extracellular antigen-binding domain comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 29. In certain embodiments, the extracellular antigen-binding domain comprises a V_{H} comprising the amino sequence set forth in SEQ ID NO: 29.

In certain embodiments, the extracellular antigen-binding domain comprises a V_{L} comprising an amino acid sequence that is at least about 80% (e.g., at least about 85%, at least about 90%, or at least about 95%) homologous or identical to the amino acid sequence set forth in SEQ ID NO: 30. For example, the extracellular antigen-binding domain comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 30. In certain embodiments, the extracellular antigen-binding domain comprises a V_{L} comprising the amino acid sequence set forth in SEQ ID NO: 30.

In certain embodiments, the extracellular antigen-binding domain comprises a V_{H} comprising an amino acid sequence that is at least about 80% (e.g., at least about 85%, at least about 90%, or at least about 95%) homologous or identical to the amino acid sequence set forth in SEQ ID NO: 29, and a V_{L} comprising an amino acid sequence that is at least about 80% (e.g., at least about 85%, at least about 90%, or at least about 95%) homologous or identical to the amino acid sequence set forth in SEQ ID NO: 30.

In certain embodiments, the extracellular antigen-binding domain comprises a V_{H} comprising the amino acid sequence set forth in SEQ ID NO: 29. In certain embodiments, the extracellular antigen-binding domain comprises a V_{L} comprising the amino acid sequence set forth in SEQ ID NO: 30. In certain embodiments, the extracellular antigen-binding domain comprises V_{H} comprising the amino acid sequence set forth in SEQ ID NO: 29 and a V_{L} comprising the amino acid sequence set forth in SEQ ID NO: 30, optionally with (iii) a linker sequence, for example a linker peptide, between the V_{H} and the V_{L}. “Linker”, as used herein, shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker comprises the amino acid sequence set forth in SEQ ID NO: 31. SEQ ID Nos: 21-31 are provided in the following Table 2.

TABLE 2

| | | anti-human CD19 scFv | | |
|---|---|---|---|---|
| CDRs | | 1 | 2 | 3 |
| V_{H} | a.a. | GYAFSS [SEQ ID | YPGDGD [SEQ ID NO: | KTISSVVDF [SEQ ID |
| | | NO: 23] | 24] | NO: 25] |
| V_{L} | a.a. | KASQNVGTNVA [SEQ | SATYRN [SEQ ID NO: | QQYNRYPYT [SEQ ID |
| | | ID NO: 26] | 27] | NO: 28] |

| | |
|---|---|
| Full V_{H} | EVKLQQSGAE LVRPGSSVKI SCKASGYAFS SYWMNWVKQR PGQGLEWIGQ |
| | IYPGDGDTNY NGKFKGQATL TADKSSSTAY MQLSGLTSED SAVYFCARKT |
| | ISSVVDFYFD YWGQGTTVTV SS [SEQ ID NO: 29] |
| Full V_{L} | DIELTQSPKF MSTSVGDRVS VTCKASQNVG TNVAWYQQKP GQSPKPLIYS |
| | ATYRNSGVPD RFTGSGSGTD FTLTITNVQS KDLADYFCQQ YNRYPYTSGG |
| | GTKLEIKR [SEQ ID NO: 30] |
| scFv | MALPVTALLL PLALLLHAEV KLQQSGAELV RPGSSVKISC KASGYAFSSY |
| | WMNWVKQRPG QGLEWIGQIY PGDGDTNYNG KFKGQATLTA DKSSSTAYMQ |
| | LSGLTSEDSA VYFCARKTIS SVVDFYFDYW GQGTTVTVSS GGGGSGGGGS |
| | GGGGSDIELT QSPKFMSTSV GDRVSVTCKA SQNVGTNVAW YQQKPGQSPK |
| | PLIYSATYRN SGVPDRFTGS GSGTDFTLTI TNVQSKDLAD YFCQQYNRYP |
| | YTSGGGTKLE IKR [SEQ ID NO: 21] |
| DNA | ATGGCTCTCCCAGTGACTGCCCTACTGCTTCCCCTAGCGCTTCTCCTGCATGCAGAGG |
| | TGAAGCTGCAGCAGTCTGGGGCTGAGCTGGTGAGGCCTGGGTCCTCAGTGAAGATTTC |
| | CTGCAAGGCTTCTGGCTATGCATTCAGTAGCTACTGGATGAACTGGGTGAAGCAGAGG |
| | CCTGGACAGGGTCTTGAGTGGATTGGACAGATTTATCCTGGAGATGGTGATACTAACT |
| | ACAATGGAAAGTTCAAGGGTCAAGCCACACTGACTGCAGACAAATCCTCCAGCACAGC |
| | CTACATGCAGCTCAGCGGCCTAACATCTGAGGACTCTGCGGTCTATTTCTGTGCAAGA |
| | AAGACCATTAGTTCGGTAGTAGATTTCTACTTTGACTACTGGGGCCAAGGGACCACGG |
| | TCACCGTCTCCTCAGGTGGAGGTGGATCAGGTGGAGGTGGATCTGGTGGAGGTGGATC |
| | TGACATTGAGCTCACCCAGTCTCCAAAATTCATGTCCACATCAGTAGGAGACAGGGTC |
| | AGCGTCACCTGCAAGGCCAGTCAGAATGTGGGTACTAATGTAGCCTGGTATCAACAGA |
| | AACCAGGACAATCTCCTAAACCACTGATTTACTCGGCAACCTACCGGAACAGTGGAGT |
| | CCCTGATCGCTTCACAGGCAGTGGATCTGGGACAGATTTCACTCTCACCATCACTAAC |
| | GTGCAGTCTAAAGACTTGGCAGACTATTTCTGTCAACAATATAACAGGTATCCGTACA |
| | CGTCCGGAGGGGGGACCAAGCTGGAGATCAAACGG [SEQ ID NO: 22] |
| Linker | GGGGSGGGGSGGGGS [SEQ ID NO: 31] |

As used herein, the term “a conservative sequence modification” refers to an amino acid modification that does not significantly affect or alter the binding characteristics of the presently disclosed CAR (e.g., the extracellular antigen-binding domain of the CAR) comprising the amino acid sequence. Conservative modifications can include amino acid substitutions, additions and deletions. Modifications can be introduced into the human scFv of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (1) above) using the functional assays described herein. In certain embodiments, no more than one, no more than two, no more than three, no more than four, no more than five residues within a specified sequence or a CDR region are altered.

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 29 and 30) may contain substitutions (e.g., conservative substitutions), insertions, or deletions relative to the specified sequence(s), but retain the ability to bind to a target antigen (e.g., CD19). In certain embodiments, a total of 1 to 10 amino acids are substituted, inserted and/or deleted in a specific sequence (e.g., SEQ ID NOs: 29 and 30). In certain embodiments, substitutions, insertions, or deletions occur in regions outside the CDRs (e.g., in the FRs) of the extracellular antigen-binding domain. In certain embodiments, the extracellular antigen-binding domain comprises V_{H} and/or V_{L} sequence selected from SEQ ID NOs: 29 and 30, including post-translational modifications of that sequence (SEQ ID NOs: 29 and 30).

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences of scFv m903, m904, m905, m906, and m900) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In certain embodiments, the transmembrane domain of the CAR comprises a native or modified transmembrane domain of CD8, CD28, CD3ζ, CD40, 4-1BB, OX40, CD84, CD166, CD8a, CD8b, ICOS, ICAM-1, CTLA-4, CD27, CD40, or NKGD2.

In certain embodiments, the transmembrane domain of a presently disclosed CAR comprises a CD28 polypeptide. The CD28 polypeptide can have an amino acid sequence that is at least about 80%, at least about 85%, at least about 90%, at least about 95%, at least about 96%, at least about 97%, at least about 98%, at least about 99% or at least about 100% homologous or identical to the sequence having a NCBI Reference No: NP_006130 (SEQ ID NO: 32) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 32, which is at least 20, or at least 30, or at least 40, or at least 50, and up to 220 amino acids in length. Alternatively or additionally, in certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 32. In certain embodiments, the CAR comprises a transmembrane domain of CD28 (e.g., human CD28). In certain embodiments, the transmembrane domain of CD28 comprises or consists of amino acids 153 to 179 of SEQ ID NO: 32. In certain embodiments, the CAR comprises a CD28 polypeptide comprising or consisting of amino acids 153 to 179 of SEQ ID NO: 32. SEQ ID NO: 32 is provided below.

| [SEQ ID NO: 32] | |
|---|---|
| 1 | MLRLLLALNL FPSIQVTGNK ILVKQSPMLV AYDNAVNLSC KYSYNLFSRE FRASLHKGLD |
| 61 | SAVEVCVVYG NYSQQLQVYS KTGFNCDGKL GNESVTFYLQ NLYVNQTDIY FCKIEVMYPP |
| 121 | PYLDNEKSNG TIIHVKGKHL CPSPLFPGPS KPFWVLVVVG GVLACYSLLV TVAFIIFWVR |
| 181 | SKRSRLLHSD YMNMTPRRPG PTRKHYQPYA PPRDFAAYRS |

An exemplary nucleotide sequence encoding amino acids 153 to 179 of SEQ ID NO: 32 is set forth in SEQ ID NO: 33, which is provided below.

[SEQ ID NO: 33]
ttttgggtgctggtggtggttggtggagtcctggcttgctatagcttgc
tagtaacagtggcctttattattttctgggtg

In certain embodiments, the CAR additionally comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of CD8, CD28, CD3ζ, CD40, 4-1BB, OX40, CD84, CD166, CD8a, CD8b, ICOS, ICAM-1, CTLA-4, CD27, CD40, or NKGD2. The hinge/spacer region can be the hinge region from IgG1, or the CH₂CH₃ region of immunoglobulin and portions of CD3, a portion of a CD28 polypeptide (e.g., a portion of SEQ ID NO: 32), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the hinge/spacer region comprises a native or modified hinge region of CD28. In certain embodiments, the hinge/spacer region comprises or consists of amino acids 114 to 152 of SEQ ID NO: 32.

An exemplary nucleotide sequence encoding amino acids 114 to 152 of SEQ ID NO: 32 is set forth in SEQ ID NO: 34 as provided below.

[SEQ ID NO: 34]
atcgaggtgatgtacccccccccctacctggacaacgagaagagcaacg
gcaccatcatccacgtgaagggcaagcacctgtgccccagccccctgtt
ccccggccccagcaagccc

In certain embodiments, the transmembrane domain and the hinge/spacer region are derived from the same molecule. In certain embodiments, the transmembrane domain and the hinge/spacer region are derived from different molecules. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD84 polypeptide and the transmembrane domain of the CAR comprises a CD84 polypeptide. In certain embodiments, the hinge/spacer region of the CAR comprises a CD166 polypeptide and the transmembrane domain of the CAR comprises a CD166 polypeptide. In certain embodiments, the hinge/spacer region of the CAR comprises a CD8a polypeptide and the transmembrane domain of the CAR comprises a CD8a polypeptide. In certain embodiments, the hinge/spacer region of the CAR comprises a CD8b polypeptide and the transmembrane domain of the CAR comprises a CD8b polypeptide. In certain embodiments, the hinge/spacer region of the CAR comprises a CD28 polypeptide and the transmembrane domain of the CAR comprises an ICOS polypeptide.

In certain embodiments, the CAR comprises an intracellular signaling domain. In certain embodiments, the intracellular signaling domain of the CAR comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3ζ-chain is the primary transmitter of signals from endogenous TCRs.

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or has an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 35 or a fragment thereof, and/or may optionally comprise up to one or up to two or up to three conservative amino acid substitutions. SEQ ID NO: 35 is provided below:

[SEQ ID NO: 35]
RVKFSRSADA PAYQQGQNQL YNELNLGRRE EYDVLDKRRG
RDPEMGGKPR RKNPQEGLFN ELQKDKMAEA FSEIGMKGER
RRGKGHDGLF QGLSTATKDT FDALHMQALP PR

An exemplary nucleotide sequence encoding the amino acid sequence of SEQ ID NO: 35 is set forth in SEQ ID NO: 36, which is provided below.

[SEQ ID NO: 36]
agagtgaagttcagcaggagcgcagacgcccccgcgtaccagcagggcca
gaaccagctctataacgagctcaatctaggacgaagagaggagtacgatg
ttttggacaagagacgtggccgggaccctgagatggggggaaagccgaga
aggaagaaccctcaggaaggcctgtTcaatgaactgcagaaagataagat
ggcggaggcctTcagtgagattgggatgaaaggcgagcgccggaggggca
aggggcacgatggcctttTccaggggctcagtacagccaccaaggacacc
tTcgacgcccttcacatgcaggccctgccccctcgc

In certain embodiments, the intracellular signaling domain of the CAR comprises a modified CD3ζ polypeptide comprising one, two or three ITAMs. In certain embodiments, the modified CD3ζ polypeptide comprises a native ITAM1 comprising the amino acid sequence set forth in SEQ ID NO: 37, which is provided below. QNQLYNELNLGRREEYDVLDKR [SEQ ID NO: 37]

An exemplary nucleotide sequence encoding the amino acid sequence of SEQ ID NO: 37 is set forth in SEQ ID NO: 38, which is provided below.

[SEQ ID NO: 38]
cagaaccagctctataacgagctcaatctagga
cgaagagaggagtacgatgttttggacaagaga

In certain embodiments, the modified CD3ζ polypeptide comprises an ITAM1 variant comprising one or more loss-of-function mutations. In certain embodiments, the modified CD3ζ polypeptide consists of one ITAM1 variant comprising or consisting of two loss-of-function mutations. In certain embodiments, each of the one or more (e.g., two) loss of function mutations comprises a mutation of a tyrosine residue in ITAM1. In certain embodiments, the ITAM1 variant (e.g., the variant consisting of two loss-of-function mutations) comprises or consists of the amino acid sequence set forth in SEQ ID NO: 39, which is provided below.

[SEQ ID NO: 39]
QNQLFNELNLGRREEFDVLDKR

An exemplary nucleotide sequence encoding the amino acid sequence of SEQ ID NO: 39 is set forth in SEQ ID NO: 40, which is provided below.

[SEQ ID NO: 40]
cagaaccagctctTtaacgagctcaatctagga
cgaagagaggagtTcgatgttttggacaagaga

In certain embodiments, the modified CD3ζ polypeptide comprises a native ITAM2 comprising the amino acid sequence set forth in SEQ ID NO: 41, which is provided below. QEGLYNELQKDKMAEAYSEIGMK [SEQ ID NO: 41]

An exemplary nucleotide sequence encoding the amino acid sequence of SEQ ID NO: 41 is set forth in SEQ ID NO: 42, which is provided below.

[SEQ ID NO: 42]
caggaaggcctgtacaatgaactgcagaaagataagatggcggaggcct
acagtgagattgggatgaaa

In certain embodiments, the modified CD3ζ polypeptide comprises an ITAM2 variant comprising one or more loss-of-function mutations. In certain embodiments, the modified CD3ζ polypeptide consists of one ITAM2 variant comprising or having two loss-of-function mutations. In certain embodiments, each of the one or more (e.g., two) the loss of function mutations comprises a mutation of a tyrosine residue in ITAM2. In certain embodiments, the ITAM2 variant (e.g., a variant consisting of two loss-of-function mutations) comprises or consists of the amino acid sequence set forth in SEQ ID NO: 42, which is provided below.

[SEQ ID NO: 43]
QEGLFNELQKDKMAEAFSEIGMK

An exemplary nucleotide sequence encoding the amino acid sequence of SEQ ID NO: 43 is set forth in SEQ ID NO: 44, which is provided below.

[SEQ ID NO: 44]
caggaaggcctgtTcaatgaactgcagaaagataagatggcggaggcct
Tcagtgagattgggatgaaa

In certain embodiments, the modified CD3ζ polypeptide comprises a native ITAM3 comprising the amino acid sequence set forth in SEQ ID NO: 45, which is provided below.

[SEQ ID NO: 45]
HDGLYQGLSTATKDTYDALHMQ

An exemplary nucleotide sequence encoding the amino acid sequence of SEQ ID NO: 45 is set forth in SEQ ID NO: 46, which is provided below.

[SEQ ID NO: 46]
cacgatggcctttaccagggtctcagtacagccaccaaggacacctacg
acgcccttcacatgcag

In certain embodiments, the modified CD3ζ polypeptide comprises an ITAM3 variant comprising one or more loss-of-function mutations. In certain embodiments, the modified CD3ζ polypeptide consists of one ITAM3 variant comprising or consisting of two loss-of-function mutations. In certain embodiments, each of the one or more (e.g., two) the loss of function mutations comprises a mutation of a tyrosine residue in ITAM3. In certain embodiments, the ITAM3 variant (e.g., a variant consisting of two loss-of-function mutations) comprises or consists of the amino acid sequence set forth in SEQ ID NO: 47, which is provided below.

[SEQ ID NO: 47]
HDGLFQGLSTATKDTFDALHMQ

An exemplary nucleotide sequence encoding the amino acid sequence of SEQ ID NO: 47 is set forth in SEQ ID NO: 48, which is provided below.

[SEQ ID NO: 48]
cacgatggcctttTccaggggctcagtacagccaccaaggacacctTcg
acgcccttcacatgcag

In certain embodiments, the intracellular signaling domain of the CAR comprises a modified CD3ζ polypeptide comprising or consisting essentially of or consisting of an ITAM1 variant comprising one or more loss-of-function mutations, an ITAM2 variant comprising one or more loss-of-function mutations, an ITAM3 variant comprising one or more loss-of-function mutations, or a combination thereof. In certain embodiments, the intracellular signaling domain of the CAR comprises a modified CD3ζ polypeptide comprising an ITAM2 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3ζ polypeptide comprising a native ITAM1, an ITAM2 variant comprising or having two loss-of-function mutations and an ITAM3 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 37, an ITAM2 variant consisting of the amino acid sequence set forth in SEQ ID NO: 42 and an ITAM3 variant consisting of the amino acid sequence set forth in SEQ ID NO: 47 (e.g., a construct designated as “1XX”). In certain embodiments, the modified CD3ζ polypeptide comprising or consists of the amino acid sequence set forth in SEQ ID NO: 35.

In certain embodiments, the intracellular signaling domain of the CAR comprises a modified CD3ζ polypeptide comprising an ITAM1 variant comprising one or more (e.g., two) loss-of-function mutations and an ITAM3 variant comprising one or more (e.g., two) loss-of-function mutations. In certain embodiments, the intracellular signaling domain of the CAR comprises a modified CD3ζ polypeptide comprising an ITAM1 variant comprising two loss-of-function mutations, a native ITAM2, and an ITAM3 variant comprising two loss-of-function mutations. In certain embodiments, the intracellular signaling domain of the CAR comprises a modified CD3ζ polypeptide comprising an ITAM1 variant consisting of the amino acid sequence set forth in SEQ ID NO: 39, a native ITAM2 consisting of the amino acid sequence set forth in SEQ ID NO: 41 and an ITAM3 variant consisting of the amino acid sequence set forth in SEQ ID NO: 47 (e.g., a construct designated as “X2X”).

In certain embodiments, the intracellular signaling domain of the CAR comprises a modified CD3ζ polypeptide comprising an ITAM1 variant comprising one or more (e.g., two) loss-of-function mutations and an ITAM2 variant comprising one or more (e.g., two) loss-of-function mutations. In certain embodiments, the intracellular signaling domain of the CAR comprises a modified CD3ζ polypeptide comprising an ITAM1 variant comprising or consisting of two loss-of-function mutations, an ITAM2 variant comprising or consisting of two loss-of-function mutations, and a native ITAM3. In certain embodiments, the intracellular signaling domain of the CAR comprises a modified CD3ζ polypeptide comprising an ITAM1 variant consisting of the amino acid sequence set forth in SEQ ID NO: 39, an ITAM2 variant consisting of the amino acid sequence set forth in SEQ ID NO: 42, and a native ITAM3 consisting of the amino acid sequence set forth in SEQ ID NO: 45 (e.g., a construct designated as “XX3”).

In certain embodiments, the intracellular signaling domain of the CAR comprises a modified CD3ζ polypeptide comprising an ITAM1 variant comprising one or more (e.g., two) loss-of-function mutations. In certain embodiments, the intracellular signaling domain of the CAR comprises a modified CD3ζ polypeptide comprising an ITAM1 variant comprising or consisting of two loss-of-function mutations, a native ITAM2, and a native ITAM3. In certain embodiments, the intracellular signaling domain of the CAR comprises a modified CD3ζ polypeptide comprising an ITAM1 variant consisting of the amino acid sequence set forth in SEQ ID NO: 39, a native ITAM2 consisting of the amino acid sequence set forth in SEQ ID NO: 41, and a native ITAM3 consisting of the amino acid sequence set forth in SEQ ID NO: 45 (e.g., a construct designated as “X23”).

In certain embodiments, the intracellular signaling domain of the CAR comprises a modified CD3ζ polypeptide comprising a native ITAM1, a native ITAM2, and an ITAM3 variant comprising one or more (e.g., two) loss-of-function mutations. In certain embodiments, the intracellular signaling domain of the CAR comprises a modified CD3ζ polypeptide comprising a native ITAM1, a native ITAM2, and an ITAM1 variant comprising or consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 37, a native ITAM2 consisting of the amino acid sequence set forth in SEQ ID NO: 41 and an ITAM3 variant consisting of the amino acid sequence set forth in SEQ ID NO: 47 (e.g., a construct designated as “12X”).

In certain embodiments, the intracellular signaling domain of the CAR comprises a modified CD3ζ polypeptide comprising a native ITAM1, an ITAM2 variant comprising one or more (e.g., two) loss-of-function mutations, and a native ITAM3. In certain embodiments, the intracellular signaling domain of the CAR comprises a modified CD3ζ polypeptide comprising a native ITAM1, an ITAM2 variant comprising or consisting of two loss-of-function mutations, and a native ITAM3. In certain embodiments, the intracellular signaling domain of the CAR comprises a modified CD3ζ polypeptide comprising a native ITAM1 consisting of the amino acid sequence set forth in SEQ ID NO: 37, an ITAM2 variant consisting of the amino acid sequence set forth in SEQ ID NO: 42, and a native ITAM3 variant consisting of the amino acid sequence set forth in SEQ ID NO: 45 (e.g., a construct designated as “1X3”).

In certain embodiments, the intracellular signaling domain of the CAR comprises a modified CD3ζ polypeptide comprising a deletion of one or two ITAMs. In certain embodiments, the modified CD3ζ polypeptide comprises or consists of a deletion of ITAM1 and ITAM2, e.g., the modified CD3ζ polypeptide comprises a native ITAM3 or an ITAM3 variant, and does not comprise an ITAM1 or an ITAM2. In certain embodiments, the modified CD3ζ polypeptide comprises a native ITAM3 consisting of the amino acid sequence set forth in SEQ ID NO: 45, and does not comprise an ITAM1 (native or modified), or an ITAM2 (native or modified) (e.g., a construct designated as “D12”).

In certain embodiments, the modified CD3ζ polypeptide comprises or consists of a deletion of ITAM2 and ITAM3, e.g., the modified CD3ζ polypeptide comprises a native ITAM1 or an ITAM1 variant, and does not comprise an ITAM2 or an ITAM3. In certain embodiments, the modified CD3ζ polypeptide comprises a native ITAM1 consisting of the amino acid sequence set forth in SEQ ID NO: 37, and does not comprise an ITAM2 (native or modified), or an ITAM3 (native or modified) (e.g., a construct designated as “D23”).

In certain embodiments, the modified CD3ζ polypeptide comprises or consists of a deletion of ITAM1 and ITAM3, e.g., the modified CD3ζ polypeptide comprises a native ITAM2 or an ITAM2 variant, and does not comprise an ITAM1 or an ITAM3. In certain embodiments, the modified CD3ζ polypeptide comprises a native ITAM2 consisting of the amino acid sequence set forth in SEQ ID NO: 41, and does not comprise an ITAM1 (native or modified), or an ITAM3 (native or modified) (e.g., a construct designated as “D13”).

In certain embodiments, the modified CD3ζ polypeptide comprises or consists of a deletion of ITAM1, e.g., the modified CD3ζ polypeptide comprises a native ITAM2 or an ITAM2 variant, and a native ITAM3 or an ITAM3 variant, and does not comprise an ITAM1 (native or modified).

In certain embodiments, the modified CD3ζ polypeptide comprises or consists of a deletion of ITAM2, e.g., the modified CD3ζ polypeptide comprises a native ITAM1 or an ITAM1 variant, and a native ITAM3 or an ITAM3 variant, and does not comprise an ITAM2 (native or modified).

In certain embodiments, the modified CD3ζ polypeptide comprises or consists of a deletion of ITAM3, e.g., the modified CD3ζ polypeptide comprises a native ITAM1 or an ITAM1 variant, and a native ITAM2 or an ITAM2 variant, and does not comprise an ITAM3 (native or modified).

In certain non-limiting embodiments, the intracellular signaling domain of the CAR further comprises at least a co-stimulatory signaling region. In certain embodiments, the at least one co-stimulatory signaling region comprises an intracellular domain of a co-stimulatory molecule. In certain embodiments, the co-stimulatory molecule is selected from the group consisting of CD28, 4-1BB, OX40, CD27, CD40, ICOS, DAP-10, CD2, and NKGD2.

The co-stimulatory molecule can bind to a co-stimulatory ligand. As one example, a 4-1BB ligand (i.e., 4-1BBL) may bind to 4-1BB for providing an intracellular signal that in combination with a CAR signal induces an effector cell function of the CAR⁺ T cell. CARs comprising an intracellular signaling domain that comprises a co-stimulatory signaling region comprising a 4-1BB polypeptide, an ICOS polypeptide, or a DAP-10 polypeptide are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide (e.g., an intracellular domain of CD28 or a portion thereof). In certain embodiments, the intracellular signaling domain of the CAR comprises a co-stimulatory signaling region that comprises an intracellular domain of human CD28 or a portion thereof. In certain embodiments, the intracellular signaling domain of the CAR comprises a co-stimulatory signaling region that comprises a CD28 polypeptide comprising or consisting of an amino acid sequence of amino acids 180 to 220 of SEQ ID NO: 32. In certain embodiments, the intracellular signaling domain of the CAR comprises a co-stimulatory signaling region that comprises a CD28 polypeptide comprising or consisting of an amino acid sequence of amino acids 180 to 219 of SEQ ID NO: 32.

An exemplary nucleotide sequence encoding amino acids 180 to 220 of SEQ ID NO: 32 is set forth in SEQ ID NO: 49, which is provided below.

[SEQ ID NO: 49]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the intracellular signaling domain of the CAR comprises a de-immunized intracellular domain of CD28 (e.g., human CD28) or a portion thereof. In certain embodiments, the de-immunized intracellular domain of CD28 or a portion thereof comprises or consists of an amino acid sequence that is at least about 80%, at least about 85%, at least about 90%, at least about 95%, at least about 96%, at least about 97%, at least about 98%, or at least about 99%, at least about 100% homologous or identical to SEQ ID NO: 50 or fragments thereof, and/or may optionally comprise up to one or up to two or up to three conservative amino acid substitutions. In certain embodiments, the de-immunized intracellular domain of CD28 comprises or consists of the amino acid sequence set forth in SEQ ID NO: 50. SEQ ID NO: 50 is provided below:

[SEQ ID NO: 50]
RSKRSRLLHSDYMNMTPRRPGPTRKHYQPYAPPRDFAAYRK

In certain embodiments, the intracellular signaling domain of the CAR comprises a co-stimulatory signaling region comprising a 4-1BB polypeptide, e.g., an intracellular domain of 4-1BB (e.g., human or murine 4-1BB) or a portion thereof.

In certain embodiments, the intracellular signaling domain of the CAR comprises a co-stimulatory signaling region that comprises two co-stimulatory molecules, e.g., co-stimulatory signaling regions of CD28 and 4-1BB or co-stimulatory signaling regions of CD28 and OX40.

In certain embodiments, a presently disclosed CAR further comprises an inducible promoter, for expressing nucleic acid sequences in human cells. Promoters for use in expressing CAR genes can be a constitutive promoter, such as ubiquitin C (UbiC) promoter.

In certain embodiments, a presently disclosed cell comprises a transgene encoding a CAR comprising an extracellular antigen-binding domain that binds to CD19 (e.g., human CD19), a transmembrane domain comprising a CD28 polypeptide (e.g., human CD28 polypeptide, e.g., a transmembrane domain of CD28 (e.g., human CD28) of a portion thereof), an intracellular signaling domain comprising a CD3ζ polypeptide (e.g., a human CD3ζ polypeptide, e.g., a native human CD3ζ polypeptide or a modified CD3ζ polypeptide), and a co-stimulatory signaling domain comprising a CD28 polypeptide (e.g., human CD28 polypeptide, e.g., an intracellular domain of CD28 (e.g., human CD28) or a portion thereof).

In certain embodiments, a presently disclosed cell further comprises an exogenous cytokine. Non-limiting examples of cytokines include IL-12, IL-21, IL-15, IL-7, and IL-36, IL2.

In certain embodiments, the antigen-recognizing receptor is a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The Variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/η. When a TCR complex engages with its antigen and MHC (peptide/MHC), the T cell expressing the TCR complex is activated.

In certain embodiments, the antigen-recognizing receptor is an endogenous TCR. In certain embodiments, the antigen-recognizing receptor is naturally occurring TCR.

In certain embodiments, the antigen-recognizing receptor is an exogenous TCR. In certain embodiments, the antigen-recognizing receptor is a recombinant TCR. In certain embodiments, the antigen-recognizing receptor is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the antigen-recognizing receptor is a TCR like fusion molecule. Non-limiting examples of TCR fusion molecules include HLA-Independent TCR-based Chimeric Antigen Receptor (also known as “HIT-CAR”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR like fusion molecule is a recombinant T cell receptor (TCR). In certain embodiments, the TCR like fusion molecule comprises an antigen binding chain that comprises an extracellular antigen-binding domain and a constant domain, wherein the TCR like fusion molecule binds to the first antigen in an HLA-independent manner. Thus, in certain embodiments, the TCR like fusion molecule is an HLA-independent (or non-HLA restricted) TCR (referred to as “HIT-CAR” or “HIT”). In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the constant domain is capable of forming a homodimer or a heterodimer with another constant domain. In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR like fusion molecule is capable of integrating with a CD3 complex and providing HLA-independent antigen recognition. In certain embodiments, the TCR like fusion molecule replaces an endogenous TCR in a CD3/TCR complex. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a ligand for a cell-surface receptor, a receptor for a cell surface ligand, an antigen binding portion of an antibody or a fragment thereof or an antigen binding portion of a TCR. In certain embodiments, the extracellular antigen-binding domain of the TCR like fusion molecule comprises an antigen binding portion of an antibody. In certain embodiments, the extracellular antigen-binding domain of the TCR like fusion molecule comprises an scFv. In certain embodiments, the extracellular antigen-binding domain of the TCR like fusion molecule comprises one or two immunoglobulin variable region(s). In certain embodiments, the extracellular antigen-binding domain of the TCR like fusion molecule comprises a heavy chain variable region (V_{H}) of an antibody. In certain embodiments, the extracellular antigen-binding domain of the TCR like fusion molecule comprises a light chain variable region (V_{L}) of an antibody. In certain embodiments, the extracellular antigen-binding domain of the TCR like fusion molecule is capable of dimerizing with another extracellular antigen-binding domain. In certain embodiments, the extracellular antigen-binding domain of the TCR like fusion molecule comprises a V_{H} of an antibody, wherein the V_{H} is capable of dimerizing with another extracellular antigen-binding domain comprising a V_{L} of the antibody and form a fragment variable (Fv), e.g., an scFv. In certain embodiments, the extracellular antigen-binding domain of the TCR like fusion molecule comprises a V_{L} of an antibody, wherein the V_{L} is capable of dimerizing with another extracellular antigen-binding domain comprising a V_{H} of the antibody and form a fragment variable (Fv), e.g., an scFv.

In certain embodiments, the extracellular antigen-binding domain of the TCR like fusion molecule comprises an antigen binding portion of a TCR. In certain embodiments, the extracellular antigen-binding domain of the TCR like fusion molecule comprises an antigen binding portion of an antibody or a fragment thereof. In certain embodiments, the extracellular antigen-binding domain of the TCR like fusion molecule comprises a heavy chain variable region (V_{H}) and/or a light chain variable region (V_{L}) of an antibody. In certain embodiments, the extracellular antigen-binding domain comprises a single-chain variable fragment (scFv). In certain embodiments, the extracellular antigen-binding domain comprises a heavy chain-only antibodies (VHH). In certain embodiments, the extracellular antigen-binding domain comprises a Fab, which is optionally crosslinked. In certain embodiments, the extracellular antigen-binding domain comprises a F(ab)2. In certain embodiments, any of the foregoing molecules can be comprised in a fusion protein with a heterologous sequence to form the extracellular antigen-binding domain.

In certain embodiments, the extracellular antigen-binding domain of the TCR like fusion molecule comprises a heavy chain variable region (V_{H}) and/or a light chain variable region (V_{L}) of an antibody, wherein the V_{H} or the V_{L} is capable of dimerizing with another extracellular antigen-binding domain comprising a V_{L} or a V_{H} (e.g., forming a fragment variable (Fv)). In certain embodiments, the Fv is a human Fv. In certain embodiments, the Fv is a humanized Fv. In certain embodiments, the Fv is a murine Fv. In certain embodiments, the Fv is identified by screening a Fv phage library with an antigen-Fc fusion protein. Additional extracellular antigen-binding domains targeting an interested antigen can be obtained by sequencing an existing scFv or a Fab region of an existing antibody targeting the same antigen.

In certain embodiments, the antigen binding chain of the TCR like fusion molecule further comprises a constant domain. In certain embodiments, the constant domain comprises a hinge/spacer region and a transmembrane domain. In certain embodiments, the constant domain is capable of forming a homodimer or a heterodimer with another constant domain. In certain embodiments, the constant domain dimerizes through one or more disulfide-links. In certain embodiments, the antigen binding chain of the TCR like fusion molecule is capable of forming a trimer or oligomer with one or more identical or different constant domains.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the TCR like fusion molecule comprises a hinge/spacer region that links the extracellular antigen-binding domain to the constant domain. The hinge/spacer region can be flexible enough to allow the antigen binding domain to orient in different directions to facilitate antigen recognition. In certain embodiments, the hinge/spacer region can be the hinge region from IgG1, or the CH₂CH₃ region of immunoglobulin and portions of CD3, a portion of a CD28 polypeptide, a portion of a CD8 polypeptide, a variation of any of the foregoing which is at least about 80%, at least about 85%, at least about 90%, or at least about 95% homologous or identical thereto, or a synthetic spacer sequence. In certain non-limiting embodiments, the hinge/spacer region of the CAR can comprise a native or modified hinge region of a CD3ζ polypeptide, a CD40 polypeptide, a 4-1BB polypeptide, an OX40 polypeptide, a CD166 polypeptide, a CD166 polypeptide, a CD8a polypeptide, a CD8b polypeptide, an ICOS polypeptide, an ICAM-1 polypeptide, a CTLA-4 polypeptide, a synthetic polypeptide (not based on a protein associated with the immune response), or a combination thereof.

In certain embodiments, the TCR like fusion molecule comprises an antigen binding chain, which does not comprise an intracellular domain. In certain embodiments, the antigen binding chain is capable of associating with a CD3ζ polypeptide. In certain embodiments, the antigen binding chain comprises a constant domain, which is capable of associating with a CD3ζ polypeptide. In certain embodiments, the CD3ζ polypeptide is endogenous. In certain embodiments, the CD3ζ polypeptide is exogenous. In certain embodiments, binding of the antigen binding chain to an antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the TCR like fusion molecule exhibits a greater antigen sensitivity than a CAR targeting the same antigen. In certain embodiments, the TCR like fusion molecule is capable of inducing an immune response when binding to an antigen that has a low density on the surface of a tumor cell. In certain embodiments, cells comprising the TCR like fusion molecule can be used to treat a subject having tumor cells with a low expression level of a surface antigen, e.g., from a relapse of a disease, wherein the subject received treatment which leads to residual tumor cells. In certain embodiments, the tumor cells have a low density of a target molecule on the surface of the tumor cells. In certain embodiments, a target molecule having a low density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low density on the cell surface has a density of less than about 2,000 molecules per cell. In certain embodiments, a target molecule having a low density on the cell surface has a density of less than about 1,500 molecules per cell. In certain embodiments, a target molecule having a low density on the cell surface has a density of less than about 1,000 molecules per cell. In certain embodiments, a target molecule having a low density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

Various TCR like fusion molecules are disclosed in International Patent Application Publication No. WO2019/133969, which is incorporated by reference hereby in its entirety.

In certain embodiments, the antigen-recognizing receptor is a chimeric co-stimulatory receptor (CCR). In certain embodiments, a CCR is a chimeric receptor that binds to an antigen and provides co-stimulatory signals, but does not provide a T-cell activation signal. CCR is described in Krause, et al., J. Exp. Med. (1998); 188(4):619-626, and US20020018783, the contents of which are incorporated by reference in their entireties. CCRs mimic co-stimulatory signals, but unlike, CARs, do not provide a T-cell activation signal, e.g., CCRs lack a CD3ζ polypeptide.

### 5.5. Exogenous Compositions

In certain embodiments, the exogenous composition disclosed herein comprises a transgene disclosed herein (e.g., disclosed in Section 5.4). In certain embodiments, the exogenous composition disclosed herein further comprises a promoter, a transcription factor, and/or an inducible element. In certain embodiments, the transgene is operably linked to the promoter, the transcription factor, and/or the inducible element. In certain embodiments, the promoter is an exogenous promoter. Non-limiting examples of exogenous promoters include an elongation factor (EF)-1 promoter, a CMV promoter, a SV40 promoter, a PGK promoter, and a metallothionein promoter. In certain embodiments, the promoter is an inducible promoter. Non-limiting examples of inducible promoters include a NFAT transcriptional response element (TRE) promoter, a CD69 promoter, a CD25 promoter, an IL-2 promoter, a 4-1 BB promoter, a hypoxia responsive promoter, and a beta globin promoter. In certain embodiments, the promoter is an endogenous promoter or a variant thereof.

Non-limiting examples of transcription factors include Nuclear factor of activated T-cells (NFAT), Hypoxia inducible factor 1α (HIF1α), and NF-κB (nuclear factor kappa-light-chain-enhancer of activated B cells).

Non-limiting examples of inducible elements include NFAT response elements, Hypoxia inducible factor 1α, Hypoxia responsive element, and NF-κB responsive elements.

In certain embodiments, the promoter is a constitutive promoter. Non-limiting examples of constitutive promoters include an elongation factor (EF) 1 promoter, a cytomegalovirus immediate-early promoter (CMV) promoter, a simian virus 40 early promoter (SV40) promoter, a phosphoglycerate kinase (PGK) promoter, a CAG promoter, and a metallothionein promoter.

In certain embodiments, the exogenous composition further comprises a polyadenylation signal.

In certain embodiments, the exogenous composition further comprises at least one insulator. Insulators are naturally occurring DNA elements that help from the functional boundaries between adjacent chromatin domains. In certain embodiments, the placement of insulators in the vectors described herein offer various potential benefits including, but not limited to, shielding of the vector from positional effect variegation of expression by flanking chromosomes (i.e., barrier activity, which may decrease position effects and vector silencing.)

In certain embodiments, the at least one insulator is selected from the insulators disclosed in Liu et al., Nature Biotechnology (2015), 33(2), p. 198, which is incorporated by reference in its entirety. In certain embodiments, the at least one insulator is selected from the insulators disclosed in International Patent Publication No. WO2015/138852, which is incorporated by reference in its entirety.

In certain embodiments, the at least one insulator can block the encroachment of silencing heterochromatin into adjoining regions of open chromatin that are transcriptionally permissive. In certain embodiments, the at least one insulator comprises a CTCF binding site having the nucleotide sequence set forth in SEQ ID NO: 76. In certain embodiments, the at least one insulator comprises or consists of the nucleotide sequence set forth in SEQ ID NO: 77 or a nucleotide sequence which is at least about 80%, at least about 85%, at least about 90%, at least about 95%, or at least about 99% identical to SEQ ID NO: 77. SEQ ID Nos: 76 and 77 are provided below. In certain embodiments, the at least one insulator comprises the C1 insulator disclosed in International Patent Publication No. WO2015/138852.

[SEQ ID NO: 76]
TCAGTAGAGGGCGC
[SEQ ID NO: 77]
GTCTGAATGGTGGCCGTAGTTTGCAGAGCCCTGGTTTCTTCTTGCCTCT
CAGCTTCCAACTTCCCCGTGAGTGCCTGCTCCTTGATGGACTGGACTCT
AAGCCCTTCTTTGCAGCAAGCACGATATCAAGCTTTGTCAGTAGAGGGC
GCCGGAGGGACACTGTGGAGGAAGGGGCCTTTTCATGGTCCACAGAGCT
CTGTTGTGCAATTTCTTGTTCCTGTTGCATCTTCTCTTAGGGTATGAAC
GCGGGGGGACATCCTCTGGGGCTTTTCCTCAGCTGTGCACCCAGAATGC
ATGGTCCCTCGACCACCTCATAGCCCATCCT

In certain embodiments, the at least one insulator comprises a CTCF binding site having the nucleotide sequence set forth in SEQ ID NO: 78. In certain embodiments, the at least one insulator comprises or consists of the nucleotide sequence set forth in SEQ ID NO: 79 or a nucleotide sequence which is at least about 80%, at least about 85%, at least about 90%, at least about 95%, or at least about 99% identical to SEQ ID NO: 79. SEQ ID Nos: 78 and 79 are provided below. In certain embodiments, the at least one insulator comprises the A1 insulator disclosed in International Patent Publication No. WO2015/138852.

[SEQ ID NO: 78]
CACCAGGTGGCGCT
[SEQ ID NO: 79]
ctggttctac tcattacatt ccaatcgtgg catatcctct
aaactttctt ttcccttcat aaatcctctt tctttttttt
ccccctcaca gttttcctga acaggttgac tattaattgt
gtctgcttga tgtggacacc aggtggcgct ggacatcaga
tttggagagg cagttgtcta gggaaccggg ctctgtgcca
gcgcaggagg caggctggct ctcctattcc agggatgctc
atccaggaag gaaaggttgc atgctggaca cactaacctt
gaagaattct tctgtctctc tcgtcattta gaaaggaagg

In certain embodiments, the at least one insulator comprises an insulator disclosed in International Patent Publication No. WO2016/037138. In certain embodiments, the at least one insulator comprises or consists of a nucleotide sequence which is at least about 80%, at least about 85%, at least about 90%, at least about 95%, or at least about 99% identical to SEQ ID NO: 80, SEQ ID NO: 81, SEQ ID NO: 82, or SEQ ID NO: 83. In certain embodiments, the at least one insulator comprises or consists of the nucleotide sequence set forth in SEQ ID NO: 80, SEQ ID NO: 81, SEQ ID NO: 82, or SEQ ID NO: 83.

[SEQ ID NO: 80]
TCCTTCCTTTCTAAATGACGAGAGAGACAGAAGAATTCTTCAAGGTTAGTGTGTCCAGCATGCAACCTTTCCTT
CCTGGATGAGCATCCCTGGAGTAGGAGAGCCAGCCTGCCTCCTGCGCTGGCACAGAGCCCGGTTCCCTAGACAA
CTGCCTCTCCAAATCTGATGTCCAGCGCCACCTGGTGTCCACATCAAGCAGACACAATTAATAGTCAACCTGTT
CAGGAAAACTGTGAGGGGGAAAAAAAAGAAAGAGGATTTATGAAGGGAAAAGAAAGTTTAGAGGATATGCCACG
ATTGGCTAG
[SEQ ID NO: 81]
CCAATC GTGGCATATC CTCTAAACTT TCTTTTCCCT TCATAAATCC TCTTTCTTTT TTTTCCCCCT
CACAGTTTTC CTGAACAGGT TGACTATTAA TTGTGTCTGC TTGATGTGGA CACCAGGTGG
CGCTGGACAT CAGATTTGGA GAGGCAGTTG TCTAGGGAAC CGGGCTCTGT GCCAGCGCAG
GAGGCAGGCT GGCTCTCCTA TTCCAGGGAT GCTCATCCAG GAAGGAAAGG TTGCATGCTG
GACACACTAA CCTTGAAGAA TTCTTCTGTC TCTCTCGTCA TTTAGAAAGG AAGGA
[SEQ ID NO: 82]
CTAGCCAATCGTGGCATATCCTCTAAACTTTCTTTTCCCTTCATAAATCCTCTTTCTTTTTTTTCCCCCTCACA
GTTTTCCTGAACAGGTTGACTATTAATTGTGTCTGCTTGATGTGGACACCAGGTGGCGCTGGACATCAGATTTG
GAGAGGCAGTTGTCTAGGGAACCGGGCTCTGTGCCAGCGCAGGAGGCAGGCTGGCTCTCCTACTCCAGGGATGC
TCATCCAGGAAGGAAAGGTTGCATGCTGGACACACTAACCTTGAAGAATTCTTCTGTCTCTCTCGTCATTTAGA
AAGGAAGGA
[SEQ ID NO: 83]
CCAATCGTGGCATATCCTCTAAACTTTCTTTTCCCTTCATAAATCCTCTTTCTTTTTTTTCCCCCTCACAGTTT
TCCTGAACAGGTTGACTATTAATTGTGTCTGCTTGATGTGGACACCAGGTGGCGCTGGACATCAGATTTGGAGA
GGCAGTTGTCTAGGGAACCGGGCTCTGTGCCAGCGCAGGAGGCAGGCTGGCTCTCCTATTCCAGGGATGCTCAT
CCAGGAAGGAAAGGTTGCATGCTGGACACACTAACCTTGAAGAATTCTTCTGTCTCTCTCGTCATTTAGAAAGG
AAGG

In certain embodiments, the exogenous composition further comprises two insulators. In certain embodiments, each of the insulators comprises or consists of the nucleic acid sequence set forth in SEQ ID NO: 77.

### 5.6. Vectors and Gene Editing Systems

In certain embodiments, a vector is employed for introduction of the exogenous compositions disclosed herein into the cells, e.g., to genetically modify the cells and to generate the cells disclosed herein (e.g., disclosed in Section 5.3). In certain embodiments, the vector is a viral vector. In certain embodiments, the viral vector is a retroviral vector. In certain embodiments, the retroviral vector is a gamma-retroviral vector or a lentiviral vector. Any suitable serotype of viral vectors can be used with the presently disclosed subject matter. Combinations of retroviral vector and an appropriate packaging line are also suitable, where the capsid proteins are functional for infecting cells (e.g., human cells). Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy (1990); 15-14; Friedman, Science (1989); 244:1275-1281; Eglitis et al., BioTechniques 6:608-614, 1988; Tolstoshev et al., Current Opinion in Biotechnology (1990); 1:55-61; Sharp, The Lancet (1991); 337:1277-1278; Cornetta et al., Nucleic Acid Research and Molecular Biology (1987)36:311-322; Anderson, Science (1984); 226:401-409; Moen, Blood Cells (1991); 17:407-416; Miller et al., Biotechnology (1989); 7:980-990; Le Gal La Salle et al., Science (1993); 259:988-990; and Johnson, Chest (1995); 107:775-83S). Retroviral vectors are particularly well developed and have been used in clinical settings (Rosenberg et al., N. Engl. J. Med (1990); 323:370; Anderson et al., U.S. Pat. No. 5,399,346).

Retroviral vectors can be derived from three genera of the retroviridae: the γ-retroviruses (also known as C-type murine retroviruses or oncoretroviruses), the lentiviruses, and the spumaviruses (also known as foamy viruses). Several reviews detailing molecular approaches for the generation of replication-defective retroviral particles are available (Cornetta et al. (2005); Cockrell & Kafri (2007)). The vector itself, which encodes the therapeutic transgene or cDNA, retains the minimal viral sequences needed to enable packaging in viral particles in a packaging cell line, reverse transcription, and integration. The packaging cell expresses the necessary structural proteins and enzymes that are required to assemble an infectious recombinant particle that contains the vector sequence and the machinery needed for its reverse transcription and integration in the transduced cell.

γ-retroviral, lentiviral and spumaviral vectors have been used successfully for the transduction of cytokine activated HSCs.

The exogenous compositions disclosed herein can be integrated within a presently disclosed GSH by any targeted gene delivery strategies known in the art. Targeted gene delivery strategies, utilizing a gene editing system, such as a non-naturally occurring or engineered nuclease (including, but not limited to, Zinc-finger nuclease (ZNFs), meganuclease, transcription activator-like effector nuclease (TALEN)), or a CRISPR-Cas system, can reduce or even eliminate the concern of insertional oncogenesis that is inherent to the use of retroviral vectors.

Clustered regularly-interspaced short palindromic repeats (CRISPR) system is a genome editing tool discovered in prokaryotic cells. When utilized for genome editing, the system comprises Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR, ZFNs can be used to insert the CAR into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing systems can vary depending on the need. In certain embodiments, the components of a selected genome editing system are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic nanoparticles, and cell-penetrating peptides).

The gene editing system disclosed herein can be delivered into the host cell using a viral vector, e.g., retroviral vectors such as gamma-retroviral vectors, and lentiviral vectors. Any suitable serotype of viral vectors can be used with the presently disclosed subject matter. Combinations of viral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to deliver the gene editing system to the host cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In certain embodiments, the viral vectors are oncolytic viral vectors that target cancer cell and deliver the gene editing system to the cancer cells. Non-limiting examples of oncolytic viral vectors are disclosed in Lundstrom et al., Biologics. 2018; 12: 43-60, and the content of which is incorporated by reference herein in its entirety. In certain embodiments, the oncolytic viral vectors are selected from adenoviruses, HSV, alphaviruses, rhabdoviruses, Newcastle disease virus (NDV), vaccinia viruses (VVs), and combinations thereof.

Non-viral approaches can also be employed for delivering the gene editing system to the host cell. For example, a nucleic acid molecule can be introduced into the host cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, non-viral approaches include nanotechnology-based approaches, which use non-viral vectors. The non-viral vectors can be made of a variety of materials, including inorganic nanoparticles, carbon nanotubes, liposomes, protein and peptide-based nanoparticles, as well as nanoscale polymeric materials. Riley et al., Nanomaterials (Basel). 2017 May; 7(5): 94 reviews nanotechnology-based methods for delivery of a nucleic acid molecule to a subject, the content of which is incorporated as reference in its entirety.

Transgene to be delivered into the cell using the gene editing system can be ssDNA or dsDNA, depending on the delivery methods.

In certain embodiments, the exogenous composition is integrated by a gene editing system. In certain embodiments, the gene editing system is selected from a CRISPR-Cas system, a zinc-finger nuclease (ZFN), a meganuclease, and a transcription activator-like effector nuclease (TALEN).

In certain embodiments, the gene editing system is a CRISPR-Cas system.

In certain embodiments, the exogenous composition that comprises two insulators, e.g., each of the two insulators comprises or consists of the nucleotide sequence set forth in SEQ ID NO:110. One of the two insulators is positioned at the 3′ end of the exogenous composition, and the other insulator is positioned at the 5′ end of the exogenous composition.

The presently disclosed gene editing systems (e.g., a CRISPR-Cas system) allow for targeted delivery of the exogenous composition to the cell (e.g., a T cell). In certain embodiments, a presently disclosed CRISPR-Cas system binds within a GSH disclosed herein. A CRISPR-Cas system can generate a double or a single strand break within the GSH.

The presently disclosed subject matter also provides polynucleotides encoding the above-described vectors, polynucleotides encoding the above-described gene editing system (e.g., a CRISPR-Cas system), and vectors comprising the polynucleotides encoding the above-described CRISPR-Cas system.

The gene editing systems (e.g., CRISPR-Cas system) and polynucleotides encoding the gene editing systems (e.g., a CRISPR-Cas system) can be delivered in vivo or ex vivo by any suitable means. For example, the CRISPR-Cas system described herein can be delivered to a cell (e.g., a T cell, a stem cell (e.g., a pluripotent stem cell, e.g., an embryonic stem cell or an induced pluripotent stem cell) by a vector comprising polynucleotides encoding the gene editing systems (e.g., a CRISPR-Cas system). Any vectors can be used including, but not limited to, plasmid vectors, retroviral vectors (e.g., γ-retroviral vectors, lentiviral vectors and foamy viral vectors), adenovirus vectors, poxvirus vectors; herpes virus vectors and adeno-associated virus vectors, etc. In certain embodiments, the vector comprising a polynucleotide encoding a gene editing system disclosed herein (e.g., a CRISPR-Cas system) is a lentiviral vector. In certain embodiments, the lentiviral vector is a non-integrating lentiviral vector. Examples of non-integrating lentiviral vector are described in Ory et al. (1996) Proc. Natl. A cad. Sci. USA 93:11382-11388; Dull et al., (1998) J. Viral. 72:8463-8471; Zuffery et al. (1998) J. Viral. 72:9873-9880; Follenzi et al., (2000) Nature Genetics 25:217-222; U.S. Patent Publication No 2009/054985.

Additionally, non-viral approaches can also be employed for introducing the exogenous composition into the cells. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases. Transient expression may be obtained by RNA electroporation.

The composition or nucleic acid composition disclosed herein can be placed anywhere in a genome. In certain embodiments, the composition or nucleic acid composition is placed in a locus within the genome of a T cell, including, but not limited to, a TRAC locus, a TRBC locus, a TRDC locus, and/or a TRGC locus. In certain embodiments, the placement of the composition or nucleic acid composition disrupts the expression of an endogenous T cell receptor.

### 5.7. Methods of Producing Cells

The present disclosure further provides methods for producing cells disclosed herein (e.g., disclosed in Section 5.3). In certain embodiments, the method comprises integrating the exogenous composition within a locus of the genome of the cell. In certain embodiments, the locus comprises a GSH disclosed herein (e.g., disclosed in Section 5.2).

### 5.8. Administration

The presently disclosed cells and compositions comprising thereof can be provided systemically or directly to a subject, e.g., for treating any diseases targeted by the transgene comprised by the cells.

In certain embodiments, the presently disclosed cells and compositions comprising thereof can be provided systemically or directly to a subject for treating inducing and/or enhancing an immune response to an antigen and/or treating and/or preventing a tumor or a neoplasm, pathogen infection, or infectious disease. In certain embodiments, the presently disclosed cells and compositions comprising thereof are directly injected into an organ of interest (e.g., an organ affected by a tumor or a neoplasm). Alternatively, the presently disclosed cells and compositions comprising thereof are provided indirectly to the organ of interest, for example, by administration into the circulatory system (e.g., the tumor vasculature). Expansion and differentiation agents can be provided prior to, during or after administration of the cells, compositions, or exogenous compositions to increase production of the cells (e.g., T cells (e.g., CTL cells) or NK cells) in vitro or in vivo.

The presently disclosed cells and compositions comprising thereof can be administered in any suitable routes, including but not limited to, intravenous, subcutaneous, intranodal, intratumoral, intrathecal, intrapleural, intraperitoneal. Usually, at least about 1×10⁵ cells will be administered, eventually reaching about 1×10¹⁰ or more. The presently disclosed cells can comprise a purified population of cells. Those skilled in the art can readily determine the percentage of the presently disclosed cells in a population using various well-known methods, such as fluorescence activated cell sorting (FACS). Suitable ranges of purity in populations comprising the presently disclosed cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like. The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 5.9. Formulations

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like.

The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells or their progenitors.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In certain embodiments, between about 10⁴ and about 10¹⁰, between about 10⁵ and about 10⁹, or between about 10⁶ and about 10⁸ of the presently disclosed cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD50 in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### 5.10. Methods of Treatment

The presently disclosed subject matter provides methods for inducing and/or increasing an immune response in a subject in need thereof. The presently disclosed cells, compositions, and exogenous compositions can be used in a therapy or medicament. The presently disclosed cells and compositions comprising thereof can be used for treating and/or preventing a tumor or a neoplasm in a subject. The presently disclosed cells and compositions comprising thereof can be used for prolonging the survival of a subject suffering from a tumor or a neoplasm. The presently disclosed cells and compositions comprising thereof can also be used for treating and/or preventing a pathogen infection or other infectious disease in a subject, such as an immunocompromised human subject. Such methods comprise administering the presently disclosed cells in an amount effective, a presently disclosed composition (e.g., pharmaceutical composition) to achieve the desired effect, be it palliation of an existing condition or prevention of recurrence. For treatment, the amount administered is an amount effective in producing the desired effect. An effective amount can be provided in one or a series of administrations. An effective amount can be provided in a bolus or by continuous perfusion.

An “effective amount” (or, “therapeutically effective amount”) is an amount sufficient to effect a beneficial or desired clinical result upon treatment. An effective amount can be administered to a subject in one or more doses. In terms of treatment, an effective amount is an amount that is sufficient to palliate, ameliorate, stabilize, reverse or slow the progression of the disease, or otherwise reduce the pathological consequences of the disease. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the cells administered.

For adoptive immunotherapy using antigen-specific T cells, cell doses in the range of about 10⁶-10¹⁰ (e.g., about 10⁹) are typically infused. Upon administration of the presently disclosed cells into the host and subsequent differentiation, T cells are induced that are specifically directed against the specific antigen.

The presently disclosed subject matter provides methods for treating and/or preventing a neoplasm or a tumor in a subject. The method can comprise administering an effective amount of the presently disclosed cells or compositions comprising thereof to a subject having a neoplasm.

Non-limiting examples of neoplasms or tumors include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is selected from blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells and compositions comprising thereof can be used for treating and/or preventing blood cancers (e.g., leukemias, lymphomas, and myelomas) or ovarian cancer, which are not amenable to conventional therapeutic interventions. In certain embodiments, the presently disclosed cells and compositions comprising thereof can be used for treating and/or preventing a solid tumor.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Suitable human subjects for therapy typically comprise two treatment groups that can be distinguished by clinical criteria. Subjects with “advanced disease” or “high tumor burden” are those who bear a clinically measurable tumor. A clinically measurable tumor is one that can be detected on the basis of tumor mass (e.g., by palpation, CAT scan, sonogram, mammogram or X-ray; positive biochemical or histopathologic markers on their own are insufficient to identify this population). A pharmaceutical composition is administered to these subjects to elicit an anti-tumor response, with the objective of palliating their condition. Ideally, reduction in tumor mass occurs as a result, but any clinical improvement constitutes a benefit. Clinical improvement comprises decreased risk or rate of progression or reduction in pathological consequences of the tumor.

A second group of suitable subjects is known in the art as the “adjuvant group.” These are individuals who have had a history of neoplasm, but have been responsive to another mode of therapy. The prior therapy can have included, but is not restricted to, surgical resection, radiotherapy, and traditional chemotherapy. As a result, these individuals have no clinically measurable tumor. However, they are suspected of being at risk for progression of the disease, either near the original tumor site, or by metastases. This group can be further subdivided into high-risk and low-risk individuals. The subdivision is made on the basis of features observed before or after the initial treatment. These features are known in the clinical arts, and are suitably defined for each different neoplasm. Features typical of high-risk subgroups are those in which the tumor has invaded neighboring tissues, or who show involvement of lymph nodes.

Another group have a genetic predisposition to neoplasm but have not yet evidenced clinical signs of neoplasm. For instance, women testing positive for a genetic mutation associated with breast cancer, but still of childbearing age, can wish to receive one or more of the cells described herein in treatment prophylactically to prevent the occurrence of neoplasm until it is suitable to perform preventive surgery.

As a consequence of expression of an antigen-recognizing receptor that binds to a tumor antigen, adoptively transferred cells are endowed with augmented and selective cytolytic activity at the tumor site. Furthermore, subsequent to their localization to tumor or viral infection and their proliferation, the T cells turn the tumor or viral infection site into a highly conductive environment for a wide range of immune cells involved in the physiological anti-tumor or antiviral response (tumor infiltrating lymphocytes, NK-, NKT-cells, dendritic cells, and macrophages).

Additionally, the presently disclosed subject matter provides methods for treating and/or preventing a pathogen infection (e.g., viral infection, bacterial infection, fungal infection, parasite infection, or protozoal infection) in a subject, e.g., in an immunocompromised subject. The method can comprise administering an effective amount of the presently disclosed cells or compositions comprising thereof to a subject having a pathogen infection. Exemplary viral infections susceptible to treatment include, but are not limited to, Cytomegalovirus (CMV), Epstein Barr Virus (EBV), Human Immunodeficiency Virus (HIV), and influenza virus infections.

Further modification can be introduced to the presently disclosed cells (e.g., T cells) to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T cell elimination by administering anti-EGFR monoclonal antibody (e.g., cetuximab). EGFRt can be covalently joined to the upstream of the antigen-recognizing receptor of a presently disclosed CAR. The suicide gene can be included within the vector comprising nucleic acids encoding a presently disclosed CAR. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated CAR-expressing T cells. The incorporation of a suicide gene into the a presently disclosed CAR gives an added level of safety with the ability to eliminate the majority of CART cells within a very short time period. A presently disclosed cell (e.g., a T cell) incorporated with a suicide gene can be pre-emptively eliminated at a given timepoint post CAR T cell infusion, or eradicated at the earliest signs of toxicity.

### 5.11. Kits

The presently disclosed subject matter provides kits for inducing and/or enhancing an immune response and/or treating and/or preventing a tumor or a neoplasm or a pathogen infection in a subject. In certain embodiments, the kit comprises an effective amount of presently disclosed cells or compositions comprising thereof. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

If desired, the cells or composition comprising thereof are provided together with instructions for administering the cells, composition, or exogenous composition to a subject having or at risk of developing a neoplasm or a pathogen infection, or immune disorder. The instructions generally include information about the use of the cell, composition or exogenous composition for the treatment and/or prevention of a neoplasm, or a pathogen infection or an immune disorder. In certain embodiments, the instructions include at least one of the following: description of the therapeutic agent; dosage schedule and administration for treatment or prevention of a tumor or a neoplasm, pathogen infection, or immune disorder or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 6. EXAMPLE

The presently disclosed subject matter will be better understood by reference to the following Example, which is provided as exemplary of the presently disclosed subject matter, and not by way of limitation.

### Example 1: Selecting GSHs for Targeted Integration

Genomic Safe Harbors (GSHs) are candidates for targeted integration. Extragenic genomic safe harbors provide safe and stable therapeutic transgene expression levels. Thus, there is a need to find genomic safe harbors for highly efficient and reproducible specific targeting in cells.

Candidate GSHs were determined if they met the following criteria: (a) are located at a distance of more than 50 kb from 5′ end of any gene, (b) are located at a distance of more than 300 kb from any cancer-related genes, (c) are located at a distance of more than 300 kb from any miRNA, (d) are located outside of a gene transcription unit, (e) are located outside of ultra-conserved regions (UCRs), and (f) are located outside of non-coding RNAs. Further criteria for selecting candidate GSHs included efficient cleavability and optimal transgene expression, both of which are governed by DNA accessibility. In addition, chromatin accessibility was used to select candidate GSHs, e.g., whether the locus was proximate to ATAC-seq peaks.

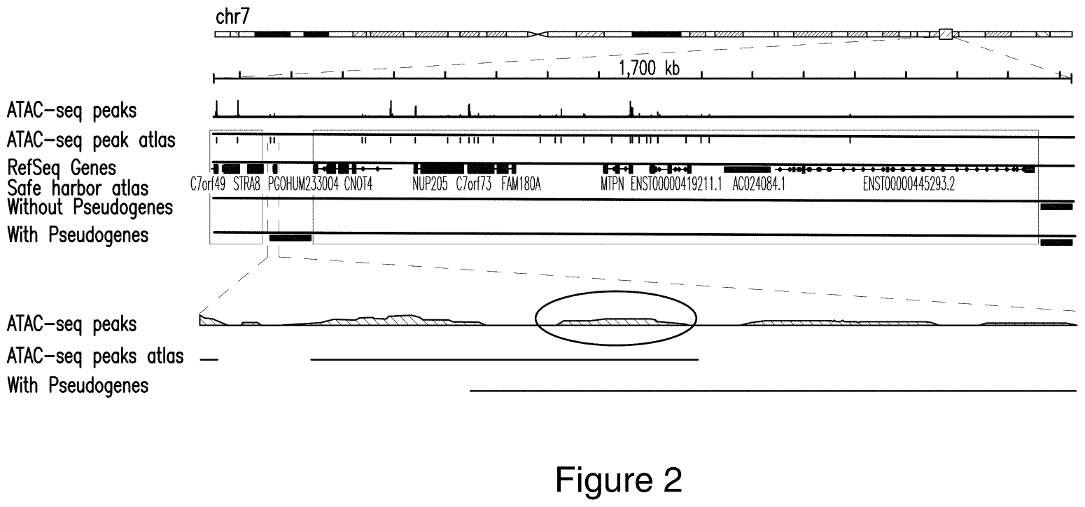

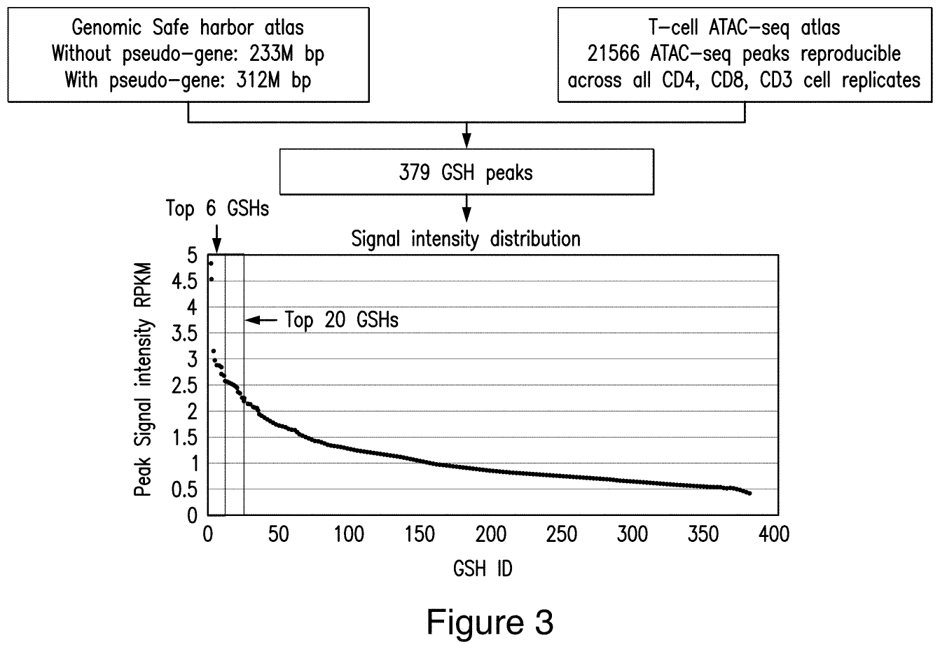

Human T cells were used to identify genomic safe harbors by employing methods disclosed herein. The ATAC-seq atlas was overlaid with GSH atlas with pseudogenes and/or GSH atlas without pseudogenes to identify GSHs (FIG. 2). The human T-cell ATAC-seq atlas comprised 21566 ATAC-seq peaks reproducible across all CD4, CD8, and CD3 cell replicates (FIG. 3). The GSH atlas without pseudogene comprised DNA regions of 233M bp in length, and the GSH atlas with pseudogene comprised DNA regions of 312M bp in length. The GSH atlas (with pseudogene) and the ATAC-seq atlas were overlaid to identify GSHs that are associated with ATAC-seq peaks. ATAC-seq peaks that had a GSH within 5 kb were identified through a custom code, and were considered as GSH peaks. GSH peaks were then scored based on peak signal intensity as replicates per million averaged across all cell types and replicates. The GSH peaks were then ranked by the average peak signal intensity scores. Loci associated with top GSH peaks were selected as top GSHs. The present example selected top 6-20 GSHs for further testing.

The top 20 GSH peaks along with their coordinates in hg19 and hg38 and the corresponding average ATAC-seq peak signal intensities were disclosed in Table 3.

TABLE 3
List of Top 20 GSHs with their coordinates
in human genome assemblies hg19 and hg38

| | | | | Average |
|---|---|---|---|---|
| | | | SEQ ID | ATAC-seq |
| GSH ID | hg19 coordinates for peak | hg38 coordinates for peak | NO | peak signal |
| GSH1 | chr3: 115502796-115503649 | chr3: 115783949-115784802 | 1 | 4.85 |
| GSH2 | chr7: 17273689-17275085 | chr7: 17234065-17235461 | 2 | 4.54 |
| GSH3 | chr5: 87436882-87437724 | chr5: 88141065-88141907 | 3 | 3.16 |
| GSH4 | chr3: 110532783-110533865 | chr3: 110813936-110815018 | 4 | 2.99 |
| GSH5 | chr13: 45491691-45492993 | chr13: 44917556-44918858 | 5 | 2.90 |
| GSH6 | chr7: 149321176-149322360 | chr7: 149624085-149625269 | 6 | 2.90 |
| GSH7 | chr1: 232765494-232766702 | chr1: 232629748-232630956 | 7 | 2.88 |
| GSH8 | chr3: 106147345-106148130 | chr3: 106428498-106429283 | 8 | 2.87 |
| GSH9 | chr7: 12296845-12297778 | chr7: 12257219-12258152 | 9 | 2.72 |
| GSH10 | chr3: 137784804-137785736 | chr3: 138065962-138066894 | 10 | 2.70 |
| GSH11 | chr5: 90458122-90459221 | chr5: 91162305-91163404 | 11 | 2.59 |
| GSH12 | chr6: 141647521-141648419 | chr6: 141326384-141327282 | 12 | 2.58 |
| GSH13 | chrX: 147461745-147463483 | chrX: 148380225-148381963 | 13 | 2.57 |
| GSH14 | chr12: 31004108-31004917 | chr12: 30851174-30851983 | 14 | 2.56 |
| GSH15 | chr3: 105895555-105896259 | chr3: 106176708-106177412 | 15 | 2.56 |
| GSH16 | chr10: 90030671-90031784 | chr10: 88270914-88272027 | 16 | 2.54 |
| GSH17 | chr13: 105791495-105792974 | chr13: 105139144-105140623 | 17 | 2.50 |
| GSH18 | chr18: 36520619-36521489 | chr18: 38940655-38941525 | 18 | 2.50 |
| GSH19 | chr5: 44875678-44876400 | chr5: 44875576-44876298 | 19 | 2.48 |
| GSH20 | chr10: 43047433-43048823 | chr10: 42551985-42553375 | 20 | 2.37 |

Guide RNA (gRNA) Design and Testing

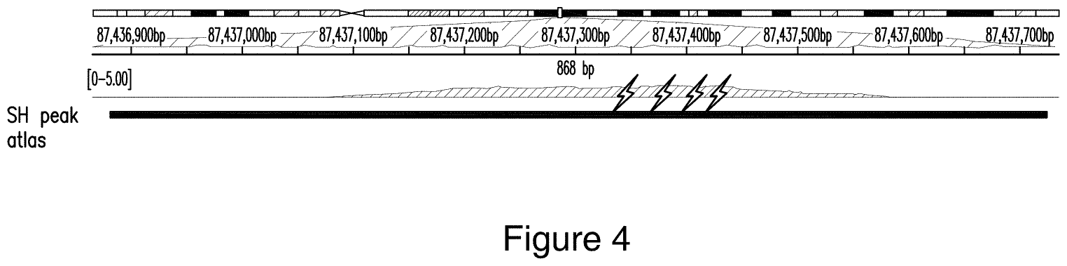

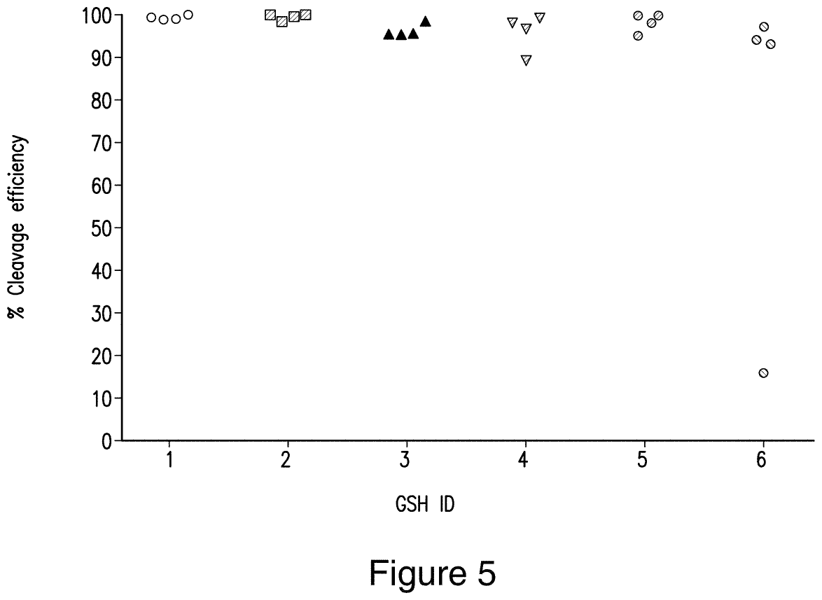

Cleavage efficiencies of the top six GSHs were analyzed by using CRISPR/Cas9 gene editing system. Cleavage efficiencies were determined through analysis of the sequencing data after PCR amplification of the site after transfecting peripheral blood derived human T cells with Cas9 mRNA and gRNAs targeting the selected six GSHs (FIG. 4). Selected top six GSHs showed high cleavage efficiencies (FIG. 5).

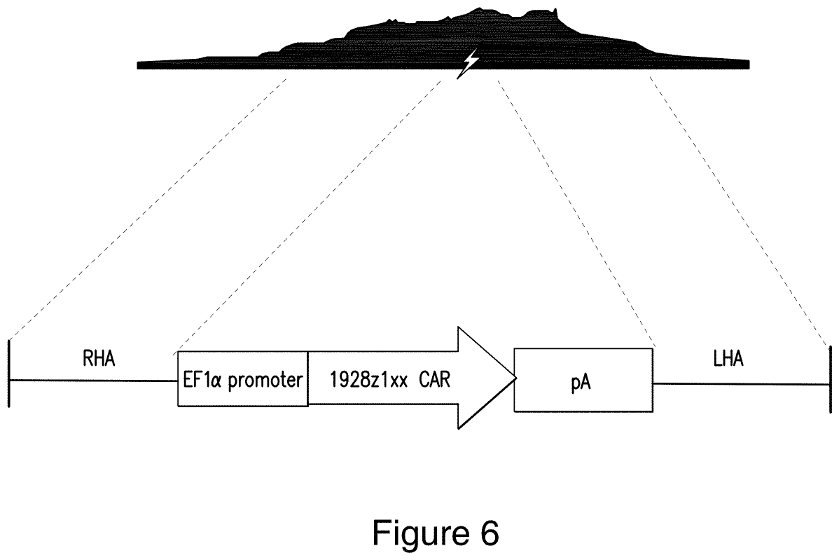

Three GSHs, GSH 1, GSH2, and GSH3, were selected as the sites for transgene integration (FIG. 6). CRISPR/Cas9-targeted CAR gene cassette was integrated into the GSHs. The cassette comprised a 1928z1xxCAR (Feucht et al., Nat. Med. 2019; 25(1):82-88.) driven by an elongation factor 1 alpha (EF1α) promoter, both of which flanked by homology arms for the GSH peaks.

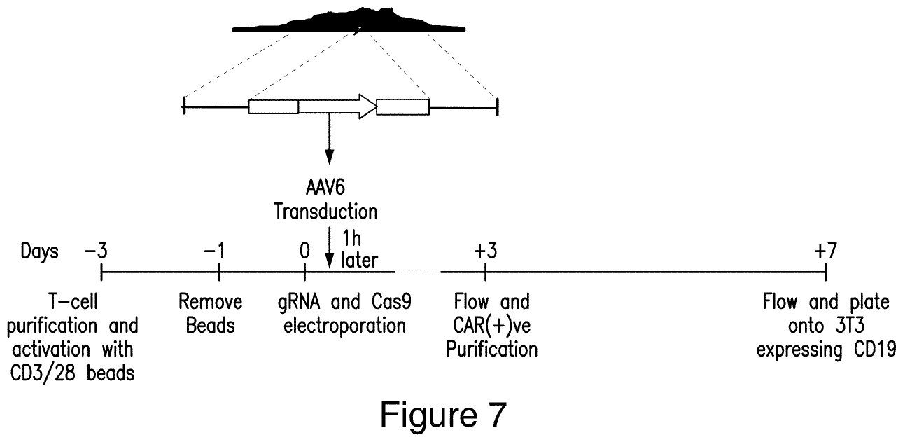

Experimental scheme was depicted in FIG. 7. Briefly, on Day −3, T cells were purified and activated with anti-CD3/CD28 beads. On Day −1, anti-CD3/CD28 beads were removed. On Day 0, gRNA and Cas9 were electroporated into the activated cells. Two hours after the electroporation, AAV6 were also transduced into cells.

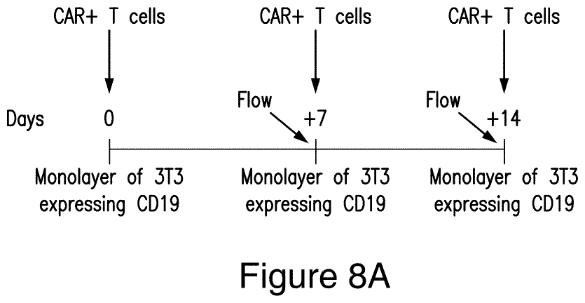

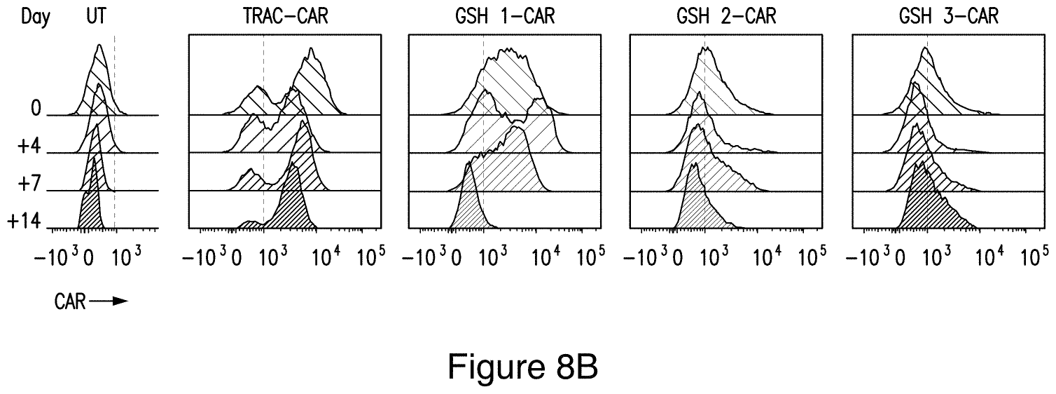

As shown in FIG. 8A weekly antigenic stimulation was applied to CAR+ T cells having CAR expressing cassette integrated at GSHs and TRAC. Untransduced cells (UT) were used as control. CAR+ T cells were plated onto 3T3 cells expressing CD19 at day 7 after transduction and profiled for CAR expression at day 0, 4, 7 and 14 days after initial stimulation. Flow cytometry for CAR expression on day 0, 7 and 14 was performed just before plating onto 3T3 cells. During the first week of stimulation, an increased CAR expression was observed on the surface of GSH-CAR T cells (FIG. 8B).

Materials and Methods

GSH Atlas Generation

All eight properties of candidate GSHs disclosed herein were applied to build a Genomic safe harbor atlas (GSH) atlasbased on the Human GRCh37/hg19 assembly. Gene data for gene transcription units and 5′ end of any gene were obtained from GENCODE release 19 and RefSeq_NM database from NCBI. The 5′ end of a gene was calculated from the transcription start site (TSS). Data for cancer-related genes were obtained by combining oncogene lists from Bushman group allOnco list (v2) (http://www.bushmanlab.org/links/genelists), COSMIC Cancer gene census v78 (https://cancer.sanger.ac.uk/cosmic) and Cancer GeneticsWeb (http://www.cancer-genetics.org/). miRNA data was obtained from hg19 sno/miRNA track in UCSC Genome Browser and also GENCODE release 19 entries for miRNAs. UCRs in the human genome were obtained from Bejerano et al., Science 2004; 304(5675):1321-1325. And the data were downloaded from http://users.soe.ucsc.edu/˜jill/ultra.html. As the genomic coordinates used in the publication were from an older assembly, the coordinates were converted to the hg19 using UCSC lift genome annotations tool. Data for non-coding RNA (ncRNA) list were obtained from NONCODE v5 (www.noncode.org) and GENCODE ncRNA entries. Pseudogene annotation from GENCODE was used to either include or exclude pseudogenes from the gene list to create two atlases—Without pseudogenes and With pseudogenes. The assembly gaps as mentioned on the UCSC Genome Browser for the hg19 genome were excluded.

ATAC-Seq Atlas for Human T Cell Genome

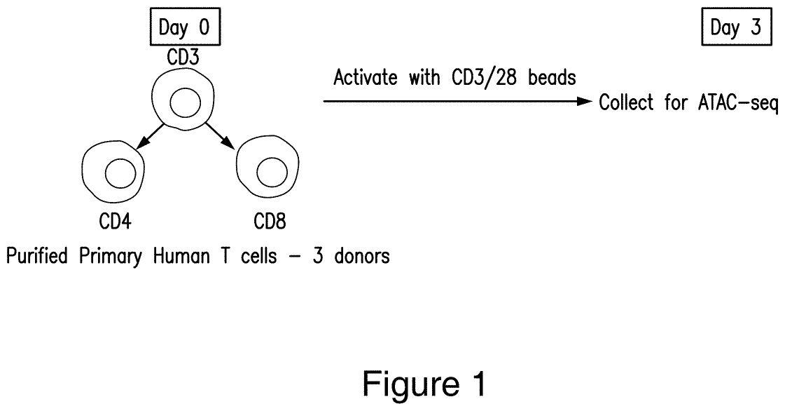

Human T cell genome was profiled for accessibility through ATAC-seq to build ATAC-seq atlas (FIG. 1). Peripheral blood mononuclear cells were obtained by density gradient centrifugation from peripheral blood of three healthy adult human volunteers. Three days after isolation and activation, the T cells were sorted into CD4 and CD8 fractions from two donors by magnetic separation through negative selection using Human CD4-biotin and Human CD8-biotin beads (Miltenyi Biotec) and anti-biotin beads (Miltenyi Biotech). CD3, CD4 and CD8 cells from two donors and only CD3 cells from third donor were collected and 50,000 cells were frozen in freezing medium (10% DMSO in FBS) for ATAC-seq analysis. ATAC-seq was performed by the Memorial Sloan Kettering Cancer Center (MSKCC) IGO core. The method used for ATAC-seq was performed as described in Buenrostro et al., Curr. Protoc. Mol. Biol. 2015; 109:21.29.1-9, with certain modifications. For example, the transposition reaction was performed at 42° C. for 45 mins for a better library preparation. All ATAC libraries were sequenced using paired-end, dual-index sequencing on a HiSeq instrument with 2×50 bp reads for at least 30 million read pairs.

Raw FASTQ reads were trimmed with trimmomatic and aligned to hg19 using Bowtie2. Bam files were filtered based on map quality and PE concordance. Duplicated reads were removed and tn5 specific read shift was performed. To identify peaks, data were aggregated by each cell type, and peak summits were identified using MACS2 and filtered using a custom blacklist. IDR analysis was performed for all replicate pairs. Peaks with global IDR <0.05 were considered as reproducible peaks. 21566 ATAC-seq peaks were found to be reproducible across all cell types and replicates tested.

Guide RNA (gRNA) Design and Testing

Four gRNAs were designed and tested for each of the top 6 GSH peaks. They were designed to fall within the ATAC-seq peak and at the summit of the peak. gRNAs that had the cleavage efficiency scores (Doench scores) of more than 50, and the off-target specificity scores more than 0.2 were chosen. The sequences of the gRNAs used to test the top six GSH peaks were listed (Table 4).

TABLE 4
gRNAs used for testing the top six GSH peaks

| GSH | gRNA | | |
|---|---|---|---|
| ID | name | SEQ ID NO | gRNA sequence |
| GSH1 | 1-1-1 | 51 | CCCGGTGTCACCCGTCACCG |
| | 1-1-2 | 52 | CGCGTCCCAGGCGAGCACGG |
| | 1-2-1 | 53 | TGACGGCAGGGTGAACTCGG |
| | 1-2-2 | 54 | GCAGGGTGAACTCGGCGGCA |
| GSH2 | 2-1-1 | 55 | GCCTACTTCCTACCACACGA |
| | 2-1-2 | 56 | TTTTATAGCCGTCGTGTGGT |
| | 2-2-1 | 57 | GAGTCAAGGTATTGGCAATG |
| | 2-2-2 | 58 | AGATAAGTGAGTCAAGGTAT |
| GSH3 | 3-1 | 59 | CATTCGACTAAAGAAAGCCG |
| | 3-2 | 60 | GCCCCTGAATGGTTTAACGG |
| | 3-3 | 61 | TCGACTAAAGAAAGCCGGGG |
| | 3-4 | 62 | TATGCAGCCTGTCCGCAAGG |
| GSH4 | 4-1 | 63 | GTTAGACAGATACTTCTAAG |
| | 4-2 | 64 | TAGCTAAAACAAGCAGCTTG |
| | 4-3 | 65 | GAATTTGCTCAACCGCCTGT |
| | 4-4 | 66 | GGAATTTGCTCAACCGCCTG |
| GSH5 | 5-1 | 67 | AATTCCATTTCAAAGCACGA |
| | 5-2 | 68 | GAACCAAGGAGCTACCCCTG |
| | 5-3 | 69 | GATGAACGCGATGGAGTGGG |
| | 5-4 | 70 | ACGCCTACGGATGAACGCGA |
| GSH6 | 6-1 | 71 | CTGTATCCTGGGAGCCGGCG |
| | 6-2 | 72 | TAAAGGCGTTGACGCAACTG |
| | 6-3 | 73 | GCGGCCGACGAAGGCACATG |
| | 6-4 | 74 | GGATACAGTGCGGACCGGCC |

2′-O-methyl 3′ phosphorothioate end modified guide RNAs (gRNAs) were synthesized by Synthego and Cas9 mRNA was synthesized by TriLink Biotechnologies. gRNAs were reconstituted at 1 μg·μl⁻¹ in sterile TE buffer.

To measure CRISPR/Cas9 mediated cleavage efficiency, CD3/CD28 beads were magnetically removed 48 hours after T cell activation was initiated. About 60-72 hours after the initial isolation and activation of T cells, T cells were electroporated with Cas9 mRNA and modified gRNA (1 μg each for 2×10⁶ cells) using the Amaxa 4D nucleofector P3 Primary Cell XKitS system (Lonza). Three days after electroporation, the cells were pelleted. gDNA was extracted from the cell pellets for PCR amplification and sequencing of respective sites for cleavage efficiency testing. Analysis of PCR amplicon sequencing data for cleavage efficiency determination was performed using CRISPresso online tool for the deep sequencing data and the ICE online tool (Synthego) for the Sanger sequencing data.

CAR Targeting

T cells were electroporated with Cas9 mRNA and gRNA in accordance with the methods described above. Recombinant AAV6 donor vectors were added to the culture one hour after electroporation at a MOI of 5×10⁵. The culture medium was changed every 2 days and was replaced with fresh medium containing 5 ng/ml interleukin-7 (IL-7) and 5 ng/ml IL-15. The cells were cultured at a concentration of 10⁶ cells per ml.

Antigen Stimulation and In Vitro Proliferation Assays

In the weekly proliferation assay, 3 days after AAV6 transduction, CAR targeted cells were purified using magnetic Biotin-SP (long spacer) AffiniPure F(ab′)₂ Fragment Goat Anti-Mouse IgG, F(ab′)2 Fragment Specific antibody (Jackson ImmunoResearch), anti-biotin microbeads and MS columns (Miltenyi Biotec). The CAR+ purified cells were cultured for 4 days as described before. NIH/3T3 expressing human CD19 cells were used as artificial antigen-presenting cells (AAPCs). For weekly stimulations, 3×10⁵ irradiated CD19+ AAPCs were plated in 24-well plates 12 h before the addition of 5×10⁵ CAR+ T cells in X-vivo15 containing human serum, 5 ng ml⁻¹ interleukin-7 (IL7) and 5 ng ml⁻¹ IL15 (Peprotech). Every 2 days, cells were counted, and media was added to reach a concentration of 2×10⁶ cells per ml. For each condition, T cells were analyzed by FACS for CAR expression at time points mentioned in the respective figures. The antibody used for CAR staining was Alexa Fluor 647 AffiniPure F(ab′)2 Fragment Goat Anti-Mouse IgG, F(ab′)2 Fragment Specific (Jackson ImmunoResearch). For setting CAR MFI, Rainbow Fluorescent Particles were used (BD Biosciences).

### Example 2: Use of Chromatin Insulator to Improve CAR Expression

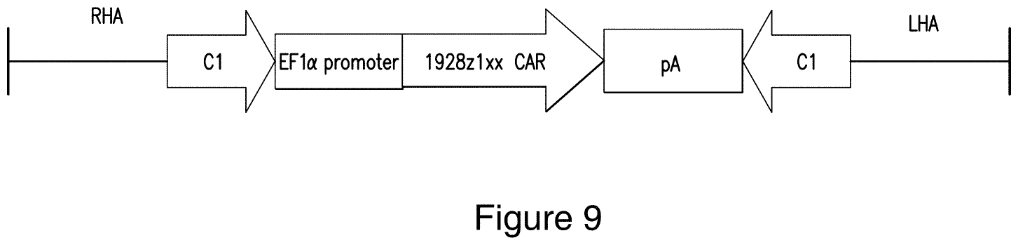

Chromatin insulator element (C.I.) C1 (Liu et al., Nat. Biotechnol. 2015; 33(2):198-203) was incorporated into the CAR gene cassette by flanking the gene cassette with the C.I. C1 was located on both sides in an opposing, convergent orientation to rescue CAR expression levels overtime in culture, possibly as a result of heterochromatinization of the locus (FIG. 9).

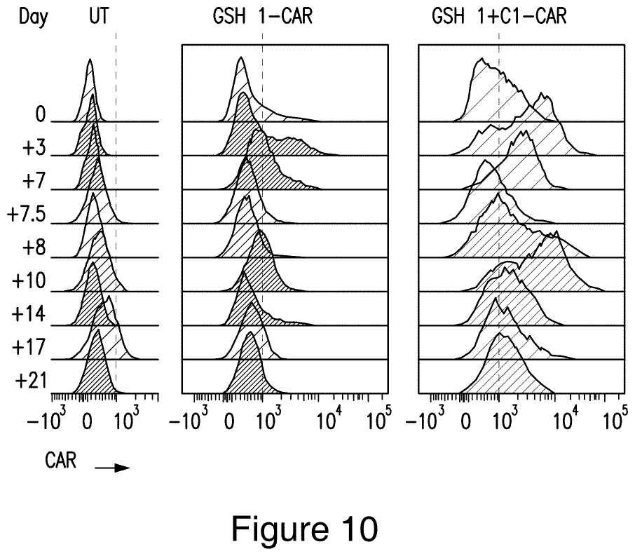

Purified CAR+ T cells with CAR integrated at GSH1 without and with C1 were exposed to antigenic stimulation for 3 weeks (FIG. 10). Similar to FIG. 7, GSH1-CAR T cells without insulator were unable to express CAR on their surface after the second week of stimulation. Upon introduction of the C1, GSH1-CAR T cells were able to express CAR on surface up to week 3. Further, GSH1-CAR T cells having chromatin insulator C1 were able to lyse 3T3 cells in culture. Thus, introduction of chromatin insulator (C.I.) may help improve the expression of CAR in GSH-CAR T cells.

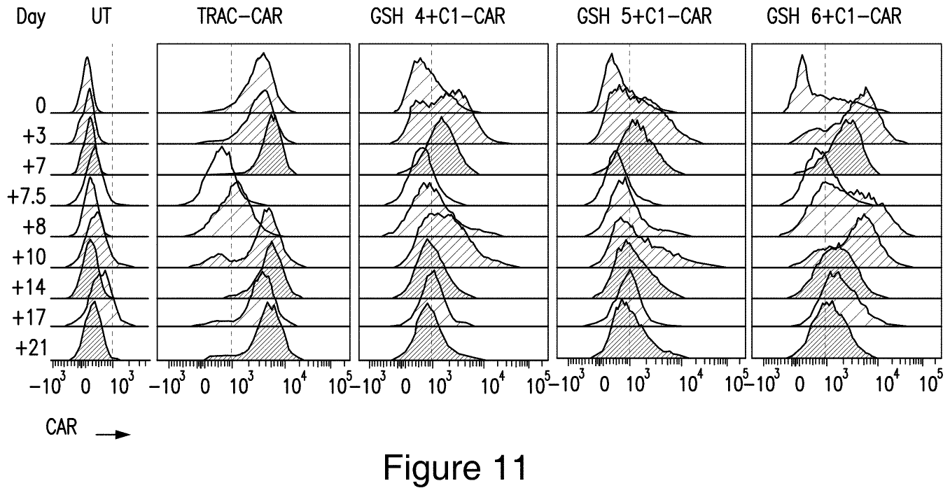

GSH-CAR T cells were created by integrating CAR and C.I. C1 at GSH4, GSH5, or GSH6 of the genome of the T cells. Purified GSH-CAR T cells were exposed to antigenic stimulation for 3 weeks (FIG. 11). Similar to GSH1 CAR T cells in FIG. 10, GSH4, GSH5, and GSH6-CAR T cells had upregulated CAR expression on day 3 after the antigenic stimulation as compared to day 0. The CAR expression was downregulated until the GSH-CAR T cells were re-exposed to antigens again.

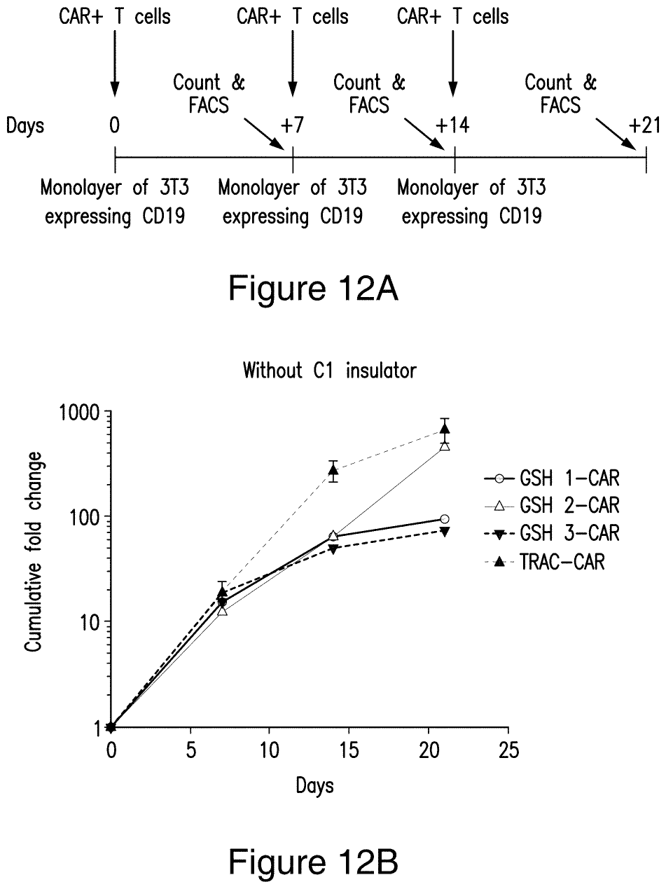

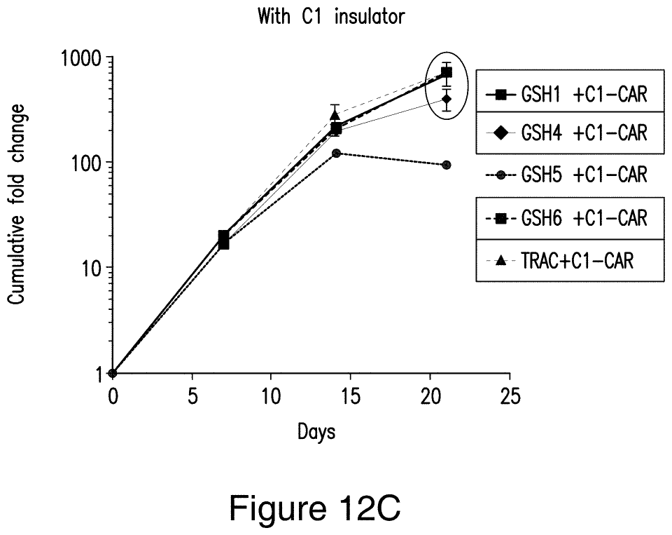

Purified GSH-CAR T cells with or without C.I. C1 were exposed to antigenic stimulation for 3 weeks and proliferation of those cells were monitored (FIG. 12A). GSH1-CAR T cells and GSH3-CAR T cells proliferated slower than those with TRAC-CAR T cells (FIG. 12B). On day 21, GSH2-CAR T cells exhibited comparable proliferation rate to TRAC-CAR T cells (FIG. 12B). GSH5-CAR T cells having integrated C.I. C1 proliferated slower than TRAC-CAR T cells (FIG. 12C). On day 21, GSH1-CAR, GSH4-CAR, and GSH6-CAR T cells having C.I. C1 exhibited comparable proliferation rate to those with TRAC-CAR (FIG. 12C).

### Example 3: In-Vivo Assessment of CARs Integrated at GSHS in CD19-CAR Stress Test Model of B-All

In-vivo assessment of GSH-CAR T cells in CD19-CAR stress test model of B-ALL 6 to 12-week-old NOD/SCID/IL-2Rγ null male mice were used. Mice were inoculated with 0.5×10⁶ CD19-FFLuc-GFP NALM6 cells by tail vein injection, followed by 0.4×10⁶ CAR T cells injected 4 days later. Bioluminescence imaging was performed using the IVIS Imaging System with the Living Image software for the acquisition of imaging datasets. Tumor burden was assessed as described in Gade et al., Cancer Res. 2005; 65(19):9080-9088.

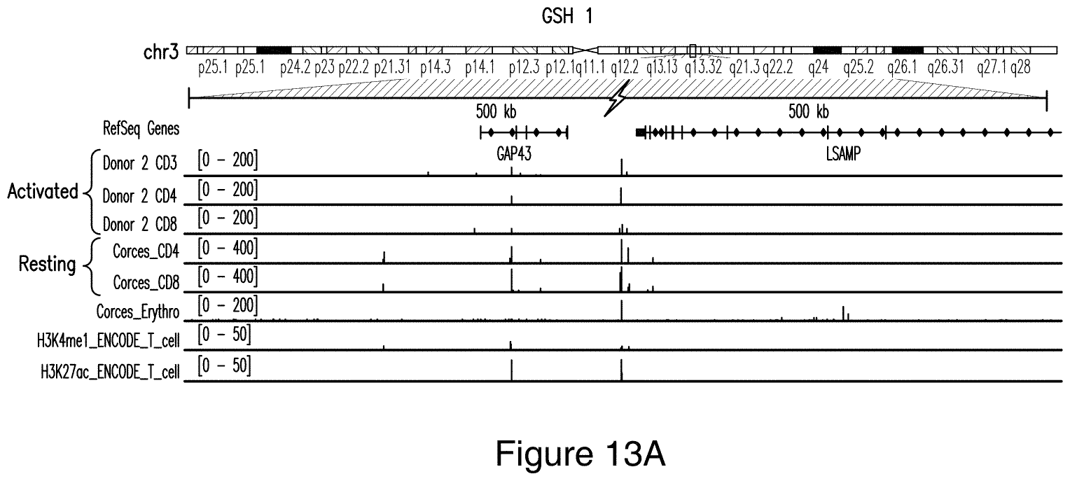

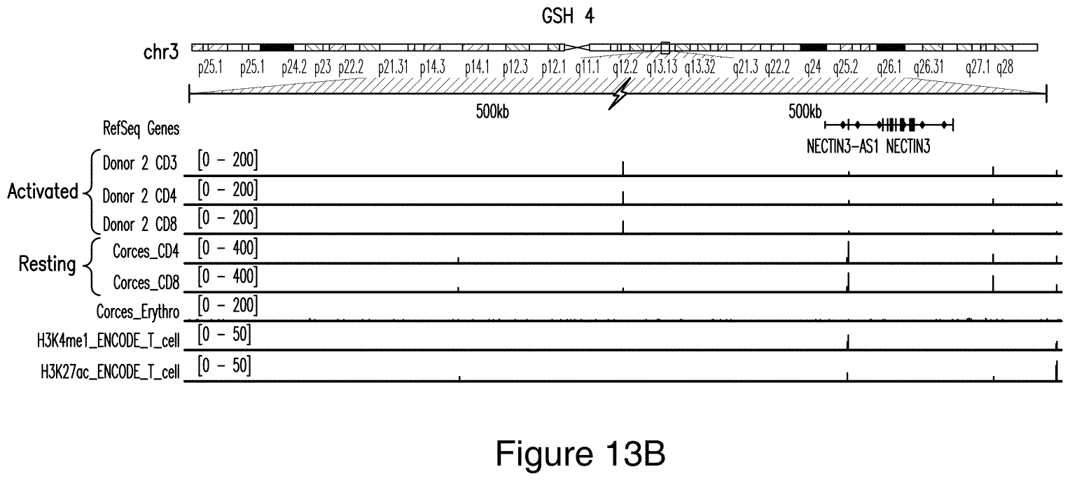

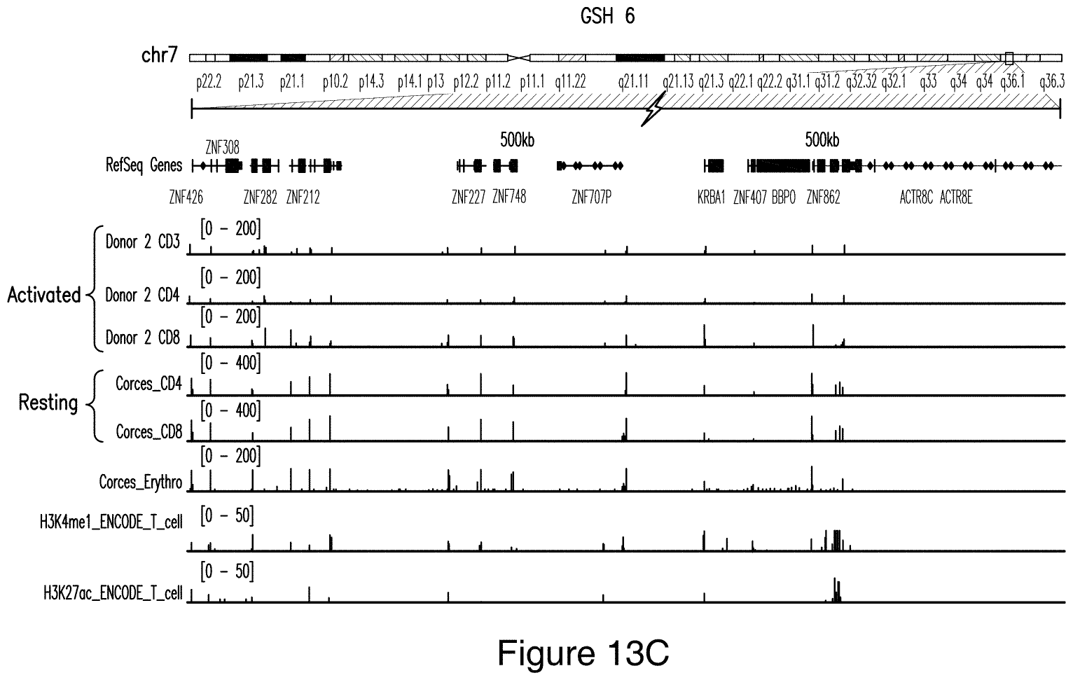

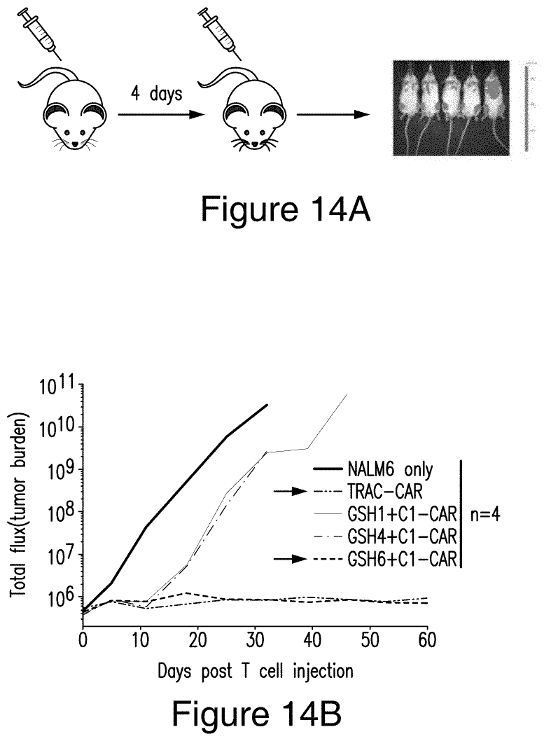

GSH1, GSH4, an GSH6 were used for the in-vivo assessment. A 1000 kb region centered on the gRNA cleavage site for each of GSH1, GSH4, an GSH6 were shown (FIGS. 13A-13C). Median tumor burden for 4 mice per group administered T cells with CAR and C.I. C1 integrated at GSH1, GSH4, an GSH6 as well as TRAC-CAR was monitored for 60 days post T cell injection (FIGS. 14A and 14B). The median tumor burden for groups of GSH 1 integrated with C1 insulator and GSH2 integrated with C1 insulator grew at a much slower rate compared to the NALM6 group. But tumors still grew over time. The median tumor burden for GSH6 with C1 insulator barely grew compared to the NALM6 group. GSH6-CAR showed effective tumor control similar to TRAC-CAR for 60 days post T cell injection.

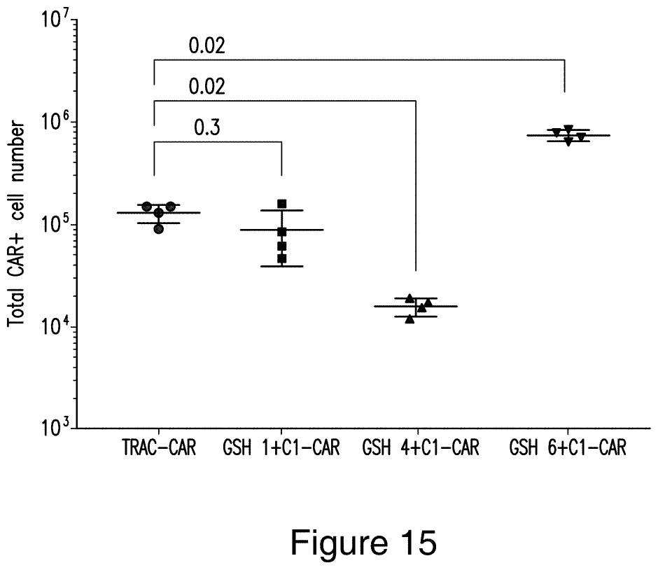

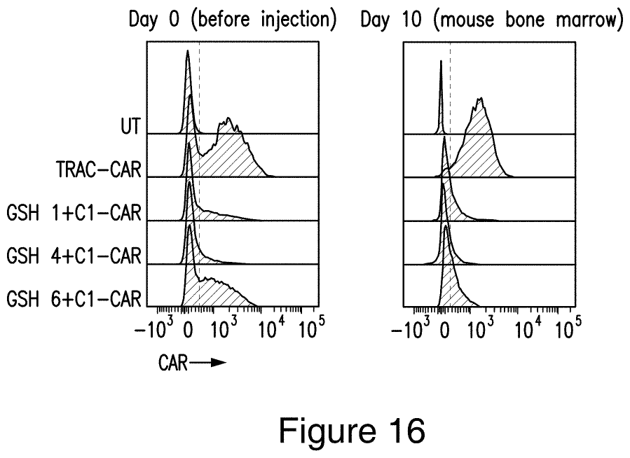

CAR+ cells in mouse bone marrow on day 10 post injection were analyzed (FIG. 15). Total CAR+ cell numbers were counted. The number of CAR+ cell with CAR integrated at GSH6 with C.I. C1 was more than that of CAR+ cell with TRAC-CAR while the numbers of CAR+ cell with CAR integrated at GSH1 and GSH4 with C.I. C1 were less than that of CAR+ cell with TRAC-CAR. The CAR+ cells with TRAC-CAR and CAR integrated at GSH1, GSH4, an GSH6 with C.I. C1 were analyzed by flow cytometry on day 10 post injection (FIG. 16). The CAR+ cells with CAR integrated at GSH6 with C.I. C1 exhibited a higher expression level than those with CAR integrated at GSH1 and GSH4 with C.I. C1.

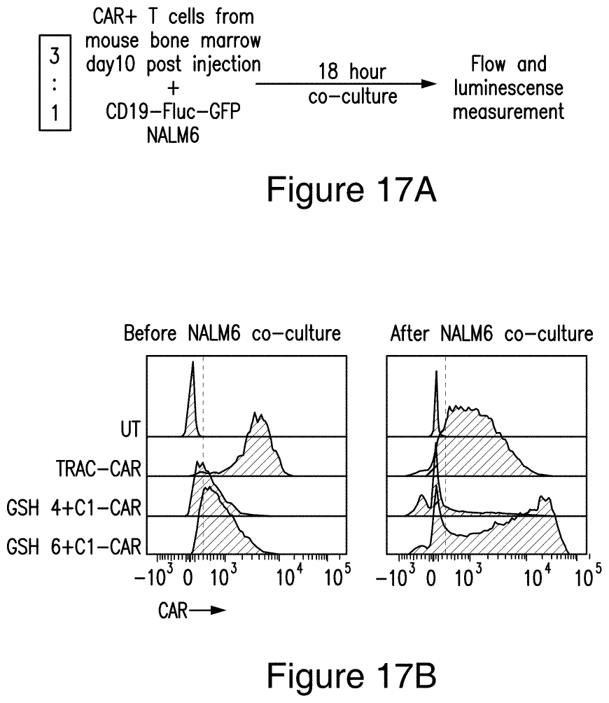

CAR+ T cells from mouse bone marrow on day 10 post injection were mixed with CD19-Fluc-GFP NALM6 at a ratio of 3:1 and co-cultured for eighteen hours and measured by flow cytometry and luminescence (FIG. 17A). CAR expressions in CAR+ T cells with CAR integrated at GSH6 with C.I. C1 and with TRAC-CAR were upregulated upon re-exposure to antigen (FIG. 17B).

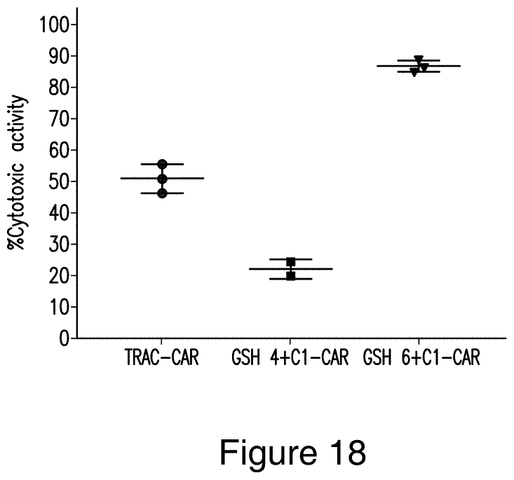

Cytotoxic activity was determined by 18-hour luciferase assay at day 10 post T cell injection in cells taken from the bone marrow of mice. The method was disclosed hereunder. GSH6-CAR with C.I. C1 exhibited high cytotoxic activity at days 10 and such cytotoxic activity was higher than that exhibited by the TRAC-CARs at this timepoint (FIG. 18). GSH4-CAR with C.I. C1 exhibited low cytotoxic activity at days 10 and such cytotoxic activity was lower than that exhibited by the TRAC-CARs at this timepoint.

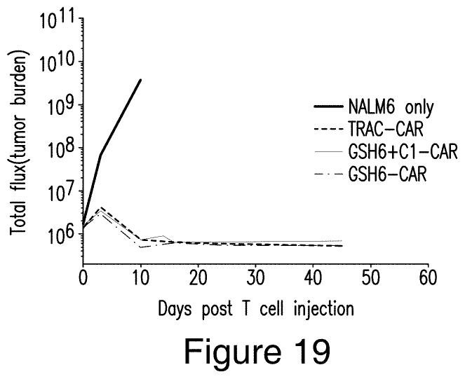

Median tumor burden for 6 mice per group administered T cells with CAR and GSH6 with and without integrated C.I. C1 as well as TRAC-CAR was monitored for 45 days post T cell injection (FIG. 19). GSH6-CAR showed effective tumor control with as well as without C.I. C1 for at least 45 days in vivo.

Cytotoxic Activity Measurement

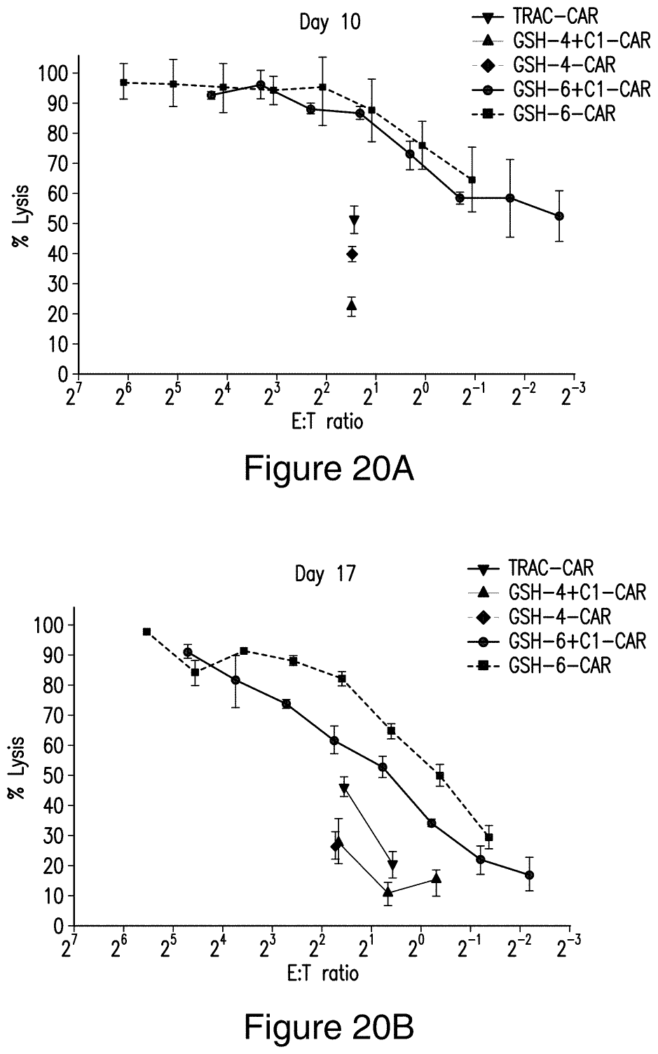

Cytotoxic activity was determined by 18 h luciferase assay at day 10 (FIG. 20A) and day 17 (FIG. 20B) post T cell injection in cells taken from the bone marrow of mice. Each group contained cells taken from 4 independent mice of the same group and pooled together. Nalm6-expressing CD19-FFLuc-GFP served as target cells. The effector and tumor target cells were cocultured in triplicates at the indicated effector/target ratio using black-walled 96-well plates with 15000 target cells in a total volume of 100 μl per well in Nalm6 medium. Target cells alone were plated at the same cell density to determine the maximal luciferase expression (relative light units (RLU)); 18 h later, 100 μl luciferase substrate was directly added to each well. Emitted light was detected in a luminescence plate reader. Lysis was determined as (1−(RLUsample)/(RLUmax))×100.

Different CAR: NALM6=Effector: Target (E:T) ratios were analyzed based on cell numbers available. GSH6-CAR with and without C.I. C1 exhibited high cytotoxic activity at days 10 and 17 and such cytotoxic activity was higher than the cytotoxic activity exhibited by the TRAC-CARs at this timepoint (FIGS. 20A and 20B). GSH4-CARs with and without C.I. C1 exhibited poor cytotoxic activity at days 10 and 17 and such cytotoxic activity was lower than the TRAC-CAR.

### Example 4: Genomic Safe Harbors for CAR T Cell Engineering

The therapeutic use of genetically engineered human cells is rapidly expanding beyond gene therapy for inherited monogenic disorders to acquired disorders. Alterations of the human genome may thus not only serve to compensate for or correct mutations (Dunbar, C. E. et al., Science 359, eaan4672 (2018)) as is the case in severe combined immune deficiencies and the thalassemias, but also introduce natural or synthetic genes to reprogram cell function, as is the case for chimeric antigen receptor (CAR) therapy (June, C. H. & Sadelain, M., N. Engl. J. Med. 379, 64-73 (2018); Sadelain, M., Rivière, I. & Riddell, S., Nature 545, 423-431 (2017)). An ideal genetic treatment should provide for predictable and dependable expression of the transgene in the intended cell type, at an optimal level and stably over time, without incurring genetic adverse events. γ-Retroviral, lentiviral and transposon-based vectors are commonly used to achieve stable genetic modifications. Albeit effective ((Dunbar, C. E. et al., Science 359, eaan4672 (2018))), they all afford semi-random integration, potentially resulting in insertional mutagenesis (Craigie, R. & Bushman, F. D. Cold Spring Harb. Perspect. Med. 2, a006890 (2012); Bushman, F., Lewinski, M., Ciuffi, A., Barr, S. & Leipzig, J. Nat. Rev. Microbiol. 3, 848-858 (2005); Schwarzwaelder, K. et al. Gammaretrovirus-mediated correction of SCID-X1 is associated with skewed vector integration site distribution in vivo. 117, 2241-2249 (2007); Singh, P. K. et al. Genes Dev. 29, 2287-2297 (2015)) and variegated transgene expression (Rivella, S. & Sadelain, M. Semin. Hematol. 35, 112-125 (1998); Ellis, J. Hum. Gene Ther. 16, 1241-1246 (2005)). Furthermore, the integration of γ-retroviral and lentiviral vectors is biased towards gene loci (Craigie, R. & Bushman, F. D. Cold Spring Harb. Perspect. Med. 2, a006890 (2012); Bushman, F., Lewinski, M., Ciuffi, A., Barr, S. & Leipzig, J. Nat. Rev. Microbiol. 3, 848-858 (2005); Dunbar, C. E. Ann. N. Y. Acad. Sci. 1044, 178-182 (2005)) increasing the probability of transgene expression and also the potential to disrupt the function or expression of endogenous genes. The most dreaded consequence is oncogene activation, which may ultimately promote malignant transformation (Stein, S. et al. Nat. Med. 16, 198-204 (2010)). A prominent example of such serious adverse events are reports of leukemia occurring in patients treated with retroviral-mediated gene therapy for X-linked severe combined immunodeficiency (X-SCID) (Kohn, D. B., Sadelain, M. & Glorioso, J. C. Nat. Rev. Cancer 3, 477-488 (2003); Hacein-Bey-Abina, S. et al. J Clin Invest 118, 3132-3142 (2008); Howe, S. J. et al. J. Clin. Invest. 118, 3143-50 (2008)). Clonal expansions stopping short of leukemic transformation have occurred in both hematopoietic stem cell therapies (Cavazzana-Calvo, M. et al. Nature 467, 318-22 (2010)) and CART cell therapies (Shah, N. N. et al. Blood Adv. 3, 2317-2322 (2019); Fraietta, J. A. et al. Nature 558, 307-312 (2018)). The other major detrimental consequence of semi-random integration that limits the efficacy of some gene therapies is variegated and hence unpredictable transgene expression, which comprises transcriptional silencing due to chromosomal position effects and heterochromatinization (Ellis, J. Hum. Gene Ther. 16, 1241-1246 (2005)).

In principle, these challenges could be overcome if the transgene were integrated at a defined genomic site that reliably provides safe and stable gene expression. Such “genomic safe harbors” (GSH) may be intra or extra-genic. Three intra- or juxta-genic sites have been proposed as potential GSH in human cells: the adeno-associated virus site 1 (AAVS1), the chemokine (CC motif) receptor 5 (CCRS) locus and the human orthologue of the mouse ROSA26 locus (Sadelain, M., Papapetrou, E. P. & Bushman, F. D. Nat. Rev. Cancer 12, 51-58 (2011); Kotin, R. M., Linden, R. M. & Berns, K. I. The EMBO journal. 11, 5071-5078 (1992); Irion, S. et al. Nat. Biotechnol. 25, 1477-1482 (2007); Lombardo, A. et al. Nat. Biotechnol. 25, 1298-1306 (2007); DeKelver, R. C. et al. Genome Res. 20, 1133-1142 (2010); Papapetrou, E. P. & Schambach, A. Mol. Ther. 24, 678-684 (2016)). These lie either within a gene thought to be dispensable or in close proximity to genes that are deemed not to pose an oncogenic threat. Their vicinity is indeed gene-rich, which may be favorable to support transgene expression but raises the risk of their trans-activation following integration of ectopic enhancer/promoter elements.

Alternatively, one may search for remote extragenic GSH (Sadelain, M., Papapetrou, E. P. & Bushman, F. D. Nat. Rev. Cancer 12, 51-58 (2011)). The presently disclosed criteria are for the retrospective identification of safe viral vector integrations at candidate GSH. The advent of site-specific nucleases now makes it possible to direct transgene integration to GSH, provided that the latter are accessible. Focusing on T cell engineering to advance cancer immunotherapy (Sadelain, M., Rivière, I. & Riddell, S. Nature 545, 423-431 (2017)), the presently disclosed subject matter showed the use of CRISPR/Cas9 to target candidate GSH, efficiently undergo homologous recombination using AAV6 vectors (Eyquem, J. et al. Targeting a CAR to the TRAC locus with CRISPR/Cas9 enhances tumor rejection. Nature 543, 113-117 (2017); Schumann, K. et al. PNAS 112, 10437-10442 (2015); Roth, T. L. et al. Nature 559, 405-409 (2018); Sather, B. D. et al. Sci. Transl. Med. 7, 307ra156 (2015)) and support sustained transgene expression. Using a CAR specific for CD19, it was demonstrated herein that one such site, termed GSH6, directed CAR expression that was as effective as the TRAC locus, an optimal locus for CAR T cell engineering (Eyquem, J. et al. Targeting a CAR to the TRAC locus with CRISPR/Cas9 enhances tumour rejection. Nature 543, 113-117 (2017)). The identification of accessible GSH in primary T cells can facilitate the generation of T cells that predictably and homogeneously express their therapeutic gene cargo, thereby enhancing the safety and efficacy of cancer immunotherapy (June, C. H. & Sadelain, M. N. Engl. J. Med. 379, 64-73 (2018)).

### Results

Identification and Targeting of GSHs

A set of 5 safety criteria previously proposed to define extragenic genomic safe harbors (GSH) based on the avoidance of chromosomal integrations posing a risk of insertional oncogenesis (Papapetrou, E. P. et al. Nat. Biotechnol. 29, 73-78 (2011)). Based on recent findings on the role of non-coding RNAs (ncRNAs) in regulating cell function (Beermann, J., Piccoli, M. T., Viereck, J. & Thum, T. Physiol. Rev. 96, 1297-1325 (2016); Esteller, M. Nat. Rev. Genet. 12, 861-874 (2011)), a sixth criterion was added to exclude disruption of known ncRNA (Table 5). Two additional criteria were added to achieve efficient site-specific transgene integration at the selected sites, requiring dependable cleavage by nucleases like Cas9 and subsequent homologous recombination, and the further need to achieve dependable and sustained transgene function (Table 5).

TABLE 5
Criteria for identification of GSH

| | Criteria for GSH |
|---|---|
| 1. | Distance of >50 kb from 5′ end of any gene |
| 2. | Distance of >300 kb from any cancer-related gene |
| 3. | Distance of >300 kb from any miRNA |
| 4. | Outside a gene transcription unit |
| 5. | Outside of Ultra-conserved regions |
| 6. | Outside of Non coding RNAs |
| 7. | Efficient cleavability |
| 8. | Reliable transgene expression and regulation |

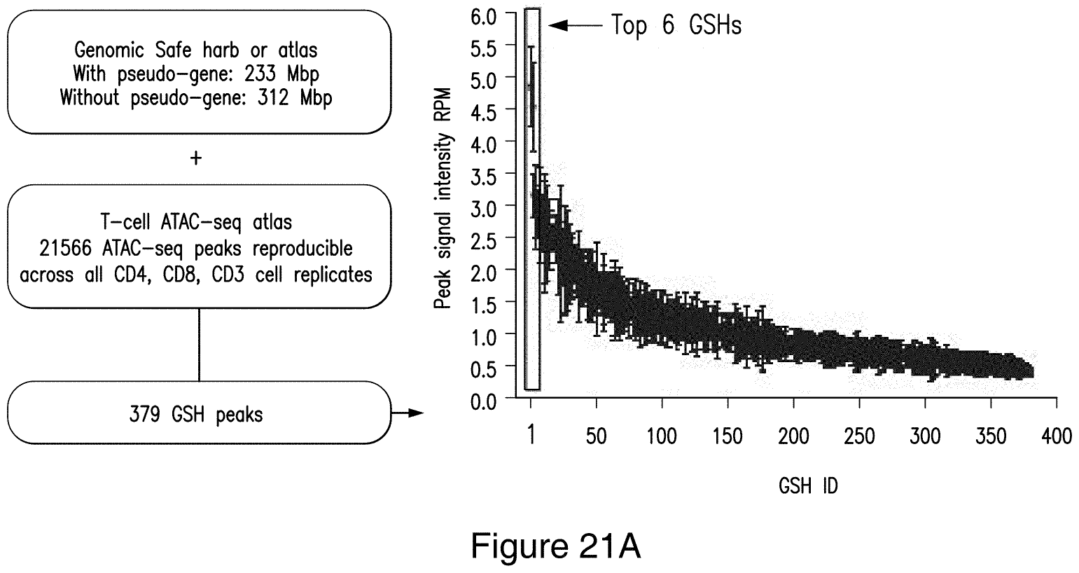

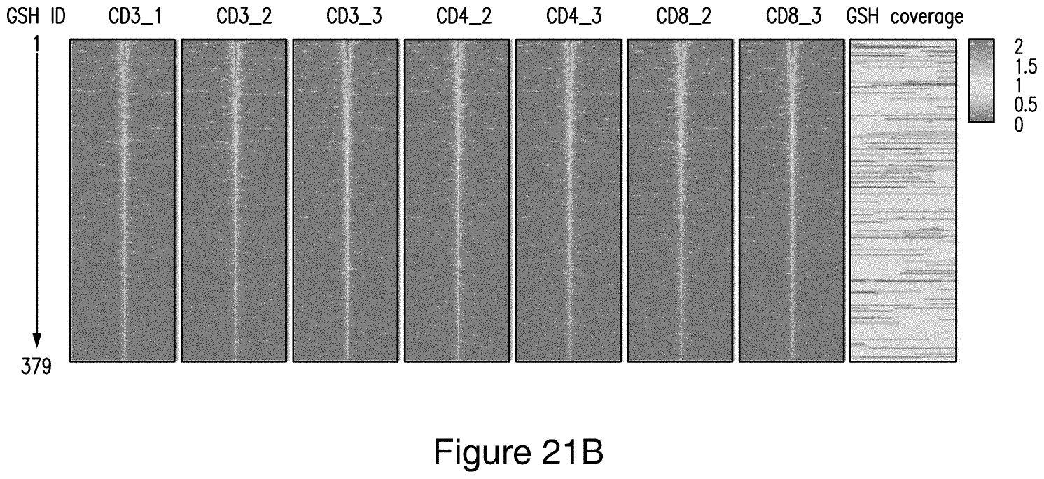

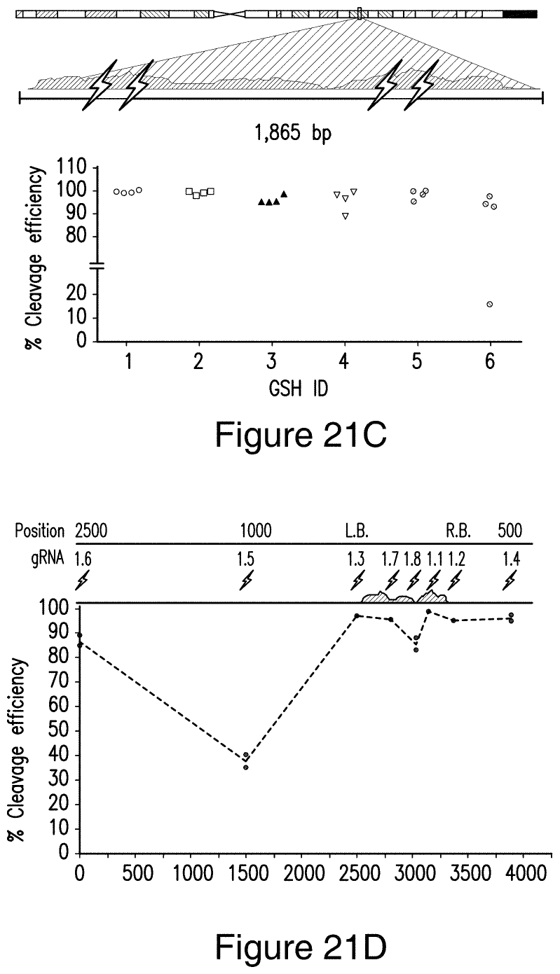

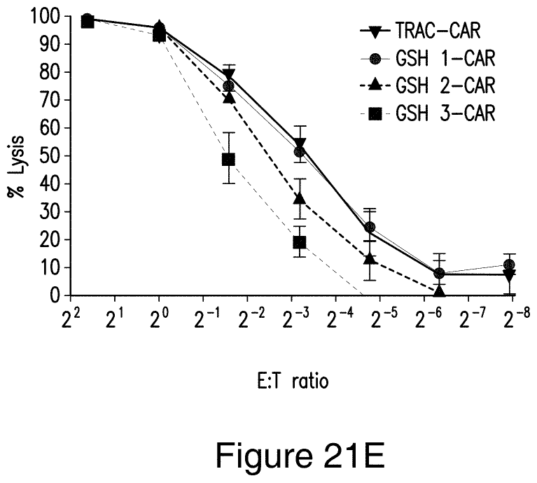

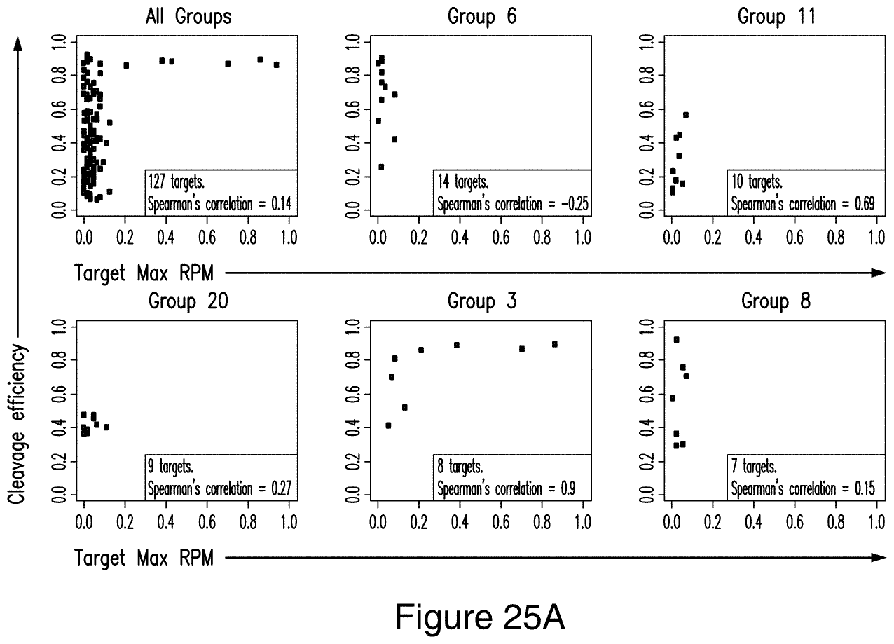

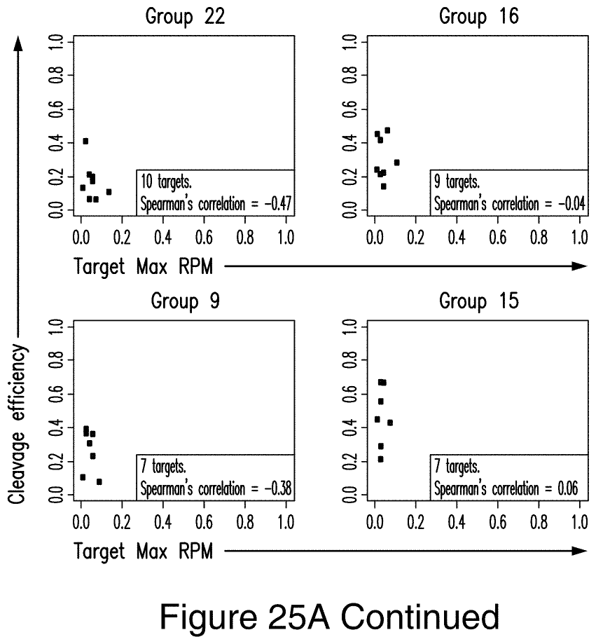

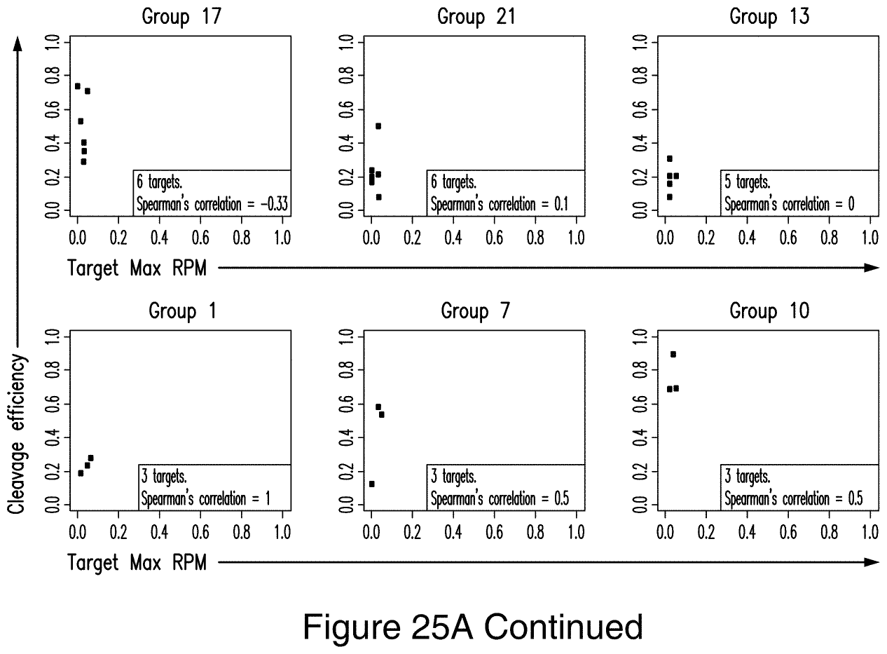

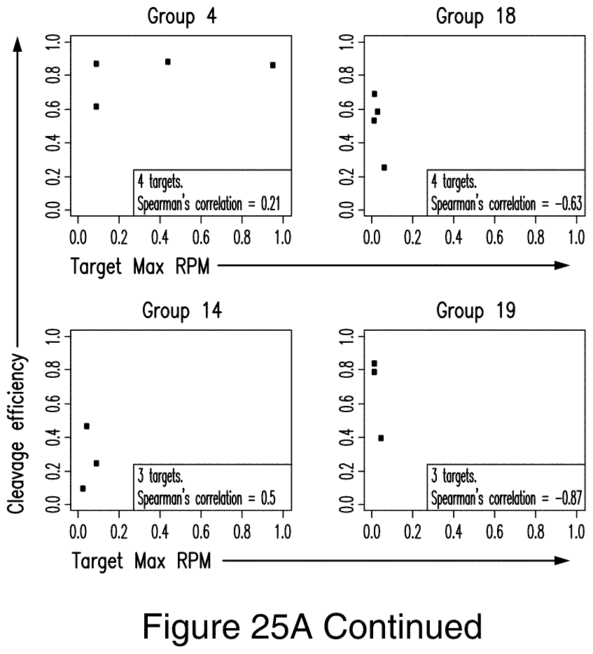

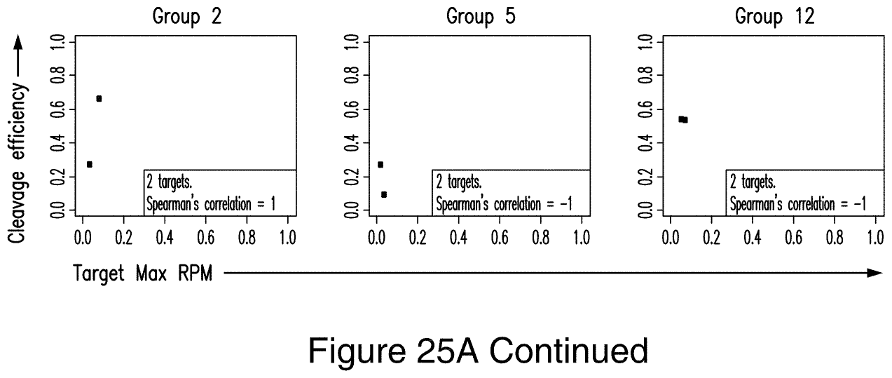

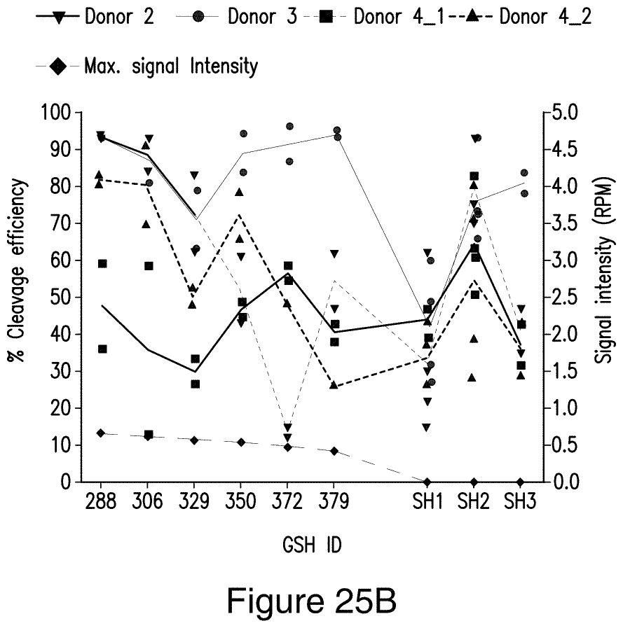

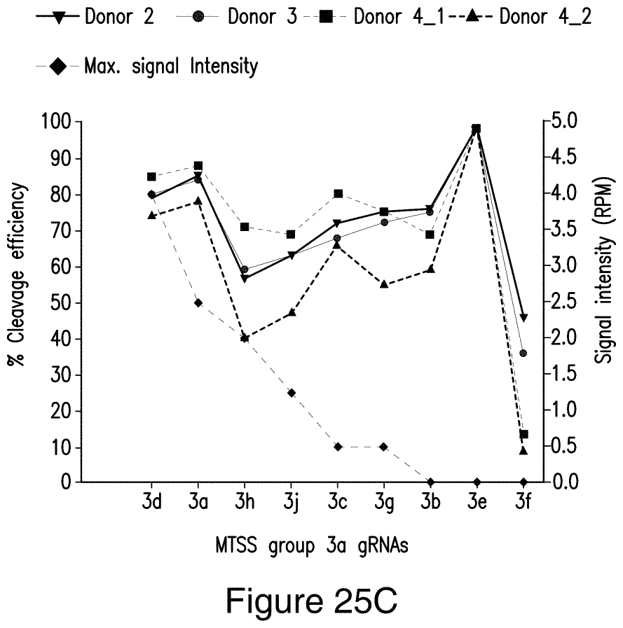

To date, the cleavage efficiencies predicted by software that use features of the gRNA sequence alone have been inaccurate in estimating cleavage efficiencies in a living cell (Verkuijl, S. A. & Rots, M. G. Curr. Opin. Biotechnol. 55, 68-73 (2019)). Given the very specific and dynamic chromatin environment of chromosomal DNA in living cells, the chromatin context of a genomic locus governs DNA accessibility and hence cleavability and subsequent transgene expression from that site. Analysis of data from Van Overbeek et al. (Van Overbeek, M. et al. Mol. Cell 63, 633-646 (2016)) on the activity of Cas9 suggested that a site possessing accessible chromatin indeed had a higher probability of displaying efficient cleavage (FIG. 25A). Candidate GSHs that conform to the safety and accessibility GSH criteria integrating measurable chromatin accessibility were identified. The technique of ATAC-seq (Assay for transposase accessible chromatin) was utilized to assess the genome wide chromatin accessibility of primary human T cells (Buenrostro, J. D., Wu, B., Chang, H. Y. & Greenleaf, W. J. Curr. Protoc. Mol. Biol. 109, 21.29.1-21.29.9 (2015)). ATAC-seq was performed three days after isolation and activation of primary human T cells obtained from healthy donors, a time point at which GSHs would eventually be targeted for transgene delivery. An ATAC-seq atlas was generated with the reproducible ATAC-seq peaks shared across all cell types and replicates (details in Methods) along with a GSH atlas constructed by computing regions that satisfy the first six GSH criteria. Pseudogenes were excluded from the gene list since pseudogenes are thought to be non-functional genes and used this ‘without pseudogene’ GSH atlas hereafter (FIG. 21A). Merging of the ATAC-seq atlas and GSH atlas resulted in the identification of 379 GSHs, which have at least one ATAC-seq peak as a part of the GSH or within 5 kb of the GSH boundaries, a region around an ATAC-seq peak which would likely be maintained open. These 379 GSHs were then ordered by average ATAC-seq signal intensity at the summit of the associated ATAC-seq peak across all seven samples (FIGS. 21A and 21B).

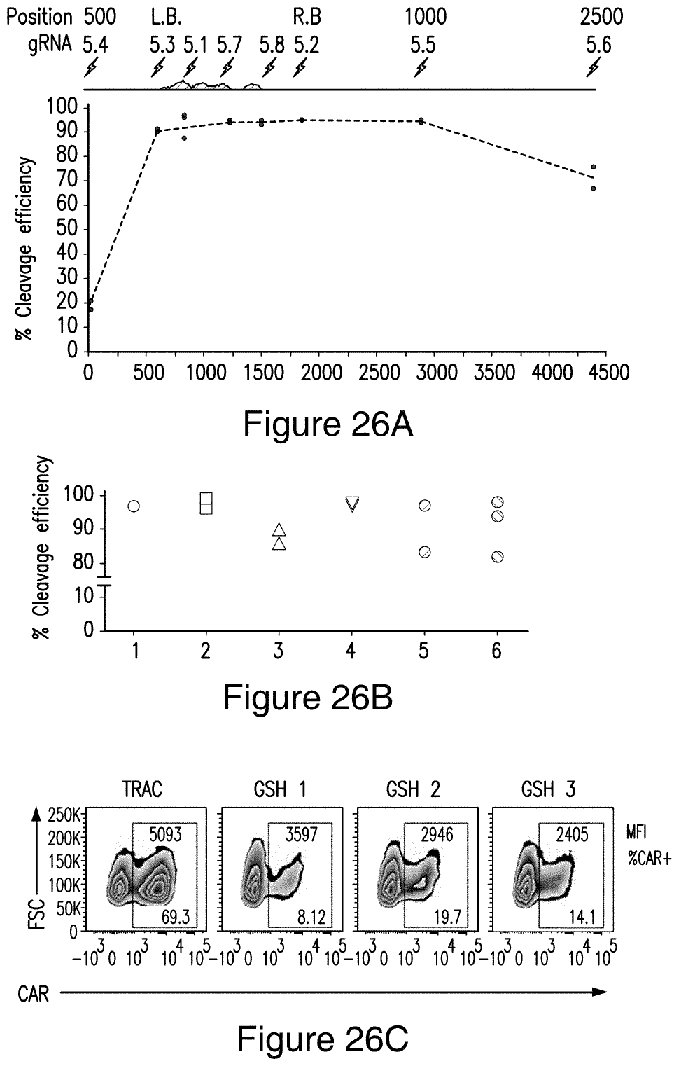

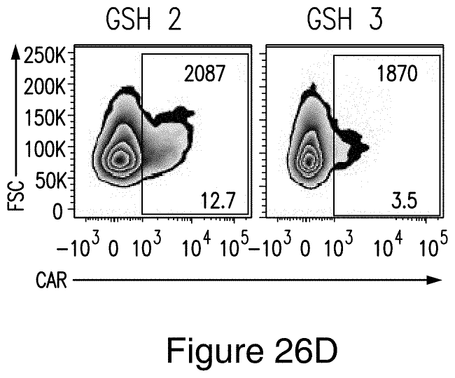

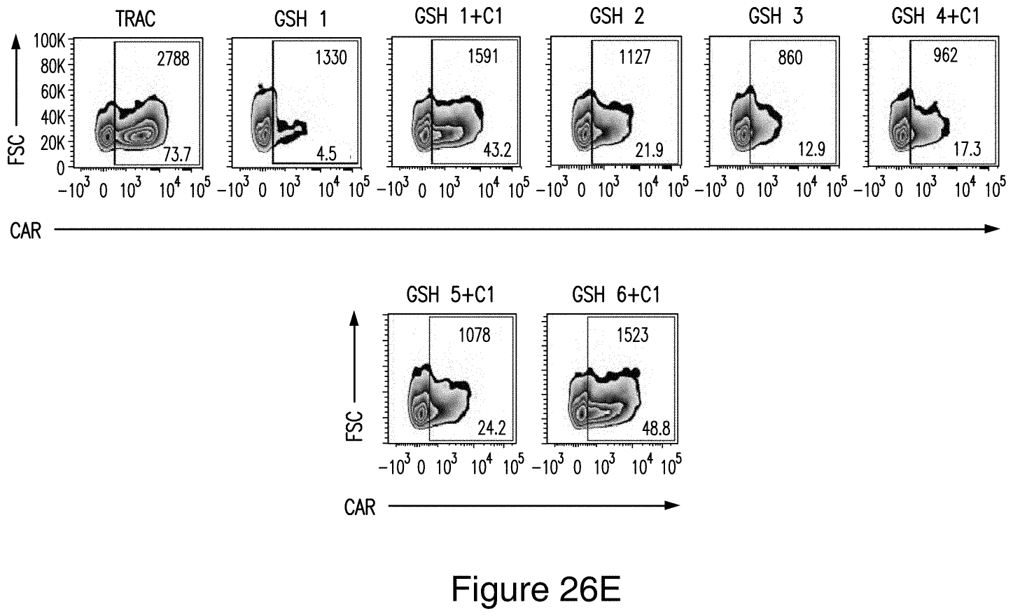

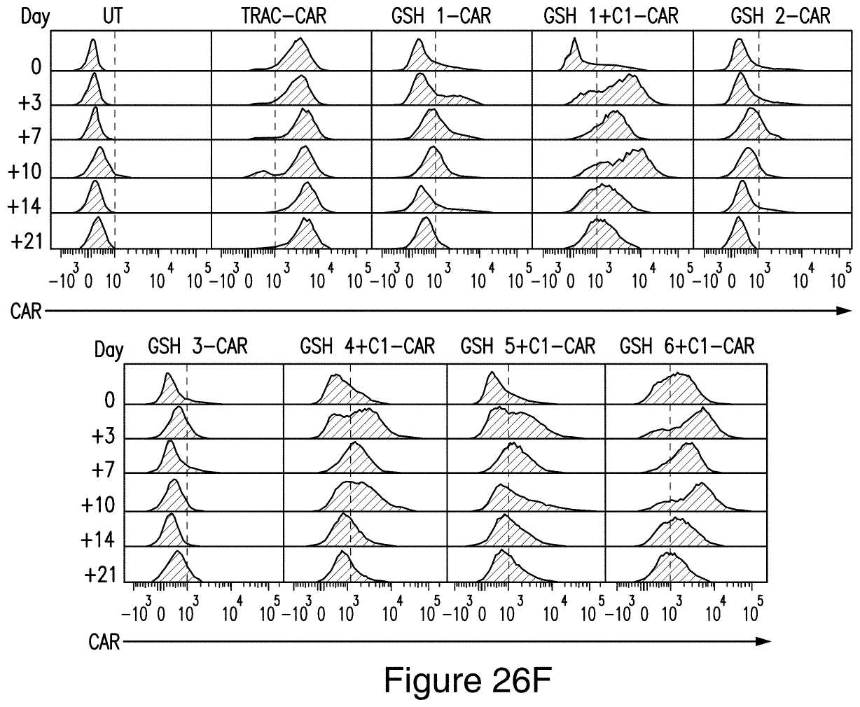

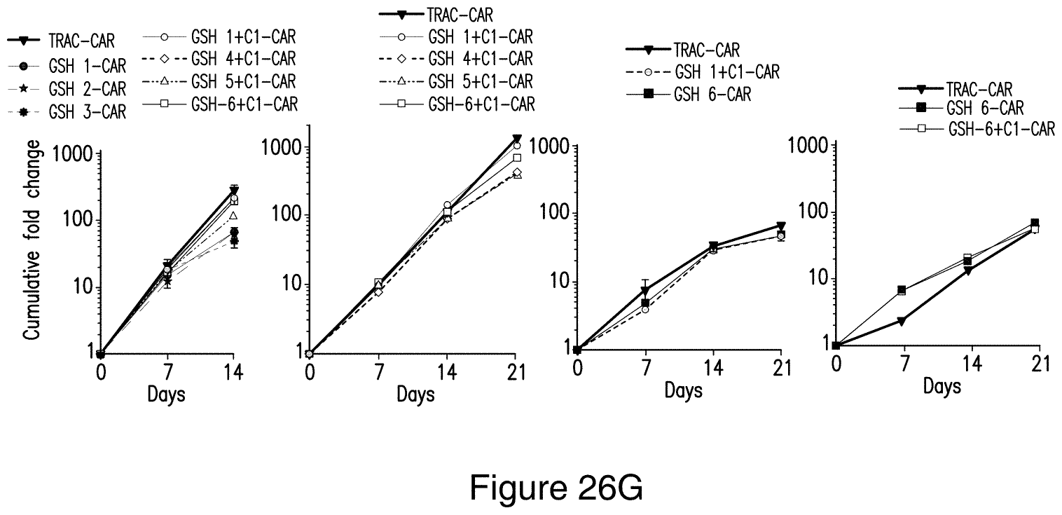

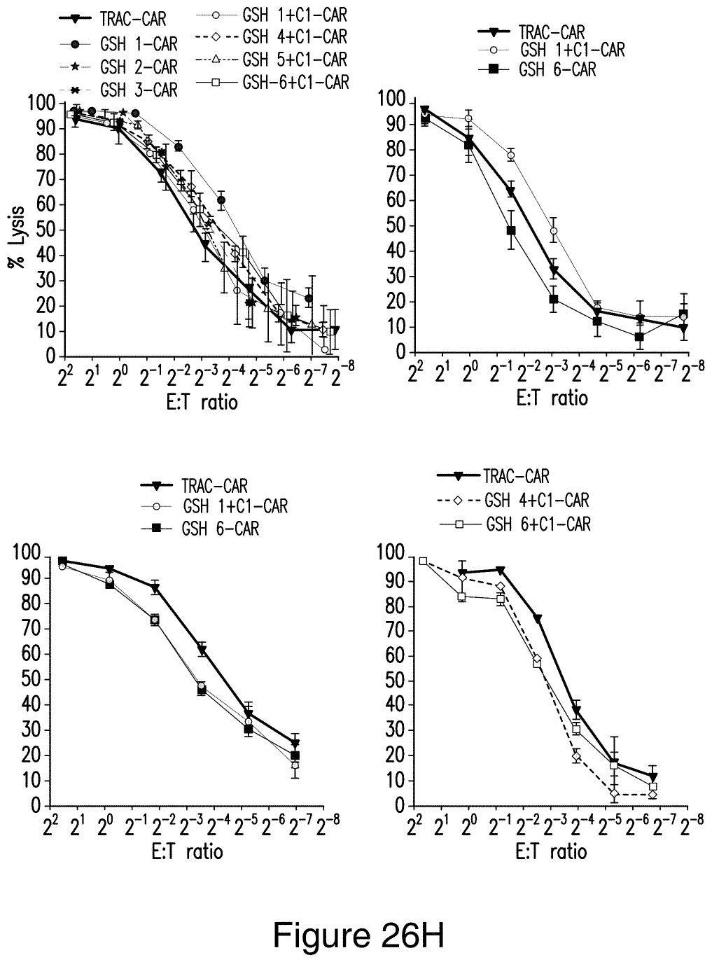

The 6 most accessible GSHs were then selected to test their cleavage efficiency. Four gRNAs per site were designed at the summit of the peak for all 6 GSHs such that all gRNAs possessed a Doench score >1=50 and specificity score>0.2 (Doench, J. G. et al. Nat. Biotechnol. 34, 1-12 (2016); Perez, A. R. et al. Nat. Biotechnol. 35, 347-349 (2017)). Electroporation of Cas9 mRNA and chemically modified sgRNAs (Hendel, A. et al. Nat. Biotechnol. 33, 985-989 (2015)) resulted in >90% cleavage efficiencies at all six GSHs tested at day 3 after electroporation (FIG. 21C). These high editing efficiencies supported that association of a GSH with a high intensity ATAC-seq peak affords a higher cleavage efficiency. However, it was still unclear if targeting only at the ATAC-seq peak summit would afford a high cleavage efficiency or whether targeting anywhere within the peak or even at a certain distance away from the peak could afford the same degree of cleavage. To test this, two GSHs were randomly chosen among the top 6, GSHs 1 and 5, to design gRNAs throughout the width of the peak and at specific distances away from the peak edges (up to 2.5 kb away) and analyzed the cleavage efficiencies at these sites in T cells from two independent donors (FIG. 21D and FIG. 26A). Although cleavage efficiency dropped slightly at one site at a distance of 2.5 kb away from one edge (FIG. 26A), high efficiency was generally maintained anywhere within the peak and at least up to about 500 bp away from the peak's edges.

Two gRNAs per GSH at the peak summit were further tested for four GSHs that had low ATAC-seq peak signal intensities and 3 GSHs identified previously (Papapetrou, E. P. et al. Nat. Biotechnol. 29, 73-78 (2011)) that had no associated ATAC-seq peaks. A multiple target site specific (MTSS) gRNA32 that targets 9 different loci which have different associated ATAC-seq peak signal intensities (FIGS. 25B and 25C) was additionally included. These controls further corroborated that an extragenic site with an associated high signal intensity ATAC-seq peak had a higher probability of efficient cleavage.

Expression of GSH-Encoded CAR and In Vitro Function

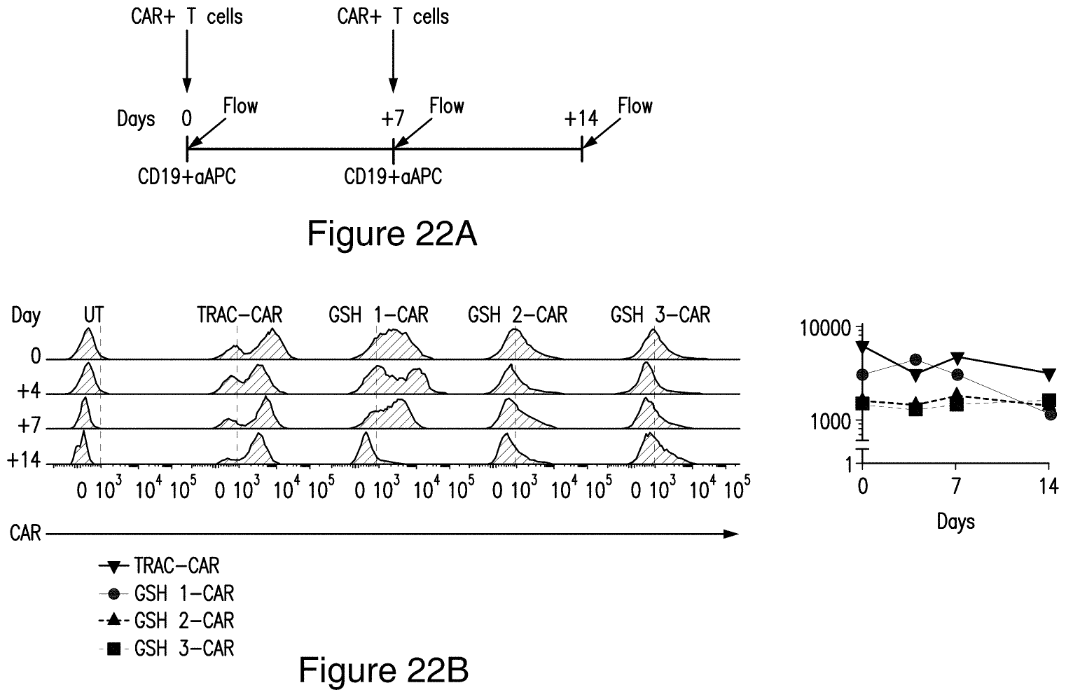

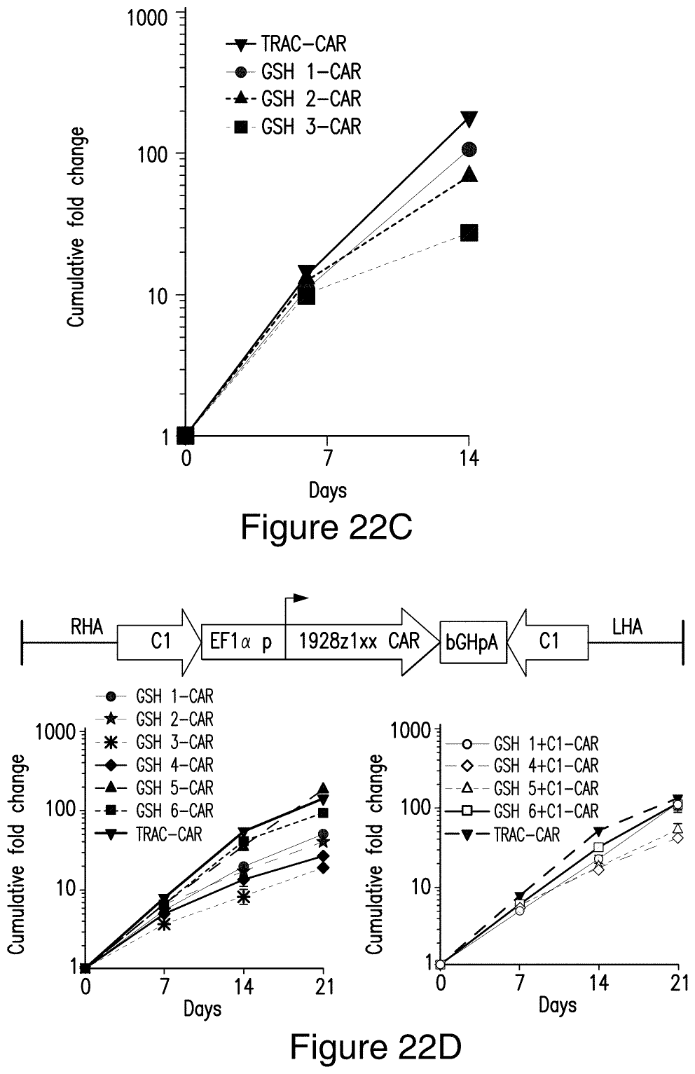

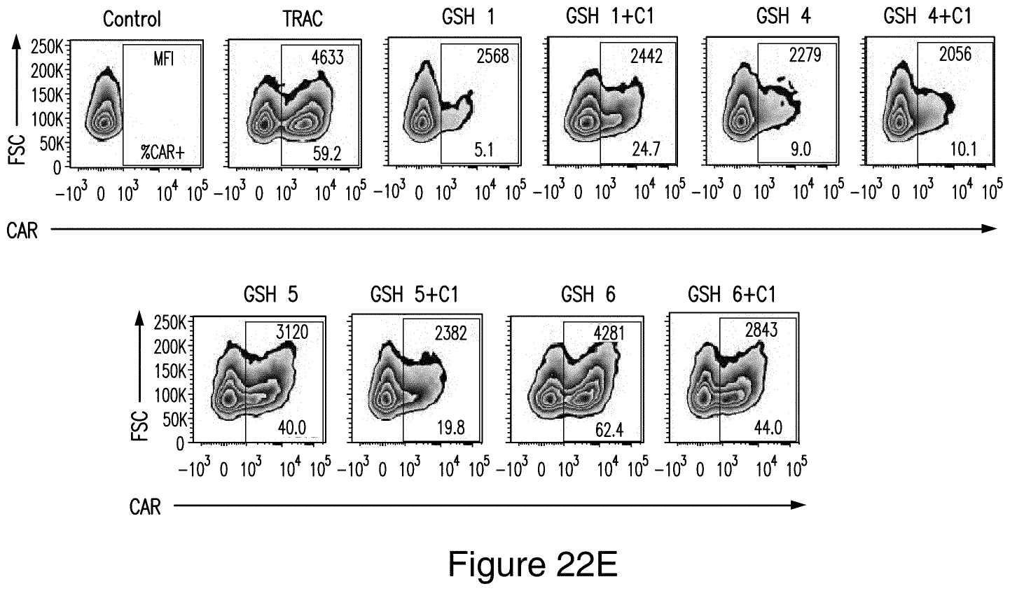

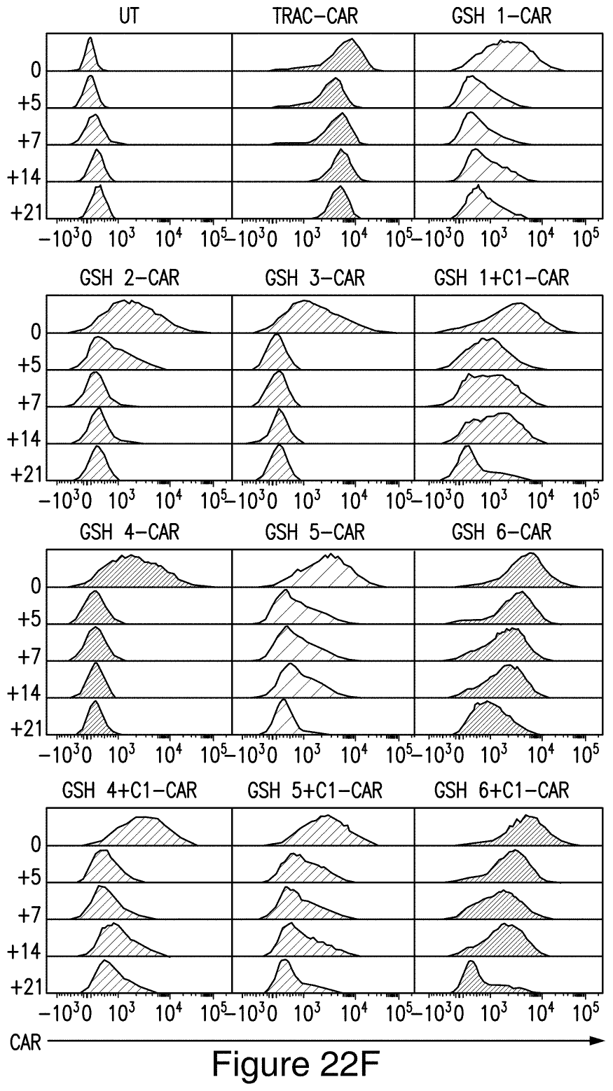

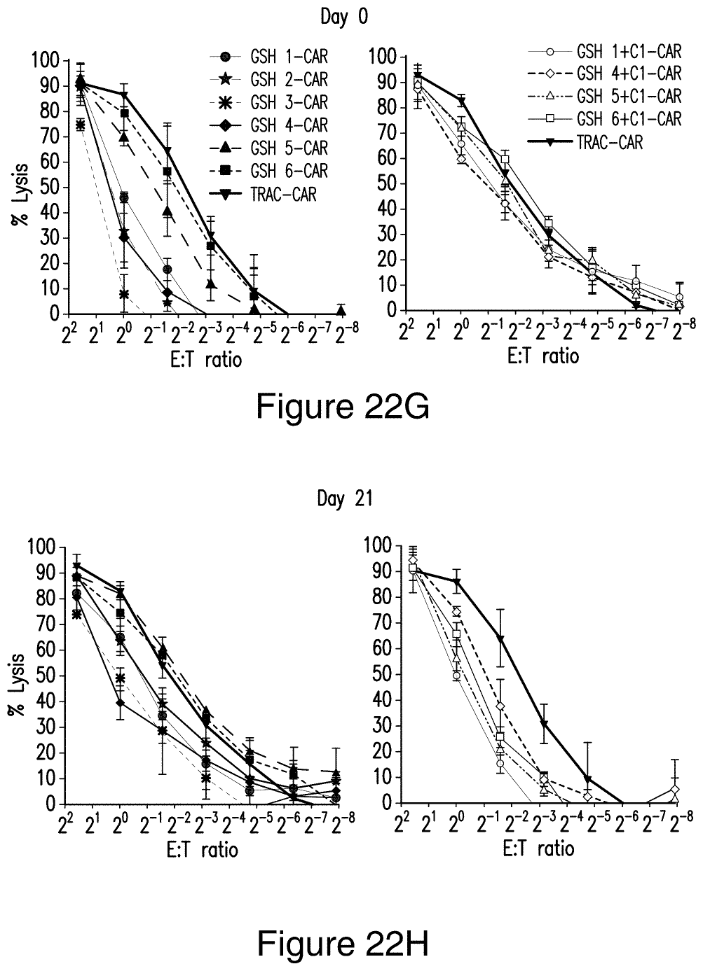

rAAV6 vectors were first designed encoding the 1928ζ-1xx CAR (Feucht, J. et al. et al. Nat. Med. 25, 82-88 (2018)) driven by the EF1α promoter (Eyquem, J., Poirot, L., Galetto, R., Scharenberg, A. M. & Smith, J. Biotechnol. Bioeng. (2013)) flanked by homology arms initially for GSHs-1, 2 and 3 (FIGS. 6 and 7). CAR targeting to the TRAC locus served as control and gold standard (Eyquem, J. et al. Nature 543, 113-117 (2017)). Expression of CAR was highest at GSH-1 among the GSHs, followed by GSH-2 and GSH-3, but lower than at TRAC (FIG. 26C). Commensurate with the CAR expression, GSH-1-CAR T cells displayed the highest cytolytic activity against CD19+ NALM6 leukemia cells equivalent to TRAC-CAR T cells, while GSH-2 and GSH-3 CAR-T cells showed reduced killing (FIG. 21E). To further analyze the functional capacity of the CAR-T cells and the effect of CAR expression on T-cell function, the proliferation of the CAR-T cells was measured over two weeks upon repeated encounter with antigen and examined cell-surface CAR expression at regular time intervals during this antigenic stimulation (FIGS. 22A and 22B). An upregulation of CAR expression was observed on the surface of GSH-1 CAR-T cells after the first exposure to antigen, reaching similar levels to CAR expression in TRAC targeted cells while the expression at GSH-2 and GSH-3 remained unchanged. This similarity in CAR expression levels at TRAC and GSH-1 upon antigen exposure explains the comparable cytolytic activity of these CAR-T cells (FIG. 21E). Upon the second exposure however, GSH-1 CAR-T cells failed to show the same level of CAR upregulation. This indicated that CAR expression silenced over time at all three GSHs, more rapidly at GSH-2 and 3 and gradually at GSH-1. The proliferation capacity of the GSH-CAR-T cells was lower in comparison to TRAC-CAR-T cells and was proportional to the CAR expression levels during the first week after transduction (FIG. 22C).

A chromatin insulator element with barrier function may limit heterochromatinization and thus sustain CAR expression (FIG. 22D). The CAR transcription unit was flanked at GSH-1 with the C1 insulator (Liu, M. et al. Nat. Biotechnol. 33, 1-21 (2015)) and 3 additional GSHs were tested (4-6, FIG. 21A). Initial CAR expression levels varied between the sites, with GSH6 yielding expression most similar to TRAC-encoded CAR, both with and without the insulator (FIGS. 22E and 22F).

CAR-T cells with uninsulated CAR at GSHs 1, 2 and 3, along with GSH-4, showed a poor proliferation over 3 weeks as compared to TRAC-CAR-T cells, but at GSHs 5 and 6, the proliferation capacity of CAR-T cells was similar to TRAC-CAR-T cells (FIG. 22D). The incorporation of insulator C1 at GSH-1 greatly improved the proliferation capacity by the end of week 3, and to a lesser extent at GSH-4 (FIG. 22F). On the other hand, at GSHs 5 and 6, the insulator negatively impacted expansion potential, with a greater decrease in proliferation capacity for GSH-5 than GSH-6.

To directly assess the long-term cytolytic activity of the CAR-T cells upon antigen exposure, which would be most similar to activity observed in an in vivo setting, cytotoxicity assays were performed at the end of three weeks of antigenic stimulation in addition to before antigen exposure (FIGS. 22G, 22H and FIG. 26G). Before antigen exposure, uninsulated GSH-CAR-T cells could be seen to fall into three categories of activity, highest activity equivalent to TRAC-CAR-T cells with GSH-5 and GSH-6 CAR-T cells, moderate activity with GSH-1 and GSH-2 CAR-T cells and lowest activity with GSH-3 and GSH-4 CAR-T cells. The presence of the insulator improved the activity of GSH-4 CAR while activity of GSH-1, GSH-5 and GSH-6 CAR was unaffected. However, after 3 weeks of antigen exposure, uninsulated GSH-6 CAR-T cells alone maintained cytolytic activity while all other GSH-CAR-T cells had a reduced cytolytic activity with, as well as, without the insulator. Similar to the proliferation assay, incorporation of insulator C1 negatively impacted the activity of GSH-5 and GSH-6 CARs while it helped improve the activity of GSH-4 CAR. The activity of GSH-1 CAR, on the other hand was unaffected. This pattern of activity between the CARs correlated well with the CAR expression levels before and after every antigen exposure where GSH-6 alone showed sustained CAR expression levels over time.

For further functional testing in vivo, GSH 1, 4 and 6 CAR were then selected with and without insulator along with the TRAC-CAR as control since these constructs represented the three categories of intermediate, low and high functional capacity respectively, based on the cytotoxicity assay in FIG. 22G.

In Vivo Assessment of GSH-CAR T Cells

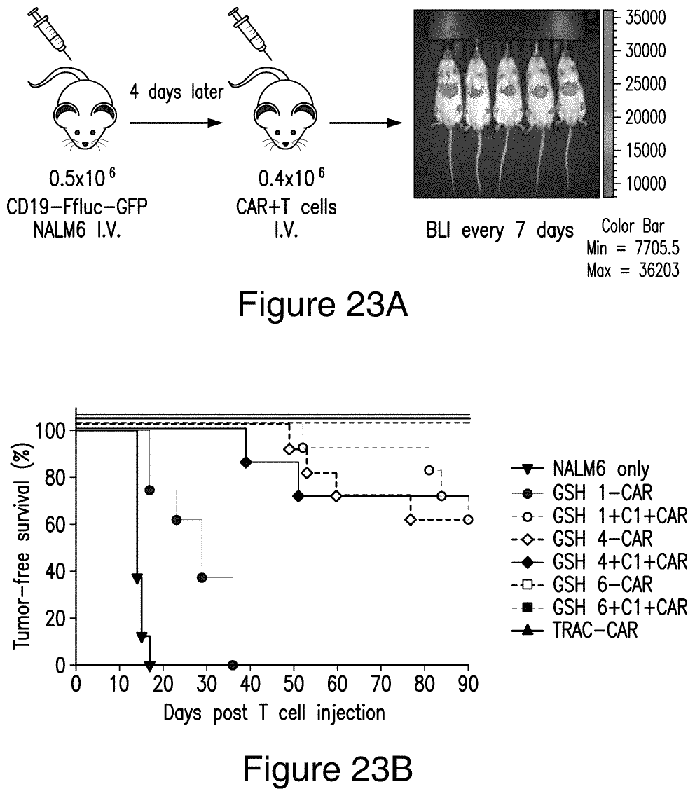

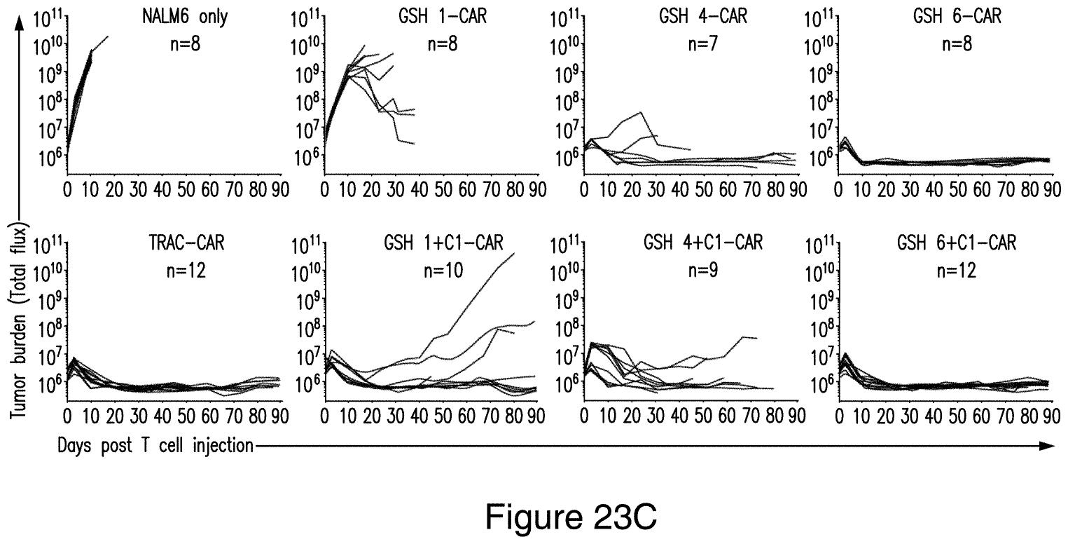

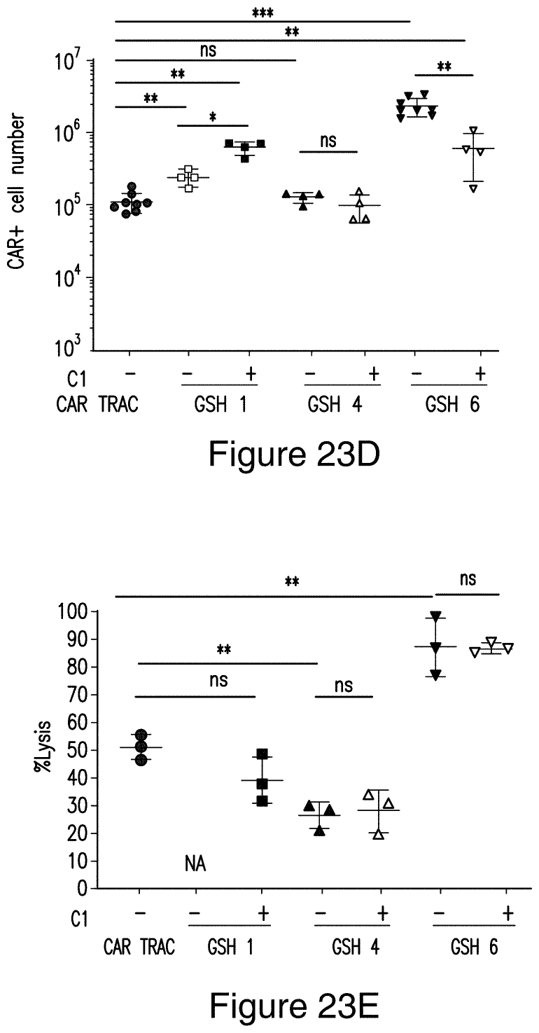

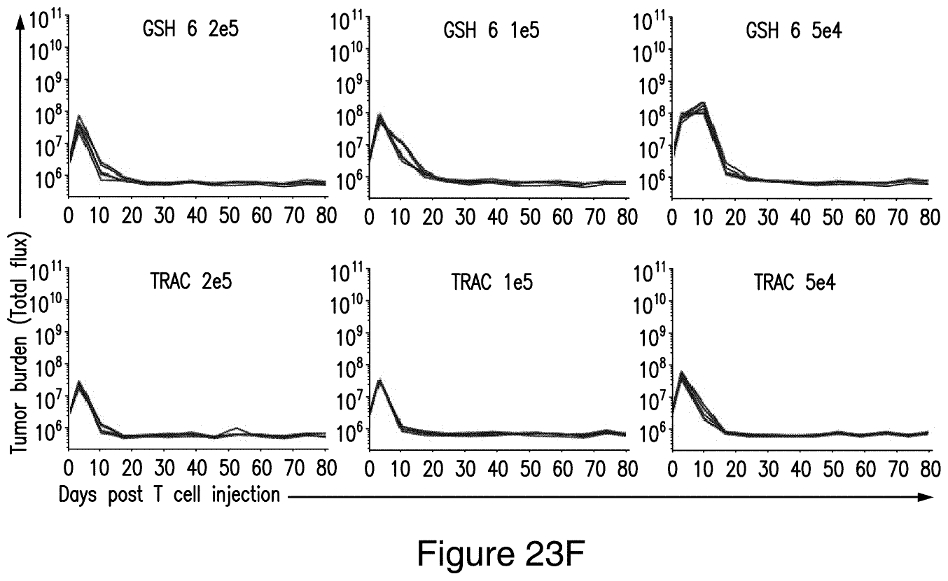

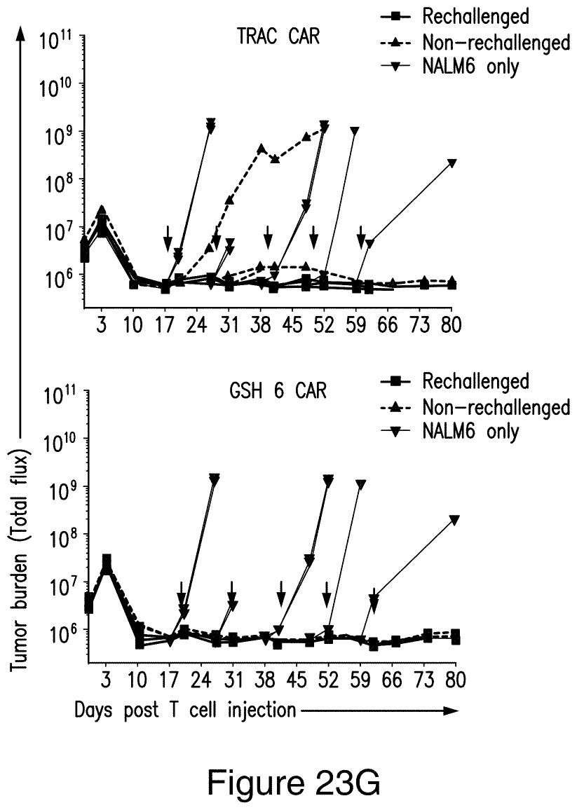

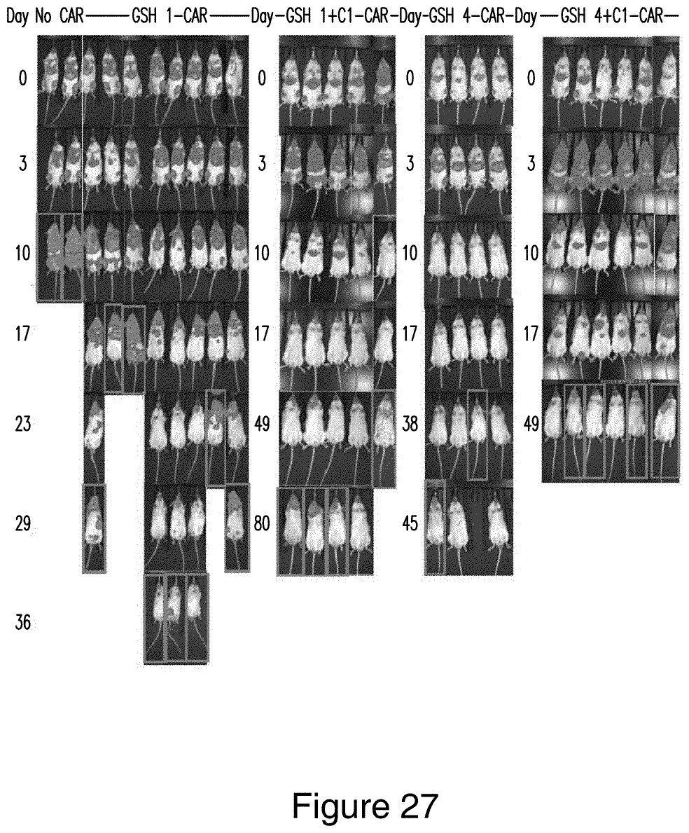

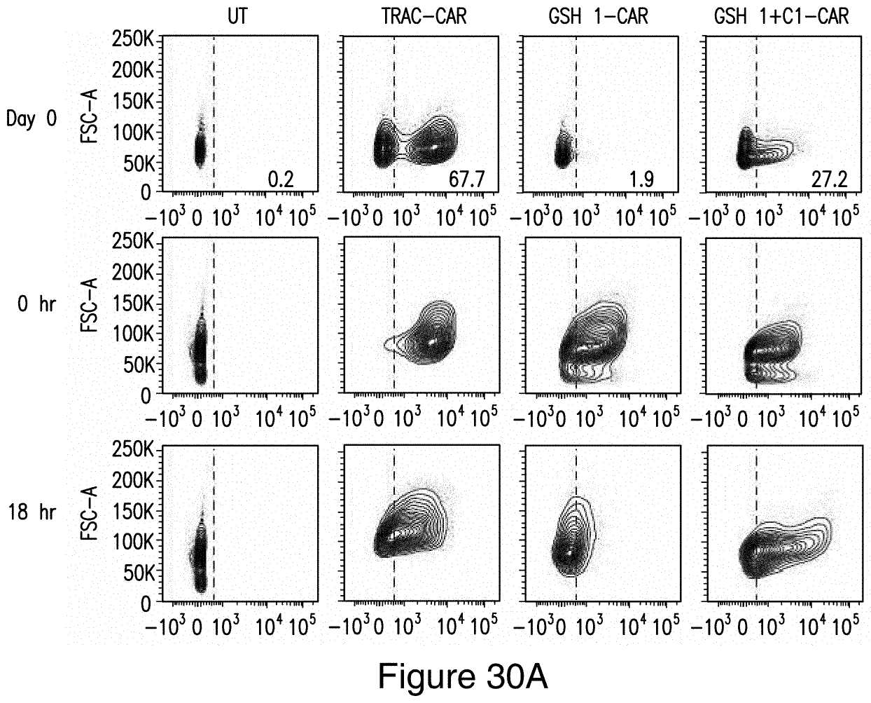

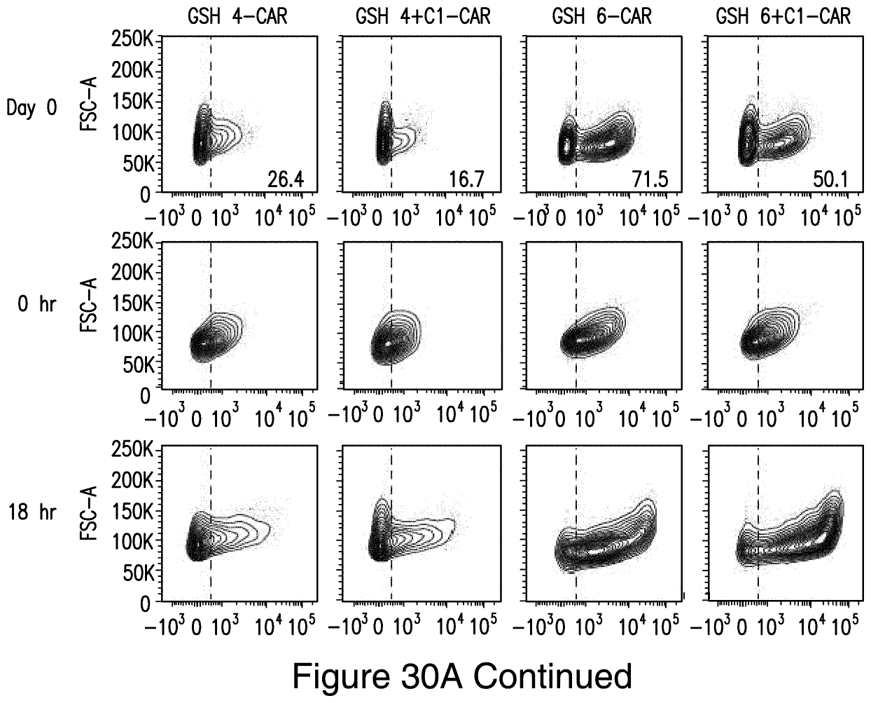

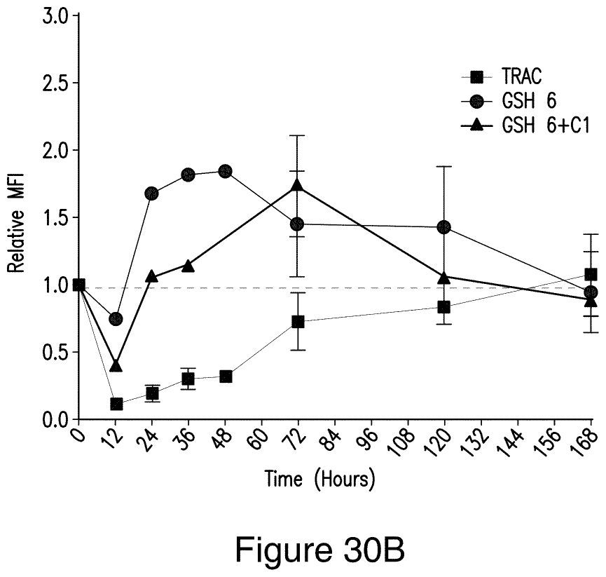

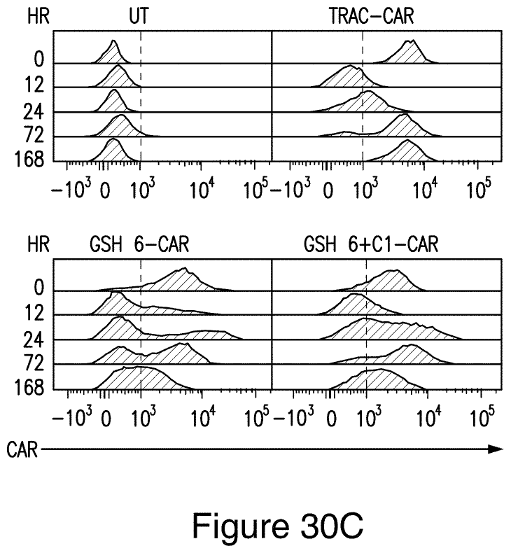

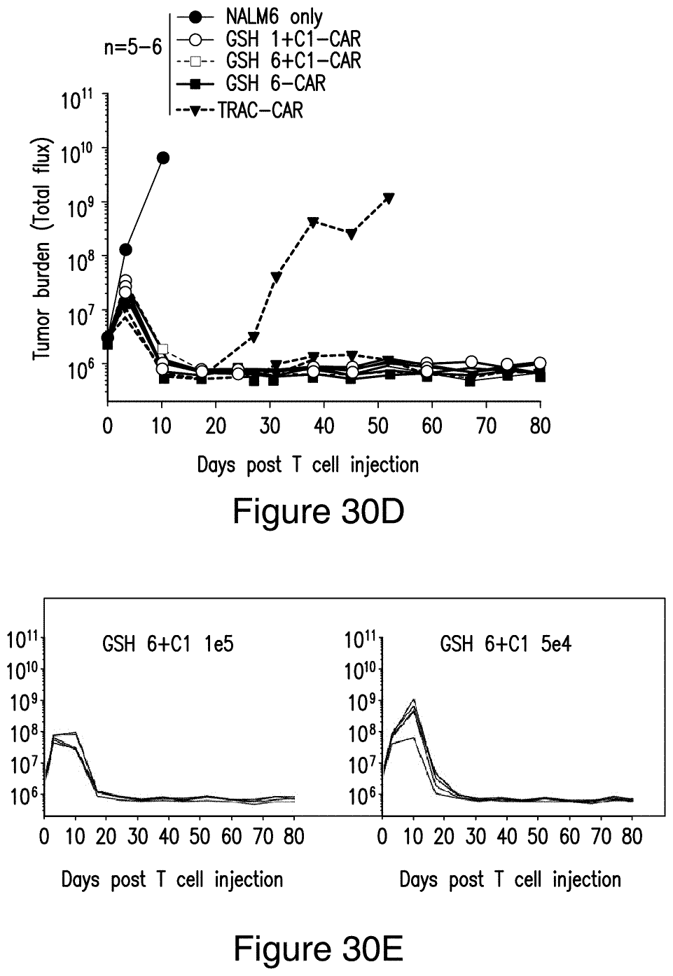

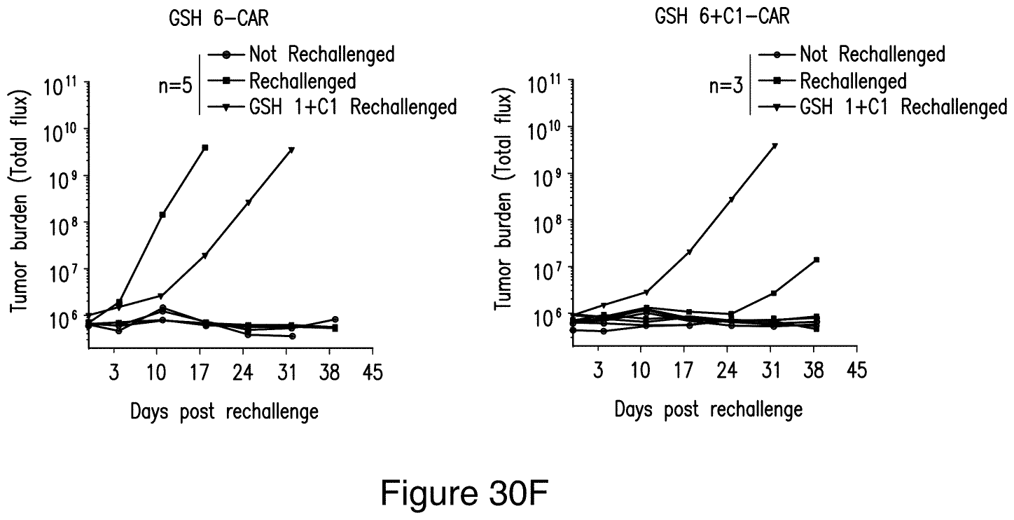

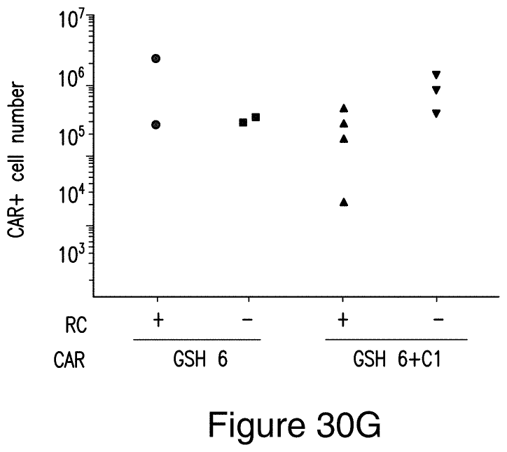

GSH-CAR T cells were assessed in vivo in a pre-B acute lymphoblastic leukemia (B-ALL) NALM-6 mouse model using the ‘CAR stress test’ (Zhao, Z. et al. Cancer Cell 28, 415-428 (2015)), wherein CAR T-cell dosage is lowered to reveal the functional limits of the CAR T-cells (FIG. 23A). GSH-6 CAR-T cells both with and without the insulator C1 were observed to display a similar functional potency as TRAC-CAR-T cells and were able to induce long-term tumor control (FIGS. 23B-23C). On the other hand, although GSH-1+C1 and 4±C1 CAR-T cells were initially able to control tumor burden, relapse was seen to occur at a later time point, suggesting T cell dysfunction or poor persistence (FIGS. 23B-23C). Interestingly, the relapses that occurred with GSH-1+C1 and GSH-4±C1 CAR-T cells were not in the bone marrow but at distant sites like brain, lung etc. and hence a lower tumor burden resulted in death (FIG. 27). GSH-1-CAR-T cells could control the tumor in the bone marrow to some extent in most mice but failed at controlling brain metastasis. Similar to the observed benefit of the insulator at GSH-1 seen in in vitro assays, GSH-1+C1-CAR-T cells seemed to have a greatly improved functional capacity in vivo (FIGS. 23B-23C and FIG. 30D). GSH-4 on the other hand, did not seem to benefit from the incorporation of the insulator which was consistent with in vitro findings that show no difference in CAR expression.

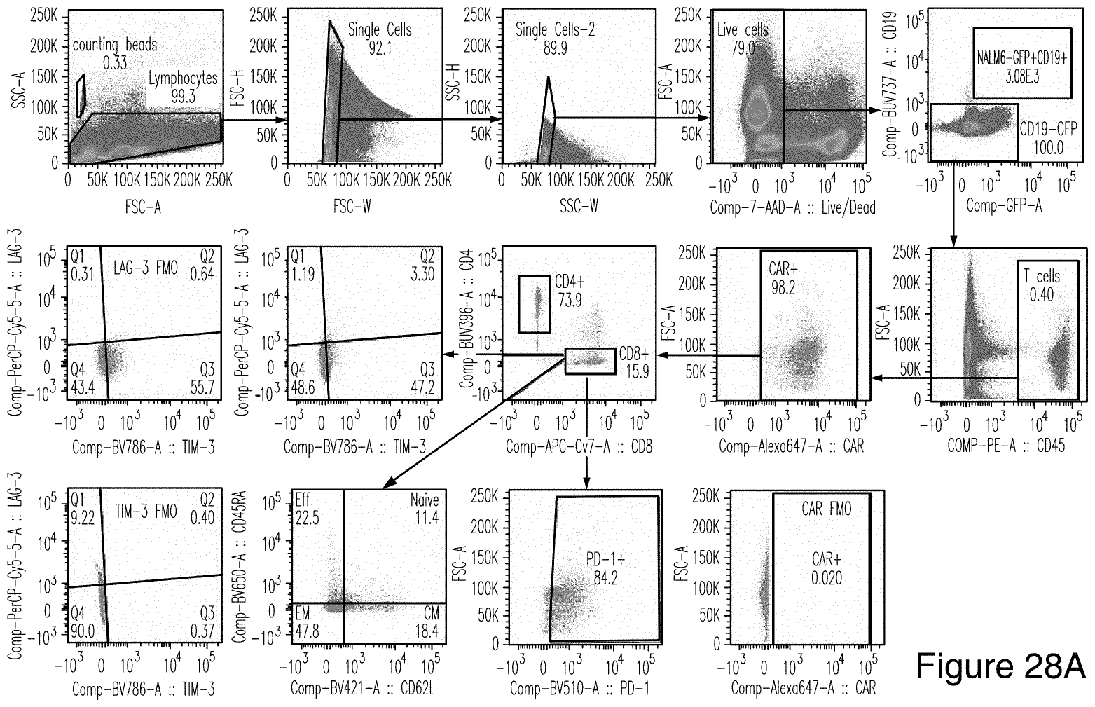

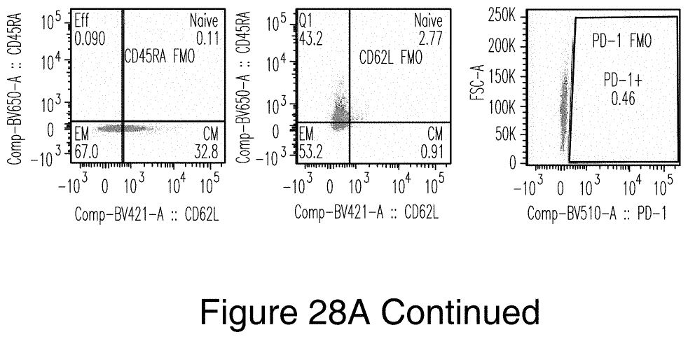

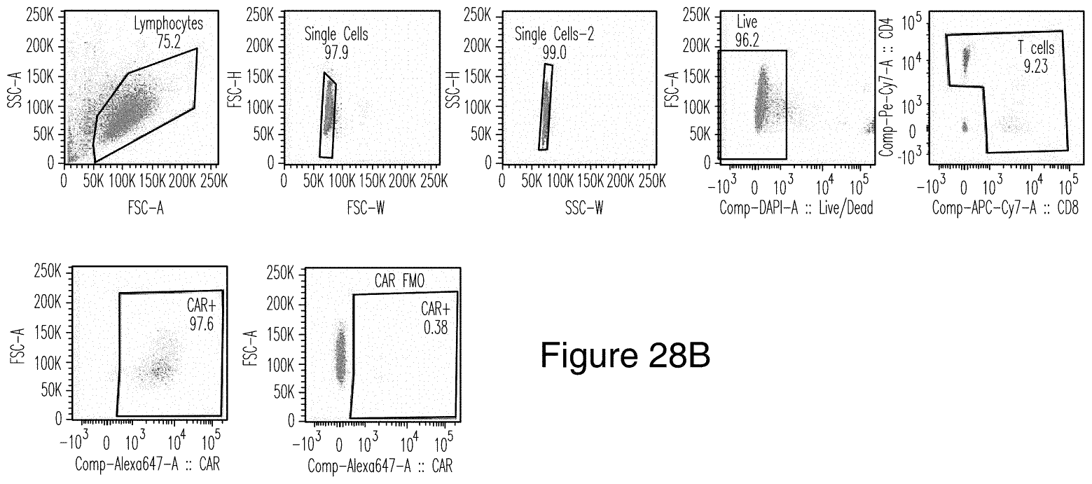

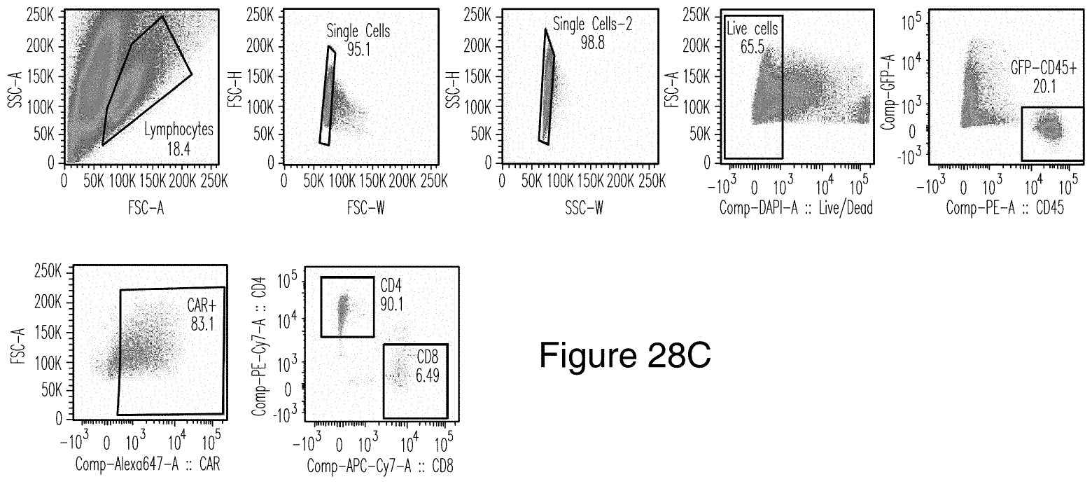

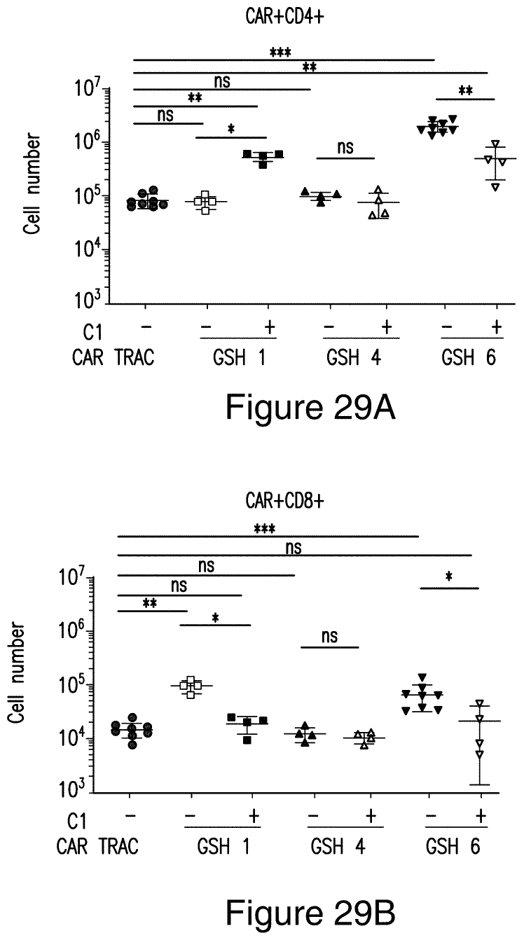

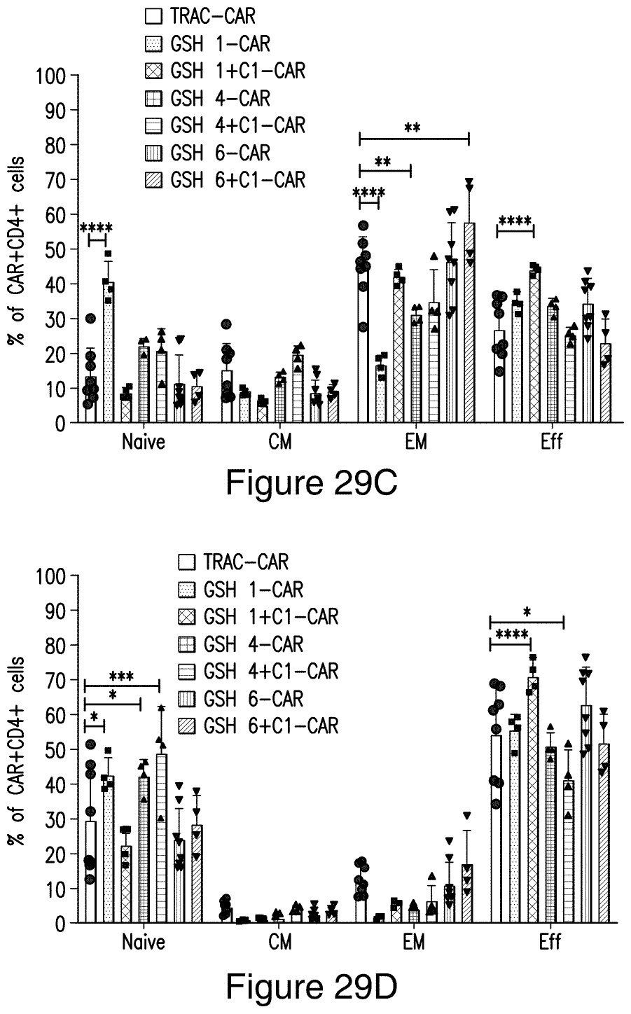

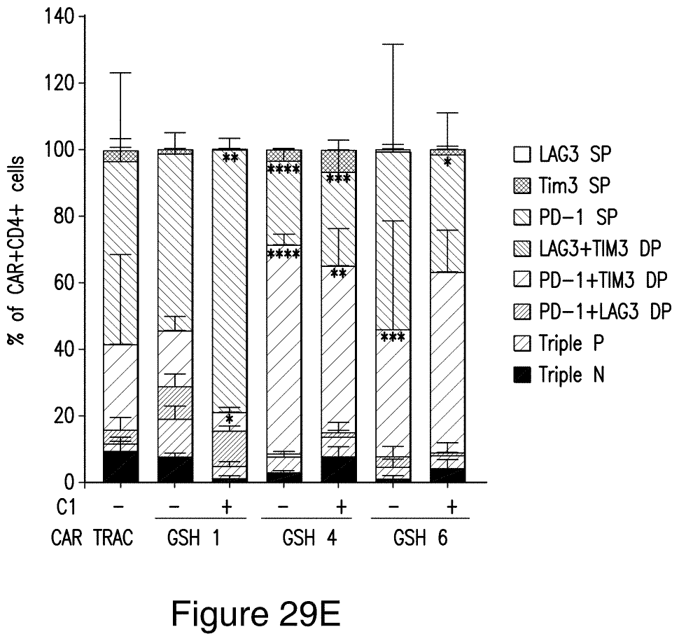

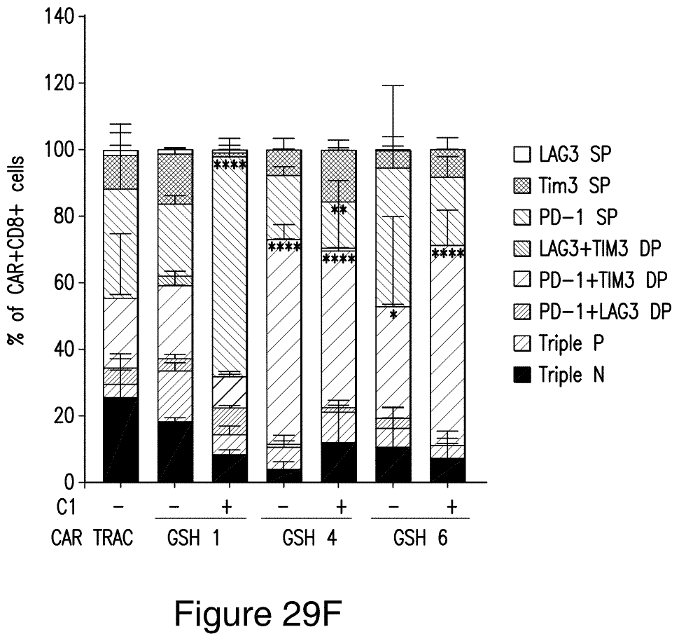

In order to study the properties of these CAR-T cells which afforded differential functional capabilities, CAR-T cells were isolated from the bone marrow of the mice at day 10 after T cell injection and analyzed for their CAR T cell numbers, CAR expression, differentiation and exhaustion markers (FIGS. 28 and 29). The CAR T cell numbers within the different groups varied with GSH-6±C1 CARs showing significantly higher CAR T cell numbers as compared to all other constructs (FIG. 23D, FIGS. 29A, 29B). The presence of the insulator significantly improved CAR T cell numbers for GSH-1 CAR but did not impact GSH 4 CAR. With respect to the differentiation and exhaustion profile of the CAR-T cells, some differences could be seen between the groups but no distinct profile could be identified to explain the higher functional capacity of the TRAC and GSH-6±C1 CAR-T cells (FIGS. 29C-29F). The CAR expression on the cell surface of all GSH-CAR-T cells was much reduced in comparison to the CAR expression levels before injection in mice (FIG. 30A). To investigate whether re-exposure to antigen increases CAR expression and affords differential cytolytic activity, these bone marrow derived CAR T cells were co-cultured with CD19+ NALM6 leukemia cells and analyzed for CAR expression and cytolytic activity. As expected from their superior anti-tumor activity, GSH-6 CAR-T cells, both with and without the insulator were able to upregulate CAR expression on the surface and lyse the NALM6 cells most effectively (FIG. 23E, FIG. 30A). GSH-1+C1, GSH-4 and GSH-4+C1 CAR-T cells were also able to upregulate CAR expression, but to a much lesser extent as reflected in their reduced lysis capacity.

GSH6 Supports Long-Term Tumor Control

The activation-induced increase in expression of the CAR at GSH-6 was deduced to be responsible for the enhanced functional capacity of the GSH-6 CAR-T cells along with the higher proliferation of the cells in vivo contributing to their high cell numbers. The finding that CAR expression was upregulated upon antigen exposure at GSH-6 prompted us to perform a careful time course of CAR expression following in vitro exposure to antigen. CAR constructs both with and without the insulator C1 at GSH-6 downregulated CAR expression immediately following antigen exposure until about 12 hours and then upregulated it by approximately 1.75-fold afterwards until about 72 hours and subsequently returned to its baseline until re-exposure to antigen (FIGS. 30B, 30C).

The efficacy of GSH-6 CARs at even lower T cell doses of 2×10⁵, 1×10⁵ and 5×10⁴ was evaluated. GSH-6±C1 CAR-T cells could effectively control tumor burden with all T cell doses equally well with the only difference being the time taken to achieve complete tumor eradication (FIG. 23F, FIG. 30E). The latency time for the GSH-6 CAR-T cells at doses of 1×10⁵ and 5×10⁴ was longer as compared to the TRAC-CAR and a higher peak tumor burden was reached before the tumor was completely eradicated. Additionally, to test whether GSH-6 CAR-T cells remained functional even upon multiple re-challenges with tumor cells as achieved with the TRAC-CAR-T cells, re-challenged mice were treated with 4×10⁵ GSH-6 CAR-T cells multiple times with CD19+-NALM6 cells at an interval of 10 days between each re-challenge. GSH-6 CAR-T cells were able to completely control the tumor after every re-challenge, similar to TRAC-CAR-T cells (FIG. 23G).

To assess whether GSH-6 CAR-T cells could control tumor burden long after the initial tumor eradication, the long-term surviving mice were re-challenged (from FIG. 23C) with NALM6-CD19 at day 120 post T cell injection. One tumor free GSH 1+C1 mouse surviving at day 120 was also re-challenged similarly. As expected, the GSH 1+C1-CARs were ineffective in controlling tumor burden in this mouse. ⅓ and ⅕ of re-challenged uninsulated and insulated GSH 6 CAR bearing mice relapsed with tumor. All other re-challenged and control non-re-challenged mice (n=3 for GSH 6 and n=5 for GSH 6+C1) remained tumor free (FIG. 30F). Upon analysis of the CAR-T cells in the bone marrow of the mice 40 days post re-challenge, CAR-T cells were found at high numbers in the tumor-free mice confirming long-term persistence of the adoptively transferred CARs (FIG. 30G). This data correlated well with the sustained cytolytic activity of these cells (FIG. 22H) and displayed the functional capacity of GSH 6 CARs even at these late time points.

Characterization of GSHs and Association with Function

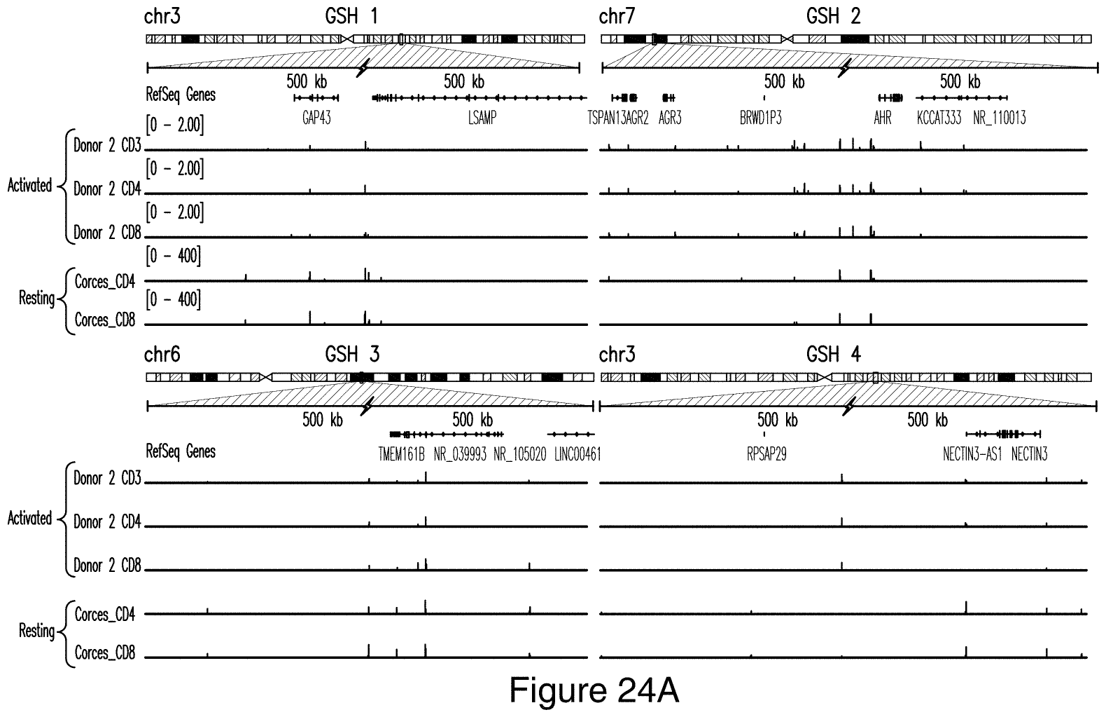

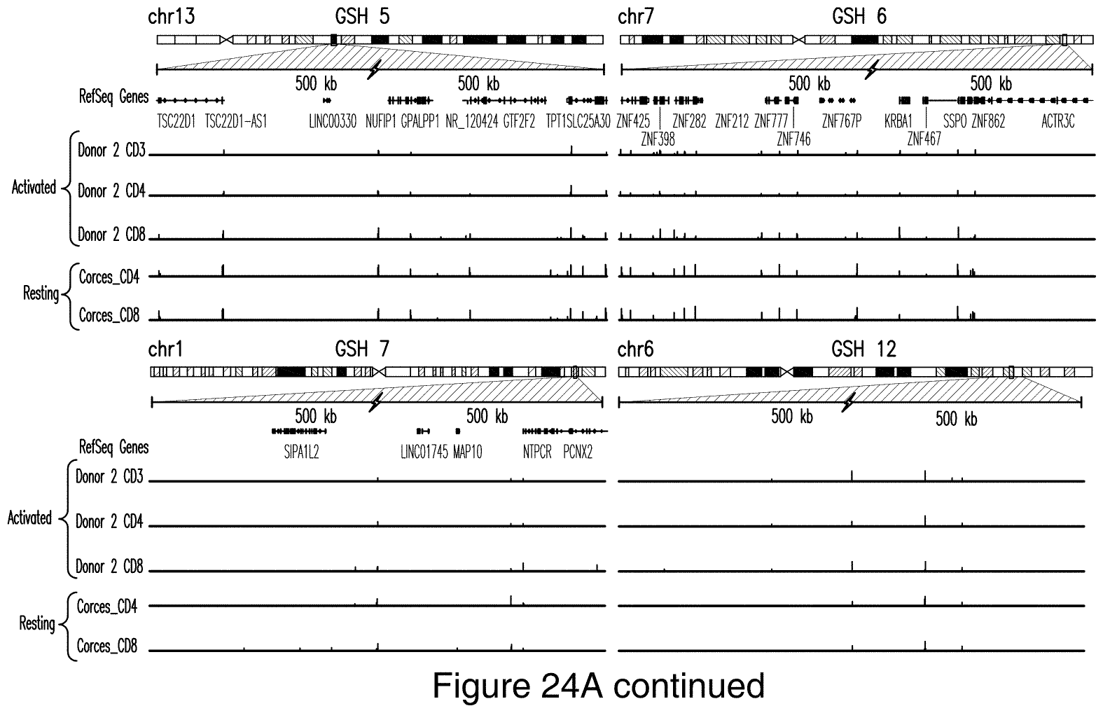

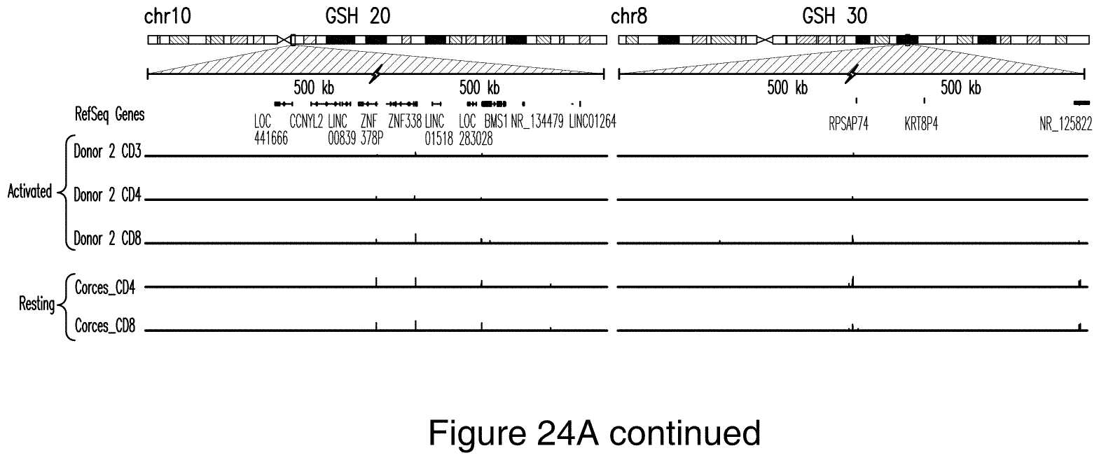

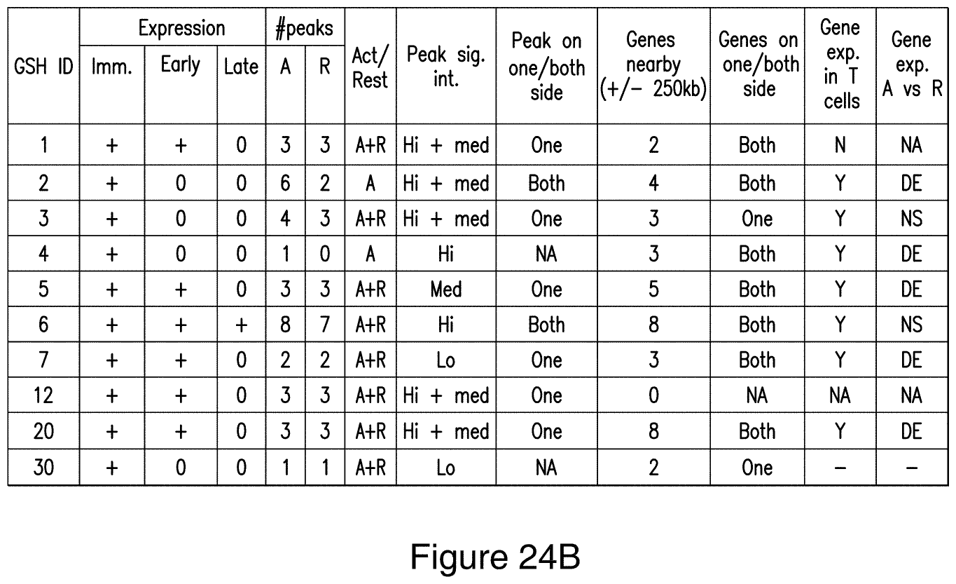

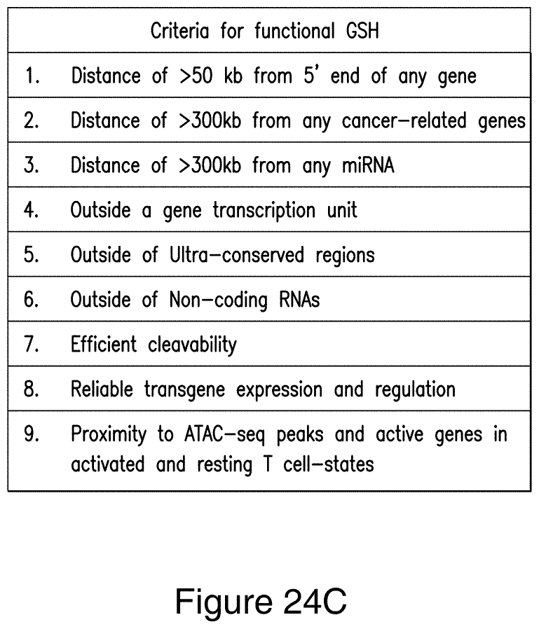

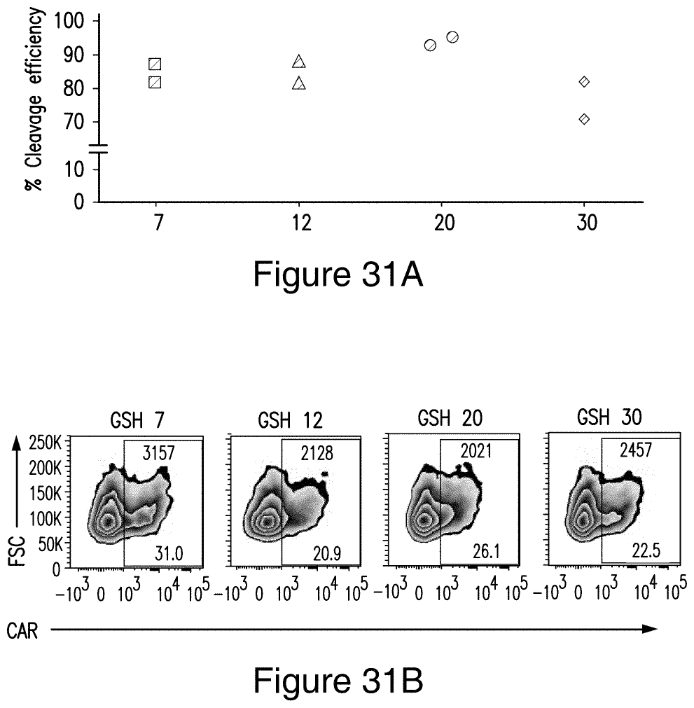

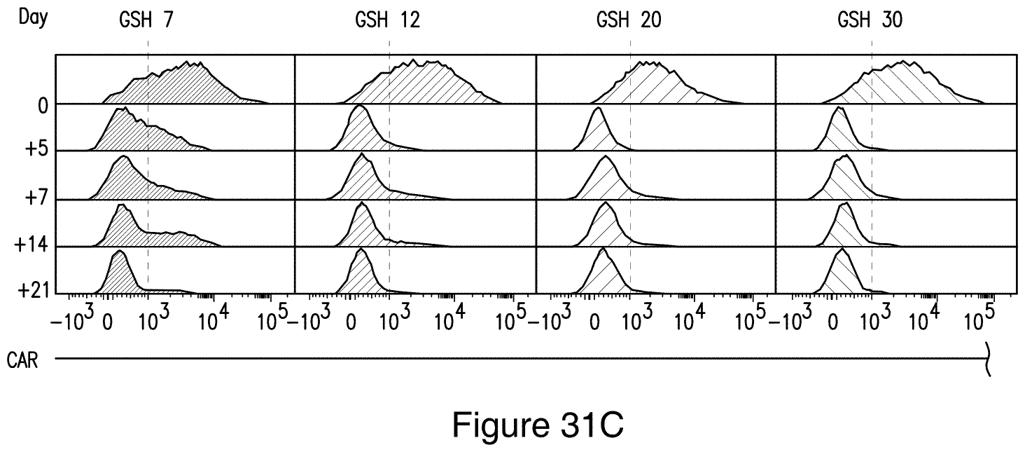

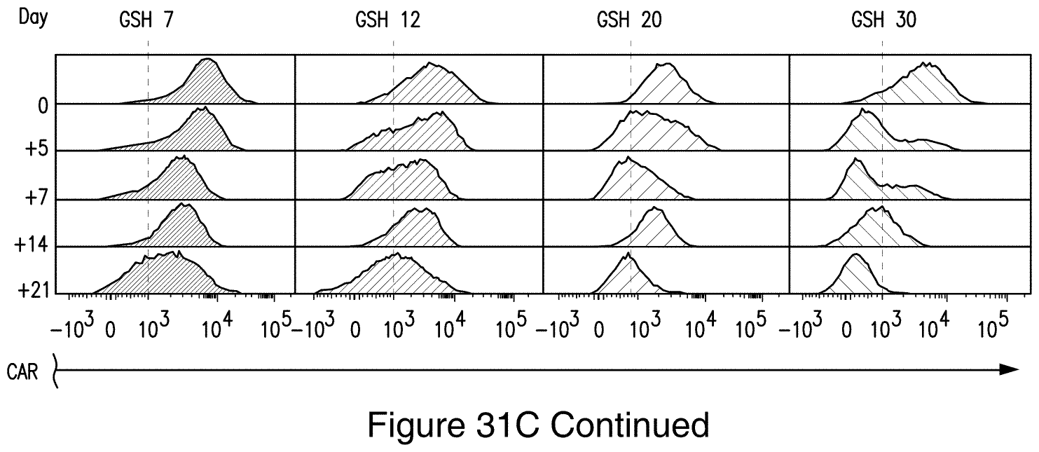

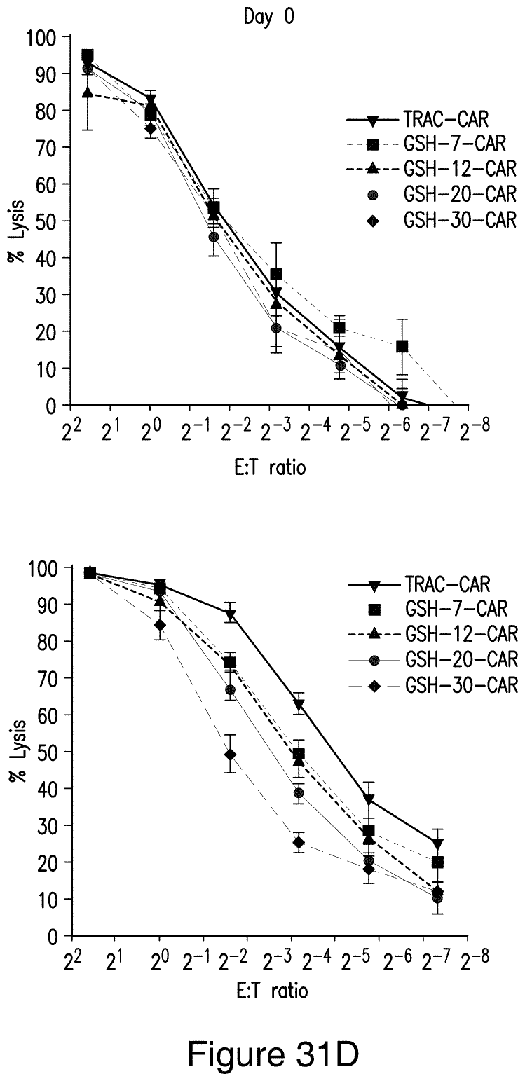

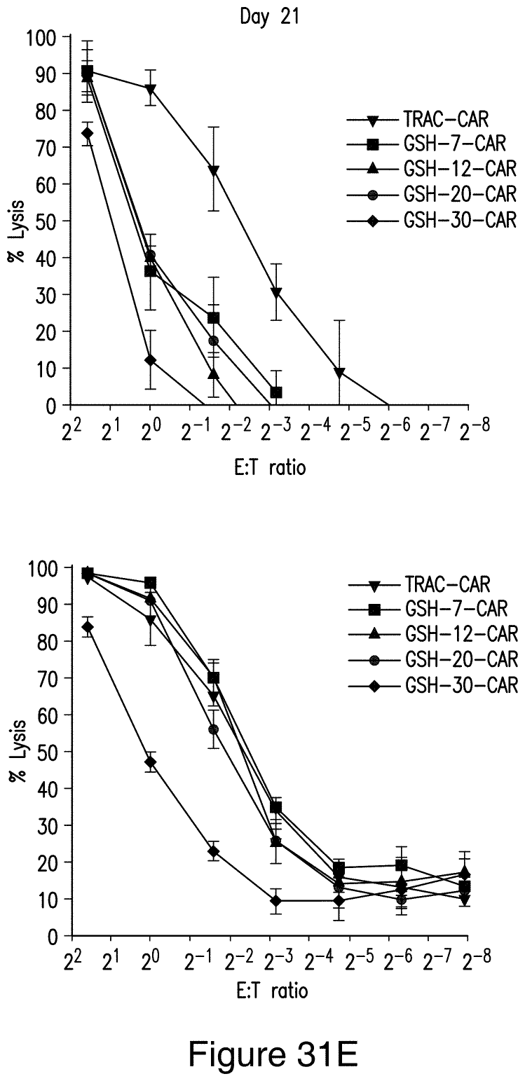

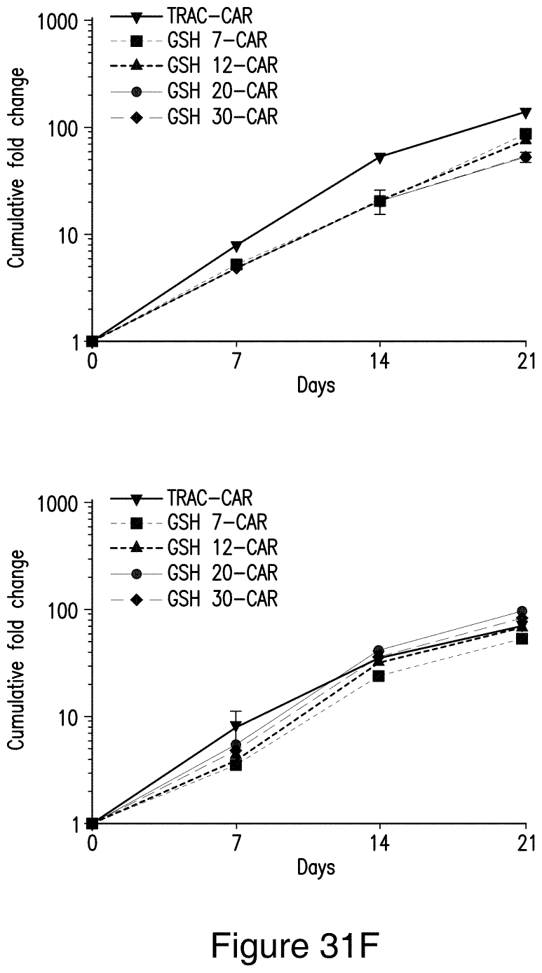

Given the widely different functional capacity of the CAR when integrated at different GSHs, it was sought to further understand the characteristics of a GSH with respect to its surrounding chromatin environment that dictate its functionality in the context of a T cell. This would help identify better functioning GSHs and these characteristics could then be integrated as part of the initial screening for GSHs. The reason for failure for most GSHs was inability or limited ability of expression upon activation which pointed to the inability of the locus to be held open in the resting state. Hence, the ATAC-seq data were analyzed at and around each of the six GSHs closely in activated and resting T cells. The activated T cell data used was the ATAC-seq data that were generated while the resting state data was obtained from Corces et al. (Corces, M. R. et al. Nat. Genet. 48, 1193-1203 (2016)). The expression of genes surrounding each of these sites in the resting and activated states was also studied. FIGS. 24A and 24B illustrate this information for all six GSHs. The best site associated with the best CAR-T cell function, GSH-6, was located within a pseudogene. To test whether the presence of the pseudogene alone granted better functionality to the GSH, 4 additional sites were tested where two of them (GSH 20 and 30) were located within a pseudogene or had a pseudogene very close to the site. The other two sites (GSH 7 and 12) were similar to intermediate (GSH 1-like) and poor performing (GSH 4-like) GSHs respectively in terms of presence of genes and ATAC-seq peaks around the sites (FIG. 24A). All 4 sites had a high intensity ATAC-seq peak and hence having a high cleavage efficiency. The cleavage efficiency, CAR integration, proliferation, expression and cytotoxicity at all these GSHs (FIG. 31) were tested. All four GSHs showed high cleavage efficiencies with two gRNAs targeted at the summit of the peak and moderate initial CAR expression levels. Surprisingly, GSH 20 which seemed most similar to GSH 6 in terms of presence of genes around the site failed to perform as well as GSH 6 over the course of the multiple stimulations. The CTLs performed at day 0 and day 21 indicated that GSHs 7, 12 and 20 seemed to be in the intermediate performing GSH group whereas GSH 30 was the poorest performing GSH, similar to GSHs 2, 3 and 4 (FIG. 31). This data indicated that the presence of a pseudogene at the site alone is not enough to grant better functionality to the GSHs. A closer examination of all GSHs, taking all data into consideration including the number of ATAC-seq peaks and genes within 250 kb around the site, respective gene expression and ATAC-seq peak signal intensity in the activated vs resting state (Tabulated in FIG. 24B) indicated that the following factors are responsible for deciding the activity of a particular GSH: 1) Proximity of peaks on either side of targeted peak 2) Intensity of proximal peaks 3) Presence of proximal and targeted peak in resting as well as activated state and 4) Presence of and expression of surrounding genes in resting and activated state. GSH 6 is characterized by the presence of high intensity ATAC-seq peaks as well as active genes in its proximity in the resting as well as activated state. These characteristics thus most likely influence the superior activity of GSH 6 over all other GSHs tested.

A number of future advances in human cell engineering based on gene addition depends on identifying safe genomic sites that afford dependable transgene expression. To achieve this goal, one may elect to target specific loci that provide desirable transgene regulation, e.g. the TRAC locus to express CARs (Eyquem, J. et al. Nature 543, 113-117 (2017)), or extragenic sites, the targeting of which does not entail disrupting an endogenous gene or known regulatory elements and may eventually accommodate large inserts encoding multiple genes. Criteria were previously proposed for the identification of such sites (Table 5, criteria 1-5 and Irion, S. et al. Nat. Biotechnol. 25, 1477-1482 (2007)), based on extensive insertional mutagenesis data accumulated in a number of clinical trials utilizing γ-retroviral and lentiviral vectors (Ellis, J. Hum. Gene Ther. 16, 1241-1246 (2005); Dunbar, C. E. Ann. N. Y. Acad. Sci. 1044, 178-182 (2005); Stein, S. et al. Nat. Med. 16, 198-204 (2010); Kohn, D. B., Sadelain, M. & Glorioso, J. C. Nat. Rev. Cancer 3, 477-488 (2003); Hacein-Bey-Abina, S. et al. J Clin Invest 118, 3132-3142 (2008); Howe, S. J. et al. J. Clin. Invest. 118, 3143-50 (2008)) and were utilized to retrospectively identify safe random integrations in clonal populations (Papapetrou, E. P. et al. Nat. Biotechnol. 29, 73-78 (2011)). Adding criteria for exclusion of non-coding RNAs, nuclease accessibility and chromatin context (criteria 7,8,9 FIG. 24C), demonstrating presently the feasibility of prospectively selected genomic regions that support therapeutic transgene expression of a CAR, matching the efficacy afforded by the reference TRAC locus.

To ensure highly efficient access to candidate GSH, a new criterion of chromatin accessibility was introduced. Cas9 would efficiently bind and cleave candidate GSH presenting with high chromatin accessibility (peak signal intensity) as assessed by ATAC-seq. It was indeed found that all 10 peaks meeting this criterion of high ATAC-seq peak signal intensity were efficiently cleaved at the center of the peak. At a distance from the peaks, accessibility was more variable, sometimes remaining high but markedly decreasing in other instances. Overlaying the safety criteria (1-6) with this one (7) reduced the number of candidate peaks in human primary T cells to 379.

The top 6 hits were further investigated for their ability to sustain transgene expression, quantifying CAR expression in vitro and evaluating their therapeutic potential in vivo in a well-established model of CD19+B-ALL. All 6 sites and another 4 that were evaluated were efficiently targeted and initially expressed the CAR, but only one maintained expression long-term. The other 9 sites eventually silenced, some within days and others after a few weeks. The incorporation of a chromatin insulator element with barrier function (Liu, M. et al. Nat. Biotechnol. 33, 1-21 (2015)) partially rescued CAR expression at one site but not the other two (FIGS. 22F, 22H and 23B). At GSH6, which allowed for sustained CAR expression without requiring an insulator element, flanking the CAR transcription unit with the barrier element in fact diminished CAR expression and CAR T cell therapeutic efficacy (FIGS. 22F-22H).

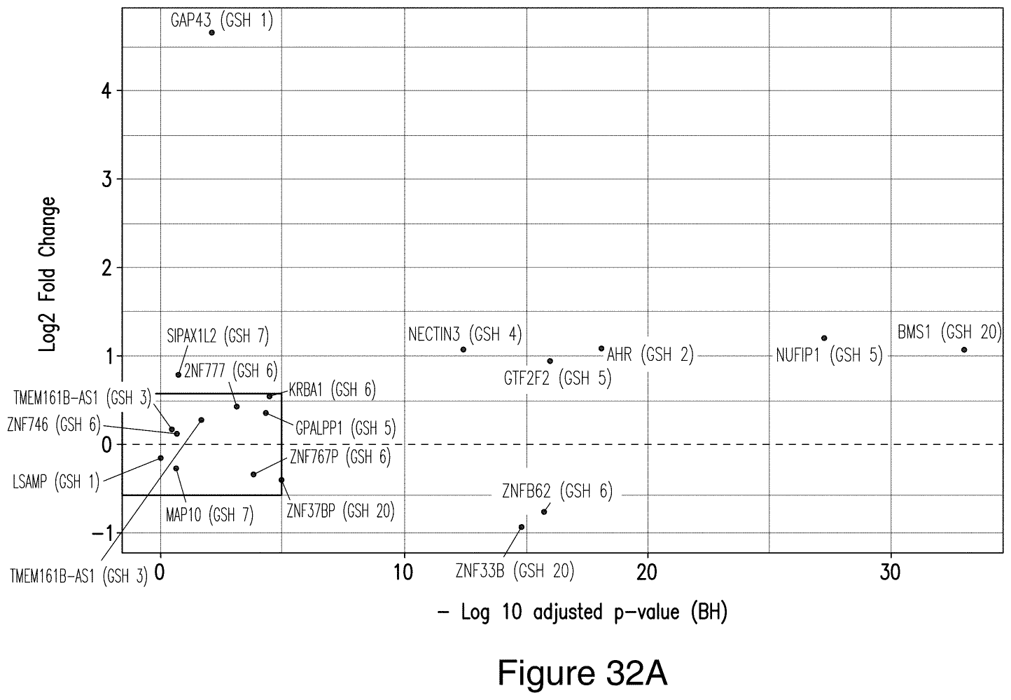

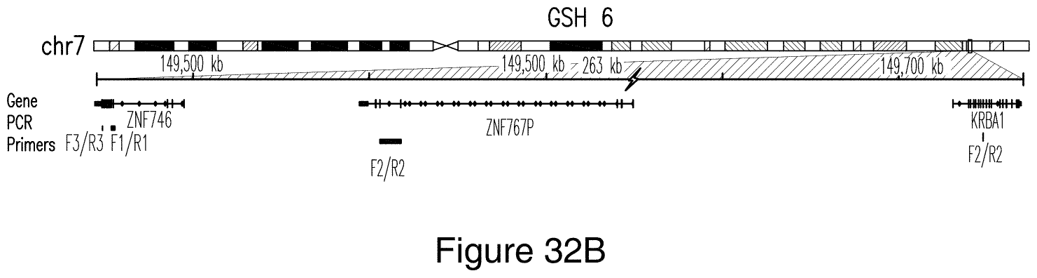

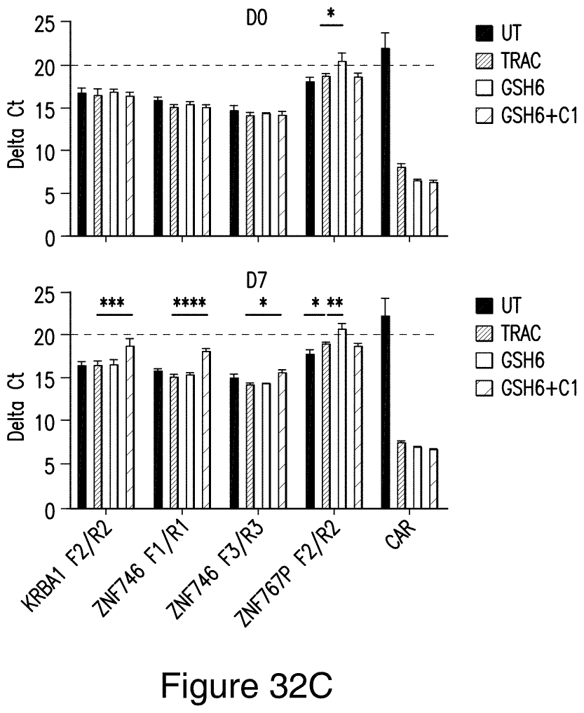

The presently disclosed subject matter highlighted the profound impact of integration site and variegated gene expression on CAR T cell function. Approaches based on the use of retroviral vectors (either γ-retroviral, lentiviral or spumaviral) and DNA transposons are all subject to such position effects (Rivella, S. & Sadelain, M. Semin. Hematol. 35, 112-125 (1998)). These approaches also expose to risk of gene disruption, as shown with CAR lentiviral vectors integrated at the TET2 or CBL-B loci (Shah, N. N. et al. Blood Adv. 3, 2317-2322 (2019); Fraietta, J. A. et al. Nature 558, 307-312 (2018)). As anticipated from the GSH criteria, integration of the CAR transcription unit at GSH6 including a strong enhancer/promoter did not perturb expression of the two endogenous genes within 150 kb on either side, neither in resting or activating T cells (FIG. 32B).

When expressed from GSH6, the CD19 CAR proved to be as effective as the TRAC-encoded CAR under stringent “stress test” conditions (Papapetrou, E. P. & Schambach, A. Mol. Ther. 24, 678-684 (2016); Feucht, J. et al. et al. Nat. Med. 25, 82-88 (2018)). Thus, following low-dose CAR T cell infusion, GSH6 CAR treated mice fared as well as TRAC-CAR treated mice and were protected from five consecutive re-challenges with tumor cells and even a late re-challenge 120 days after the initial, single CAR T cell infusion.

A close analysis of CAR expression, reflecting the function of the EF1α enhancer/promoter and the bovine growth hormone polyadenylation signal within the GSH6 chromosomal context, revealed the kinetics of CAR expression upon antigen-induced T cell activation. The CAR expression at GSH 6 without antigen exposure was downregulated, but upon antigen exposure, it peaked after the initial CAR internalization phase, eventually returning to baseline in a week (FIGS. 30B, 30C, 31D). This kinetics of CAR expression returning to baseline without overshooting is similar to the CAR expression pattern displayed by the endogenous TRAC promoter and unlike the pattern observed with viral promoters or the EF1α promoter at the TRAC locus (Eyquem, J. et al. Nature 543, 113-117 (2017)). While both TRAC-CAR and GSH6-CAR T cells performed similarly in the therapeutic model, they differ fundamentally in that TRAC-CAR T cells lack an endogenous TCR while GSH CAR retain it which may help with long-term persistence and can also be used to advantage for improving in vivo expansion of CARs as has been shown in recent studies (Ghosh, A. et al. Nat. Med. 23, 242-249 (2017); Stenger, D. et al. Blood (2020). doi:10.1182/blood.2020005185; Lapteva, N. et al. Cancer Res. 25, 7340-7350 (2019)). Other GSHs that show short-term activity could also have potential use, if the extinction of gene expression were desirable within a certain time frame. Thus, the GSHs disclosed here could be useful for the design of a variety of T cells encoding CARs, TCRs and a vast array of other immunotherapeutic molecules.

The ATAC-seq profile of the different GSHs provides some insights into what may constitute a more favorable site for sustained expression in T cells. The surrounding ATAC-seq peaks and gene expression profiles in resting and activated T cells differed slightly between the 10 GSHs where the CAR cDNA was integrated. Proximity to genes—while complying with the GSH criteria—that are active in both resting and activated T cell states and presence of ATAC-seq peaks in both states was observed at GSH6. These features were not all found at the other GSHs. These may thus represent a screening criterion to add to the presently disclosed GSH requirements for optimal T cell genome editing (FIG. 24C).

GSH 6 is located within a pseudogene, ZNF767P which is transcribed yet non-coding with no known function, but it is noteworthy that another GSH located within another pseudogene (ZNF37BP, GSH20), did not sustain CAR expression using the same expression elements (FIG. 31C).

### Methods

Generation of GSH Atlas.

The first six criteria for GSHs (Table 5) were applied to build a Genomic safe harbor atlas (GSH) atlas based on the Human GRCh37/hg19 assembly. Gene annotation information for criteria 1 and 4 were obtained from GENCODE version 25 and RefSeq_NM database from NCBI. Data for cancer-related genes were obtained by combining oncogene lists from Bushman group allOnco list (v2) (http://www.bushmanlab.org/links/genelists), COSMIC Cancer gene census v78 (https://cancer.sanger.ac.uk/cosmic) and CancerGeneticsWeb (http://www.cancer-genetics.org/). miRNA data was obtained from hg19 sno/miRNA track in UCSC Genome Browser and GENCODE entries for miRNAs. The data for UCRs in the human genome was obtained from http://users.soe.ucsc.edu/˜jill/ultra.html (Bejerano, G. et al. Science 304, 1321-1326 (2004)). As the genomic coordinates used in the publication were from an older assembly, the coordinates were converted to hg19 using UCSC lift genome annotations tool. Data for Non-coding RNA (ncRNA) list was obtained from NONCODE v5 (www.noncode.org) and GENCODE ncRNA entries. Pseudogene annotation from GENCODE was used to either include or exclude pseudogenes from the gene list to create two atlases—With pseudogenes and Without pseudogenes. The assembly gaps as mentioned on the UCSC Genome Browser for hg19 genome were excluded.

ATAC-Seq Atlas for Human T Cell Genome.

Peripheral blood mononuclear cells were obtained by density gradient centrifugation from peripheral blood of three healthy adult human volunteers. T cells were purified using the Pan T Cell Isolation Kit (Miltenyi Biotec) and stimulated with CD3/CD28 T cell Activator Dynabeads (Invitrogen) (1:1 beads:cell) and cultured in X-VIVO 15 Serum-free Hematopoietic Cell Medium (Lonza), supplemented with 5% human serum (Gemini Bio-Products) and 200 U ml⁻¹ IL-2 (Miltenyi Biotec). Cells were cultured at 10⁶ cells per ml. CD3/CD28 beads were magnetically removed 48 h after initiating T cell activation. At day 3 after isolation and activation, the T cells were sorted into CD4 and CD8 fractions from two donors by magnetic separation through negative selection using Human CD4-biotin and Human CD8-biotin beads (Miltenyi Biotec) and anti-biotin beads (Miltenyi Biotec). CD3, CD4 and CD8 cells from donors 2 and 3 and only CD3 cells from donor 1 were collected and 50,000 cells were frozen in freezing medium (10% DMSO in FBS) for ATAC-seq analysis. ATAC-seq was performed by the MSKCC IGO core. The method used for ATAC-seq was as described previously (Buenrostro, J. D., Wu, B., Chang, H. Y. & Greenleaf, W. J. Curr. Protoc. Mol. Biol. 109, 21.29.1-21.29.9 (2015)) but with the change that the transposition reaction was performed at 42° C. for 45 mins since this condition gave a better library prep. All ATAC libraries were sequenced using paired-end, dual-index sequencing on an Illumina HiSeq instrument with 2×50 bp reads for at least 30 million read pairs.

ATAC-Seq Data Processing.

Raw FASTQ reads were trimmed with Trimmomatic (Bolger, A. M., Lohse, M. & Usadel, B. Bioinformatics 30, 2114-2120 (2014)) and aligned to hg19 using Bowtie2 (Langmead, B. & Salzberg, S. L. Nat. Methods 9, 357-359 (2012)). Bam files were filtered based on map quality and PE concordance, duplicated reads were removed and tn5 specific read shift was performed. To call peaks, data were aggregated by cell type and peak calling was performed using MACS2 (Zhang, Y. et al. Genome Biol. 9, R137 (2008)) and filtered using ENCODE hg19 blacklist (Amemiya, H. M., Kundaje, A. & Boyle, A. P. Sci. Rep. 9, 9354 (2019)). Irreproducible discovery rate (IDR) analysis was performed for all replicate pairs. Peaks with global IDR <0.05 were considered as reproducible peaks. The ATAC-seq data from the Corces et al. study available publicly was also processed similarly, visualized using the IGV genome browser by setting to the same signal range to view all of the GSH regions in FIG. 24.

Identification of Candidate GSHs.

The Genomic Safe Harbor atlas (without pseudogenes) and the ATAC-seq atlas were overlaid to find GSHs associated with an ATAC-seq peak. 21,566 ATAC-seq peaks that are shared across all samples were overlapped with GSH atlas to identify 379 ATAC-seq peaks that had a GSH within 5 kb. These ATAC-seq peaks were termed as GSH peaks and were then ranked by the average signal intensity (RPM) at the summit to identify candidate GSHs for further testing.

Guide RNA (gRNA) Design and Testing.

Coordinates for each site for gRNA design were input into the Guidescan online software tool (http://www.guidescan.com/) and the gRNAs that had the best cleavage efficiency score and off-target profile (Doench score>50 and specificity score>0.2) were chosen. Sequences of all gRNAs targets used are in Table 6.

TABLE 6
Sequences of all gRNAs. In red: gRNA used for targeted integration

| | | | |
|---|---|---|---|
| gRNA name | gRNA sequence | Associated Fig. | SEQ ID NO. |

gRNAs at peak centre

| | | | |
|---|---|---|---|
| 1-1-1/1.1 | CCCGGTGTCACCCGTCACCG | FIGS. 21D, FIG. 21E | 96 |
| 1-1-2 | CGCGTCCCAGGCGAGCACGG | FIG. 21D | 97 |
| 1-2-1 | TGACGGCAGGGTGAACTCGG | FIG. 21D | 98 |
| 1-2-2 | GCAGGGTGAACTCGGCGGCA | FIG. 21D | 99 |
| 2-1-1 | GCCTACTTCCTACCACACGA | FIG. 21D | 100 |
| 2-1-2 | TTTTATAGCCGTCGTGTGGT | FIG. 21D | 101 |
| 2-2-1 | GAGTCAAGGTATTGGCAATG | FIG. 21D | 102 |
| 2-2-2 | AGATAAGTGAGTCAAGGTAT | FIG. 21D | 103 |
| 3-1 | CATTCGACTAAAGAAAGCCG | FIG. 21D | 104 |
| 3-2 | GCCCCTGAATGGTTTAACGG | FIG. 21D | 105 |
| 3-3 | TCGACTAAAGAAAGCCGGGG | FIG. 21D | 106 |
| 3-4 | TATGCAGCCTGTCCGCAAGG | FIG. 21D | 107 |
| 4-1 | GTTAGACAGATACTTCTAAG | FIG. 21D | 108 |
| 4-2 | TAGCTAAAACAAGCAGCTTG | FIG. 21D | 109 |
| 4-3 | GAATTTGCTCAACCGCCTGT | FIG. 21D | 110 |
| 4-4 | GGAATTTGCTCAACCGCCTG | FIG. 21D | 111 |
| 5-1/5.1 | AATTCCATTTCAAAGCACGA | FIG. 21D, FIG. 26A | 112 |
| 5-2 | GAACCAAGGAGCTACCCCTG | FIG. 21D | 113 |
| 5-3 | GATGAACGCGATGGAGTGGG | FIG. 21D | 114 |
| 5-4 | ACGCCTACGGATGAACGCGA | FIG. 21D | 115 |
| 6-1 | CTGTATCCTGGGAGCCGGCG | FIG. 21D | 116 |
| 6-2 | TAAAGGCGTTGACGCAACTG | FIG. 21D | 117 |
| 6-3 | GCGGCCGACGAAGGCACATG | FIG. 21D | 118 |
| 6-4 | GGATACAGTGCGGACCGGCC | FIG. 21D | 119 |
| 7-1 | CGTTGCGAAGGTTCGTGTCG | FIG. 26A | 120 |
| 7-2 | ACCTTCGCAACGCCGTCCGC | FIG. 31A | 121 |
| 12-1 | CCACGACATGAATCATCACA | FIG. 31A | 122 |
| 12-2 | CATGTGATGATTCATGTCGT | FIG. 31A | 123 |
| 20-1 | CCGAACGGCGCTGAAACCTC | FIG. 31A | 124 |
| 20-2 | CCTGAGGTTTCAGCGCCGTT | FIG. 31A | 125 |
| 30-1 | ACTGATGCCAGGATCTACAA | FIG. 31A | 126 |
| 30-2 | GATACCCCACTACTGATGCC | FIG. 31A | 127 |

Flanking gRNAs

| | | | |
|---|---|---|---|
| 1.2 | CATTCATTAGTAATCAACCT | FIG. 21E | 128 |
| 1.3 | CCCCTTTTACTCCAAAAGAG | FIG. 21E | 129 |
| 1.4 | TAATAAACCAGCACGCACAG | FIG. 21E | 130 |
| 1.5 | ACCATAAGTTTCACGAGAGA | FIG. 21E | 131 |
| 1.6 | ACATCATCCAGCATCATTGT | FIG. 21E | 132 |
| 1.7 | CTAGCTTCCTCCCGACCGGG | FIG. 21E | 133 |
| 1.8 | CTATCTGTACTAGCCCGAGG | FIG. 21E | 134 |
| 5.2 | ATGTGTAGCTTAAGCCACTG | FIG. 26A | 135 |
| 5.3 | TTGCTCATCTTCACAGTACA | FIG. 26A | 136 |
| 5.4 | CGGAGTCTAGTTATGTCGCA | FIG. 26A | 137 |
| 5.5 | ATGGTGCCCGTCCAATGGAT | FIG. 26A | 138 |
| 5.6 | GAAGGGGATTTATTGTCGTG | FIG. 26A | 139 |
| 5.7 | TCGACATGGTACCTAGCACC | FIG. 26A | 140 |
| 5.8 | ATCCACTTTCAGGGACGGGG | FIG. 26A | 141 |

Medium/low intensity gRNAs at peak center

| | | | |
|---|---|---|---|
| 288-2 | TACGATGACAATCCAGAAAG | FIG. 25B | 142 |
| 288-2 | CATCTTAGTATCTGCTCGTG | FIG. 25B | 143 |
| 306-1 | CTACGTGCAGCACGGAACCT | FIG. 25B | 144 |
| 306-2 | TGTGGATGAGCGACCTCTCG | FIG. 25B | 145 |
| 329-1 | TTACGTGACCCCTTTGCAGA | FIG. 25B | 146 |
| 329-2 | TGCAAAGGGGTCACGTAATG | FIG. 25B | 147 |
| 350-1 | CTTCAAACAGGAAATGGAGT | FIG. 25B | 148 |
| 350-2 | GTGCAATAAAACTGTCTCAC | FIG. 25B | 149 |
| 372-1 | GAACATGATGAGTCAAGCAC | FIG. 25B | 150 |
| 372-2 | ATTGGCCAAGTCAAGCCCTA | FIG. 25B | 151 |
| 379-1 | GCTCATACAGACAAACCACT | FIG. 25B | 152 |
| 379-2 | TTTTGAGTTAAATCCTAAGG | FIG. 25B | 153 |
| SH1-1 | TATTATAATAGAAGTGCCTG | FIG. 25B | 154 |
| SH1-2 | ATAATGAATGTCTCATGAGA | FIG. 25B | 155 |
| SH1-3 | CAGTTCATATCAAGCACAT | FIG. 25B | 156 |
| SH1-4 | AATGCATTACAGTAGAGCA | FIG. 25B | 157 |
| SH2-1 | ACAATACCAAGGGTATCCGG | FIG. 25B | 158 |
| SH2-2 | ATCAAGTATTTACAATACCA | FIG. 25B | 159 |
| SH2-3 | TCAAGTATTTACAATACCA | FIG. 25B | 160 |
| SH2-4 | ACTGAGAACTAGTTCATAC | FIG. 25B | 161 |
| SH3-1 | ATGAGTGCTAGAGACTGGTG | FIG. 25B | 162 |
| SH3-2 | TAACCTATGAACACTGACAC | FIG. 25B | 163 |

2′-O-methyl 3′ phosphorothioate end modified guide RNAs (gRNAs) were synthesized by Synthego and Cas9 mRNA was synthesized by TriLink Biotechnologies. gRNAs were reconstituted at 1 μgμl⁻¹ in sterile TE buffer.

To obtain cells for CRISPR/Cas9 targeting in all experiments, buffy coats from anonymous healthy donors were purchased from the New York Blood Center (institutional review board-exempted) or peripheral blood was obtained from healthy volunteers. All blood samples were handled following the required ethical and safety procedures. As mentioned previously, peripheral blood mononuclear cells were obtained by density gradient centrifugation from peripheral blood followed by Pan T cell isolation, activated with CD3/28 beads (1:1 beads:cell) and cultured in X-VIVO 15 Serum-free Hematopoietic Cell Medium (Lonza), supplemented with 5% human serum (Gemini Bio-Products) and 200 U ml⁻¹ IL-2 (Miltenyi Biotec) at 10⁶ cells per ml. CD3/CD28 beads were magnetically removed after 48 h and 60-72 h after initial T cell isolation and activation, T cells were electroporated with Cas9 mRNA and modified gRNA (1 μg each for 2×10⁶ cells) using the Amaxa 4D nucleofector P3 Primary Cell XKitS system (Lonza). 3 days after electroporation, cells were pelleted and gDNA extracted for PCR amplification and sequencing of respective sites for cleavage efficiency testing. Analysis of PCR amplicon sequencing data for cleavage efficiency determination was performed using CRISPresso online tool for the deep sequencing data and the ICE online tool (Synthego) for the Sanger sequencing data.

Car Targeting.

The same protocol as for gRNA testing was followed with the addition of recombinant AAV6 donor vector (manufactured by SignaGen Laboratories or Vigene Biosciences) to the culture 1 hour after electroporation at a MOI of 3-5×10⁵. The culture medium was changed every 2 days and replaced with fresh medium containing 5 ng ml⁻¹ interleukin-7 (IL7) and 5 ng ml⁻¹ interleukin-15 (IL15) (Peprotech) and cells were cultured at 2×10⁶ cells per ml. The pAAV-1928ζ-1xx contains 300-500 bp of genomic DNA flanking the gRNA targeting sequences of the GSHs or TRAC locus on both sides, a self-cleaving P2A peptide in-frame with the first exon of pAAV-1928ζ-TRAC-1xx construct only, followed by the 1928ζ-1xx CAR as described previously. The CAR cDNA is followed by the bovine growth hormone polyA signal in all constructs.

Antigen Stimulation and In Vitro Proliferation Assays.

For use in weekly proliferation assay, 3 days after AAV6 transduction, CAR targeted cells were purified using magnetic Biotin-SP (long spacer) AffiniPure F(ab′)2 Fragment Goat Anti-Mouse IgG, F(ab′)2 Fragment Specific antibody (Jackson ImmunoResearch, 115-066-072), anti-biotin microbeads and MS columns (Miltenyi Biotec). The purified cells were cultured for 4 days as described before. NIH/3T3 cells expressing human CD19 were used as artificial antigen-presenting cells (AAPCs). For weekly stimulations, 3×10⁵ irradiated CD19+ AAPCs were plated in 24-well plates 12 h before the addition of 5×10⁵ CAR+ purified T cells in X-VIVO 15 medium containing 5% human serum, 5 ng ml⁻¹ IL7 and 5 ng ml⁻¹ IL15 (Peprotech). Every 2 days, cells were counted and media was added to reach a concentration of 2×10⁶ cells per ml. For each condition, T cells were analyzed by flow cytometry for CAR expression at time points mentioned in the respective figures. The antibody used for CAR staining was Alexa Fluor 647 AffiniPure F(ab′)2 Fragment Goat Anti-Mouse IgG, F(ab′)2 Fragment Specific (Jackson ImmunoResearch, 115-606-072). For keeping the CAR MFI comparable across all experiments and time-points, Rainbow Fluorescent Particles (BD Biosciences, 556298) were used.

In-Vivo Assessment of GSH-CARs in CD19-CAR Stress Test Model of B-ALL.

6 to 12-week-old NOD/SCID/IL-2Rγ null male mice (The Jackson Laboratory) were used under a protocol approved by the Memorial Sloan Kettering Cancer Center (MSKCC) Institutional Animal Care and Use Committee. All relevant animal use guidelines and ethical regulations were followed. Mice were infused with 0.5×10⁶ CD19-FFLuc-GFP NALM6 cells by tail vein injection, followed by 5×10⁴, 1×10⁵, 2×10⁵ or 4×10⁵ CAR+ T cells (unpurified, CAR+ calculated by flow cytometry) injected 4 d later. Tumor re-challenge experiments were performed by intravenous administration of 1×10⁶ CD19-FFLuc-GFP Nalm6 cells at intervals of 10 d at the indicated time points. NALM6 produce very even tumor burdens, and no mice were excluded before treatment. No randomization or blinding methods were used. Bioluminescence imaging was performed using the IVIS Imaging System (PerkinElmer) with the Living Image software (PerkinElmer) for the acquisition of imaging datasets. Tumor burden was assessed as average total flux signal of dorsal and ventral images per mouse.

Luciferase Based Cytotoxicity Assays.

NALM6-expressing CD19-FFLuc-GFP served as target cells. The effector CAR+ T cells and target cells were cocultured in triplicates at the indicated effector/target ratio using black-walled 96-well plates with 15000 target cells in a total volume of 100 μl per well in NALM6 medium. Target cells alone were plated at the same cell density to determine the maximal luciferase expression (relative light units (RLU)); 18 h later, 100 μl luciferase substrate (Bright-Glo; Promega) was directly added to each well. Emitted light was detected in a luminescence plate reader (TECAN Spark Reader). Lysis was determined as (1−(RLUsample)/(RLUmax))×100.

Mouse cell depletion kit (Miltenyi Biotec) was used for mouse cell depletion from bone marrow according to manufacturer's instructions and flow-through cells were then used for the ex-vivo co-culture and cytotoxicity assay with NALM6 cells as described above.

Antibodies and Staining for Flow Cytometry.

The following fluorophore-conjugated antibodies were used. From BD Biosciences: APC-Cy7 mouse anti-human CD8; BUV395 mouse anti-human CD4; PE-Cy7 mouse anti-human CD4; BV421 mouse anti-human CD62L; BV650 mouse anti-human CD45RA; BV510 mouse anti-human CD279 (PD-1); BUV737 mouse anti-human CD19. From BioLegend: PE mouse anti-human CD45; BV785 mouse anti-human TIM3 (CD366); BV421 mouse anti-human CD19. From eBioscience: PerCP-eFluor 710 CD223 (LAG-3) Monoclonal Antibody (3DS223H). 7-AAD (BD Biosciences) and DAPI solution (BD Biosciences) were used as viability dyes. For CAR staining, an Alexa Fluor 647 AffiniPure F(ab′)2 Fragment Goat Anti-Mouse IgG, F(ab′)2 fragment specific antibody was used (Jackson ImmunoResearch). For cell counting, CountBright Absolute Counting Beads were added (Invitrogen) according to the manufacturer's instructions. For in vivo experiments, Normal mouse serum (EMD Millipore) and FcR Blocking Reagent, mouse (Miltenyi Biotec) were used to block mouse Fc receptors.

Flow cytometry was performed on an LSRII or LSRFortessa instrument (BD Biosciences). Data were analyzed with the FlowJo software v.10.1 (FlowJo LLC).

Statistical Analysis.

All statistical analyses were performed using the Prism 7 (GraphPad) software. No statistical methods were used to predetermine sample size. Statistical comparisons between two groups were determined by two-tailed parametric or nonparametric (Mann-Whitney U-test) t-tests for unpaired data or by two-way Anova for multiple comparisons. For in-vivo experiments, the overall survival was depicted by a Kaplan-Meier curve. P values <0.05 were considered to be statistically significant. The statistical test used for each figure is described in the corresponding figure legend.

RNA Extraction and Real Time Quantitative PCR.

Total RNA was extracted from T cells by using the RNeasy plus mini kit (QIAGEN) combined with QIAshredder (QIAGEN), following the manufacturer's instructions. RNA concentration and quality were assessed by UV spectroscopy using the NanoDrop spectrophotometer (Themo Fisher Scientific). One hundred to 200 ng total RNA was used to prepare cDNA using the SuperScript III First-Strand Synthesis SuperMix for qRTPCR (Invitrogen). Completed cDNA synthesis reactions were treated with 2U RNase H for 20 min at 37° C. Quantitative PCR was performed using the ABsolute Blue qPCR SYBR Green Low ROX Mix., and the following primer sets: Ribosomal 18S:

forward
(SEQ ID NO. 84)
5′-AACCCGTTGAACCCCATT-3′,
reverse
(SEQ ID NO. 85)
5′-CCATCCAATCGGTAGTAGCG-3′;
1928z:
forward
(SEQ ID NO. 86)
5′-CGTGCAGTCTAAAGACTTGG-3′,
reverse
(SEQ ID NO. 87)
5′-ATAGGGGACTTGGACAAAGG-3′;
ZNF746:
forward1
(SEQ ID NO. 88)
5′-GCCCCAGACCTCTTGATGC-3′,
reverse1
(SEQ ID NO. 89)
5′-GAAAAGTTGGAATGGACACCAGA-3′;
ZNF746:
forward3
(SEQ ID NO. 90)
5′-CACCTCCTGATCCCTTCAAGA-3′,
reverse3
(SEQ ID NO. 91)
5′-CACAAGTCCAGTCGGTCACA-3′;
KRBA1:
forward2
(SEQ ID NO. 92)
5′-GAGGCGGTTTCAGGGGATTG-3′,
reverse2
(SEQ ID NO. 93)
5′-CTGGATTCTCCTGTAGCCGT-3′;
ZNF767P:
forward2
(SEQ ID NO. 94)
5′-TAATTCCAGAGCGGGGCAA-3′,
reverse2
(SEQ ID NO. 95)
5′-GGACTTGCACACAGGAGACAA-3′.

PCR assays were run on the QuantStudio7 Flex System and each sample was run in duplicate or triplicate in two or three independent runs. The difference between the Ct value of each replicate of a primer set and mean Ct value of 18S rRNA replicates was computed and used as ΔCt values.

RNA-Seq Analysis.

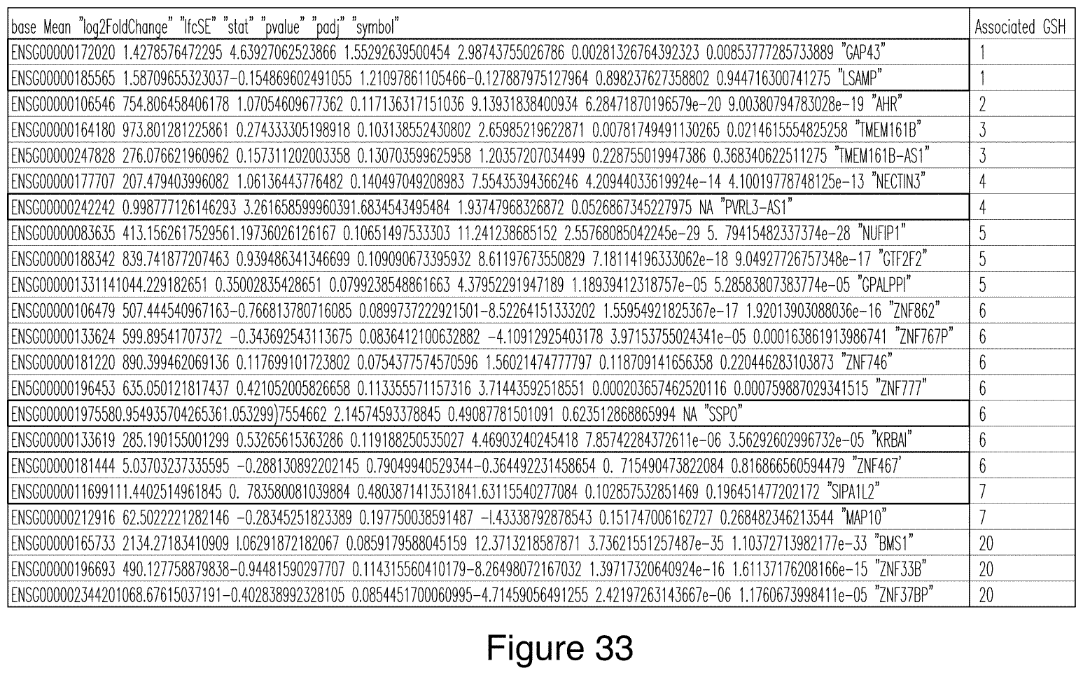

Peripheral blood mononuclear cells were obtained by density gradient centrifugation from peripheral blood of three healthy adult human volunteers. PBMCs were stimulated with CD3/CD28 T cell Activator Dynabeads (Invitrogen) (1:1 beads:cell) and cultured at 10⁶ cells per ml in RPMI (Sigma Aldrich), supplemented with 5% human serum (Gemini Bio-Products) and 100 U ml⁻¹ IL-2 (Miltenyi Biotec). After 2 days, cells were fed with 100 U ml⁻¹ IL-2 and at day 3 after isolation, CD3/CD28 beads were magnetically removed and cells were replated at 10⁶ cells per ml. 5 days after isolation, the T cells were replated again at 10⁶ cells per ml with 80 U ml⁻¹ IL-2 and either activated with CD3/CD28 Dynabeads at a 1:2 ratio (cells:beads) or left untreated. 24 hours later the T cells were collected for RNA extraction. RNA was isolated using TRIzol (Invitrogen) according to manufacturer's instructions, followed by RNeasy MinElute Cleanup kit (Qiagen). After RiboGreen RNA quantification and quality control using an Agilent Bioanalyzer, 500 ng of total RNA underwent polyA selection and TruSeq RNA library preparation according to the instructions provided by the manufacturer (TruSeq Stranded mRNA LT Kit; Illumina), with eight cycles of PCR. Samples were barcoded and run on a HiSeq 4000 (Illumina) in a 50 base-pair (bp)/50 bp paired-end run, using the HiSeq 3000/4000 SBS Kit (Illumina). An average of 35.9 million paired reads was generated per sample. At the most, the ribosomal reads represented 6.32% of the total reads generated; the percentage of mRNA bases averaged 64.5%. The output FASTQ data were mapped to the reference genome GRCh38 using the STAR 2 pass analysis (version 2.7.1a). The reference annotation was Ensembl v86. The output for each sample (read counts) was used as input for differential gene expression analysis using DESeq2 in R (ver 3.6.0). Data for GSHs analyzed is in FIG. 33.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A cell comprising an exogenous composition, wherein the exogenous composition is integrated up to about 10 kb upstream or downstream or within a locus of the genome of the cell, wherein the locus
(i) comprises a nucleotide sequence that is at least about 80% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75;
(ii) comprises or consists of the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75;
(iii) comprises or consists of the nucleotide sequence set forth in SEQ ID NO: 1, SEQ ID NO: 2, SEQ ID NO: 3, SEQ ID NO: 4, SEQ ID NO: 5, or SEQ ID NO: 6; and/or
(iv) comprises or consists of the nucleotide sequence set forth in SEQ ID NO: 6.

2. The cell of claim 1, wherein the cell is
(i) an immunoresponsive cell;
(ii) a cell of the lymphoid lineage; and/or
(iii) a T cell.

3. The cell of claim 3, wherein the cell of the lymphoid lineage is selected from the group consisting of T cells, Natural Killer (NK) cells, B cells, dendritic cells, and, stem cell from which lymphoid cells may be differentiated.

4. The cell of claim 1, wherein the exogenous composition comprises a transgene.

5. The cell of claim 4, wherein the transgene encodes an antigen-recognizing receptor that binds to an antigen.

6. The cell of claim 5, wherein the antigen is (i) a tumor antigen or a pathogen antigen; and/or (ii) CD19.

7. The cell of claim 1, wherein the antigen-recognizing receptor is selected from the group consisting of a chimeric antigen receptor (CAR), a T-cell receptor (TCR), a chimeric co-stimulating receptor (CCR), and a TCR like fusion molecule.

8. The cell of claim 7, wherein the CAR comprises an extracellular antigen binding domain, and an intracellular signaling domain.

9. The cell of claim 8, wherein the intracellular signaling domain comprises a CD3ζ polypeptide.

10. The cell of claim 9, wherein the CD3ζ polypeptide comprises a CD3ζ chain signaling domain.

11. The cell of claim 8, wherein intracellular signaling domain further comprises a co-stimulatory signaling region.

12. The cell of claim 11, wherein co-stimulatory signaling region comprises
(i) a signaling domain of CD28 or a portion thereof; and/or
(ii) a signaling domain of 4-1BB or a portion thereof.

13. The cell of claim 1, wherein the exogenous composition further comprises a promoter, wherein a transgene is operably linked to the promoter.

14. The cell of claim 13, wherein the promoter is an inducible promoter, or a constitutive promoter.

15. The cell of claim 14, wherein:
(i) the inducible promoter is selected from the group consisting of a tetracycline-responsive element promoter (TRE) promoter, a CD69 promoter, a CD25 promoter, an IL-2 promoter, a 4-1 BB promoter, a hypoxia responsive promoter, and a beta globin promoter; or
(ii) the constitutive promoter is selected from the group consisting of an elongation factor (EF) 1 promoter, a cytomegalovirus immediate-early promoter (CMV) promoter, a simian virus 40 early promoter (SV40) promoter, a phosphoglycerate kinase (PGK) promoter, a CAG promoter, and a metallothionein promoter.

16. The cell of claim 1, wherein the exogenous composition further comprises at least one insulator.

17. The cell of claim 16, wherein
(i) the at least one insulator exhibits barrier function;
(ii) the at least one insulator comprises a CTCF binding site consisting of the nucleotide sequence set forth in SEQ ID NO: 76 or SEQ ID NO: 78;
(iii) the at least one insulator comprises or consists of the nucleotide sequence set forth in SEQ ID NO: 77, SEQ ID NO: 79, SEQ ID NO: 80, SEQ ID NO: 81, SEQ ID NO: 82, or SEQ ID NO: 83;
(iv) the at least one insulator comprises two insulators.

18. The cell of claim 17, wherein each of the two insulators comprises or consists of the nucleic acid sequence set forth in SEQ ID NO: 77.

19. The cell of claim 1, wherein the exogenous composition is integrated by a gene editing system to the cell.

20. The cell of claim 19, wherein the gene editing system is selected from the group consisting of a CRISPR-Cas system, a zinc-finger nuclease (ZFN), a meganuclease, and a transcription activator-like effector nuclease (TALEN).

21. The cell of claim 1, wherein the locus is (a) located at a distance of more than about 50 kb from the 5′ end of each gene of the genome; (b) located at a distance of more than about 300 kb from each cancer-related gene of the genome; (c) located outside each gene transcription unit of the genome; (d) locate outside of each ultra-conserved region of the genome; (e) located outside of each non-coding RNA region of the genome; and (f) located at a distance more than about 300 kb from each microRNA (miRNA) gene of the genome.

22. A composition comprising a cell of claim 1.

23. A method of reducing tumor burden in a subject, the method comprising administering to the subject an effective amount of the cells of claim 1.

24. A method of treating and/or preventing a tumor or a neoplasm in a subject, the method comprising administering to the subject an effective amount of the cells of claim 1.

25. A method of treating and/or preventing a pathogen infection in a subject, the method comprising administering to the subject an effective amount of the cells of claim 1.

26. A method of treating and/or preventing an infectious disease in a subject, the method comprising administering to the subject an effective amount of the cells of claim 1.

27. A method of producing a cell that comprises an exogenous composition, comprising: integrating the exogenous composition up to about 10 kb upstream or downstream or within a locus of the genome of the cell, wherein the locus comprises a nucleotide sequence that is at least about 80% identical to the nucleotide sequence set forth in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, or SEQ ID NO: 75.


SEQUENCE LISTING (163 sequences)

SEQ ID NO: 1 - DNA, 853 bp, Homo sapiens
ggggcggaatcgcaacaagaccccaggaccccgtgtgcgcgcgcgtgtcgggggagctgacctatgacaaacatgtgcagagctctagtacccagccacgcggaggctgccggttgacagaagcgttgccgccccctgccgccgagttcaccctgccgtcactgctggaagccgcacccctaggaaaggctgcaaacccaccctgcccccaacacacccgcgctccctctctgcaggccggctccatccccgcccccaggtggccaagcaacaccgccttctagcttcctcccgaccgggcggcgcctgcagctgcctggggaaggagccccgcagcagcgcgagcgggtgggcggggggtgcgcgggccgcagggcaggagggagggcgcaggtaggcgggcggggagaagctaggcggccgggccgagcggtcgcgcccagccctgcctctctcccttctcgactcacaggcggcctgactcactcctttgtttctggaaaagatctatctgtactagcccgaggcggaattcaccccgcccgcactcatcagaaatcgtttgcttattctctgcgcccctccgtgctcgcctgggacgcgggcggaatcctgacgcaggctgtcagccgcggtgacgggtgacaccgggtgagcagccgtccgcgccgccttccctgcgccgacgggcagcgcggcgttcggcgcgcactgctgccccctcgtggcaggagcaagcatcgcagcagcgccgacaccaccgcccacctgcagcccggggagcgtcacttctgggtgtttttcaaagggagcatttttacagctccattcaactgctgggattaggagatgt

SEQ ID NO: 2 - DNA, 1396 bp, Homo sapiens
atatttttatgttgcttgataaaagatgattgtcaaaagagatccacacagacagtggctataagagtgaagctgcacaggtccctttgtagcagtcagtctctctcctactccctgtctcatctccttcccctgccacaaggacccagagaagcaggcaggctggcagtgtgatgacggtatcttaccctggctctgaggttgacacagatttcacaggtcacttctatcactgcaaaagcacattggtctggagtttatgaagaacatacaatttctgttggcatgttaggtacattatataaaatgccccgcataaagaaagcagtgtagttaaaagcaaatgaaagataaaaagagtaacaacgtggtttctcagtatttcatggtaccggtagatatgaccttcacagacaaacctaaatctttcttatgtaactgacatttagtccgttagccatttttaataaaagagtagagaagttactctactgcggcttttgctggtactttttatagccgtcgtgtggtaggaagtaggccccaagttcccttggcaacaagtagagtagaagcgatacacagaaaactgagtcatcggtgaatcatctgaggatttctgtttccaatccttggaaataatttaccgattgttttttatttgtgcagtgtttgctgactctgagtggacacctcagaaactgtaacatctttggtcaatttggtcaatcgtttaagaaacattctggatgctccaagtagaccttaagattgatatccaaacaaaacaaacctctgcttgctttaagatgacactcactccccatctgaggaggaacagataagtgagtcaaggtattggcaatggggaagtcccctttctacaaagaaatctgatgtccccagaaaaatgtttcatgacattgtcacatttaaaatgttagtcacaacaaattttcagacacctaaaaagagaacaaatatggtctcatacattttctctctctcactctgtctgagacttatgttcatcagttactcaattcccccatttttttttttacaaatacatctcttatataatccctttatctccacaattgttgcttctaccttatatcaaggtttaccatttcatttatctaggctcagattgtcctttacttcattgcgtgtacaaggagaaagaaaaaacctcccctaaaaatgctttttggccaggtgcggtagctcaaacatgtaatcccagcactttgggaggctgaggtgggtggatcacctgaggtcagaagcttgagaccagcatggccaacacggcgaaaccccatctgtactaaaaatgcaaaaattagctgggtgtagtgttgcactcctgtaatcccagctactcaggagg

SEQ ID NO: 3 - DNA, 842 bp, Homo sapiens
tagggggagaaaggcatttccaaccagtctccgtgcagaaccttcccgcaaaccctgtagcggcatttttcgacacttttctcctcctttcggcaagcctcaaacttgaccttcgagaggattttaagggtagagccttcctccctttccctcgtctgctccaagagctcacgtaggccttcttctgggaaacagcggctcaggaccagagctcggagaactgcgcgtccaggagcgcgggcgcggccggggaaaagggaggaggcggcaggagggggcaggaggtggagggaggaggaggatcggtctgaatatgcatgaggcacccagcactgccctcttccccggcagctgctcgggcgggggcgaaggagggaaggggaggggaggggaggaaatgaggctggggcggggtggccagagcagccgcagaggctgacgcgggtttggagaacggaagagatgatttgcatggcgcctggtgattggctgatggccgggggctgggtctgagaggcggcccctgaatggtttaacggaggaaattacaagattcattcgactaaagaaagccggggcgggcctggtcggcgcaatatgcagcctgtccgcaagggggcgcgatggcctatctttgttctggttaaaggctccttttccctcctcccccgcccccgcctccgcccccgcccccgcccccaccccccacacacagacacacacgcagaaagtgcagagaaaccctgtgatccagtcaactggctctgccccatctattaagaaagatgaacactcctttctcttccatctccttcatgggagaccacttttcatttcagatt

SEQ ID NO: 4 - DNA, 1082 bp, Homo sapiens
atttccctactcactcaccacacaatcatttcctaattgctgtctgtgtttgacttgcacctgtattaggtaataaagattcagaattttattcacactggttccactaccaattttactattcaattgagaaacagataccattagctgctaattccaaaatcatcagtttggtgactataacttgggtcagaaagatttagcactgcttctccttccacatccacaacttctagctcacagtttgccccttagaagtatctgtctaacattcatagtttagcctgcaagctgttgcttagtctagtaatgacacatacagaatggttttatggtcaagcactgagtcattccccaagctgcttgttttagctatattctccaaatgtatacaatatagatttataaaaatgattatgctgaggttataatttcaaaatgagccacaaagtatgtttgaggtactcatctggccttttcattttacagatgaggaaatacccacaggcggttgagcaaattcctgaagagcatacaacttgtaagatgatgacttcaaagctctttgagtgctatcagaatttgtgagtaacagaggcttcagcctatggatggggatggaggctacacaatttgaggtagggaagggtgggagtttgggaaccaaaagtagttactagcttgttaccaaagatagattttggttgcaaagaggcaaagtaaactgccagtgacctcaccacaaggcaggggcagacttcctgctaaggcattaagaccttctcagtcatctggggaacaaagtggcattcacaaggaaccgcctctgaccgcagacctactgggtagtggaggtttttcattcttaagtttgttctttcattcctatcaacttgcagattttctactgtcatcacccacctcctctcaaaccctcatttctattcagcttagatctcctcttttccttttgttttctcctcgattatttttatcttattttatgccatcatgccccatttcaaagaaatgttcttgttttcttaaagccttctacgtaagatccatttagctcctcattc

SEQ ID NO: 5 - DNA, 1302 bp, Homo sapiens
aaaagagcactcttttggtcatttgctcatcttcacagtacaaggagaaaatactggagaaaaaccagcgggtgtatttcggcatgcgaccgcttggttcccccgctttccttcacctctgacctaatctctcacttcaaaaactgacttcaagctgttgccagatacgtcccttccgccgcctgccgcaggtggctagggtctgagcacacttgaacctcacacccgccccaggggtagctccttggttcccttcagccccgatgtgtccctcgtgctttgaaatggaattacagttttggttaaaaacatgccttttccgagttaggaagaatctaaatcgactgaacgccagtctaaaatttcggcgttcccacaccgggagtcgaacccgggccgcctgggtgaaaaccaggaatcctaaccgctagaccatgtgggagacggcaatagcgactccaagcctagacaaattgagtcttctcggtcggcttccgcccactccatcgcgttcatccgtaggcgtcaaacctgctcctgcgcctgcgcggagtctgcagcggtttaaaccgttcaggttcgcattctactgtttctctccttgcaggggcccttgaatctttcttcaatctactctcgcgtctttcgtgatctcatccagtctcctggtgctaggtaccatgtcgacaccaatgtagacactagattggatgagtcattgttcacaattcctcacaaaactcctgcaaagtccaaaagaactggggtttgaatgttagaggctggtgaagaagtgaggaccacggggcgtcataacaccgcgcagtggaggccacgctgcactggccggtgtccgccgacagggtgtgtgccacagacgcgcagtgccttgcccgaatctgtccctcccctcagcgctggtccaggctggccacaggcaccaccccgtccctgaaagtggatcttttcctctgaaaaaacaggaatgagaactgcccggaagcctggaacattccaggttctgatgactcagcggctgaaggcaggatttgtgtcaccgaaagcagccagagtggcggtctgtaaatgtagcttagaaaagtggagaaagtctcttactgcaggcgttcatgatagatggaaactccgtgtggtttccaaggccctcctcgatttgaagcatttcaagggagcggttgatgccttagcagtctactgtttgaattagggaaatgcaaagagtcagccattggggatagccggcggagaggagctgtggctctggtgtcctcagtggcttaa

SEQ ID NO: 6 - DNA, 1184 bp, Homo sapiens
atattcccttccgcgccgaccctggggagctgggcacgtaaggggtgtcggtatacgcggattcgaagcctcacaactcccaggtaccactaagccgctggggggagctgggggagggacccacaggccgagcgccaagcagagaaaagagcctcgggtggggggacgcagatgtagggttcagcggctggggcgggggcaggaagccccggaggggccaaggatcttggcgtggggcggcccggggcggggacaaccgttccgccagcgctcgggctcggcccggagtggggggcccggctggcaagcgcagtaggccccggcagacgccgcggccctgttccccggagccccaggcgcggtagtacggctcggctcccgcagcccccgtgtgccgggcccgggtcgcccggggaatccaggccgcgagttcgtgcgcgcgctggtcgcccttaccggagctgcggccgcctcctccatggccctgcgctgtcccccgcggcccagataaagtcgtcgccgctgctgcgcgcggccccacgcagggcccgcctcccggtgctctccgcaggcggcgcccgcccagccctgtctctgccgccgctgctccctgactgcccctgccctgcgccgcccgcagttgcgtcaacgcctttagcgcccggccggtccgcactgtatcctgggagccggcgcggccgacgaaggcacatgaggcttcctggaggcaggcagccgcacagccccacctgcagctccggaggcgaactgcaggcgcgaccccgcctggcccgcgcccgggagctgaccccgcgacccctggccgacccctcgccccttccccgactgccgcgtgtgcgcgcgcagtcactgcgctggccccgcgacccaggccgagggactcggttaccgaaagagtccccctcataagacccgcctggccgcgcccgcgggcaagcaggggcttccaggcagggccctccccggtgaccgcgggcagtgtggccccctcgcccagctgggccccggcgcggccgaacaggcacttctgccgcccgaggtcctgggcctggaggtttgggctgttttcggggggcagctgtaactcatccccatctgtgtttgaagtggaataaatacttcgaacaataggaatataccggtagatgctacttagcattttc

SEQ ID NO: 7 - DNA, 1208 bp, Homo sapiens
cgtcagccccccaaagcgccccgaagggcatccccttccccttcgggacagtccccgcctggccgcaggcatcccgaaccgaccctcgccacgctgcccgtcggattccccgacacgaaccttcgcaacgccgtccgccggaacctgctacagcctgtgcgcgccgggcggcgcgtacccgggctgccgcctcgccgcccgccgctgcccggggctgcccatcggcccggactctccccgcgccgggctccggcggaggcggcccggacccgctggctgcggctggagctctcggcctgcgctcgggcggccggcgggggcgcggtgctcctcctccgtcctcctcctcctctcgctccgccagctcctcccgggctcccagtctgccgcgccggctcccgatgccaccgcccgcccgcccagcggcaccgccccccgcccgctctgcgggctcccattggtcgctgcctgcaggccccctcggccccgccccacgattgggcgcggacgccgtcagtctcaccgtccgcccggctccgggcgcagccggtgcgctgttcccaggccccgcggcgcgcgggagctgcgcgtccaggaggggcccggacgcccttcttggggagggggctgcggggcgcatcatgagcggcgctgggcgagctcgagggccgggcagcctacctggaccccggcttctagagagccgtcgccgtccttgtcgagactcgctttcgcccctctgaggaataagttaaaaaccgcgagtctggttctgagccgccccctggcgcaggcgcgaccccacccggccctcccgtctcccaacttgggacagacgcggcggccgggcggtcggatccgtgagcctcggccgctggggctacatacacagcccaggacgccacagaatcgccgaggccccgcgaacgcccaaacggccgcaagtcgaagtgatcccatctgaagaagacagcgcgaggaagcgagaccctgctgtgtgccggcccccggagggcgccccgggccctgtgggtcggagggtggggaagcacagagaaacacgcaggcagtaaaacgttaggaggacagggggtgccagcgtgcaggaaatgctgctaagagacctttaggagccaaggggaaaacggagagattggcagttccaggcacccacatcccagcgcggtccgctcaccccttcctccctg

SEQ ID NO: 8 - DNA, 785 bp, Homo sapiens
tgaaaataaaatcaaaaatttgctttttgtcattctggtaatgctgttcttcatcatagacaataacttggagataacttcaagccagagatggtagctttgctcatagttgctttggatggtaatcaaatgagactttcgtttatgtcacagatggaagcttgcaagtcaaaacattcaaaagaaatgaagatttctacatacccaagaactaatgagctagccagtgagtgagtcattatccatttatgtcacagatggaagcttctaagtcaaaacattcagaagaaatgaagatttctacatacccaagaaccactcagctagccagtgagtgagtcatcatccctcacttggcctgctctttggctccatactggcttcttggctcctgttctttacctgctacagtttttgtccacatagtagccagaatgattctattgaaatgaaacagactatgtcatttaagctcaaaaacctcgaacaacttttcatctcactcagaatactatgattgtgtggaacccatattatatgacctctggctaacttgattgaatttatacctcaccattctttccttctcaaagttcactccatcagccctgactttttggtggtgaaaccaagtatgcttccattcactgcctctgcacctacatagcttctgcttgtgaagctcttcctggagacatttacttggctcacactctccaatcaagtctttgttagaatgtcccttatttgcgtgttgctcctacctaatctttataaaatggcac

SEQ ID NO: 9 - DNA, 933 bp, Homo sapiens
aaggaagagaaggaaatattgctaacgcagagcctagaccaactgggagggatgtgatatagagcccagaggagcaatcacttcaaatgggaacatggatggctcatttataggcccaagtgggaaggcaaaagttgtgagtgggaatggtggtaagtgagtacaagtcgtggtggaagactatgccacttatgtattgctacagtttttaagaaatataggaagcaaggccattggctaagagtgatgatagaggagatttggaggtgtgagaagagagtcttttaaggggatgcttcccaaacttaaatacactatgcagaagaatctcctagtatcttgataaaactcaggttcgtattcagtaggtctaggatggggtctgagatctgtatttctcagaatatctaggtaatatcaatagtgttggcccatgggctacacatggagtagcaaagttctatgagaaatgagaaagttgagtgaatcaggaaagtactgagtgcgtaacagcattaaacatctactggagttcattgctgttaactgaaagtgaaactaatcagaatatttgtgtgttttgttccaactactctgggcttaaatgaggccaaatgtggctggaagtgaaagtgaaaatgatgaaggaagagagatatgagtcaagggtgtgtgcactggagaggttataattggtaaatttaagctgggttaggagggagaagtgttacagagtgaatatatcctgccgcaactcatttgttgaagctctaacccccagtgcaatggaatttagaggtaaggcctaagggagataattagagctagatggggtcatgtaagtgggtccctcatgatgagattagtggccttataaatagagtatgagagagacctctcctctctttacccaaaaaaaaaaaaaaaacaaaa

SEQ ID NO: 10 - DNA, 932 bp, Homo sapiens
ttttcattccacttaccatgacagttagctcaggtctctacaatgccctttccaccaacaagttgaatttctaagtctcttggctgaaaagggcaaagagctttcctgtatgatgaaacaggctgcatggcccaaaggcagcctgctataaaggttgactgtggctgcctcctctgtctcaagctttgctgggaagccacatttttcacaattgagttgttgcaaaaagttatgtgtgttacactctatgtgttcttgaaaactatatttgtcaaaatgttcatggtaggaaactgttttcccttcaagttgtaactgaggtttaccttggaggctactttgaccactgtgatatttaactctatgaccaccaactcttgatctggtgctaatgaactcaaacagcattaaagactatggggatattcttaacattgggaacacagctaaaggcagctctttctggggggaaaaatggtttacaagtaaataaataaacagtgactctaacacattaacttcaaaaacatcatacagaatctcaacttgccatgacatcgttctgtggtcttgggccagtcataatgtctatgaggcttagtttcaacttctgtcacgatgtgtgtggctcagccttccctcatcagtgctgtgaggagtaaatgaggagacttgtgcgaagagatccacacagagctgagcacgcggtgagcacccagcaacaccaacatagaggggagagccttctctcacacgcctcgagcttccacagaagaaggaacttctgctctactcaccgagatggacaaggataggagaaacaccctcccagtttcttccccacatactaccctctctcacagaagaagccaagcagaggggtgggcctacaacacaccctgggccacggcgcacctgaaggagggccgctg

SEQ ID NO: 11 - DNA, 1099 bp, Homo sapiens
aatcatcatgtttcaagggatagcagagttgatattggtaggaagagtggaggcagccagatgaaggaggtgggaagtgagaagagcaccccaggcagaggtgccagcataagaacgtcgtgtgaaaaggcagtgacctgctgctccgtgtcctgagcttggaaaccctagtgagacattaggagagagtgcaggcaagaggatcctgcaggccttgagacagggcagaaaacaaggcctccagaaatgaggcagcaggaggaagtgtatgagaaagcagaatagatctagaaagagccagcagatcagaggataagatgggcaagtaccatgtcacagaagcaaagagaggaagtggagtttcaacagcggcctttaacagagagagtgcgaaggtcagagacccagaagaaggagggcttggctagaaggtcactagtaggctctggcttccatctccagataggctaagattaagccaattcaccattttcccacaactgtcaactctgcagaattgccttttcttcctctcagacagaaagggaaatcccagctgcctgagcaaccggtttttcactttcctccttgtttgcagagtctagtggggctggccctggtaacctctggtgattcatccagcacaccctatgaccacatccccattgtgtgtgtgtagagaggatgagagtttatgtgttcctctgaacgttattacaaaagaaaagaacattgttctctagaaatatgaaagaaaaagaaaagaacaaggtgttactctagcatagttttttctcaattatctgagttgtgaaaaaaaaagtaatgaatagtggagcttcctcaaaacagattctgagcgctgtcagtagttaacagtaactactgtggtggaattcctgtgtggatctattttggcactgtgttacgtaattatttttatcttaggatcagatctgaatcattcaaagccaatgtcttcagggcactggtgtgactccaggtgttctcacatgcctgtctttcttctcagatcactgcccaacactgaatctcaagtgatggacctcagtgcatatgaattcggggcctatatgtt

SEQ ID NO: 12 - DNA, 898 bp, Homo sapiens
tctttctctttttcaaaatatcttgtcgtactgcacacttagatgtgtagtataataagatatttccttgttatgatgtgagatgacacaatgcctatgtgatgtgatgaagtgaggtgaatgaggtaggcattgtagcattaggttaccagtcaccttcttacaatagtcacaaagagatcatttgctttgagtgatcctgcatcatcgagctgtgatgatgttgatgatgcggatgactaataggcaagtaatgtacgcagcatggatacactaaatgaaagaatgactcatgtctcgggtgggacaaaacacccacgacatgaatcatcacatggtacagtgtttcttcagggtattcagaatgacacacaatttaaaacttacaaattgtttatttattgaattttctatttaaaaatttcaaactgcagttgactgtggtaaacttaaactggagaaagtgaaactgggtaagaatacgttttgcatctgtgtatcctgacaatttcaaggaacacacattaaaattagcttattaaggtatgcacatgtttaactttattggatattatcttagtatgttcaggctgctgttaaaagaaaaacaaaagctgggtagcttacaaataacatacatttattttttacagttctggatgctgggaagcctaagatcatgggaaaggtaaatttaaagtctggttagagttcactttcaggtttatagatggtgccttctagctgtgtcctcacatgatgaaaggggttagtggtctctttggggccttttcaaaaggcactatggaaccacacacacaaacacacacacacacacacacacacacacacaccctggaatagccaaggcacttgtgagaaaaagaacaaagagtta

SEQ ID NO: 13 - DNA, 1738 bp, Homo sapiens
cggggacacactcagagtctggatctgattcaccaaggcgctaacactgaaacagtggtccagagtgccagcttttgttcatgtctggtcagacacctccatcctctgggcaatctcactcactctcctgggacctaaaaggagagcctcacctggggtgccacaaaatgttaccagcatcatggattcttgtgattgcccaggatagaaaccaaaagagataaccaaacatagcagcaaaggaaagtttattccgcttgtgcacaggggagtcagcaccaagaagggaaaagaaatgggttgcttatcgagggtagtctgtgagttaggtttacaggagttttctacaggaaagtattacattcgagcatgtataggagtaggagggatttttctagtgcttgtgcattggctcaacatgctttttcatacatcatatgtagaattagcattttacatctccacctctgggcatgatttttaacattaaaataaggaaggggtaactgtagttcgaagtttaactctaactgcacatgcagggccctggggaagtccctagcccccgaagcaggaatttgtggttaacagcttcttgggtcttttattactgattggttgagagttaggtaagctacagcttgagtaaggaacttgcattcttttactccagaccatattaaaacagggaaccaaccagcctgcctgtctcatagctagtttgctttaatctgttcccagcttaccttccttttctcagatgaaatgagactcatgttaaccaaaatgccatcctaccttttgtcttttgactagacacattgtagaggtttggatttgttaacctggaatgggaaaggctgagaggggaaatgatcctgggtatgctaatgagcagaaagaaaacaaggatggcacggatacctctctctcccccttctatgtgtgtcctctacctgttgtacattcgtatgagataggtattaatataacctacacttcgtaggtgacgaaacagtctcagagagtgaaagggacttgaccaaagtcacagagccagtgagtgacagggtcagggagagaagccacgtctgcatgactcaaagcttatgtctctaattaccctacatactaccccgccgcctttgcaagttataaataggtcatgctggctttactcattaagtcctgggttgctaaagtgaaagaaaaacttccttcccagtatgccttgtacacatcctgaaactgaaagaaagcactttagaacaaattaaaggcagttgtcttctgtgtcttagggaacaaaaatactaattgtatttttaacctcaaattgtagagcctgaaaagactggcttgagaaggagcctaggtcattccctggacacctagaggtctagagggcataatagaggtccacaagtttttctcaatgtgaaacaaacaataatcctcccaaccagacagagaatttacatacgctccctttcagaccgggcaggcagacaaagtcacaagtgctttgactttgggctaaggattctgtcaggtccatcaatcctttaacctcaccttcacagccatgatagctaaccctgactgaacactttctgtatgccagccactgttccacttcctttactcctcataacaaccctgaggttatgactagtatcacctctattttgcagaaacttgagggcatctcatgctgacaagaaactaattgggagtt

SEQ ID NO: 14 - DNA, 809 bp, Homo sapiens
aaaccaggacatacagagataggagctgaagggacatggtgagaagtgaccagaagactagtgtgagccctctgtcacacccggacagggccactagagggctccttggtctagtggtaacgctagtgcctgggaaggcacccgttacttagcagatcgggaaagggagtctccctttccccggtggagttagagaagactctgctccaccacctcttgtggaaggcctgacatcagtcaggcccgcccgcagccatccggagacctaaacgtctcgctgtgatgctgtgcttcagaggtcacgctcctgtttcactttcatgttctgctctgtacacctggctccgccttctagatagcaatagcagaattagtgaaagtattaaagtctttgatctttctgagaagagcatagaagaaataatgacgtaagctgtcctctctccagctcggctacctaaaagggaaaggccccctgtccggtggacacgtgactcacatgaccttattaatcactggagatgactcacactccttaccctgcccctttgccttgtacacaataaataacagcgcgaccaggcattcggggccactgccagtctccacatctaggtggtagtggtcccccgggcccagctgtcttttcttctatctctttgtcttgtgtctttatttctacgacctctcatctccgcacaccaggagaaaaacccacagacccagtagggctggaccctacagctctatgcaaatcagatactgcctcctccagcctcccatataaccgaccacttttctgccctccc

SEQ ID NO: 15 - DNA, 704 bp, Homo sapiens
ttttaagtgtgtttttaaaaaaaagtctgtgctactagaaagaaaaagaaaacaactgttcttgctatcatacattacatggttaaaggggcacctcccttaactgactggaggaagttagctgttggtttcaaaatcactctttccagaaaagcagggtgtgtgactcagatttccacccgaaggtggtccttgagagaaggtcttagatgtttagaagaggttatgttacattctttggtatcacttattatttccttcaggcaaaccacatgtcagaggacttcaatctgaaaatgacgtgagttacatgggttccttcctcttcgttttttaaaggtcctcttgtctttctgttttcatgtgtgctcttcactggcagctttctataaacaaatgcacttatcagaataaaagagtcatttcacttctctagggagagatttttatatccatcttttgaagagatgagatgaaacgtagtttgaaattgaatgtatacaaattagtgtatagatctccacattttaaaagaccctgagtggtcaaaatgatgaaacaaatgagaaagaaaaataagaggacaagaactaacattaattggaaacccagtgtgtatctcacaaccttataggtattttgcacacagcatttaatttgatcctcctattaaggattaaatagacattttatctccatgttat

SEQ ID NO: 16 - DNA, 1113 bp, Homo sapiens
gtctcaagtttctagagagccaggctctcgcaggctgaggtggcttccacactgttcatgaactgctcttggcgggctggtggcctgtttgatattctgaggtctagcaaagatttagctccagggggtcatcaagaggtcatggtcttgatcatttcctctgttatttatctttggcttgtggtactggagcctgcctttgatacagggcatttctaccacagctgcacagaatcaagcatttcagtgatttggtttgaggtgaaggaggcttggcttctaatgctgcgaaaatgtcactgggtacatctgagaatatctagctctgtccttcactgatgagcaaattcatttctgggggtttaccagtggcacttcccagacttgggtcaggaaaggaatcctcctggtgaggacagcagttttcagtttgagtaattccattttcagttccttctgagacatttttcttgctctgagctggagataaaagcagcatgtctcaggcctctagggtggatctctacttttcaacactgtttatgactcacctgtgactgccctcagtgcagccaaggggaccatttatgctgaattagcctttggatttttcttcttagtgacagcaaaacttccgtgacagcaaacaagtaaaccacaggaacatccctggtctgctcagcacagcaggctcttgggttctcagagagcctgggaggacttttcagcttgtcttatgtgggtttgttcaccccgtccggtttcatttttatttaaaacaattggtatctttcatcttggttgccaagtatgggttgcatatggttttgacctgatatgaagggcccaaatcaaatacccaggacttggcctggaaaattggttgtataagacctgctctcttgttcccctaaaaagcagcctagcaaatggaagcaatgggtcccaggagttcgtttctgtatgcggctctaactctgccacgaaggccaggacagatagtgtgcagtgctctgtcatgtctaccagagaaaatgatgcaacatgtgctggagactttaccatctgtgcaagagtgcagccctaggctcaatccaaaagaaaaaggaagtg

SEQ ID NO: 17 - DNA, 1479 bp, Homo sapiens
attggcatacagctgtttcctgctactgccttactctagagagaggatgacttatcactcaactgaagctcccaaaagaagagctgtaggaggccttgttacctcacacagctggctaaactcataacctgccctgtcatgattgctattctggggcttgatcgataaagactcgggtgttctcaattctgttcttttctttgcaatcgcaacactcaacaatttcttgcttgataactatatgcagacactgccctcacttccccaaggaattgcactgtgcctgcacatccctgtcactttcacaaagaggcagataatttcctctcactggatagggtgctatgtggaaatgacgtgaaatccgctcaacctgatttccaacatgtgcgttcgttttctccctattcaggtcatagtgattcatgagagagagaggcagagcgggaactgaagcagtttcagcctcaccaaagtacctttcccctagttcctcttactgtgctcattatcctttctttcctgcttaccatacatcgtttaccaaccctttgaaaatttaaactaccaggttcatctagctctggcactgctagaaccgagactacagaagatgtctgagtcttccaagctatttgggtcaaacaaaacaaacaaaaccacacacagccaccgagttgggttattttaaggacttagagctacagaaggcaatctgtcagcatccgaggctgtttcaaaaccacctcctccctccgtgcttgagtcaagagctgcagtctttctatcgcccatagccatgctgcagcacttcctcaatcaccctcttctcgctgcacgcattgactttcggtttcttcacagagtcactgacagcttgcagctttggcttatcgatagccccgtggttctctccatccatgacctaccagatggcattttagtttagttcccaccactcactgccttatttaattggtatttcttgccccacatttccaaagcaagcccggtctggattctgtttggttcagccatctcatgggtctccagctgcttccctgtcctgtaggcggcctgcgtgaggcgcagttcctacttccaaggtattgtggggcagtggctatttttgcaggctattgtgaggcagcagaggtaaagtatgagttgaaacatacaaacaaaatatggcccctcagcattgtagaacttttgcagataatgaattttagaaccaaatagagcaggacatttgttttcacagtgagtcttatggctctacttagaatcttgtcactctgttttgcagtcacctctccaagtcattgtcttttattgtacccttgagaaacaatactgtttccattccaaccactttactgagtatttttactcagtggtcaggtcacaaataatctgttaagttttaaattcagttgtcttgtattgctgatttctctgtcttatttaatgtattat

SEQ ID NO: 18 - DNA, 870 bp, Homo sapiens
cagcaaagagaacaaacttttgcagatgccctgtgtgataagaagcacactcattcaacaagtttagaacatccatttaatcctgctcttttcttgctaatattcaaatttctttagttgacttgtatttcaggaccttgttgaaaaaataacaatattctagattctagagtctagaaaagtaggatcaacctttgacatatatcagaattgagggactaagtggctaagttggctggacttcctgggtcaacagggacttccccagggggactttcccctaagccaaaatgagtcatagctgcaaggtacgggattgaaacttcacccaatcaacagggactttcccctaagccaaaatgagtcatagctgcaagcgaagggattaaaacttcaaccaatgaaaggggactttcccctaaaccaaaatgagtcatagctgcaagctaagggattgaaatttcaagcaatcaaaggggactttcttctaagccaaaatgagtcacagctgcaaactaaggcattgaaacttcaaccaatcatatagggagtttaagctctagctgcagcctgatgtttttaaccaagcaggcccaccaacccacaagcggatagaaaataagctaatcctacaggacaggaaaaggaaaaggggaggagtcataaggggacataagcctaagacacccaagccagaaacggcagccattccggatccccatggaagctttactttctcttttgctttactttcgcttttgctttacttccgcttttgctttactttcgcttttgctttactttcactttcgctttaataaatcttgccgcagcacactctttgggtctgcgcgtttctctaatcgagct

SEQ ID NO: 19 - DNA, 722 bp, Homo sapiens
atagtaaagaaaaagttttgttttcagatataattgaataccgaagaaaatcagaaaattcaactgaaaacctatttaaagtaaaaagaaaacttactaagatggctgcatacaaaaatcaagagttttccttttatagtagtataacaggatgtgaaaatgaaagctttgactgaactctgaattacagtgtgctatgagtcagatcaacaagctgaaatttcaaagaaggtgtttgtgaacctgctagatgaatcatctacatcaaggatctaaaacggtcaggtggctggtgattcaacaggatttggtgaagaggactggtttggaaaatctgtgttcttaatcctgtcttccatcccagagaaagggaggggggatgattgacccttctttctcaaatctagggagaaagtttcatgagggctgcttgcagagcttgatatatgtttcttgtagtcgtggggcattgtgaattggtttctgattcatgcctgtgaaatttgagacaaaaagatcatggctggaagtttcgtgtgactaggtgactaagttccttccaataagaaataatgaatgcagatgtcttgttctccttttctctccttcttcctgtttctagttggccctggaacagtggcctccaggcctgagataatcgaagcaactggagaagcttgaaggagtctgggtccctgatcctgacaaagccgctattccac

SEQ ID NO: 20 - DNA, 1390 bp, Homo sapiens
tcaccccacaacagatgttttgctccaagacagctgggcctaagcccttcagttcgaatcctcatccaagactgaaagctcctacatcttcctgtcactttagccacaagctgcaccgtctggaaatgcccactgctactagggacacaggcgaggccggacagtgccggaggacccggggtgcctccctccacgtgcgcggcctcacaggtcagtggaaacaagaaatcccccaatccacagccacgatgacttcaaaatacttcctgaaggcatcaggccatgaagctgcgacctgcgaggacacagcaagcggggtgacgctgagcagaggcttggtgtggagtccgcgcccgcagggtccctccccgccccgcgggtgcttcccgccacctcgctcgcctctgcgtcgcagtgacaacgcggggatgccgaccacaggtggttggaaggagacagcgagattctgaggggaaagaataaggagagaagcagcatcttttgggcagaaggcccgtttccgcacggtcctgccgaggcctccgggcatccttcacctcagggaagagaaaatccaaccttacttatcacaggcgcgggacctacgttcccttcgggagcagaagaaccaggacaggattaggacgcaggccaggcggcaaagccgctccctctctcccacctccaggacaagcggcacctccagtccaccctcgctccagccctcccgtactctacccccggcatcctctgccccgaagcagggagatgccgggagccctccctaccaggctgtaccggagaaaaacaaacctggagccacaccgcgcacatgcgcagaaagcccaaagcggctcccgcgaccggggccaaaaagtgcacactacatttcccacaagtcctcgcgccgctcccttagaaagtacacgctacatttcccacaagccctcgcgccgctccctgagaaagtgaagactacatttcccagaagtcctcgcgccgctcctttagaaacggcctgaggtttcagcgccgttcggtagttagttcccagatgcaccgagtgcaatactgcaattttattcgttagctgaaataaattatgagtatttaaattacacaggtttcttatgaattacatctccctgatgatttggctgtcctcaacacttcctcgtttggcagaattcaggagtggaatttctccagacacggtcggttcctgtggccccgctgcgccgcggcggcaccacctccacttgcgcctgtggctccacttgcttggttctggtccactggacgctcaccctgagaaaacacgctggacatgagcattaccagcctgtacactgttgtaaagataactcggaattgctcttcaaattttatt

SEQ ID NO: 21 - protein, 263 aa, Artificial Sequence
  Synthetic
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 22 - DNA, 789 bp, Artificial Sequence
  Synthetic
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgg

SEQ ID NO: 23 - protein, 6 aa, Artificial Sequence
  Synthetic
GYAFSS

SEQ ID NO: 24 - protein, 6 aa, Artificial Sequence
  Synthetic
YPGDGD

SEQ ID NO: 25 - protein, 9 aa, Artificial Sequence
  Synthetic
KTISSVVDF

SEQ ID NO: 26 - protein, 11 aa, Artificial Sequence
  Synthetic
KASQNVGTNVA

SEQ ID NO: 27 - protein, 6 aa, Artificial Sequence
  Synthetic
SATYRN

SEQ ID NO: 28 - protein, 9 aa, Artificial Sequence
  Synthetic
QQYNRYPYT

SEQ ID NO: 29 - protein, 122 aa, Artificial Sequence
  Synthetic
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 30 - protein, 108 aa, Artificial Sequence
  Synthetic
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 31 - protein, 15 aa, Artificial Sequence
  Synthetic
GGGGSGGGGSGGGGS

SEQ ID NO: 32 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 33 - DNA, 81 bp, Artificial Sequence
  Synthetic
ttttgggtgctggtggtggttggtggagtcctggcttgctatagcttgctagtaacagtggcctttattattttctgggtg

SEQ ID NO: 34 - DNA, 117 bp, Artificial Sequence
  Synthetic
atcgaggtgatgtacccccccccctacctggacaacgagaagagcaacggcaccatcatccacgtgaagggcaagcacctgtgccccagccccctgttccccggccccagcaagccc

SEQ ID NO: 35 - protein, 112 aa, Artificial Sequence
  Synthetic
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 36 - DNA, 336 bp, Artificial Sequence
  Synthetic
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 37 - protein, 22 aa, Artificial Sequence
  Synthetic
QNQLYNELNLGRREEYDVLDKR

SEQ ID NO: 38 - DNA, 66 bp, Artificial Sequence
  Synthetic
cagaaccagctctataacgagctcaatctaggacgaagagaggagtacgatgttttggacaagaga

SEQ ID NO: 39 - protein, 22 aa, Artificial Sequence
  Synthetic
QNQLFNELNLGRREEFDVLDKR

SEQ ID NO: 40 - DNA, 66 bp, Artificial Sequence
  Synthetic
cagaaccagctctttaacgagctcaatctaggacgaagagaggagttcgatgttttggacaagaga

SEQ ID NO: 41 - protein, 23 aa, Artificial Sequence
  Synthetic
QEGLYNELQKDKMAEAYSEIGMK

SEQ ID NO: 42 - DNA, 69 bp, Artificial Sequence
  Synthetic
caggaaggcctgtacaatgaactgcagaaagataagatggcggaggcctacagtgagattgggatgaaa

SEQ ID NO: 43 - protein, 23 aa, Artificial Sequence
  Synthetic
QEGLFNELQKDKMAEAFSEIGMK

SEQ ID NO: 44 - DNA, 69 bp, Artificial Sequence
  Synthetic
caggaaggcctgttcaatgaactgcagaaagataagatggcggaggccttcagtgagattgggatgaaa

SEQ ID NO: 45 - protein, 22 aa, Artificial Sequence
  Synthetic
HDGLYQGLSTATKDTYDALHMQ

SEQ ID NO: 46 - DNA, 66 bp, Artificial Sequence
  Synthetic
cacgatggcctttaccagggtctcagtacagccaccaaggacacctacgacgcccttcacatgcag

SEQ ID NO: 47 - protein, 22 aa, Artificial Sequence
  Synthetic
HDGLFQGLSTATKDTFDALHMQ

SEQ ID NO: 48 - DNA, 66 bp, Artificial Sequence
  Synthetic
cacgatggccttttccaggggctcagtacagccaccaaggacaccttcgacgcccttcacatgcag

SEQ ID NO: 49 - DNA, 123 bp, Artificial Sequence
  Synthetic
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 50 - protein, 41 aa, Artificial Sequence
  Synthetic
RSKRSRLLHSDYMNMTPRRPGPTRKHYQPYAPPRDFAAYRK

SEQ ID NO: 51 - DNA, 20 bp, Artificial Sequence
  Synthetic
cccggtgtcacccgtcaccg

SEQ ID NO: 52 - DNA, 20 bp, Artificial Sequence
  Synthetic
cgcgtcccaggcgagcacgg

SEQ ID NO: 53 - DNA, 20 bp, Artificial Sequence
  Synthetic
tgacggcagggtgaactcgg

SEQ ID NO: 54 - DNA, 20 bp, Artificial Sequence
  Synthetic
gcagggtgaactcggcggca

SEQ ID NO: 55 - DNA, 20 bp, Artificial Sequence
  Synthetic
gcctacttcctaccacacga

SEQ ID NO: 56 - DNA, 20 bp, Artificial Sequence
  Synthetic
ttttatagccgtcgtgtggt

SEQ ID NO: 57 - DNA, 20 bp, Artificial Sequence
  Synthetic
gagtcaaggtattggcaatg

SEQ ID NO: 58 - DNA, 20 bp, Artificial Sequence
  Synthetic
agataagtgagtcaaggtat

SEQ ID NO: 59 - DNA, 20 bp, Artificial Sequence
  Synthetic
cattcgactaaagaaagccg

SEQ ID NO: 60 - DNA, 20 bp, Artificial Sequence
  Synthetic
gcccctgaatggtttaacgg

SEQ ID NO: 61 - DNA, 20 bp, Artificial Sequence
  Synthetic
tcgactaaagaaagccgggg

SEQ ID NO: 62 - DNA, 20 bp, Artificial Sequence
  Synthetic
tatgcagcctgtccgcaagg

SEQ ID NO: 63 - DNA, 20 bp, Artificial Sequence
  Synthetic
gttagacagatacttctaag

SEQ ID NO: 64 - DNA, 20 bp, Artificial Sequence
  Synthetic
tagctaaaacaagcagcttg

SEQ ID NO: 65 - DNA, 20 bp, Artificial Sequence
  Synthetic
gaatttgctcaaccgcctgt

SEQ ID NO: 66 - DNA, 20 bp, Artificial Sequence
  Synthetic
ggaatttgctcaaccgcctg

SEQ ID NO: 67 - DNA, 20 bp, Artificial Sequence
  Synthetic
aattccatttcaaagcacga

SEQ ID NO: 68 - DNA, 20 bp, Artificial Sequence
  Synthetic
gaaccaaggagctacccctg

SEQ ID NO: 69 - DNA, 20 bp, Artificial Sequence
  Synthetic
gatgaacgcgatggagtggg

SEQ ID NO: 70 - DNA, 20 bp, Artificial Sequence
  Synthetic
acgcctacggatgaacgcga

SEQ ID NO: 71 - DNA, 20 bp, Artificial Sequence
  Synthetic
ctgtatcctgggagccggcg

SEQ ID NO: 72 - DNA, 20 bp, Artificial Sequence
  Synthetic
taaaggcgttgacgcaactg

SEQ ID NO: 73 - DNA, 20 bp, Artificial Sequence
  Synthetic
gcggccgacgaaggcacatg

SEQ ID NO: 74 - DNA, 20 bp, Artificial Sequence
  Synthetic
ggatacagtgcggaccggcc

SEQ ID NO: 75 - DNA, 695 bp, Homo sapiens
ttcactcaaattcctcccaaatagttttgagagtctccagatgtctcacatctactttacatacaagttccctcagatctccttctctcatgatcccatttcagctctctgtggtgaatctgataccccactactgatgccaggatctacaatggttaaggttccactgcctgacaaacatgttagtgatactggtgcttgctgttgtctgcattgaggcttctgatatctgttactgaggatattgaggtttacccacagagaactcttgcctggtgctgctgaagctcttcgtataccatctcctctgaacagagtcaacatatgacttgtcttcaattatagccaaagctgtaagtcccaatatcactaatgcttttctttgaagctcttaaatgcatttaattcttaatgcatagcagttctatttgactgtacaccctagtactcaaaattgattctaagaaagctgtatgggaatacaaactaaaacaatgaatgctataccaccttaagaaccagttgatccctccatccttggccactgttcttgaaaacaaaagtgaattgactccctttttctctagaatgatttgctccagaacaaagtttaattctttatgtatgaagacccataattgtttctatcaccctgcctacatattcctaactagtaaggtaaataaactaaattt

SEQ ID NO: 76 - DNA, 14 bp, Artificial Sequence
  Synthetic
tcagtagagggcgc

SEQ ID NO: 77 - DNA, 325 bp, Homo sapiens
gtctgaatggtggccgtagtttgcagagccctggtttcttcttgcctctcagcttccaacttccccgtgagtgcctgctccttgatggactggactctaagcccttctttgcagcaagcacgatatcaagctttgtcagtagagggcgccggagggacactgtggaggaaggggccttttcatggtccacagagctctgttgtgcaatttcttgttcctgttgcatcttctcttagggtatgaacgcggggggacatcctctggggcttttcctcagctgtgcacccagaatgcatggtccctcgaccacctcatagcccatcct

SEQ ID NO: 78 - DNA, 14 bp, Artificial Sequence
  Synthetic
caccaggtggcgct

SEQ ID NO: 79 - DNA, 320 bp, Homo sapiens
ctggttctactcattacattccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagg

SEQ ID NO: 80 - DNA, 305 bp, Homo sapiens
tccttcctttctaaatgacgagagagacagaagaattcttcaaggttagtgtgtccagcatgcaacctttccttcctggatgagcatccctggagtaggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattggctag

SEQ ID NO: 81 - DNA, 301 bp, Homo sapiens
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagga

SEQ ID NO: 82 - DNA, 305 bp, Artificial Sequence
  Synthetic
ctagccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctactccagggatgctcatccaggaaggaaaggttgcatgctggacacactaaccttgaagaattcttctgtctctctcgtcatttagaaaggaagga

SEQ ID NO: 83 - DNA, 300 bp, Homo sapiens
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagg

SEQ ID NO: 84 - DNA, 18 bp, Artificial Sequence
  Synthetic
aacccgttgaaccccatt

SEQ ID NO: 85 - DNA, 20 bp, Artificial Sequence
  Synthetic
ccatccaatcggtagtagcg

SEQ ID NO: 86 - DNA, 20 bp, Artificial Sequence
  Synthetic
cgtgcagtctaaagacttgg

SEQ ID NO: 87 - DNA, 20 bp, Artificial Sequence
  Synthetic
ataggggacttggacaaagg

SEQ ID NO: 88 - DNA, 19 bp, Artificial Sequence
  Synthetic
gccccagacctcttgatgc

SEQ ID NO: 89 - DNA, 23 bp, Artificial Sequence
  Synthetic
gaaaagttggaatggacaccaga

SEQ ID NO: 90 - DNA, 21 bp, Artificial Sequence
  Synthetic
cacctcctgatcccttcaaga

SEQ ID NO: 91 - DNA, 20 bp, Artificial Sequence
  Synthetic
cacaagtccagtcggtcaca

SEQ ID NO: 92 - DNA, 20 bp, Artificial Sequence
  Synthetic
gaggcggtttcaggggattg

SEQ ID NO: 93 - DNA, 20 bp, Artificial Sequence
  Synthetic
ctggattctcctgtagccgt

SEQ ID NO: 94 - DNA, 19 bp, Artificial Sequence
  Synthetic
taattccagagcggggcaa

SEQ ID NO: 95 - DNA, 21 bp, Artificial Sequence
  Synthetic
ggacttgcacacaggagacaa

SEQ ID NO: 96 - DNA, 20 bp, Artificial Sequence
  Synthetic
cccggtgtcacccgtcaccg

SEQ ID NO: 97 - DNA, 20 bp, Artificial Sequence
  Synthetic
cgcgtcccaggcgagcacgg

SEQ ID NO: 98 - DNA, 20 bp, Artificial Sequence
  Synthetic
tgacggcagggtgaactcgg

SEQ ID NO: 99 - DNA, 20 bp, Artificial Sequence
  Synthetic
gcagggtgaactcggcggca

SEQ ID NO: 100 - DNA, 20 bp, Artificial Sequence
  Synthetic
gcctacttcctaccacacga

SEQ ID NO: 101 - DNA, 20 bp, Artificial Sequence
  Synthetic
ttttatagccgtcgtgtggt

SEQ ID NO: 102 - DNA, 20 bp, Artificial Sequence
  Synthetic
gagtcaaggtattggcaatg

SEQ ID NO: 103 - DNA, 20 bp, Artificial Sequence
  Synthetic
agataagtgagtcaaggtat

SEQ ID NO: 104 - DNA, 20 bp, Artificial Sequence
  Synthetic
cattcgactaaagaaagccg

SEQ ID NO: 105 - DNA, 20 bp, Artificial Sequence
  Synthetic
gcccctgaatggtttaacgg

SEQ ID NO: 106 - DNA, 20 bp, Artificial Sequence
  Synthetic
tcgactaaagaaagccgggg

SEQ ID NO: 107 - DNA, 20 bp, Artificial Sequence
  Synthetic
tatgcagcctgtccgcaagg

SEQ ID NO: 108 - DNA, 20 bp, Artificial Sequence
  Synthetic
gttagacagatacttctaag

SEQ ID NO: 109 - DNA, 20 bp, Artificial Sequence
  Synthetic
tagctaaaacaagcagcttg

SEQ ID NO: 110 - DNA, 20 bp, Artificial Sequence
  Synthetic
gaatttgctcaaccgcctgt

SEQ ID NO: 111 - DNA, 20 bp, Artificial Sequence
  Synthetic
ggaatttgctcaaccgcctg

SEQ ID NO: 112 - DNA, 20 bp, Artificial Sequence
  Synthetic
aattccatttcaaagcacga

SEQ ID NO: 113 - DNA, 20 bp, Artificial Sequence
  Synthetic
gaaccaaggagctacccctg

SEQ ID NO: 114 - DNA, 20 bp, Artificial Sequence
  Synthetic
gatgaacgcgatggagtggg

SEQ ID NO: 115 - DNA, 20 bp, Artificial Sequence
  Synthetic
acgcctacggatgaacgcga

SEQ ID NO: 116 - DNA, 20 bp, Artificial Sequence
  Synthetic
ctgtatcctgggagccggcg

SEQ ID NO: 117 - DNA, 20 bp, Artificial Sequence
  Synthetic
taaaggcgttgacgcaactg

SEQ ID NO: 118 - DNA, 20 bp, Artificial Sequence
  Synthetic
gcggccgacgaaggcacatg

SEQ ID NO: 119 - DNA, 20 bp, Artificial Sequence
  Synthetic
ggatacagtgcggaccggcc

SEQ ID NO: 120 - DNA, 20 bp, Artificial Sequence
  Synthetic
cgttgcgaaggttcgtgtcg

SEQ ID NO: 121 - DNA, 20 bp, Artificial Sequence
  Synthetic
accttcgcaacgccgtccgc

SEQ ID NO: 122 - DNA, 20 bp, Artificial Sequence
  Synthetic
ccacgacatgaatcatcaca

SEQ ID NO: 123 - DNA, 20 bp, Artificial Sequence
  Synthetic
catgtgatgattcatgtcgt

SEQ ID NO: 124 - DNA, 20 bp, Artificial Sequence
  Synthetic
ccgaacggcgctgaaacctc

SEQ ID NO: 125 - DNA, 20 bp, Artificial Sequence
  Synthetic
cctgaggtttcagcgccgtt

SEQ ID NO: 126 - DNA, 20 bp, Artificial Sequence
  Synthetic
actgatgccaggatctacaa

SEQ ID NO: 127 - DNA, 20 bp, Artificial Sequence
  Synthetic
gataccccactactgatgcc

SEQ ID NO: 128 - DNA, 20 bp, Artificial Sequence
  Synthetic
cattcattagtaatcaacct

SEQ ID NO: 129 - DNA, 20 bp, Artificial Sequence
  Synthetic
ccccttttactccaaaagag

SEQ ID NO: 130 - DNA, 20 bp, Artificial Sequence
  Synthetic
taataaaccagcacgcacag

SEQ ID NO: 131 - DNA, 20 bp, Artificial Sequence
  Synthetic
accataagtttcacgagaga

SEQ ID NO: 132 - DNA, 20 bp, Artificial Sequence
  Synthetic
acatcatccagcatcattgt

SEQ ID NO: 133 - DNA, 20 bp, Artificial Sequence
  Synthetic
ctagcttcctcccgaccggg

SEQ ID NO: 134 - DNA, 20 bp, Artificial Sequence
  Synthetic
ctatctgtactagcccgagg

SEQ ID NO: 135 - DNA, 20 bp, Artificial Sequence
  Synthetic
atgtgtagcttaagccactg

SEQ ID NO: 136 - DNA, 20 bp, Artificial Sequence
  Synthetic
ttgctcatcttcacagtaca

SEQ ID NO: 137 - DNA, 20 bp, Artificial Sequence
  Synthetic
cggagtctagttatgtcgca

SEQ ID NO: 138 - DNA, 20 bp, Artificial Sequence
  Synthetic
atggtgcccgtccaatggat

SEQ ID NO: 139 - DNA, 20 bp, Artificial Sequence
  Synthetic
gaaggggatttattgtcgtg

SEQ ID NO: 140 - DNA, 20 bp, Artificial Sequence
  Synthetic
tcgacatggtacctagcacc

SEQ ID NO: 141 - DNA, 20 bp, Artificial Sequence
  Synthetic
atccactttcagggacgggg

SEQ ID NO: 142 - DNA, 20 bp, Artificial Sequence
  Synthetic
tacgatgacaatccagaaag

SEQ ID NO: 143 - DNA, 20 bp, Artificial Sequence
  Synthetic
catcttagtatctgctcgtg

SEQ ID NO: 144 - DNA, 20 bp, Artificial Sequence
  Synthetic
ctacgtgcagcacggaacct

SEQ ID NO: 145 - DNA, 20 bp, Artificial Sequence
  Synthetic
tgtggatgagcgacctctcg

SEQ ID NO: 146 - DNA, 20 bp, Artificial Sequence
  Synthetic
ttacgtgacccctttgcaga

SEQ ID NO: 147 - DNA, 20 bp, Artificial Sequence
  Synthetic
tgcaaaggggtcacgtaatg

SEQ ID NO: 148 - DNA, 20 bp, Artificial Sequence
  Synthetic
cttcaaacaggaaatggagt

SEQ ID NO: 149 - DNA, 20 bp, Artificial Sequence
  Synthetic
gtgcaataaaactgtctcac

SEQ ID NO: 150 - DNA, 20 bp, Artificial Sequence
  Synthetic
gaacatgatgagtcaagcac

SEQ ID NO: 151 - DNA, 20 bp, Artificial Sequence
  Synthetic
attggccaagtcaagcccta

SEQ ID NO: 152 - DNA, 20 bp, Artificial Sequence
  Synthetic
gctcatacagacaaaccact

SEQ ID NO: 153 - DNA, 20 bp, Artificial Sequence
  Synthetic
ttttgagttaaatcctaagg

SEQ ID NO: 154 - DNA, 20 bp, Artificial Sequence
  Synthetic
tattataatagaagtgcctg

SEQ ID NO: 155 - DNA, 20 bp, Artificial Sequence
  Synthetic
ataatgaatgtctcatgaga

SEQ ID NO: 156 - DNA, 19 bp, Artificial Sequence
  Synthetic
cagttcatatcaagcacat

SEQ ID NO: 157 - DNA, 19 bp, Artificial Sequence
  Synthetic
aatgcattacagtagagca

SEQ ID NO: 158 - DNA, 20 bp, Artificial Sequence
  Synthetic
acaataccaagggtatccgg

SEQ ID NO: 159 - DNA, 20 bp, Artificial Sequence
  Synthetic
atcaagtatttacaatacca

SEQ ID NO: 160 - DNA, 19 bp, Artificial Sequence
  Synthetic
tcaagtatttacaatacca

SEQ ID NO: 161 - DNA, 19 bp, Artificial Sequence
  Synthetic
actgagaactagttcatac

SEQ ID NO: 162 - DNA, 20 bp, Artificial Sequence
  Synthetic
atgagtgctagagactggtg

SEQ ID NO: 163 - DNA, 20 bp, Artificial Sequence
  Synthetic
taacctatgaacactgacac